(19) United States
(12) Patent Application Publication
(10) Pub. No.: US 2017/0210723 A1
(43) Pub. Date: Jul. 27, 2017

(54) MOLECULES HAVING PESTICIDAL UTILITY, AND INTERMEDIATES, COMPOSITIONS, AND PROCESSES, RELATED THERETO

(71) Applicant: Dow AgroSciences LLC, Indianapolis, IN (US)

(72) Inventors: Paul R. LePlae, JR., Brownsburg, IN (US); Thomas Barton, Indianapolis, IN (US); Xin Gao, Carmel, IN (US); Jim Hunter, Indianapolis, IN (US); William C. Lo, Fishers, IN (US); Joshodeep Boruwa, Noida, IN (US); Raghuram Tangirala, Bengaluru, IN (US); Gerald B. Watson, Zionsville, IN (US); John Herbert, Fishers, IN (US)

(73) Assignee: Dow AgroSciences LLC, Indianapolis, IN (US)

(21) Appl. No.: 15/408,503

(22) Filed: Jan. 18, 2017

Related U.S. Application Data
(60) Provisional application No. 62/286,535, filed on Jan. 25, 2016.
(60) Provisional application No. 62/286,573, filed on Jan. 25, 2016.

(51) Int. Cl. C07D 317/62; A01N 37/20; C07C 317/28; A01N 37/34; C07D 305/08; A01N 43/20; A01N 43/30; C07C 323/42; A01N 41/10

(52) U.S. Cl. CPC C07D 317/62; C07C 323/42; A01N 37/20; C07C 317/28; A01N 41/10; C07D 305/08; A01N 43/20; A01N 43/30; A01N 37/34; C07C 2101/02

(57) ABSTRACT

This disclosure relates to the field of molecules having pesticidal utility against pests in Phyla Arthropoda, Mollusca, and Nematoda, processes to produce such molecules, intermediates used in such processes, compositions containing such molecules, and processes of using such molecules and compositions against such pests. These molecules and compositions may be used, for example, as acaricides, insecticides, miticides, molluscicides, and nematicides. This document discloses molecules having the following formula (“Formula One”).

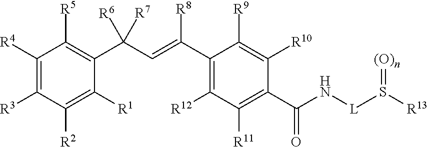

### CROSS REFERENCE TO RELATED APPLICATIONS

This application claims the benefit of, and priority from, U.S. Provisional Patent Application Ser. Nos. 62/286,535 and 62/286,573 both filed Jan. 25, 2016, each of which are expressly incorporated by reference herein.

### FIELD OF THIS DISCLOSURE

This disclosure relates to the field of molecules having pesticidal utility against pests in Phyla Arthropoda, Mollusca, and Nematoda, processes to produce such molecules, intermediates used in such processes, pesticidal compositions containing such molecules, and processes of using such pesticidal compositions against such pests. These pesticidal compositions may be used, for example, as acaricides, insecticides, miticides, molluscicides, and nematicides.

### BACKGROUND OF THIS DISCLOSURE

“Many of the most dangerous human diseases are transmitted by insect vectors” (Rivero et al.). “Historically, malaria, dengue, yellow fever, plague, filariasis, louse-borne typhus, trypanosomiasis, leishmaniasis, and other vector borne diseases were responsible for more human disease and death in the 17^{th} through the early 20^{th} centuries than all other causes combined” (Gubler). Vector-borne diseases are responsible for about 17% of the global parasitic and infectious diseases. Malaria alone causes over 800,000 deaths a year, 85% of which occur in children under five years of age. Each year there are about 50 to about 100 million cases of dengue fever. A further 250,000 to 500,000 cases of dengue hemorrhagic fever occur each year (Matthews). Vector control plays a critical role in the prevention and control of infectious diseases. However, insecticide resistance, including resistance to multiple insecticides, has arisen in all insect species that are major vectors of human diseases (Rivero et al.). Recently, more than 550 arthropod pest species have developed resistance to at least one pesticide (Whalon et al.).

Each year insects, plant pathogens, and weeds, destroy more than 40% of all food production. This loss occurs despite the application of pesticides and the use of a wide array of non-chemical controls, such as, crop rotations, and biological controls. If just some of this food could be saved, it could be used to feed the more than three billion people in the world who are malnourished (Pimental).

Plant parasitic nematodes are among the most widespread pests, and are frequently one of the most insidious and costly. It has been estimated that losses attributable to nematodes are from about 9% in developed countries to about 15% in undeveloped countries. However, in the United States of America a survey of 35 States on various crops indicated nematode-derived losses of up to 25% (Nicol et al.).

It is noted that gastropods (slugs and snails) are pests of less economic importance than other arthropods or nematodes, but in certain places they may reduce yields substantially, severely affecting the quality of harvested products, as well as, transmitting human, animal, and plant diseases. While only a few dozen species of gastropods are serious regional pests, a handful of species are important pests on a world-wide scale. In particular, gastropods affect a wide variety of agricultural and horticultural crops, such as, arable, pastoral, and fiber crops; vegetables; bush and tree fruits; herbs; and ornamentals (Speiser).

Termites cause damage to all types of private and public structures, as well as, to agricultural and forestry resources. In 2005, it was estimated that termites cause over US$50 billion in damage world-wide each year (Korb).

Consequently, for many reasons, including those mentioned above, there is an on-going need for the costly (estimated to be about US$256 million per pesticide in 2010), time-consuming (on average about 10 years per pesticide), and difficult, development of new pesticides (CropLife America).

DeMassey et al. discloses the following structure. For more detail, refer to US 2002/0068838.

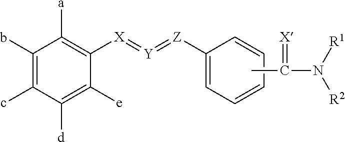

### CERTAIN REFERENCES CITED IN THIS DISCLOSURE

CropLife America, The Cost of New Agrochemical Product Discovery, Development & Registration, and Research & Development predictions for the Future, 2010.
Gubler, D., Resurgent Vector-Borne Diseases as a Global Health Problem, Emerging Infectious Diseases, Vol. 4, No. 3, p. 442-450, 1998.
Korb, J., Termites, Current Biology, Vol. 17, No. 23, 2007.
Matthews, G., Integrated Vector Management: Controlling Vectors of Malaria and Other Insect Vector Borne Diseases, Ch. 1, p. 1-2011.
Nicol, J., Turner S.; Coyne, L.; den Nijs, L., Hocksland, L., Tahna-Maafi, Z., Current Nematode Threats to World Agriculture, Genomic and Molecular Genetics of Plant—Nematode Interactions, p. 21-43, 2011).
Pimental, D., Pest Control in World Agriculture, Agricultural Sciences—Vol. II, 2009.
Rivero, A., Vezilier, J., Weill, M., Read, A., Gandon, S., Insect Control of Vector-Borne Diseases: When is Insect Resistance a Problem? Public Library of Science Pathogens, Vol. 6, No. 8, p. 1-9, 2010.
Speiser, B., Molluscicides, Encyclopedia of Pest Management, Ch. 219, p. 506-508, 2002.
Whalon, M., Mota-Sanchez, D., Hollingworth, R., Analysis of Global Pesticide Resistance in Arthropods, Global Pesticide Resistance in Arthropods, Ch. 1, p. 5-33, 2008.

### Definitions Used in this Disclosure

The examples given in these definitions are generally non-exhaustive and must not be construed as limiting the disclosure. It is understood that a substituent should comply with chemical bonding rules and steric compatibility constraints in relation to the particular molecule to which it is attached. These definitions are only to be used for the purposes of this disclosure.

“active ingredient” means a material having activity useful in controlling pests, and/or that is useful in helping other materials have better activity in controlling pests, examples of such materials include, but are not limited to, acaricides, algicides, avicides, bactericides, fungicides, herbicides, insecticides, molluscicides, nematicides, rodenticides, virucides, antifeedants, bird repellents, chemosterilants, herbicide safeners, insect attractants, insect repellents, mammal repellents, mating disrupters, plant activators, plant growth regulators, and synergists. Specific examples of such materials include, but are not limited to, the materials listed in active ingredient group alpha.

“active ingredient group alpha” (hereafter “AIGA”) means collectively the following materials:

(1) (3-ethoxypropyl)mercury bromide, 1,2-dibromoethane, 1,2-dichloroethane, 1,2-dichloropropane, 1,3-dichloropropene, 1-MCP, 1-methylcyclopropene, 1-naphthol, 2-(octylthio)ethanol, 2,3,3-TPA, 2,3,5-tri-iodobenzoic acid, 2,3,6-TBA, 2,4,5-T, 2,4,5-TB, 2,4,5-TP, 2,4-D, 2,4-DB, 2,4-DEB, 2,4-DEP, 2,4-DES, 2,4-DP, 2,4-MCPA, 2,4-MCPB, 2iP, 2-methoxyethylmercury chloride, 2-phenylphenol, 3,4-DA, 3,4-DB, 3,4-DP, 3,6-dichloropicolinic acid, 4-aminopyridine, 4-CPA, 4-CPB, 4-CPP, 4-hydroxyphenethyl alcohol, 8-hydroxyquinoline sulfate, 8-phenylmercurioxyquinoline, abamectin, abamectin-aminomethyl, abscisic acid, ACC, acephate, acequinocyl, acetamiprid, acethion, acetochlor, acetofenate, acetophos, acetoprole, acibenzolar, acifluorfen, aclonifen, ACN, acrep, acrinathrin, acrolein, acrylonitrile, acypetacs, afidopyropen, afoxolaner, alachlor, alanap, alanycarb, albendazole, aldicarb, aldicarb sulfone, aldimorph, aldoxycarb, aldrin, allethrin, allicin, allidochlor, allosamidin, alloxydim, allyl alcohol, allyxycarb, alorac, alpha-cypermethrin, alpha-endosulfan, alphamethrin, altretamine, aluminium phosphide, aluminum phosphide, ametoctradin, ametridione, ametryn, ametryne, amibuzin, amicarbazone, amicarthiazol, amidithion, amidoflumet, amidosulfuron, aminocarb, aminocyclopyrachlor, aminopyralid, aminotriazole, amiprofos-methyl, amiprophos, amiprophos-methyl, amisulbrom, amiton, amitraz, amitrole, ammonium sulfamate, amobam, amorphous silica gel, amorphous silicon dioxide, ampropylfos, AMS, anabasine, ancymidol, anilazine, anilofos, anisuron, anthraquinone, antu, apholate, aramite, arprocarb, arsenous oxide, asomate, aspirin, asulam, athidathion, atraton, atrazine, aureofungin, avermectin B1, AVG, aviglycine, azaconazole, azadirachtin, azafenidin, azamethiphos, azidithion, azimsulfuron, azinphosethyl, azinphos-ethyl, azinphosmethyl, azinphos-methyl, aziprotryn, aziprotryne, azithiram, azobenzene, azocyclotin, azothoate, azoxystrobin, bachmedesh, barban, barbanate, barium hexafluorosilicate, barium polysulfide, barium silicofluoride, barthrin, basic copper carbonate, basic copper chloride, basic copper sulfate, BCPC, beflubutamid, benalaxyl, benalaxyl-M, benazolin, bencarbazone, benclothiaz, bendaqingbingzhi, bendiocarb, bendioxide, benefin, benfluralin, benfuracarb, benfuresate, benmihuangcaoan, benodanil, benomyl, benoxacor, benoxafos, benquinox, bensulfuron, bensulide, bensultap, bentaluron, bentazon, bentazone, benthiavalicarb, benthiazole, benthiocarb, bentranil, benzadox, benzalkonium chloride, benzamacril, benzamizole, benzamorf, benzene hexachloride, benzfendizone, benzimine, benzipram, benzobicyclon, benzoepin, benzofenap, benzofluor, benzohydroxamic acid, benzomate, benzophosphate, benzothiadiazole, benzovindiflupyr, benzoximate, benzoylprop, benzthiazuron, benzuocaotong, benzyl benzoate, benzyladenine, berberine, beta-cyfluthrin, beta-cypermethrin, bethoxazin, BHC, bialaphos, bicyclopyrone, bifenazate, bifenox, bifenthrin, bifujunzhi, bilanafos, binapacryl, bingqingxiao, bioallethrin, bioethanomethrin, biopermethrin, bioresmethrin, biphenyl, bisazir, bismerthiazol, bismerthiazol-copper, bisphenylmercury methylenedi(x-naphthalene-y-sulphonate), bispyribac, bistrifluron, bisultap, bitertanol, bithionol, bixafen, blasticidin-S, borax, Bordeaux mixture, boric acid, boscalid, BPPS, brassinolide, brassinolide-ethyl, brevicomin, brodifacoum, brofenprox, brofenvalerate, broflanilide, brofluthrinate, bromacil, bromadiolone, bromchlophos, bromethalin, bromethrin, bromfenvinfos, bromoacetamide, bromobonil, bromobutide, bromociclen, bromocyclen, bromo-DDT, bromofenoxim, bromofos, bromomethane, bromophos, bromophos-ethyl, bromopropylate, bromothalonil, bromoxynil, brompyrazon, bromuconazole, bronopol, BRP, BTH, bucarpolate, bufencarb, buminafos, bupirimate, buprofezin, Burgundy mixture, busulfan, busulphan, butacarb, butachlor, butafenacil, butam, butamifos, butane-fipronil, butathiofos, butenachlor, butene-fipronil, butethrin, buthidazole, buthiobate, buthiuron, butifos, butocarboxim, butonate, butopyronoxyl, butoxycarboxim, butralin, butrizol, butroxydim, buturon, butylamine, butylate, butylchlorophos, butylene-fipronil, cacodylic acid, cadusafos, cafenstrole, calciferol, calcium arsenate, calcium chlorate, calcium cyanamide, calcium cyanide, calcium polysulfide, calvinphos, cambendichlor, camphechlor, camphor, captafol, captan, carbam, carbamorph, carbanolate, carbaril, carbaryl, carbasulam, carbathion, carbendazim, carbendazol, carbetamide, carbofenotion, carbofuran, carbon disulfide, carbon tetrachloride, carbonyl sulfide, carbophenothion, carbophos, carbosulfan, carboxazole, carboxide, carboxin, carfentrazone, carpropamid, cartap, carvacrol, carvone, CAVP, CDAA, CDEA, CDEC, cellocidin, CEPC, ceralure, cerenox, cevadilla, Cheshunt mixture, chinalphos, chinalphos-mdthyl, chinomethionat, chinomethionate, chiralaxyl, chitosan, chlobenthiazone, chlomethoxyfen, chloralose, chloramben, chloramine phosphorus, chloramphenicol, chloraniformethan, chloranil, chloranocryl, chlorantraniliprole, chlorazifop, chlorazine, chlorbenside, chlorbenzuron, chlorbicyclen, chlorbromuron, chlorbufam, chlordane, chlordecone, chlordimeform, chlorempenthrin, chloretazate, chlorethephon, chlorethoxyfos, chloreturon, chlorfenac, chlorfenapyr, chlorfenazole, chlorfenethol, chlorfenidim, chlorfenprop, chlorfenson, chlorfensulphide, chlorfenvinphos, chlorfenvinphos-methyl, chlorfluazuron, chlorflurazole, chlorflurecol, chlorfluren, chlorflurenol, chloridazon, chlorimuron, chlorinate, chlor-IPC, chlormephos, chlormequat, chlormesulone, chlormethoxynil, chlornidine, chlornitrofen, chloroacetic acid, chlorobenzilate, chlorodinitronaphthalenes, chlorofdnizon, chloroform, chloromebuform, chloromethiuron, chloroneb, chlorophacinone, chlorophos, chloropicrin, chloropon, chloropropylate, chlorothalonil, chlorotoluron, chloroxifenidim, chloroxuron, chloroxynil, chlorphonium, chlorphoxim, chlorprazophos, chlorprocarb, chlorpropham, chlorpyrifos, chlorpyrifos-methyl, chlorquinox, chlorsulfuron, chlorthal, chlorthiamid, chlorthiophos, chlortoluron, chlozolinate, chltosan, cholecalciferol, choline chloride, chromafenozide, cicloheximide, cimectacarb, cimetacarb, cinerin I, cinerin II, cinerins, cinidon-ethyl, cinmethylin, cinosulfuron, cintofen, ciobutide, cisanilide, cismethrin, clacyfos, clefoxydim, clenpirin, clenpyrin, clethodim, climbazole, cliodinate, clodinafop, cloethocarb, clofencet, clofenotane, clofentezine, clofenvinfos, clofibric acid, clofop, clomazone, clomeprop, clonitralid, cloprop, cloproxydim, clopyralid, cloquintocet, cloransulam, closantel, clothianidin, clotrimazole, cloxyfonac, cloxylacon, clozylacon, CMA, CMMP, CMP, CMU, codlelure, colecalciferol, colophonate, copper 8-quinolinolate, copper acetate, copper acetoarsenite, copper arsenate, copper carbonate, basic, copper hydroxide, copper naphthenate, copper oleate, copper oxychloride, copper silicate, copper sulfate, copper sulfate, basic, copper zinc chromate, coumachlor, coumafene, coumafos, coumafuryl, coumaphos, coumatetralyl, coumethoxystrobin, coumithoate, coumoxystrobin, CPMC, CPMF, CPPC, credazine, cresol, cresylic acid, crimidine, crotamiton, crotoxyfos, crotoxyphos, crufomate, cryolite, cue-lure, cufraneb, cumyleron, cumyluron, cuprobam, cuprous oxide, curcumenol, CVMP, cyanamide, cyanatryn, cyanazine, cyanofenphos, cyanogen, cyanophos, cyanthoate, cyantraniliprole, cyanuric acid, cyazofamid, cybutryne, cyclafuramid, cyclanilide, cyclaniliprole, cyclethrin, cycloate, cycloheximide, cycloprate, cycloprothrin, cyclopyrimorate, cyclosulfamuron, cycloxydim, cycluron, cyenopyrafen, cyflufenamid, cyflumetofen, cyfluthrin, cyhalofop, cyhalothrin, cyhexatin, cymiazole, cymoxanil, cyometrinil, cypendazole, cypermethrin, cyperquat, cyphenothrin, cyprazine, cyprazole, cyproconazole, cyprodinil, cyprofuram, cypromid, cyprosulfamide, cyromazine, cythioate, cytrex, daimuron, dalapon, daminozide, dayoutong, dazomet, DBCP, d-camphor, DCB, DCIP, DCPA, DCPTA, DCU, DDD, DDPP, DDT, DDVP, debacarb, decafentin, decamethrin, decarbofuran, deet, dehydroacetic acid, deiquat, delachlor, delnav, deltamethrin, demephion, demephion-O, demephion-S, demeton, demeton-methyl, demeton-O, demeton-O-methyl, demeton-S, demeton-S-methyl, demeton-S-methyl sulphone, demeton-S-methylsulphon, DEP, depallthrine, derris, desmedipham, desmetryn, desmetryne, d-fanshiluquebingjuzhi, diafenthiuron, dialifor, dialifos, diallate, diamidafos, dianat, diatomaceous earth, diatomite, diazinon, dibrom, dibutyl phthalate, dibutyl succinate, dicamba, dicapthon, dichlobenil, dichlofenthion, dichlofluanid, dichlone, dichloralurea, dichlorbenzuron, dichlorfenidim, dichlorflurecol, dichlorflurenol, dichlormate, dichlormid, dichloromethane, dicloromezotiaz, dichlorophen, dichlorprop, dichlorprop-P, dichlorvos, dichlozolin, dichlozoline, diclobutrazol, diclocymet, diclofop, diclomezine, dicloran, diclosulam, dicofol, dicophane, dicoumarol, dicresyl, dicrotophos, dicryl, dicumarol, dicyclanil, dicyclonon, dieldrin, dienochlor, diethamquat, diethatyl, diethion, didthion, diethofencarb, dietholate, didthon, diethyl pyrocarbonate, diethyltoluamide, difenacoum, difenoconazole, difenopenten, difenoxuron, difenzoquat, difethialone, diflovidazin, diflubenzuron, diflufenican, diflufenicanil, diflufenzopyr, diflumetorim, dikegulac, dilor, dimatif, dimefluthrin, dimefox, dimefuron, dimehypo, dimepiperate, dimetachlone, dimetan, dimethacarb, dimethachlone, dimethachlor, dimethametryn, dimethenamid, dimethenamid-P, dimethipin, dimethirimol, dimethoate, dimethomorph, dimethrin, dimethyl carbate, dimethyl disulfide, dimethyl phthalate, dimethylvinphos, dimetilan, dimexano, dimidazon, dimoxystrobin, dimpylate, dimuron, dinex, dingjunezuo, diniconazole, diniconazole-M, dinitramine, dinitrophenols, dinobuton, dinocap, dinocap-4, dinocap-6, dinocton, dinofenate, dinopenton, dinoprop, dinosam, dinoseb, dinosulfon, dinotefuran, dinoterb, dinoterbon, diofenolan, dioxabenzofos, dioxacarb, dioxathion, dioxation, diphacin, diphacinone, diphenadione, diphenamid, diphenamide, diphenyl sulfone, diphenylamine, diphenylsulphide, diprogulic acid, dipropalin, dipropetryn, dipterex, dipymetitrone, dipyrithione, diquat, disodium tetraborate, disosultap, disparlure, disugran, disul, disulfiram, disulfoton, ditalimfos, dithianon, dithicrofos, dithioether, dithiomdton, dithiopyr, diuron, dixanthogen, d-limonene, DMDS, DMPA, DNOC, dodemorph, dodicin, dodine, dofenapyn, doguadine, dominicalure, doramectin, DPC, drazoxolon, DSMA, d-trans-allethrin, d-trans-resmethrin, dufulin, dymron, EBEP, EBP, ebufos, ecdysterone, echlomezol, EDB, EDC, EDDP, edifenphos, eglinazine, emamectin, EMPC, empenthrin, enadenine, endosulfan, endothal, endothall, endothion, endrin, enestroburin, enilconazole, enoxastrobin, ephirsulfonate, EPN, epocholeone, epofenonane, epoxiconazole, eprinomectin, epronaz, EPTC, erbon, ergocalciferol, erlujixiancaoan, esddpalldthrine, esfenvalerate, ESP, esprocarb, etacelasil, etaconazole, etaphos, etem, ethaboxam, ethachlor, ethalfluralin, ethametsulfuron, ethaprochlor, ethephon, ethidimuron, ethiofencarb, ethiolate, ethion, ethiozin, ethiprole, ethirimol, ethoate-methyl, ethobenzanid, ethofumesate, ethohexadiol, ethoprop, ethoprophos, ethoxyfen, ethoxyquin, ethoxysulfuron, ethychlozate, ethyl formate, ethyl pyrophosphate, ethylan, ethyl-DDD, ethylene, ethylene dibromide, ethylene dichloride, ethylene oxide, ethylicin, ethylmercury 2,3-dihydroxypropyl mercaptide, ethylmercury acetate, ethylmercury bromide, ethylmercury chloride, ethylmercury phosphate, etinofen, ETM, etnipromid, etobenzanid, etofenprox, etoxazole, etridiazole, etrimfos, etrimphos, eugenol, EXD, famoxadone, famphur, fenac, fenamidone, fenaminosulf, fenaminstrobin, fenamiphos, fenapanil, fenarimol, fenasulam, fenazaflor, fenazaquin, fenbuconazole, fenbutatin oxide, fenchlorazole, fenchlorphos, fenclofos, fenclorim, fenethacarb, fenfluthrin, fenfuram, fenhexamid, fenidin, fenitropan, fenitrothion, fenizon, fenjuntong, fenobucarb, fenolovo, fenoprop, fenothiocarb, fenoxacrim, fenoxanil, fenoxaprop, fenoxaprop-P, fenoxasulfone, fenoxycarb, fenpiclonil, fenpirithrin, fenpropathrin, fenpropidin, fenpropimorph, fenpyrazamine, fenpyroximate, fenquinotrione, fenridazon, fenson, fensulfothion, fenteracol, fenthiaprop, fenthion, fenthion-ethyl, fentiaprop, fentin, fentrazamide, fentrifanil, fenuron, fenuron-TCA, fenvalerate, ferbam, ferimzone, ferric phosphate, ferrous sulfate, fipronil, flamprop, flamprop-M, flazasulfuron, flocoumafen, flometoquin, flonicamid, florasulam, fluacrypyrim, fluazifop, fluazifop-P, fluazinam, fluazolate, fluazuron, flubendiamide, flubenzimine, flubrocythrinate, flucarbazone, flucetosulfuron, fluchloralin, flucofuron, flucycloxuron, flucythrinate, fludioxonil, fludndthyl, fluenetil, fluensulfone, flufenacet, flufenerim, flufenican, flufenoxuron, flufenoxystrobin, flufenprox, flufenpyr, flufenzine, flufiprole, fluhexafon, flumethrin, flumetover, flumetralin, flumetsulam, flumezin, flumiclorac, flumioxazin, flumipropyn, flumorph, fluometuron, fluopicolide, fluopyram, fluorbenside, fluoridamid, fluoroacetamide, fluoroacetic acid, fluorochloridone, fluorodifen, fluoroglycofen, fluoroimide, fluoromide, fluoromidine, fluoronitrofen, fluoroxypyr, fluothiuron, fluotrimazole, fluoxastrobin, flupoxam, flupropacil, flupropadine, flupropanate, flupyradifurone, flupyrsulfuron, fluquinconazole, fluralaner, flurazole, flurecol, flurenol, fluridone, flurochloridone, fluromidine, fluroxypyr, flurprimidol, flursulamid, flurtamone, flusilazole, flusulfamide, flutenzine, fluthiacet, fluthiamide, flutianil, flutolanil, flutriafol, fluvalinate, fluxapyroxad, fluxofenim, folpel, folpet, fomesafen, fonofos, foramsulfuron, forchlorfenuron, formaldehyde, formetanate, formothion, formparanate, fosamine, fosetyl, fosmethilan, fospirate, fosthiazate, fosthietan, frontalin, fthalide, fuberidazole, fucaojing, fucaomi, fujunmanzhi, fulumi, fumarin, funaihecaoling, fuphenthiourea, furalane, furalaxyl, furamethrin, furametpyr, furan tebufenozide, furathiocarb, furcarbanil, furconazole, furconazole-cis, furethrin, furfural, furilazole, furmecyclox, furophanate, furyloxyfen, gamma-BHC, gamma-cyhalothrin, gamma-HCH, genit, gibberellic acid, gibberellin A3, gibberellins, gliftor, glitor, glucochloralose, glufosinate, glufosinate-P, glyodin, glyoxime, glyphosate, glyphosine, gossyplure, grandlure, griseofulvin, guanoctine, guazatine, halacrinate, halauxifen, halfenprox, halofenozide, halosafen, halosulfuron, haloxydine, haloxyfop, haloxyfop-P, haloxyfop-R, HCA, HCB, HCH, hemel, hempa, HEOD, heptachlor, heptafluthrin, heptenophos, heptopargil, herbimycin, herbimycin A, heterophos, hexachlor, hexachloran, hexachloroacetone, hexachlorobenzene, hexachlorobutadiene, hexachlorophene, hexaconazole, hexaflumuron, hexafluoramin, hexaflurate, hexalure, hexamide, hexazinone, hexylthiofos, hexythiazox, HHDN, holosulf, homobrassinolide, huancaiwo, huanchongjing, huangcaoling, huanjunzuo, hydramethylnon, hydrargaphen, hydrated lime, hydrogen cyanamide, hydrogen cyanide, hydroprene, hydroxyisoxazole, hymexazol, hyquincarb, IAA, IBA, IBP, icaridin, imazalil, imazamethabenz, imazamox, imazapic, imazapyr, imazaquin, imazethapyr, imazosulfuron, imibenconazole, imicyafos, imidacloprid, imidaclothiz, iminoctadine, imiprothrin, inabenfide, indanofan, indaziflam, indoxacarb, inezin, infusorial earth, iodobonil, iodocarb, iodofenphos, iodomethane, iodosulfuron, iofensulfuron, ioxynil, ipazine, IPC, ipconazole, ipfencarbazone, iprobenfos, iprodione, iprovalicarb, iprymidam, ipsdienol, ipsenol, IPSP, IPX, isamidofos, isazofos, isobenzan, isocarbamid, isocarbamide, isocarbophos, isocil, isodrin, isofenphos, isofenphos-methyl, isofetamid, isolan, isomethiozin, isonoruron, isopamphos, isopolinate, isoprocarb, isoprocil, isopropalin, isopropazol, isoprothiolane, isoproturon, isopyrazam, isopyrimol, isothioate, isotianil, isouron, isovaledione, isoxaben, isoxachlortole, isoxadifen, isoxaflutole, isoxapyrifop, isoxathion, isuron, ivermectin, ixoxaben, izopamfos, izopamphos, japonilure, japothrins, jasmolin I, jasmolin II, jasmonic acid, jiahuangchongzong, jiajizengxiaolin, jiaxiangjunzhi, jiecaowan, jiecaoxi, Jinganmycin A, jodfenphos, juvenile hormone I, juvenile hormone II, juvenile hormone III, kadethrin, kappa-bifenthrin, kappa-tefluthrin, karbutilate, karetazan, kasugamycin, kejunlin, kelevan, ketospiradox, kieselguhr, kinetin, kinoprene, kiralaxyl, kresoxim-methyl, kuicaoxi, lactofen, lambda-cyhalothrin, latilure, lead arsenate, lenacil, lepimectin, leptophos, lianbenjingzhi, lime sulfur, lindane, lineatin, linuron, lirimfos, litlure, looplure, lufenuron, lixiancaolin, Ivdingjunzhi, Ivfumijvzhi, Ivxiancaolin, lythidathion, M-74, M-81, MAA, magnesium phosphide, malathion, maldison, maleic hydrazide, malonoben, maltodextrin, MAMA, mancopper, mancozeb, mandestrobin, mandipropamid, maneb, matrine, mazidox, MCC, MCP, MCPA, MCPA-thioethyl, MCPB, MCPP, mebenil, mecarbam, mecarbinzid, mecarphon, mecoprop, mecoprop-P, medimeform, medinoterb, medlure, mefenacet, mefenoxam, mefenpyr, mefluidide, megatomoic acid, melissyl alcohol, melitoxin, MEMC, menazon, MEP, mepanipyrim, meperfluthrin, mephenate, mephosfolan, mepiquat, mepronil, meptyldinocap, mercaptodimethur, mercaptophos, mercaptophos thiol, mercaptothion, mercuric chloride, mercuric oxide, mercurous chloride, merphos, merphos oxide, mesoprazine, mesosulfuron, mesotrione, mesulfen, mesulfenfos, mesulphen, metacresol, metaflumizone, metalaxyl, metalaxyl-M, metaldehyde, metam, metamifop, metamitron, metaphos, metaxon, metazachlor, metazosulfuron, metazoxolon, metconazole, metepa, metflurazon, methabenzthiazuron, methacrifos, methalpropalin, metham, methamidophos, methasulfocarb, methazole, methfuroxam, methibenzuron, methidathion, methiobencarb, methiocarb, methiopyrisulfuron, methiotepa, methiozolin, methiuron, methocrotophos, metholcarb, methometon, methomyl, methoprene, methoprotryn, methoprotryne, methoquin-butyl, methothrin, methoxychlor, methoxyfenozide, methoxyphenone, methyl apholate, methyl bromide, methyl eugenol, methyl iodide, methyl isothiocyanate, methyl parathion, methylacetophos, methylchloroform, methyldithiocarbamic acid, methyldymron, methylene chloride, methyl-isofenphos, methylmercaptophos, methylmercaptophos oxide, methylmercaptophos thiol, methylmercury benzoate, methylmercury dicyandiamide, methylmercury pentachlorophenoxide, methylneodecanamide, methylnitrophos, methyltriazothion, metiozolin, metiram, metiram-zinc, metobenzuron, metobromuron, metofluthrin, metolachlor, metolcarb, metometuron, metominostrobin, metosulam, metoxadiazone, metoxuron, metrafenone, metriam, metribuzin, metrifonate, metriphonate, metsulfovax, metsulfuron, mevinphos, mexacarbate, miechuwei, mieshuan, miewenjuzhi, milbemectin, milbemycin oxime, milneb, mima2nan, mipafox, MIPC, mirex, MNAF, moguchun, molinate, molosultap, momfluorothrin, monalide, monisuron, monoamitraz, monochloroacetic acid, monocrotophos, monolinuron, monomehypo, monosulfiram, monosulfuron, monosultap, monuron, monuron-TCA, morfamquat, moroxydine, morphothion, morzid, moxidectin, MPMC, MSMA, MTMC, muscalure, myclobutanil, myclozolin, myricyl alcohol, N-(ethylmercury)-p-toluenesulphonanilide, NAA, NAAm, nabam, naftalofos, naled, naphthalene, naphtha leneacetamide, naphthalic anhydride, naphthalophos, naphthoxyacetic acids, naphthylacetic acids, naphthylindane-1,3-diones, naphthyloxyacetic acids, naproanilide, napropamide, napropamide-M, naptalam, natamycin, NBPOS, neburea, neburon, nendrin, neonicotine, nichlorfos, niclofen, niclosamide, nicobifen, nicosulfuron, nicotine, nicotine sulfate, nifluridide, nikkomycins, NIP, nipyraclofen, nipyralofen, nitenpyram, nithiazine, nitralin, nitrapyrin, nitrilacarb, nitrofen, nitrofluorfen, nitrostyrene, nitrothal-isopropyl, nobormide, nonanol, norbormide, norea, norflurazon, nornicotine, noruron, novaluron, noviflumuron, NPA, nuarimol, nuranone, OCH, octachlorodipropyl ether, octhilinone, o-dichlorobenzene, ofurace, omethoate, o-phenylphenol, orbencarb, orfralure, orthobencarb, ortho-dichlorobenzene, orthosulfamuron, oryctalure, orysastrobin, oryzalin, osthol, osthole, ostramone, ovatron, ovex, oxabetrinil, oxadiargyl, oxadiazon, oxadixyl, oxamate, oxamyl, oxapyrazon, oxapyrazone, oxasulfuron, oxathiapiprolin, oxaziclomefone, oxine-copper, oxine-Cu, oxolinic acid, oxpoconazole, oxycarboxin, oxydemeton-methyl, oxydeprofos, oxydisulfoton, oxyenadenine, oxyfluorfen, oxymatrine, oxytetracycline, oxythioquinox, PAC, paclobutrazol, paichongding, pallthrine, PAP, para-dichlorobenzene, parafluron, paraquat, parathion, parathion-methyl, parinol, Paris green, PCNB, PCP, PCP-Na, p-dichlorobenzene, PDJ, pebulate, pédinex, pefurazoate, pelargonic acid, penconazole, pencycuron, pendimethalin, penfenate, penflufen, penfluron, penoxalin, penoxsulam, pentachlorophenol, pentachlorophenyl laurate, pentanochlor, penthiopyrad, pentmethrin, pentoxazone, perchlordecone, perfluidone, permethrin, pethoxamid, PHC, phenamacril, phenamacril-ethyl, phdnaminosulf, phenazine oxide, phénétacarbe, phenisopham, phenkapton, phenmedipham, phenmedipham-ethyl, phenobenzuron, phenothiol, phenothrin, phenproxide, phenthoate, phenylmercuriurea, phenylmercury acetate, phenylmercury chloride, phenylmercury derivative of pyrocatechol, phenylmercury nitrate, phenylmercury salicylate, phorate, phosacetim, phosalone, phosametine, phosazetim, phosazetin, phoscyclotin, phosdiphen, phosethyl, phosfolan, phosfolan-methyl, phosglycin, phosmet, phosnichlor, phosphamide, phosphamidon, phosphine, phosphinothricin, phosphocarb, phosphorus, phostin, phoxim, phoxim-methyl, phthalide, phthalophos, phthalthrin, picarbutrazox, picaridin, picloram, picolinafen, picoxystrobin, pimaricin, pindone, pinoxaden, piperalin, piperazine, piperonyl butoxide, piperonyl cyclonene, piperophos, piproctanly, piproctanyl, piprotal, pirimetaphos, pirimicarb, piriminil, pirimioxyphos, pirimiphos-ethyl, pirimiphos-methyl, pival, pivaldione, plifenate, PMA, PMP, polybutenes, polycarbamate, polychlorcamphene, polyethoxyquinoline, polyoxin D, polyoxins, polyoxorim, polythialan, potassium arsenite, potassium azide, potassium cyanate, potassium ethylxanthate, potassium naphthenate, potassium polysulfide, potassium thiocyanate, pp′-DDT, prallethrin, precocene I, precocene II, precocene III, pretilachlor, primidophos, primisulfuron, probenazole, prochloraz, proclonol, procyazine, procymidone, prodiamine, profenofos, profluazol, profluralin, profluthrin, profoxydim, profurite-aminium, proglinazine, prohexadione, prohydrojasmon, promacyl, promecarb, prometon, prometryn, prometryne, promurit, pronamide, propachlor, propafos, propamidine, propamocarb, propanil, propaphos, propaquizafop, propargite, proparthrin, propazine, propetamphos, propham, propiconazole, propidine, propineb, propisochlor, propoxur, propoxycarbazone, propyl isome, propyrisulfuron, propyzamide, proquinazid, prosuler, prosulfalin, prosulfocarb, prosulfuron, prothidathion, prothiocarb, prothioconazole, prothiofos, prothoate, protrifenbute, proxan, prymidophos, prynachlor, psoralen, psoralene, pydanon, pyflubumide, pymetrozine, pyracarbolid, pyraclofos, pyraclonil, pyraclostrobin, pyraflufen, pyrafluprole, pyramat, pyrametostrobin, pyraoxystrobin, pyrasulfotole, pyraziflumid, pyrazolate, pyrazolynate, pyrazon, pyrazophos, pyrazosulfuron, pyrazothion, pyrazoxyfen, pyresmethrin, pyrethrin I, pyrethrin II, pyrethrins, pyribambenz-isopropyl, pyribambenz-propyl, pyribencarb, pyribenzoxim, pyributicarb, pyriclor, pyridaben, pyridafol, pyridalyl, pyridaphenthion, pyridaphenthione, pyridate, pyridinitril, pyrifenox, pyrifluquinazon, pyriftalid, pyrimdtaphos, pyrimethanil, pyrimicarbe, pyrimidifen, pyriminobac, pyriminostrobin, pyrimiphos-ethyl, pyrimiphos-methyl, pyrimisulfan, pyrimitate, pyrinuron, pyriofenone, pyriprole, pyripropanol, pyriproxyfen, pyrisoxazole, pyrithiobac, pyrolan, pyroquilon, pyroxasulfone, pyroxsulam, pyroxychlor, pyroxyfur, qincaosuan, qingkuling, quassia, quinacetol, quinalphos, quinalphos-methyl, quinazamid, quinclorac, quinconazole, quinmerac, quinoclamine, quinomethionate, quinonamid, quinothion, quinoxyfen, quintiofos, quintozene, quizalofop, quizalofop-P, quwenzhi, quyingding, rabenzazole, rafoxanide, R-diniconazole, rebemide, reglone, renriduron, rescalure, resmethrin, rhodethanil, rhodojaponin-III, ribavirin, rimsulfuron, rizazole, R-metalaxyl, roddthanil, ronnel, rotenone, ryania, sabadilla, saflufenacil, saijunmao, saisentong, salicylanilide, salifluofen, sanguinarine, santonin, S-bioallethrin, schradan, scilliroside, sebuthylazine, secbumeton, sedaxane, selamectin, semiamitraz, sesamex, sesamolin, sesone, sethoxydim, sevin, shuangjiaancaolin, shuangjianancaolin, S-hydroprene, siduron, sifumijvzhi, siglure, silafluofen, silatrane, silica aerogel, silica gel, silthiofam, silthiopham, silthiophan, silvex, simazine, simeconazole, simeton, simetryn, simetryne, sintofen, S-kinoprene, slaked lime, SMA, S-methoprene, S-metolachlor, sodium arsenite, sodium azide, sodium chlorate, sodium cyanide, sodium fluoride, sodium fluoroacetate, sodium hexafluorosilicate, sodium naphthenate, sodium o-phenylphenoxide, sodium orthophenylphenoxide, sodium pentachlorophenate, sodium pentachlorophenoxide, sodium polysulfide, sodium silicofluoride, sodium tetrathiocarbonate, sodium thiocyanate, solan, sophamide, spinetoram, spinosad, spirodiclofen, spiromesifen, spirotetramat, spiroxamine, stirofos, streptomycin, strychnine, sulcatol, sulcofuron, sulcotrione, sulfallate, sulfentrazone, sulfiram, sulfluramid, sulfodiazole, sulfometuron, sulfosate, sulfosulfuron, sulfotep, sulfotepp, sulfoxaflor, sulfoxide, sulfoxime, sulfur, sulfuric acid, sulfuryl fluoride, sulglycapin, sulphosate, sulprofos, sultropen, swep, tau-fluvalinate, tavron, tazimcarb, TBTO, TBZ, TCA, TCBA, TCMTB, TCNB, TDE, tebuconazole, tebufenozide, tebufenpyrad, tebufloquin, tebupirimfos, tebutam, tebuthiuron, tecloftalam, tecnazene, tecoram, tedion, teflubenzuron, tefluthrin, tefuryltrione, tembotrione, temefos, temephos, tepa, TEPP, tepraloxydim, teproloxydim, terallethrin, terbacil, terbucarb, terbuchlor, terbufos, terbumeton, terbuthylazine, terbutol, terbutryn, terbutryne, terraclor, terramicin, terramycin, tetcyclacis, tetrachloroethane, tetrachlorvinphos, tetraconazole, tetradifon, tetradisul, tetrafluron, tetramethrin, tetramethylfluthrin, tetramine, tetranactin, tetraniliprole, tetrapion, tetrasul, thallium sulfate, thallous sulfate, thenylchlor, theta-cypermethrin, thiabendazole, thiacloprid, thiadiazine, thiadifluor, thiamethoxam, thiameturon, thiapronil, thiazafluron, thiazfluron, thiazone, thiazopyr, thicrofos, thicyofen, thidiazimin, thidiazuron, thiencarbazone, thifensulfuron, thifluzamide, thimerosal, thimet, thiobencarb, thiocarboxime, thiochlorfenphim, thiochlorphenphime, thiocyanatodinitrobenzenes, thiocyclam, thiodan, thiodiazole-copper, thiodicarb, thiofanocarb, thiofanox, thiofluoximate, thiohempa, thiomersal, thiometon, thionazin, thiophanate, thiophanate-ethyl, thiophanate-methyl, thiophos, thioquinox, thiosemicarbazide, thiosultap, thiotepa, thioxamyl, thiram, thiuram, thuringiensin, tiabendazole, tiadinil, tiafenacil, tiaojiean, TIBA, tifatol, tiocarbazil, tioclorim, tioxazafen, tioxymid, tirpate, TMTD, tolclofos-methyl, tolfenpyrad, tolprocarb, tolpyralate, tolyfluanid, tolylfluanid, tolylmercury acetate, tomarin, topramezone, toxaphene, TPN, tralkoxydim, tralocythrin, tralomethrin, tralopyril, transfluthrin, transpermethrin, tretamine, triacontanol, triadimefon, triadimenol, triafamone, triallate, tri-allate, triamiphos, triapenthenol, triarathene, triarimol, triasulfuron, triazamate, triazbutil, triaziflam, triazophos, triazothion, triazoxide, tribasic copper chloride, tribasic copper sulfate, tribenuron, tribufos, tributyltin oxide, tricamba, trichlamide, trichlopyr, trichlorfon, trichlormetaphos-3, trichloronat, trichloronate, trichlorotrinitrobenzenes, trichlorphon, triclopyr, triclopyricarb, tricresol, tricyclazole, tricyclohexyltin hydroxide, tridemorph, tridiphane, trietazine, trifenmorph, trifenofos, trifloxystrobin, trifloxysulfuron, trifludimoxazin, triflumezopyrim, triflumizole, triflumuron, trifluralin, triflusulfuron, trifop, trifopsime, triforine, trihydroxytriazine, trimedlure, trimethacarb, trimeturon, trinexapac, triphenyltin, triprene, tripropindan, triptolide, tritac, trithialan, triticonazole, tritosulfuron, trunc-call, tuoyelin, uniconazole, uniconazole-P, urbacide, uredepa, valerate, validamycin, validamycin A, valifenalate, valone, vamidothion, vangard, vaniliprole, vernolate, vinclozolin, vitamin D3, warfarin, xiaochongliulin, xinjunan, xiwojunan, xiwojunzhi, XMC, xylachlor, xylenols, xylylcarb, xymiazole, yishijing, zarilamid, zeatin, zengxiaoan, zengxiaolin, zeta-cypermethrin, zinc naphthenate, zinc phosphide, zinc thiazole, zinc thiozole, zinc trichlorophenate, zinc trichlorophenoxide, zineb, ziram, zolaprofos, zoocoumarin, zoxamide, zuoanjunzhi, zuocaoan, zuojunzhi, zuomihuanglong, α-chlorohydrin, α-ecdysone, α-multistriatin, α-naphthaleneacetic acids, and β-ecdysone;

(2) the following molecule

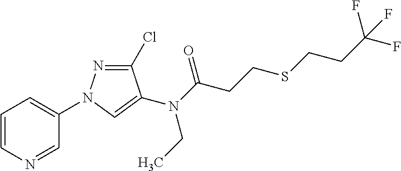 [SMILES: CCN(C(=O)CCSCCC(F)(F)F)c1cn(-c2cccnc2)nc1Cl]

### N-(3-chloro-1-(pyridin-3-yl)-1H-pyrazol-4-yl)-N-ethyl-3-((3,3,3-trifluoropropyl)thio)propanamide

In this document, this molecule, for ease of use, is named as “AI-1;”

(3) a molecule known as Lotilaner which has the following

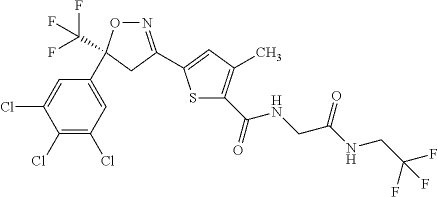 [SMILES: Cc1cc(C2=NO[C@@](c3cc(Cl)c(Cl)c(Cl)c3)(C(F)(F)F)C2)sc1C(=O)NCC(=O)NCC(F)(F)F]

and

(4) the following molecules in Table A

TABLE A
Structure of M-active ingredients

| Name | Structure |
|---|---|
| M1 | 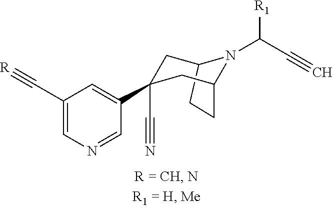 |
| M2 | 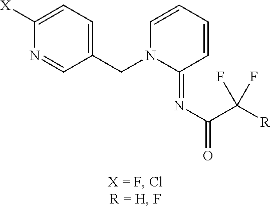 |
| M3 | 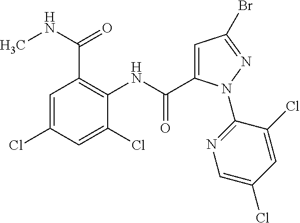 [SMILES: CNC(=O)c1cc(Cl)cc(Cl)c1NC(=O)c1cc(Br)nn1-c1ncc(Cl)cc1Cl] |
| M4 | 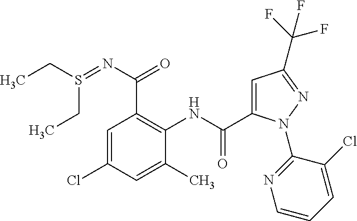 [SMILES: CCS(CC)=NC(=O)c1cc(Cl)cc(C)c1NC(=O)c1cc(C(F)(F)F)nn1-c1ncccc1Cl] |
| M5 | 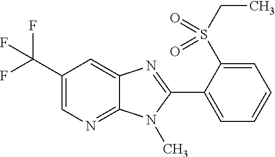 [SMILES: CCS(=O)(=O)c1ccccc1-c1nc2cc(C(F)(F)F)cnc2n1C] |
| M6 | 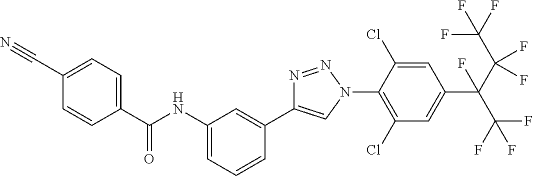 [SMILES: N#Cc1ccc(C(=O)Nc2cccc(-c3cn(-c4c(Cl)cc(C(F)(C(F)(F)F)C(F)(F)C(F)(F)F)cc4Cl)nn3)c2)cc1] |

As used in this disclosure, each of the above is an active ingredient, and two or more are active ingredients. For more information consult the “COMPENDIUM OF PESTICIDE COMMON NAMES” located at Alanwood.net and various editions, including the on-line edition, of “THE PESTICIDE MANUAL” located at bcpcdata.com.

The term “alkenyl” means an acyclic, unsaturated (at least one carbon-carbon double bond), branched or unbranched, substituent consisting of carbon and hydrogen, for example, vinyl, allyl, butenyl, pentenyl, and hexenyl.

The term “alkenyloxy” means an alkenyl further consisting of a carbon-oxygen single bond, for example, allyloxy, butenyloxy, pentenyloxy, hexenyloxy.

The term “alkoxy” means an alkyl further consisting of a carbon-oxygen single bond, for example, methoxy, ethoxy, propoxy, isopropoxy, butoxy, isobutoxy, and tert-butoxy.

The term “alkyl” means an acyclic, saturated, branched or unbranched, substituent consisting of carbon and hydrogen, for example, methyl, ethyl, propyl, isopropyl, butyl, and tert-butyl.

The term “alkynyl” means an acyclic, unsaturated (at least one carbon-carbon triple bond), branched or unbranched, substituent consisting of carbon and hydrogen, for example, ethynyl, propargyl, butynyl, and pentynyl.

The term “alkynyloxy” means an alkynyl further consisting of a carbon-oxygen single bond, for example, pentynyloxy, hexynyloxy, heptynyloxy, and octynyloxy.

The term “aryl” means a cyclic, aromatic substituent consisting of hydrogen and carbon, for example, phenyl, naphthyl, and biphenyl.

The term “biopesticide” means a microbial biological pest control agent which, in general, is applied in a similar manner to chemical pesticides. Commonly they are bacterial, but there are also examples of fungal control agents, including Trichoderma spp. and Ampelomyces quisqualis. One well-known biopesticide example is Bacillus thuringiensis, a bacterial disease of Lepidoptera, Coleoptera, and Diptera, Biopesticides include products based on:

(1) entomopathogenic fungi (e.g. Metarhizium anisopliae);

(2) entomopathogenic nematodes (e.g. Steinernema feltiae); and

(3) entomopathogenic viruses (e.g. Cydia pomonella granulovirus).

Other examples of entomopathogenic organisms include, but are not limited to, baculoviruses, protozoa, and Microsporidia. For the avoidance of doubt biopesticides are considered to be active ingredients.

The term “cycloalkenyl” means a monocyclic or polycyclic, unsaturated (at least one carbon-carbon double bond) substituent consisting of carbon and hydrogen, for example, cyclobutenyl, cyclopentenyl, cyclohexenyl, norbornenyl, bicyclo[2.2.2]octenyl, tetrahydronaphthyl, hexahydronaphthyl, and octahydronaphthyl.

The term “cycloalkenyloxy” means a cycloalkenyl further consisting of a carbon-oxygen single bond, for example, cyclobutenyloxy, cyclopentenyloxy, norbornenyloxy, and bicyclo[2.2.2]octenyloxy.

The term “cycloalkyl” means a monocyclic or polycyclic, saturated substituent consisting of carbon and hydrogen, for example, cyclopropyl, cyclobutyl, cyclopentyl, norbornyl, bicyclo[2.2.2]octyl, and decahydronaphthyl.

The term “cycloalkoxy” means a cycloalkyl further consisting of a carbon-oxygen single bond, for example, cyclopropyloxy, cyclobutyloxy, cyclopentyloxy, norbornyloxy, and bicyclo[2.2.2]octyloxy.

The term “halo” means fluoro, chloro, bromo, and iodo.

The term “haloalkoxy” means an alkoxy further consisting of, from one to the maximum possible number of identical or different, halos, for example, fluoromethoxy, trifluoromethoxy, 2,2-difluoropropoxy, chloromethoxy, trichloromethoxy, 1,1,2,2-tetrafluoroethoxy, and pentafluoroethoxy.

The term “haloalkyl” means an alkyl further consisting of, from one to the maximum possible number of, identical or different, halos, for example, fluoromethyl, trifluoromethyl, 2,2-difluoropropyl, chloromethyl, trichloromethyl, and 1,1,2,2-tetrafluoroethyl.

The term “heterocyclyl” means a cyclic substituent that may be aromatic, fully saturated, or partially or fully unsaturated, where the cyclic structure contains at least one carbon and at least one heteroatom, where said heteroatom is nitrogen, sulfur, or oxygen. Examples are:

(1) aromatic heterocyclyl substituents include, but are not limited to, benzofuranyl, benzoisothiazolyl, benzoisoxazolyl, benzoxazolyl, benzothienyl, benzothiazolyl cinnolinyl, furanyl, indazolyl, indolyl, imidazolyl, isoindolyl, isoquinolinyl, isothiazolyl, isoxazolyl, oxadiazolyl, oxazolinyl, oxazolyl, phthalazinyl, pyrazinyl, pyrazolinyl, pyrazolyl, pyridazinyl, pyridyl, pyrimidinyl, pyrrolyl, quinazolinyl, quinolinyl, quinoxalinyl, tetrazolyl, thiazolinyl, thiazolyl, thienyl, triazinyl, and triazolyl;

(2) fully saturated heterocyclyl substituents include, but are not limited to, piperazinyl, piperidinyl, morpholinyl, pyrrolidinyl, tetrahydrofuranyl, and tetrahydropyranyl;

(3) partially or fully unsaturated heterocyclyl substituents include, but are not limited to, 1,2,3,4-tetrahydro-quinolinyl, 4,5-dihydro-oxazolyl, 4,5-dihydro-1H-pyrazolyl, 4,5-dihydro-isoxazolyl, and 2,3-dihydro-[1,3,4]-oxadiazolyl; and

(4) Additional examples of heterocyclyls include the following: thietanyl thietanyl-oxide and thietanyl-dioxide.

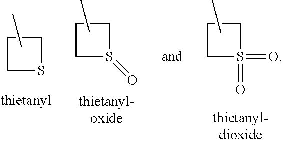

The term “locus” means a habitat, breeding ground, plant, seed, soil, material, or environment, in which a pest is growing, may grow, or may traverse, for example, a locus may be: where crops, trees, fruits, cereals, fodder species, vines, turf, and/or ornamental plants are growing; where domesticated animals are residing; the interior or exterior surfaces of buildings (such as places where grains are stored); the materials of construction used in buildings (such as impregnated wood); and the soil around buildings.

The phrase “MoA Material” means a material having a mode of action (“MoA”) as indicated in IRAC MoA Classification v. 7.3, located at irac-online.org., which describes:

(1) Acetylcholinesterase (AChE) inhibitors;

(2) GABA-gated chloride channel antagonists;

(3) Sodium channel modulators;

(4) Nicotinic acetylcholine receptor (nAChR) agonists;

(5) Nicotinic acetylcholine receptor (nAChR) allosteric activators;

(6) Chloride channel activators;

(7) Juvenile hormone mimics;

(8) Miscellaneous nonspecific (multi-site) inhibitors;

(9) Modulators of Chordotonal Organs;

(10) Mite growth inhibitors;

(11) Microbial disruptors of insect midgut membranes;

(12) Inhibitors of mitochondrial ATP synthase;

(13) Uncouplers of oxidative phosphorylation via disruption of the proton gradient;

(14) Nicotinic acetylcholine receptor (nAChR) channel blockers;

(15) Inhibitors of chitin biosynthesis, type 0;

(16) Inhibitors of chitin biosynthesis, type 1;

(17) Moulting disruptor, Dipteran;

(18) Ecdysone receptor agonists;

(19) Octopamine receptor agonists;

(20) Mitochondrial complex III electron transport inhibitors;

(21) Mitochondrial complex I electron transport inhibitors;

(22) Voltage-dependent sodium channel blockers;

(23) Inhibitors of acetyl CoA carboxylase;

(24) Mitochondrial complex IV electron transport inhibitors;

(25) Mitochondrial complex II electron transport inhibitors; and

(28) Ryanodine receptor modulators.

The phrase “MoA material group alpha” (hereafter “MoAMGA”) means collectively the following materials, abamectin, acephate, acequinocyl, acetamiprid, acrinathrin, alanycarb, aldicarb, allethrin, alpha-cypermethrin, aluminium phosphide, amitraz, azamethiphos, azinphos-ethyl, azinphos-methyl, azocyclotin, bendiocarb, benfuracarb, bensultap, beta-cyfluthrin, beta-cypermethrin, bifenthrin, bioallethrin, bioallethrin S-cyclopentenyl isomer, bioresmethrin, bistrifluron, borax, buprofezin, butocarboxim, butoxycarboxim, cadusafos, calcium phosphide, carbaryl, carbofuran, carbosulfan, cartap hydrochloride, chlorantraniliprole, chlordane, chlorethoxyfos, chlorfenapyr, chlorfenvinphos, chlorfluazuron, chlormephos, chloropicrin, chlorpyrifos, chlorpyrifos-methyl, chromafenozide, clofentezine, clothianidin, coumaphos, cyanide, cyanophos, cyantraniliprole, cycloprothrin, cyenopyrafen, cyflumetofen, cyfluthrin, cyhalothrin, cyhexatin, cypermethrin, cyphenothrin, cyromazine, d-cis-trans-allethrin, DDT, deltamethrin, demeton-S-methyl, diafenthiuron, diazinon, dichlorvos/DDVP, dicrotophos, diflovidazin, diflubenzuron, dimethoate, dimethylvinphos, dinotefuran, disulfoton, DNOC, d-trans-allethrin, emamectin benzoate, empenthrin, endosulfan, EPN, esfenvalerate, ethiofencarb, ethion, ethoprophos, etofenprox, etoxazole, famphur, fenamiphos, fenazaquin, fenbutatin oxide, fenitrothion, fenobucarb, fenoxycarb, fenpropathrin, fenpyroximate, fenthion, fenvalerate, flonicamid, fluacrypyrim, flubendiamide, flucycloxuron, flucythrinate, flufenoxuron, flumethrin, flupyradifurone, formetanate, fosthiazate, furathiocarb, gamma-cyhalothrin, halfenprox, halofenozide, heptenophos, hexaflumuron, hexythiazox, hydramethylnon, hydroprene, imicyafos, imidacloprid, imiprothrin, indoxacarb, isofenphos, isoprocarb, isoxathion, kadethrin, kinoprene, lambda-cyhalothrin, lepimectin, lufenuron, malathion, mecarbam, metaflumizone, methamidophos, methidathion, methiocarb, methomyl, methoprene, (methoxyaminothio-phosphoryl) salicylate, methoxychlor, methoxyfenozide, methyl bromide, metolcarb, mevinphos, milbemectin, monocrotophos, naled, nicotine, nitenpyram, novaluron, noviflumuron, oxamyl, oxydemeton-methyl, parathion, parathion-methyl, permethrin, phenothrin, phenthoate, phorate, phosalone, phosmet, phosphamidon, phosphine, phoxim, pirimicarb, pirimiphos-methyl, prallethrin, profenofos, propargite, propetamphos, propoxur, prothiofos, pymetrozine, pyraclofos, pyrethrin, pyridaben, pyridaphenthion, pyrimidifen, pyriproxyfen, quinalphos, resmethrin, rotenone, silafluofen, spinetoram, spinosad, spirodiclofen, spiromesifen, spirotetramat, sulfluramid, sulfotep, sulfoxaflor, sulfuryl fluoride, tartar emetic, tau-fluvalinate, tebufenozide, tebufenpyrad, tebupirimfos, teflubenzuron, tefluthrin, temephos, terbufos, tetrachlorvinphos, tetradifon, tetramethrin, tetramethrin, theta-cypermethrin, thiacloprid, thiamethoxam, thiocyclam, thiodicarb, thiofanox, thiometon, thiosultap-sodium, tolfenpyrad, tralomethrin, transfluthrin, triazamate, triazophos, trichlorfon, triflumuron, trimethacarb, vamidothion, XMC, xylylcarb, zeta-cypermethrin, and zinc phosphide. For the avoidance of doubt, each of the foregoing materials is an active ingredient.

The term “pest” means an organism that is detrimental to humans, or human concerns (such as, crops, food, livestock, etc.), where said organism is from Phyla Arthropoda, Mollusca, or Nematoda, particular examples are ants, aphids, beetles, bristletails, cockroaches, crickets, earwigs, fleas, flies, grasshoppers, leafhoppers, lice (including sea lice), locusts, mites, moths, nematodes, scales, symphylans, termites, thrips, ticks, wasps, and whiteflies, additional examples are pests in:

(1) Subphyla Chelicerata, Myriapoda, Crustacea, and Hexapoda;

(2) Classes of Arachnida, Maxillopoda, Symphyla, and Insecta;

(3) Order Anoplura. A non-exhaustive list of particular genera includes, but is not limited to, Haematopinus spp., Hoplopleura spp., Linognathus spp., Pediculus spp., and Polyplax spp. A non-exhaustive list of particular species includes, but is not limited to, Haematopinus asini, Haematopinus suis, Linognathus setosus, Linognathus ovillus, Pediculus humanus capitis, Pediculus humanus humanus, and Pthirus pubis.

(4) Order Coleoptera. A non-exhaustive list of particular genera includes, but is not limited to, Acanthoscelides spp., Agriotes spp., Anthonomus spp., Apion spp., Apogonia spp., Aulacophora spp., Bruchus spp., Cerosterna spp., Cerotoma spp., Ceutorhynchus spp., Chaetocnema spp., Colaspis spp., Ctenicera spp., Curculio spp., Cyclocephala spp., Diabrotica spp., Hypera spp., Ips spp., Lyctus spp., Megascelis spp., Meligethes spp., Otiorhynchus spp., Pantomorus spp., Phyllophaga spp., Phyllotreta spp., Rhizotrogus spp., Rhynchites spp., Rhynchophorus spp., Scolytus spp., Sphenophorus spp., Sitophilus spp., and Tribolium spp. A non-exhaustive list of particular species includes, but is not limited to, Acanthoscelides obtectus, Agrilus planipennis, Anoplophora glabripennis, Anthonomus grandis, Ataenius spretulus, Atomaria linearis, Bothynoderes punctiventris, Bruchus pisorum, Callosobruchus maculatus, Carpophilus hemipterus, Cassida vittata, Cerotoma trifurcata, Ceutorhynchus assimilis, Ceutorhynchus napi, Conoderus scalaris, Conoderus stigmosus, Conotrachelus nenuphar, Cotinis nitida, Crioceris asparagi, Cryptolestes ferrugineus, Cryptolestes pusillus, Cryptolestes turcicus, Cylindrocopturus adspersus, Deporaus marginatus, Dermestes lardarius, Dermestes maculatus, Epilachna varivestis, Faustinus cubae, Hylobius pales, Hypera postica, Hypothenemus hampei, Lasioderma serricorne, Leptinotarsa decemlineata, Liogenys fuscus, Liogenys suturalis, Lissorhoptrus oryzophilus, Maecolaspis joliveti, Melanotus communis, Meligethes aeneus, Melolontha melolontha, Oberea brevis, Oberea linearis, Oryctes rhinoceros, Oryzaephilus mercator, Oryzaephilus surinamensis, Oulema melanopus, Oulema oryzae, Phyllophaga cuyabana, Popillia japonica, Prostephanus truncatus, Rhyzopertha dominica, Sitona lineatus, Sitophilus granarius, Sitophilus oryzae, Sitophilus zeamais, Stegobium paniceum, Tribolium castaneum, Tribolium confusum, Trogoderma variabile, and Zabrus tenebrioides.

(5) Order Dermaptera. A non-exhaustive list of particular species includes, but is not limited to, Forficula auricularia.

(6) Order Blattaria. A non-exhaustive list of particular species includes, but is not limited to, Blattella germanica, Blatta orientalis, Parcoblatta pennsylvanica, Periplaneta americana, Periplaneta australasiae, Periplaneta brunnea, Periplaneta fuliginosa, Pycnoscelus surinamensis, and Supella longipalpa.

(7) Order Diptera. A non-exhaustive list of particular genera includes, but is not limited to, Aedes spp., Agromyza spp., Anastrepha spp., Anopheles spp., Bactrocera spp., Ceratitis spp., Chrysops spp., Cochliomyia spp., Contarinia spp., Culex spp., Dasineura spp., Delia spp., Drosophila spp., Fannia spp., Hylemyia spp., Liriomyza spp., Musca spp., Phorbia spp., Tabanus spp., and Tipula spp. A non-exhaustive list of particular species includes, but is not limited to, Agromyza frontella, Anastrepha suspensa, Anastrepha ludens, Anastrepha obliqa, Bactrocera cucurbitae, Bactrocera dorsalis, Bactrocera invadens, Bactrocera zonata, Ceratitis capitata, Dasineura brassicae, Delia platura, Fannia canicularis, Fannia scalaris, Gasterophilus intestinalis, Gracillia perseae, Haematobia irritans, Hypoderma lineatum, Liriomyza brassicae, Melophagus ovinus, Musca autumnalis, Musca domestica, Oestrus ovis, Oscinella frit, Pegomya betae, Psila rosae, Rhagoletis cerasi, Rhagoletis pomonella, Rhagoletis mendax, Sitodiplosis mosellana, and Stomoxys calcitrans.

(8) Order Hemiptera. A non-exhaustive list of particular genera includes, but is not limited to, Adelges spp., Aulacaspis spp., Aphrophora spp., Aphis spp., Bemisia spp., Ceroplastes spp., Chionaspis spp., Chrysomphalus spp., Coccus spp., Empoasca spp., Lepidosaphes spp., Lagynotomus spp., Lygus spp., Macrosiphum spp., Nephotettix spp., Nezara spp., Philaenus spp., Phytocoris spp., Piezodorus spp., Planococcus spp., Pseudococcus spp., Rhopalosiphum spp., Saissetia spp., Therioaphis spp., Toumeyella spp., Toxoptera spp., Trialeurodes spp., Triatoma spp. and Unaspis spp. A non-exhaustive list of particular species includes, but is not limited to, Acrosternum hilare, Acyrthosiphon pisum, Aleyrodes proletella, Aleurodicus dispersus, Aleurothrixus floccosus, Amrasca biguttula biguttula, Aonidiella aurantii, Aphis gossypii, Aphis glycines, Aphis pomi, Aulacorthum solani, Bemisia argentifolii, Bemisia tabaci, Blissus leucopterus, Brachycorynella asparagi, Brevennia rehi, Brevicoryne brassicae, Calocoris norvegicus, Ceroplastes rubens, Cimex hemipterus, Cimex lectularius, Dagbertus fasciatus, Dichelops furcatus, Diuraphis noxia, Diaphorina citri, Dysaphis plantaginea, Dysdercus suturellus, Edessa meditabunda, Eriosoma lanigerum, Eurygaster maura, Euschistus heros, Euschistus servus, Helopeltis antonii, Helopeltis theivora, Icerya purchasi, Idioscopus nitidulus, Laodelphax striatellus, Leptocorisa oratorius, Leptocorisa varicornis, Lygus hesperus, Maconellicoccus hirsutus, Macrosiphum euphorbiae, Macrosiphum granarium, Macrosiphum rosae, Macrosteles quadrilineatus, Mahanarva frimbiolata, Metopolophium dirhodum, Mictis longicornis, Myzus persicae, Nephotettix cinctipes, Neurocolpus longirostris, Nezara viridula, Nilaparvata lugens, Parlatoria pergandii, Parlatoria ziziphi, Peregrinus maidis, Phylloxera vitifoliae, Physokermes piceae, Phytocoris californicus, Phytocoris relativus, Piezodorus guildinii, Poecilocapsus lineatus, Psallus vaccinicola, Pseudacysta perseae, Pseudococcus brevipes, Quadraspidiotus perniciosus, Rhopalosiphum maidis, Rhopalosiphum padi, Saissetia oleae, Scaptocoris castanea, Schizaphis graminum, Sitobion avenae, Sogatella furcifera, Trialeurodes vaporariorum, Trialeurodes abutiloneus, Unaspis yanonensis, and Zulia entrerriana.

(9) Order Hymenoptera. A non-exhaustive list of particular genera includes, but is not limited to, Acromyrmex spp., Atta spp., Camponotus spp., Diprion spp., Formica spp., Monomorium spp., Neodiprion spp., Pogonomyrmex spp., Polistes spp., Solenopsis spp., Vespula spp., and Xylocopa spp. A non-exhaustive list of particular species includes, but is not limited to, Athalia rosae, Atta texana, Iridomyrmex humilis, Monomorium minimum, Monomorium pharaonis, Solenopsis invicta, Solenopsis geminata, Solenopsis molesta, Solenopsis richtery, Solenopsis xyloni, and Tapinoma sessile.

(10) Order Isoptera. A non-exhaustive list of particular genera includes, but is not limited to, Coptotermes spp., Cornitermes spp., Cryptotermes spp., Heterotermes spp., Kalotermes spp., Incisitermes spp., Macrotermes spp., Marginitermes spp., Microcerotermes spp., Procornitermes spp., Reticulitermes spp., Schedorhinotermes spp., and Zootermopsis spp. A non-exhaustive list of particular species includes, but is not limited to, Coptotermes curvignathus, Coptotermes frenchi, Coptotermes formosanus, Heterotermes aureus, Microtermes obesi, Reticulitermes banyulensis, Reticulitermes grassei, Reticulitermes flavipes, Reticulitermes hageni, Reticulitermes hesperus, Reticulitermes santonensis, Reticulitermes speratus, Reticulitermes tibialis, and Reticulitermes virginicus.

(11) Order Lepidoptera. A non-exhaustive list of particular genera includes, but is not limited to, Adoxophyes spp., Agrotis spp., Argyrotaenia spp., Cacoecia spp., Caloptilia spp., Chilo spp., Chrysodeixis spp., Colias spp., Crambus spp., Diaphania spp., Diatraea spp., Earias spp., Ephestia spp., Epimecis spp., Feltia spp., Gortyna spp., Helicoverpa spp., Heliothis spp., Indarbela spp., Lithocolletis spp., Loxagrotis spp., Malacosoma spp., Peridroma spp., Phyllonorycter spp., Pseudaletia spp., Sesamia spp., Spodoptera spp., Synanthedon spp., and Yponomeuta spp. A non-exhaustive list of particular species includes, but is not limited to, Achaea janata, Adoxophyes orana, Agrotis ipsilon, Alabama argillacea, Amorbia cuneana, Amyelois transitella, Anacamptodes defectaria, Anarsia lineatella, Anomis sabulifera, Anticarsia gemmatalis, Archips argyrospila, Archips rosana, Argyrotaenia citrana, Autographa gamma, Bonagota cranaodes, Borbo cinnara, Bucculatrix thurberiella, Capua reticulana, Carposina niponensis, Chlumetia transversa, Choristoneura rosaceana, Cnaphalocrocis medinalis, Conopomorpha cramerella, Cossus cossus, Cydia caryana, Cydia funebrana, Cydia molesta, Cydia nigricana, Cydia pomonella, Darna diducta, Diatraea saccharalis, Diatraea grandiosella, Earias insulana, Earias vittella, Ecdytolopha aurantianum, Elasmopalpus lignosellus, Ephestia cautella, Ephestia elutella, Ephestia kuehniella, Epinotia aporema, Epiphyas postvittana, Erionota thrax, Eupoecilia ambiguella, Euxoa auxiliaris, Grapholita molesta, Hedylepta indicata, Helicoverpa armigera, Helicoverpa zea, Heliothis virescens, Hellula undalis, Keiferia lycopersicella, Leucinodes orbonalis, Leucoptera coffeella, Leucoptera malifoliella, Lobesia botrana, Loxagrotis albicosta, Lymantria dispar, Lyonetia clerkella, Mahasena corbetti, Mamestra brassicae, Maruca testulalis, Metisa plana, Mythimna unipuncta, Neoleucinodes elegantalis, Nymphula depunctalis, Operophtera brumata, Ostrinia nubilalis, Oxydia vesulia, Pandemis cerasana, Pandemis heparana, Papilio demodocus, Pectinophora gossypiella, Peridroma saucia, Perileucoptera coffeella, Phthorimaea operculella, Phyllocnistis citrella, Pieris rapae, Plathypena scabra, Plodia interpunctella, Plutella xylostella, Polychrosis viteana, Prays endocarpa, Prays oleae, Pseudaletia unipuncta, Pseudoplusia includens, Rachiplusia nu, Scirpophaga incertulas, Sesamia inferens, Sesamia nonagrioides, Setora nitens, Sitotroga cerealella, Sparganothis pilleriana, Spodoptera exigua, Spodoptera frugiperda, Spodoptera eridania, Thecla basilides, Tineola bisselliella, Trichoplusia ni, Tuta absoluta, Zeuzera coffeae, and Zeuzera pyrina;

(12) Order Mallophaga. A non-exhaustive list of particular genera includes, but is not limited to, Anaticola spp., Bovicola spp., Chelopistes spp., Goniodes spp., Menacanthus spp., and Trichodectes spp. A non-exhaustive list of particular species includes, but is not limited to, Bovicola bovis, Bovicola caprae, Bovicola ovis, Chelopistes meleagridis, Goniodes dissimilis, Goniodes gigas, Menacanthus stramineus, Menopon gallinae, and Trichodectes canis.

(13) Order Orthoptera. A non-exhaustive list of particular genera includes, but is not limited to, Melanoplus spp., and Pterophylla spp. A non-exhaustive list of particular species includes, but is not limited to, Anabrus simplex, Gryllotalpa africana, Gryllotalpa australis, Gryllotalpa brachyptera, Gryllotalpa hexadactyla, Locusta migratoria, Microcentrum retinerve, Schistocerca gregaria, and Scudderia furcata.

(14) Order Siphonaptera. A non-exhaustive list of particular species includes, but is not limited to, Ceratophyllus gallinae, Ceratophyllus niger, Ctenocephalides canis, Ctenocephalides felis, and Pulex irritans.

(15) Order Siphonostomatoida. A non-exhaustive list of particular species includes, but is not limited to, Lepeophtheirus salmonis, Lepeophtheirus pectoralis, Caligus elongatus, and Caligus clemensi.

(16) Order Thysanoptera. A non-exhaustive list of particular genera includes, but is not limited to, Caliothrips spp., Frankliniella spp., Scirtothrips spp., and Thrips spp. A non-exhaustive list of particular species includes, but is not limited to, Frankliniella fusca, Frankliniella occidentalis, Frankliniella schultzei, Frankliniella williamsi, Heliothrips haemorrhoidalis, Rhipiphorothrips cruentatus, Scirtothrips citri, Scirtothrips dorsalis, Taeniothrips rhopalantennalis, Thrips hawaiiensis, Thrips nigropilosus, Thrips orientalis, and Thrips tabaci.

(17) Order Thysanura. A non-exhaustive list of particular genera includes, but is not limited to, Lepisma spp. and Thermobia spp.

(18) Order Acarina. A non-exhaustive list of particular genera includes, but is not limited to, Acarus spp., Aculops spp., Boophilus spp., Demodex spp., Dermacentor spp., Epitrimerus spp., Eriophyes spp., Ixodes spp., Oligonychus spp., Panonychus spp., Rhizoglyphus spp., and Tetranychus spp. A non-exhaustive list of particular species includes, but is not limited to, Acarapis woodi, Acarus siro, Aceria mangiferae, Aculops lycopersici, Aculus pelekassi, Aculus schlechtendali, Amblyomma americanum, Brevipalpus obovatus, Brevipalpus phoenicis, Dermacentor variabilis, Dermatophagoides pteronyssinus, Eotetranychus carpini, Notoedres cati, Oligonychus coffeae, Oligonychus ilicis, Panonychus citri, Panonychus ulmi, Phyllocoptruta oleivora, Polyphagotarsonemus latus, Rhipicephalus sanguineus, Sarcoptes scabiei, Tegolophus perseaflorae, Tetranychus urticae, and Varroa destructor.

(19) Order Symphyla. A non-exhaustive list of particular species includes, but is not limited to, Scutigerella immaculata.

(20) Phylum Nematoda. A non-exhaustive list of particular genera includes, but is not limited to, Aphelenchoides spp., Belonolaimus spp., Criconemella spp., Ditylenchus spp., Heterodera spp., Hirschmanniella spp., Hoplolaimus spp., Meloidogyne spp., Pratylenchus spp., and Radopholus spp. A non-exhaustive list of particular sp. includes, but is not limited to, Dirofilaria immitis, Heterodera zeae, Meloidogyne incognita, Meloidogyne javanica, Onchocerca volvulus, Radopholus similis, and Rotylenchulus reniformis.

The phrase “pesticidally effective amount” means the amount of a pesticide needed to achieve an observable effect on a pest, for example, the effects of necrosis, death, retardation, prevention, removal, destruction, or otherwise diminishing the occurrence and/or activity of a pest in a locus, this effect may come about when, pest populations are repulsed from a locus, pests are incapacitated in, or around, a locus, and/or pests are exterminated in, or around, a locus. Of course, a combination of these effects can occur. Generally, pest populations, activity, or both are desirably reduced more than fifty percent, preferably more than 90 percent, and most preferably more than 99 percent. In general a pesticidally effective amount, for agricultural purposes, is from about 0.0001 grams per hectare to about 5000 grams per hectare, preferably from about 0.0001 grams per hectare to about 500 grams per hectare, and it is even more preferably from about 0.0001 grams per hectare to about 50 grams per hectare.

### DETAILED DESCRIPTION OF THE DISCLOSURE

This document discloses molecules of Formula One

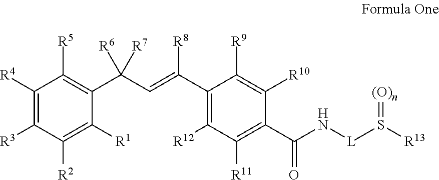

wherein:

(A) R¹, R⁵, R⁶, R⁹, and R¹² are each independently selected from the group consisting of H, F, Cl, Br, I, CN, (C₁-C₄)alkyl, (C₁-C₄)haloalkyl, (C₁-C₄)alkoxy, and (C₁-C₄)haloalkoxy;

(B) R² is selected from the group consisting of H, F, Cl, Br, I, CN, (C₁-C₄)alkyl, (C₁-C₄)haloalkyl, (C₁-C₄)alkoxy, and (C₁-C₄)haloalkoxy;

(C) R³ and R⁴ are each independently selected from the group consisting of (D), H, F, Cl, Br, I, CN, C(O)H, (C₁-C₄)alkyl, (C₂-C₄)alkenyl, (C₂-C₄)alkynyl, (C₁-C₄)haloalkyl, (C₁-C₄)alkoxy, and (C₁-C₄)haloalkoxy;

(D) R³ and R⁴ together can optionally form a 3- to 5-membered saturated or unsaturated, heterohydrocarbyl link, which may contain one or more heteroatoms selected from the group consisting of nitrogen, sulfur, and oxygen,

wherein said heterohydrocarbyl link may optionally be substituted with one or more substituents independently selected from the group consisting of H, F, Cl, Br, I, CN, and OH;

(E) R⁷ is (C₁-C₆)haloalkyl;

(F) R⁸ is selected from the group consisting of H, (C₁-C₄)alkyl, (C₁-C₄)haloalkyl, and (C₁-C₄)alkoxy;

(G) R¹⁰ is selected from the group consisting of F, Cl, Br, I, (C₁-C₄)alkyl, (C₂-C₄)alkenyl, (C₂-C₄)alkynyl, (C₁-C₄)haloalkyl, (C₁-C₄)alkoxy, and (C₁-C₄)haloalkoxy;

(H) R¹¹ is selected from the group consisting of H, F, Cl, Br, I, (C₁-C₄)alkyl, or (C₁-C₄)haloalkyl;

(I) L is a linker that is selected from the group consisting of (C₁-C₈)alkyl, (C₁-C₄)alkoxy, (C₃-C₆)cycloalkyl-(C₁-C₄)alkyl, (C₁-C₄)alkyl-(C₃-C₆)cycloalkoxy, (C₁-C₄)alkyl-S—(C₁-C₄)alkyl, (C₁-C₄)alkyl-S(O)—(C₁-C₄)alkyl, and (C₁-C₄)alkyl-S(O)₂—(C₁-C₄)alkyl, wherein each alkyl, alkoxy, and cycloalkyl may optionally be substituted with one or more substituents independently selected from the group consisting of F, Cl, Br, I, CN, OH, oxetanyl, C(═O)NH(C₁-C₄)haloalkyl, and (C₁-C₄)alkoxy;

(J) n is selected from the group consisting of 0, 1, and 2;

(K) R¹³ is selected from the group consisting of (C₁-C₄)alkyl, (C₂-C₄)alkenyl, (C₁-C₄)haloalkyl, (C₁-C₄)alkoxy, (C₁-C₄)haloalkoxy, phenyl, benzyl, (C₁-C₄)alkyl-(C₃-C₆)cycloalkyl, and NH(C₁-C₄)haloalkyl,

wherein each alkyl, alkenyl, haloalkyl, alkoxy, haloalkoxy, phenyl, and cycloalkyl, may optionally be substituted with one or more substituents independently selected from the group consisting of F, Cl, Br, I, CN, and OH; and

agriculturally acceptable acid addition salts, salt derivatives, solvates, ester derivatives, crystal polymorphs, isotopes, resolved stereoisomers, and tautomers, of the molecules of Formula One.

In another embodiment R¹, R⁵, R⁶, R⁹, and R¹² are H. This embodiment may be used in combination with the other embodiments R², R³, R⁴, R⁷, R⁸, R¹⁰, R¹¹, L, n, and R¹³.

In another embodiment R² is Cl or Br. This embodiment may be used in combination with the other embodiments R¹, R³, R⁴, R⁵, R⁶, R⁷, R⁸, R⁹, R¹⁰, R¹¹, R¹², L, n, and R¹³.

In another embodiment R³ is H, F, Cl, or CN. This embodiment may be used in combination with the other embodiments R¹, R², R⁴, R⁵, R⁶, R⁷, R⁸, R⁹, R¹⁰, R¹¹, R¹², L, n, and R¹³.

In another embodiment R⁴ is Cl, Br, or C(O)H. This embodiment may be used in combination with the other embodiments R¹, R², R³, R⁵, R⁶, R⁷, R⁸, R⁹, R¹⁰, R¹¹, R¹², L, n, and R¹³.

In another embodiment R³ and R⁴ together are —OCH₂O—. This embodiment may be used in combination with the other embodiments R¹, R², R⁵, R⁶, R⁷, R⁸, R⁹, R¹⁰, R¹¹, R¹², L, n, and R¹³.

In another embodiment R², R³, and R⁴ are Cl. This embodiment may be used in combination with the other embodiments R¹, R⁵, R⁶, R⁷, R⁸, R⁹, R¹⁰, R¹¹, R¹², L, n, and R¹³.

In another embodiment R⁷ is CF₃ or CF₂CH₃. This embodiment may be used in combination with the other embodiments R¹, R², R³, R⁴, R⁵, R⁶, R⁸, R⁹, R¹⁰, R¹¹, R¹², L, n, and R¹³.

In another embodiment R⁸ is H, OCH₃, or OCH₂CH₃. This embodiment may be used in combination with the other embodiments R¹, R², R³, R⁴, R⁵, R⁶, R⁷, R⁹, R¹⁰, R¹¹, R¹², L, n, and R¹³.

In another embodiment R¹⁰ is F, Cl, Br, CH₃, CH₂CH₃, CHF₂, or CF₃. This embodiment may be used in combination with the other embodiments R¹, R², R³, R⁴, R⁵, R⁶, R⁷, R⁸, R⁹, R¹⁰, R¹¹, R¹², L, n, and R¹³.

In another embodiment R¹¹ is H or CH₃. This embodiment may be used in combination with the other embodiments R¹, R², R³, R⁴, R⁵, R⁶, R⁷, R⁸, R⁹, R¹⁰, R¹², L, n, and R¹³.

In another embodiment L is —CH₂CH₂—, —CH(CH₃)CH₂—, —CH(CH₂CH₃)CH₂—, —CH(CH(CH₃)₂)CH₂—, —C(CH₃)₂CH₂—, —CH(CH₃)CH₂CH₂—, —CH(CH₂OCH₃)CH₂—, —C(cyclopropyl)CH₂—, —CH₂C(3,3-oxetanyl)-, or —CH₂CH(SCH₂CH₃)—. This embodiment may be used in combination with the other embodiments R¹, R², R³, R⁴, R⁵, R⁶, R⁷, R⁸, R⁹, R¹⁰, R¹¹, R¹², n, and R¹³.

In another embodiment n is 0, 1, or 2. This embodiment may be used in combination with the other embodiments R¹, R², R³, R⁴, R⁵, R⁶, R⁷, R⁸, R⁹, R¹⁰, R¹¹, R¹², L, and R¹³.

In another embodiment R¹³ is CH₃, CH₂CH₃, CH₂CH₂CH₃, CH(CH₃)₂, CH₂CH(CH₃)₂, CH₂CH═CH₂, CH₂CF₃, CH₂CH₂CF₃, phenyl, CH₂phenyl, CH₂cyclopropyl, or NHCH₂CF₃, wherein each phenyl and cyclopropyl is optionally substituted with one or more substituents selected from the group consisting of F, Cl, Br, and CN. This embodiment may be used in combination with the other embodiments R¹, R², R³, R⁴, R⁵, R⁶, R⁷, R⁸, R¹, R¹⁰, R¹¹, R¹², L, and n.

In another embodiment

(A) R¹, R⁵, R⁶, R⁹, and R¹² are H;

(B) R² is selected from the group consisting of Cl and Br;

(C) R³ and R⁴ are, each independently selected from the group consisting of (D), H, F, Cl, Br, I, CN, and C(O)H;

(D) R³ and R⁴ together can optionally form a 3- to 5-membered saturated or unsaturated, heterohydrocarbyl link, which may contain one or more heteroatoms selected from the group consisting of nitrogen, sulfur, and oxygen,

wherein said heterohydrocarbyl link may optionally be substituted with one or more substituents independently selected from the group consisting of H, F, Cl, Br, I, CN, and OH;

(E) R⁷ is (C₁-C₆)haloalkyl;

(F) R⁸ is selected from the group consisting of H and (C₁-C₄)alkoxy;

(G) R¹⁰ is selected from the group consisting of F, Cl, Br, I, (C₁-C₄)alkyl, and (C₁-C₄)haloalkyl;

(H) R¹¹ is selected from the group consisting of H and (C₁-C₄)alkyl;

(I) L is a linker that is selected from the group consisting of (C₁-C₅)alkyl, (C₁-C₄)alkoxy, (C₃-C₆)cycloalkyl-(C₁-C₄)alkyl, (C₁-C₄)alkyl-(C₃-C₆)cycloalkoxy, and (C₁-C₄)alkyl-S—(C₁-C₄)alkyl,

wherein each alkyl, alkoxy, and cycloalkyl may optionally be substituted with one or more (C₁-C₄)alkoxy substituents;

(J) n is selected from the group consisting of 0, 1, and 2; and

(K) R¹³ is selected from the group consisting of (C₁-C₄)alkyl, (C₂-C₄)alkenyl, (C₁-C₄)haloalkyl, phenyl, benzyl, (C₁-C₄)alkyl-(C₃-C₆)cycloalkyl, and NH(C₁-C₄)haloalkyl,

wherein each alkyl, alkenyl, haloalkyl, phenyl, and cycloalkyl, may optionally be substituted with one or more substituents independently selected from the group consisting of F, Cl, Br, I, and CN.

In another embodiment

(A) R¹, R⁵, R⁶, R⁹, and R¹² are H;

(B) R² is selected from the group consisting of Cl and Br;

(C) R³ and R⁴ are, each independently selected from the group consisting of H, F, Cl, Br, I, and CN.

(E) R⁷ is (C₁-C₆)haloalkyl;

(F) R⁸ is H;

(G) R¹⁰ is selected from the group consisting of F, Cl, Br, I, (C₁-C₄)alkyl, and (C₁-C₄)haloalkyl;

(H) R¹¹ is selected from the group consisting of H and (C₁-C₄)alkyl;
(I) L is a linker that is selected from the group consisting of (C₁-C₈)alkyl, (C₃-C₆)cycloalkyl-(C₁-C₄)alkyl, and (C₁-C₄)alkyl-S—(C₁-C₄)alkyl;

(J) n is selected from the group consisting of 0, 1, and 2; and

(K) R¹³ is selected from the group consisting of (C₁-C₄)alkyl, (C₂-C₄)alkenyl, (C₁-C₄)haloalkyl, benzyl, (C₁-C₄)alkyl-(C₃-C₆)cycloalkyl, and NH(C₁-C₄)haloalkyl,

wherein each alkyl, alkenyl, haloalkyl, phenyl, and cycloalkyl, may optionally be substituted with one or more substituents independently selected from the group consisting of F, Cl, Br, and I.

### Preparation of Benzyl Halides

Benzyl alcohol 1-3, wherein R¹, R², R³, R⁴, R⁵, R⁶, and R⁷ are as previously disclosed, may be prepared in several ways. Ketones 1-1 may be prepared by treating bromobenzenes with a lithium base such as n-butyllithium in a polar, aprotic solvent preferably diethyl ether at temperatures from about −78° C. to about 0° C. followed by treatment with esters R⁷C(O)O(C₁-C₄)alkyl, wherein R⁷ is as previously disclosed, such as ethyl 2,2-difluoropropanoate (not shown). Treatment of ketones 1-1, wherein R¹, R², R³, R⁴, R⁵, and R⁷ are as previously disclosed, with a reducing agent such as sodium borohydride, in the presence of a base, such as aqueous sodium hydroxide, in a polar, protic solvent preferably methanol at about −10° C. to about 10° C. may provide benzyl alcohols 1-3 (Scheme 1, step a). Alternatively, aldehydes 1-2, wherein R⁶ is H and R¹, R², R³, R⁴, and R⁵ are as previously disclosed, may be allowed to react with trifluorotrimethylsilane in the presence of a catalytic amount of tetrabutylammonium fluoride in a polar, aprotic solvent preferably tetrahydrofuran (Scheme 1, step b) to provide benzyl alcohols 1-3, wherein R⁷ is CF₃. Subsequently, benzyl alcohols 1-3 may be converted into benzyl halides 1-4, wherein E is Br, Cl, or I, and R¹, R², R³, R⁴, R⁵, R⁶, and R⁷ are as previously disclosed, by treatment with a halogenating reagent, such as N-bromosuccinimide, and triethylphosphite in a solvent that does not react with the reagents preferably dichloromethane at about 40° C. to provide benzyl halides 1-4, E is Br (Scheme 1, step c). Alternatively, benzyl alcohols 1-3 may be converted into benzyl halides 1-4, where E is Br by treatment with a sulfonyl chloride such as methanesulfonyl chloride in the presence of a base such as triethylamine and subsequent treatment of the resultant sulfonate with a transition metal bromide such as iron(III) bromide. Additionally, treatment with chlorinating reagents such as thionyl chloride in the presence of a base such as pyridine in a hydrocarbon solvent such as toluene at about 110° C. may provide benzyl halides 1-4, where E is Cl (Scheme 1, step c).

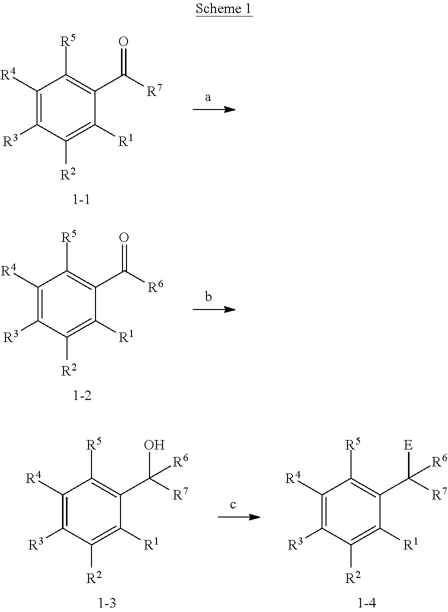

### Preparation of Vinylbenzoic Acids and Esters

Halobenzoic acids 2-1, wherein R¹, R¹⁰, R¹¹, and R¹² are as previously disclosed may be converted to vinylbenzoic acid esters 2-3, wherein R⁸, R⁹, R¹⁰, R¹¹, and R¹² are as previously disclosed or vinylbenzoic acids 2-4, wherein R⁸, R⁹, R¹⁰, R¹¹, and R¹² are as previously disclosed. Halobenzoic acids 2-1, may be treated with a base, such as n-butylithium and then N,N-dimethylformamide in a polar, aprotic solvent, such as tetrahydrofuran, at a temperature of about −78° C. (Scheme 2, step a). The resulting formyl benzoic acids may be treated with an acid, such as sulfuric acid, in the presence of an alcohol, such as ethanol, to provide formyl benzoic acid ethyl esters 2-2 (Scheme 2, step b). Vinyl benzoic acid esters 2-3 may be accessed via reaction of 2-2 with a base, such as potassium carbonate, and methyl triphenylphosphonium bromide in a polar, aprotic solvent, such as 1,4-dioxane, at about ambient temperature (Scheme 2, step c).

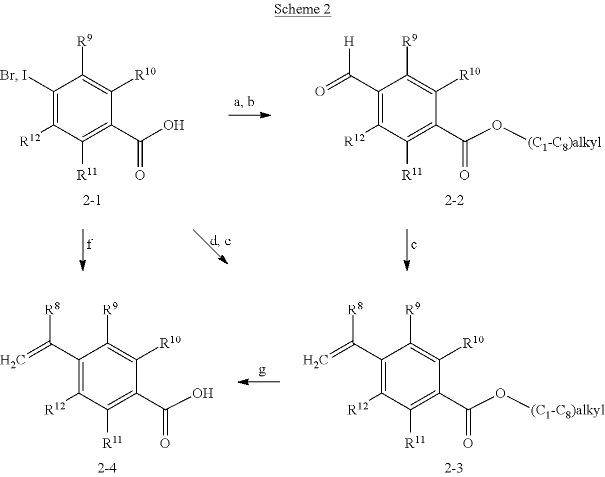

Alternatively, halobenzoic acids 2-1 may be treated with di-tert-butyl dicarbonate in the presence of a base, such as triethylamine and a catalytic amount of 4-(dimethylamino)pyridine in a polar, aprotic solvent, such as tetrahydrofuran, at about ambient temperature (Scheme 2, step d). The resulting benzoic acid tert-butyl esters may be treated with vinyl boronic anhydride pyridine complex in the presence of a palladium catalyst, such a tetrakis(triphenylphospine)palladium(0), and a base, such as potassium carbonate, in a non-reactive solvent such as toluene at about 110° C., to provide vinyl benzoic acid esters 2-3 (Scheme 2, step e).

Halobenzoic acids 2-1 may be directly treated with a vinyl borane source such as vinyltrifluoroborate or 3-hydroxy-2, 3-dimethylbutan-2-yl hydrogen vinylboronate in the presence of a palladium catalyst, such as 1,1′-bis(diphenylphosphino)ferrocene palladium(II) dichloride, and a base, such as potassium carbonate, in a non-reactive solvent such as dimethylsulfoxide at temperatures ranging from about 80° C. to about 140° C., to provide vinyl benzoic acids 2-4 (Scheme 2, step f).

Vinyl benzoic acid esters 2-3 may be treated with a metal hydroxide source such as lithium hydroxide in a solvent mixture such as tetrahydrofuran, methanol, and water at about ambient temperature to provide vinyl benzoic acids 2-4 (Scheme 2, step g).

Halobenzoic acid esters 3-1 may be treated with a vinyl stannane source such as tributyl (1-ethoxyvinyl)stannane in the presence of a palladium catalyst, such as bis(triphenylphosphine)palladium(II) dichloride, in a solvent such as N,N-dimethylformamide at temperatures ranging from about 60° C. to about 100° C., to provide vinyl benzoic acid esters 2-3 (Scheme 3, step a).

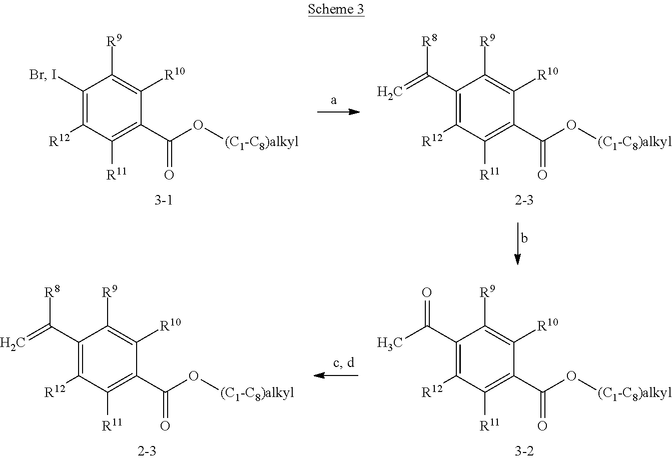

Vinyl benzoic acid esters 2-3 may be treated with an acid such as hydrogen chloride in a solvent such as tetrahydrofuran at about ambient temperature to provide methyl ketone benzoic acid esters 3-2 (Scheme 3, step b). Methyl ketone benzoic acid esters 3-2 may be treated with a latent alcohol source such as trimethoxymethane in the presence of a catalytic amount of an acid such as sulfuric acid in an alcohol solvent such as methanol at about ambient temperature (Scheme 3, step c). The resultant methyl dialkyl acetal benzoic acid esters may be further treated with trimethylchlorosilane in the presence of an organic acid such as 4-nitrobenzoic acid in a basic solvent such as pyridine at temperatures ranging from about 50° C. to about 90° C., to provide vinyl benzoic acid esters 2-3 (Scheme 3, step d).

### Preparation of Phenyl Allylbenzoic Acids

Benzyl halides 1-4 and vinylbenzoic acid esters 2-3 may be treated with a copper(I) source such as copper(I) chloride or copper(I) bromide and a pyridine ligand such as 2,2-bipyridyl in a degassed solvent, such as 1,2-dichlorobenzene, at a temperature of about 180° C. to provide phenyl allylbenzoic esters 4-1, wherein R¹, R², R³, R⁴, R⁵, R⁶, R⁷, R⁸, R⁹, R¹⁰, R¹¹, and R¹² are as previously disclosed (Scheme 4, step a). Phenyl allylbenzoic esters 4-1 may be then converted to phenyl allylbenzoic acids 4-2, wherein R¹, R², R³, R⁴, R⁵, R⁶, R⁷, R⁸, R⁹, R¹⁰, R¹¹, and R¹² are as previously disclosed. Treatment of phenyl allylbenzoic esters 4-1, with an acid, such as concentrated aqueous hydrochloric acid, in a polar, aprotic solvent, such as 1,4-dioxane, at about 100° C. may provide phenyl allylbenzoic acids 4-2 (Scheme 4, step b). Treatment of phenyl allylbenzoic esters 4-1, with a saponification reagent such as trimethylstannanol in a polar, aprotic solvent, such as 1,2-dichloroethane, at about 80° C. may also provide phenyl allylbenzoic acids 4-2 (Scheme 4, step c).

Alternatively, benzyl halides 1-4 and vinylbenzoic acids 2-4 may be treated with a copper(I) source such as copper(I) chloride or copper(I) bromide and a pyridine ligand such as 2,2-bipyridyl in a degassed solvent, such as 1,2-dichlorobenzene or N-methylpyrrolidine, at temperatures between about 60° C. and about 180° C. to provide phenyl allylbenzoic acids 4-2 (Scheme 4, step d).

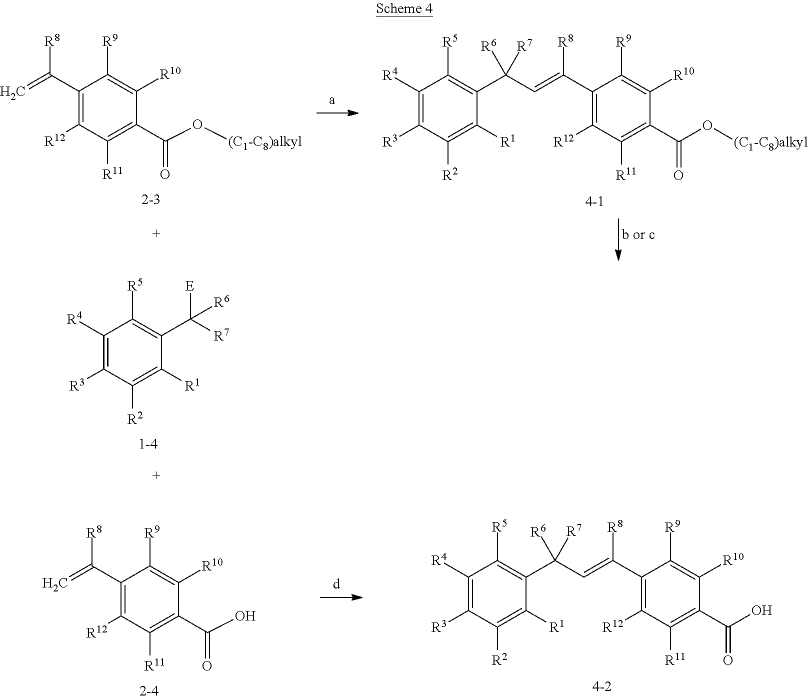

Preparation of Phenyl Allylbenzoic Amides Phenyl allylbenzoic amides 5-3, wherein R¹, R², R³, R⁴, R⁵, R⁶, R⁷, R⁸, R⁹, R¹⁰, R¹¹, R¹², L, n, and R¹³ are as previously disclosed may be prepared by treatment with amines or amine salts 5-2, wherein L, n, and R¹³ are as previously disclosed, and activated carboxylic acids 5-1, wherein A is an activating group, and R¹, R², R³, R⁴, R⁵, R⁶, R⁷, R⁸, R⁹, R¹⁰, R¹¹, and R¹² are as previously disclosed, with a base, such as triethylamine, diisopropylethylamine, or 4-methylmorpholine in an anhydrous aprotic solvent such as dichloromethane, tetrahydrofuran, 1,2-dichloroethane, N,N-dimethylformamide, or any combination thereof, at temperatures between about 0° C. and about 120° C. (Scheme 5, step a).

Activated carboxylic acids 5-1 may be an acid halide, such as an acid chloride, an acid bromide, or an acid fluoride; a carboxylic ester, such as a para-nitrophenyl ester, a pentafluorophenyl ester, an ethyl (hydroxyimino)cyanoacetate ester, a methyl ester, an ethyl ester, a benzyl ester, an N-hydroxysuccinimidyl ester, a hydroxybenzotriazol-1-yl ester, or a hydroxypyridyltriazol-1-yl ester; an O-acylisourea; an acid anhydride; or a thioester. Acid chlorides may be prepared from the corresponding carboxylic acids by treatment with a dehydrating, chlorinating reagent, such as oxalyl chloride or thionyl chloride. Activated carboxylic acids 5-1 may be prepared from carboxylic acids in situ with a uronium salt, such as 1-[bis(dimethylamino)methylene]-1H-1,2,3-triazolo[4,5-b]pyridinium 3-oxid hexafluorophosphate (HATU), O-(benzotriazol-1-yl)-N,N,N′,N′-tetramethyluronium hexafluorophosphate (HBTU), or (1-cyano-2-ethoxy-2-oxoethylidenaminooxy)dimethylamino-morpholino-carbenium hexafluorophosphate (COMU). Activated carboxylic acids 5-1 may also be prepared from carboxylic acids in situ with a phosphonium salt such as benzotriazol-1-yl-oxytripyrrolidinophosphonium hexafluorophosphate (PyBop). Activated carboxylic acids 5-1 may also be prepared from carboxylic acids in situ with a coupling reagent such as 1-(3-dimethylamino propyl)-3-ethylcarbodiimide, or dicyclohexylcarbodiimide in the presence of a triazole such as hydroxybenzotriazole-monohydrate (HOBt) or 1-hydroxy-7-azabenzotriazole (HOAt). O-Acylisoureas may be prepared with a dehydrating carbodimide such as 1-(3-dimethylamino propyl)-3-ethylcarbodiimide or dicyclohexylcarbodiimide.

Phenyl allylbenzoic amides 5-3, wherein n is 0 (sulfide), may be oxidized to the corresponding sulfoxide or sulfone by treatment with one equivalent of sodium perborate in a protic solvent such as acetic acid (sulfoxide) or two equivalents of sodium perborate (sulfone). Preferably, the oxidation will be performed at temperatures between about 40° C. to about 100° C. using 1.5 equivalents of sodium perborate to provide chromatographically separable mixtures of sulfoxide and sulfone diphenyl allylbenzoic amides 5-3.

Alternatively, phenyl allylbenzoic amides 5-3, wherein n is 0 (sulfide), may be oxidized to the corresponding sulfoxide by treatment with hydrogen peroxide in a protic solvent such as methanol or preferably hexafluoroisopropanol. Preferably, the oxidation will be performed at temperatures between about 10° C. to about 100° C.

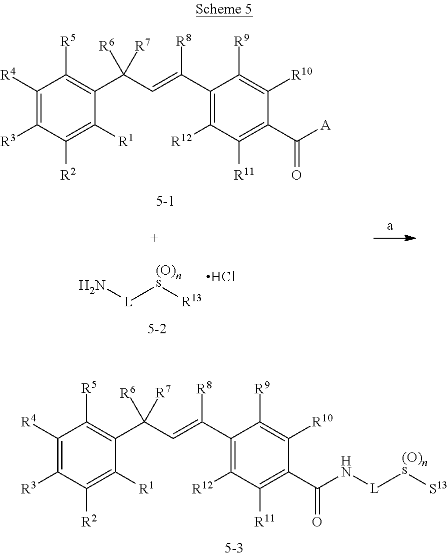

Amine salts 5-2 may be generated in situ from the corresponding N-tert-butoxycarbonyl amines by treatment with an acid such as hydrogen chloride. Additionally, amine salts 5-2 may be neutralized in situ in the presence of a base such as sodium bicarbonate, triethylamine, or 4-methylmorpholine during reaction with activated carboxylic acids 5-1 to provide phenyl allylbenzoic amides 5-3.

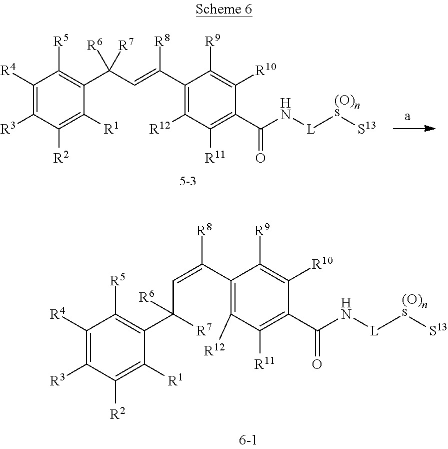

Phenyl allylbenzoic amides 5-3 may be exposed to ultraviolet irradiation in a deuterated or non-deuterated solvent such as acetone to provide phenyl allylbenzoic amides 6-1 (Scheme 6, step a).

### Preparation of Amines and Amine Salts 5-2

Amines and amine salts 5-2 may be prepared as outlined in Scheme 7. N-tert-Butoxycarbonyl aminoalcohols 7-1, wherein L is as previously disclosed, may be treated with a sulfonyl chloride such as methanesulfonyl chloride or a sulfonyl anhydride such as methanesulfonyl anhydride in the presence of a base such as triethylamine in a solvent such as dichloromethane at temperatures from about −20° C. to about 40° C. (Scheme 7, step a). The resultant N-tert-butoxycarbonyl amino sulfonates may then be treated with sodium thioacetate, prepared by treating thioacetic acid with a base such as sodium hydride, in a polar, aprotic solvent such as N,N-dimethylformamide at temperatures from about 10° C. to about 40° C. to provide N-tert-butoxycarbonyl amino thioesters 7-2, wherein L is as previously disclosed (Scheme 7, step b). Alkylation of the N-tert-butoxycarbonyl amino thioesters 7-2 may be accomplished in an oxygen free environment by first removing the acetate from the sulfur by treatment with a metal hydroxide base such as sodium hydroxide followed by treatment with halides R¹³-halo, wherein R¹³ is alkyl, or triflates R¹³-OTf, wherein R¹³ is alkyl in a polar, protic solvent such as methanol at temperatures from about −10° C. to about 40° C. to provide N-tert-butoxycarbonyl amino sulfides 7-3, wherein L and R¹³ are as previously disclosed (Scheme 7, step c). N-tert-Butoxycarbonyl amino sulfides 7-3 may then be treated with an acid such as hydrogen chloride to provide amino salts 5-2, wherein n is 0 (Scheme 7, step d). Optionally, the amine salts 5-2 may be neutralized in the presence of a base such as sodium bicarbonate or triethylamine prior to use in subsequent reactions.

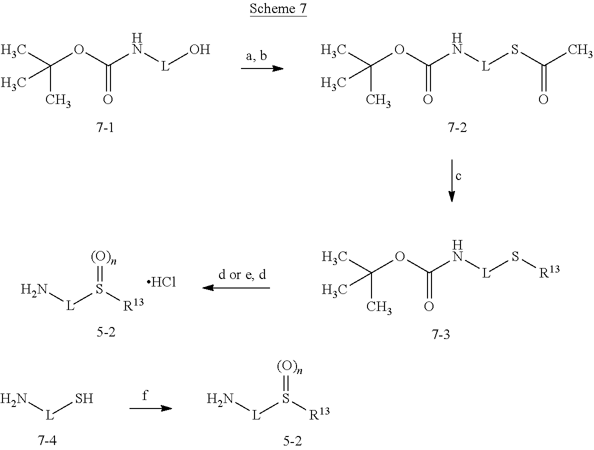

Alternatively, N-tert-butoxycarbonyl amino sulfides 7-3, may be oxidized to the corresponding sulfoxide or sulfone by treatment with one equivalent of sodium perborate in a protic solvent such as acetic acid to provide the sulfoxide; or two equivalents of sodium perborate to provide the sulfone (Scheme 7, step e). The resultant sulfones may then be treated with an acid such as hydrogen chloride to provide amine salts 5-2 (Scheme 7, step d). Optionally, the amine salts 5-2 may be neutralized in the presence of a base such as sodium bicarbonate or triethylamine prior to use in subsequent reactions.

Amines 5-2 may alternatively be prepared by treating aminothiols 7-4, wherein L is as previously disclosed with a base such as sodium hydride followed by treatment with halides R¹³-halo, wherein R¹³ is alkyl, or triflates R¹³-OTf, wherein R¹³ is alkyl in a polar, aprotic solvent such as N,N-dimethylformamide at temperatures from about 15° C. to about 50° C. (Scheme 7, step f).

Thiols 8-2, wherein R¹³ is as previously disclosed may be treated with a base such as sodium hydride followed by treatment with acids 8-1, wherein L is as previously disclosed, in a polar, aprotic solvent such as N,N-dimethylformamide at temperatures from about −10° C. to about 30° C. to provide thioacids 8-3, wherein L and R¹³ are as previously disclosed (Scheme 8, step a). Thioacids 8-3 may then be treated with an azide source such as diphenyl phosphorazidate in the presence of a base such as triethylamine in a solvent such as 1,2-dichloroethane at temperatures from about 60° C. to about 90° C. to effect a Curtius rearrangement. The resultant isocyanate may be treated with a benzyl alcohol such as (4-methoxyphenyl)methanol to provide benzyl carbamates 8-4, wherein L and R¹³ are as previously disclosed (Scheme 8, step b). Benzyl carbamates 8-4 may be treated with an acid such as trifluoroacetic acid followed by salt metathesis with hydrochloric acid to provide amino salts 5-2, wherein n is 0 (Scheme 8, step c). Optionally, the amine salts 5-2 may be neutralized in the presence of a base such as sodium bicarbonate or triethylamine prior to use in subsequent reactions.

Alternatively, benzyl carbamates 8-4, wherein n is 0 (sulfide), may be oxidized to the corresponding sulfone by treatment with two equivalents of sodium perborate (Scheme 8, step d). The resultant sulfones may then be treated with an acid such as hydrogen chloride to provide amino salts 5-2 (Scheme 8, step c). Optionally, the amine salts 5-2 may be neutralized in the presence of a base such as sodium bicarbonate or triethylamine prior to use in subsequent reactions.

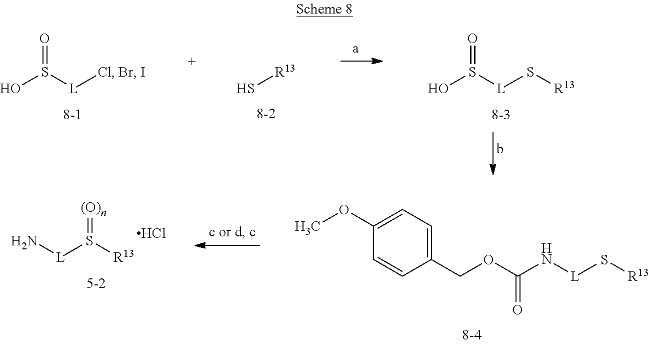

### Preparation of N-Tert-Butoxycarbonylamine Salts 9-4

N-tert-Butoxycarbonyl amine salts 9-4, wherein R¹³ is as previously disclosed, may be prepared as outlined in Scheme 9. N-tert-Butoxycarbonyl amino acids 9-1, wherein R¹³ is as previously disclosed, may be treated with an alkyl chloroformate such as isobutyl chloroformate in the presence of a base such as 4-methylmorpholine followed by treatment with (C₁-C₄)haloalkyl amine in a polar solvent such as tetrahydrofuran at temperatures from about −78° C. to about ambient temperature to provide N-tert-butoxycarbonyl amines 9-2, wherein R¹³ is as previously disclosed (Scheme 9, step a).

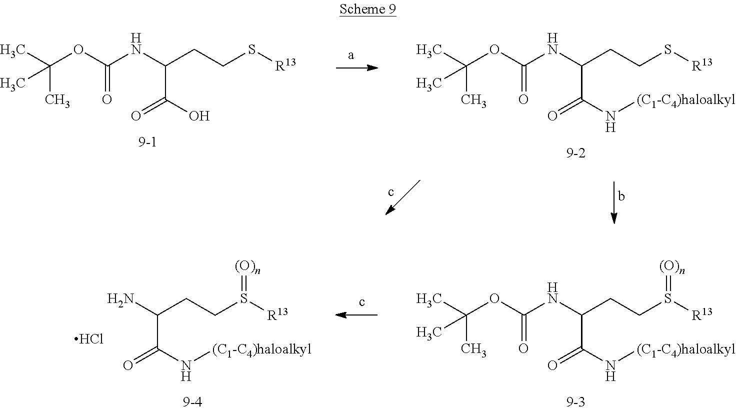

N-tert-Butoxycarbonyl amines 9-2 may be oxidized to the corresponding sulfoxide or sulfone by treatment with about 1 equivalent of sodium perborate in a protic solvent such as acetic acid to provide N-tert-butoxycarbonyl amines 9-3, wherein n is 1 (sulfoxide) or about 2 equivalents of sodium perborate to provide N-tert-butoxycarbonyl amines 9-3, wherein n is 2 (sulfone) (Scheme 9, step b). The sulfur oxidation of N-tert-butoxycarbonyl amines 9-2 may also be accomplished by treatment with about 1 or about 2 equivalents of meta-chloroperbenzoic acid. N-tert-Butoxycarbonyl amines 9-2 may be treated with an acid such as hydrogen chloride to provide N-tert-butoxycarbonyl amine salts 9-4, wherein n is 0 (Scheme 9, step c). Likewise, N-tert-butoxycarbonyl amines 9-3 may be treated with an acid such as hydrogen chloride to provide N-tert-butoxycarbonyl amine salts 9-4, wherein n is 1 or 2 (Scheme 9, step c).

### Preparation of Amines 10-4

Benzylcarbamate thioesters 10-1, wherein L is as previously disclosed, may be treated with an oxidant such as hydrogen peroxide in a solvent such as acetic acid at about ambient temperature. Subsequent chlorination of the resultant thiospecies with a chlorine source such as oxalyl chloride in a solvent such as dichloromethane at about ambient temperature may provide sulfonyl chlorides 10-2, wherein L is as previously disclosed (Scheme 10, step a).

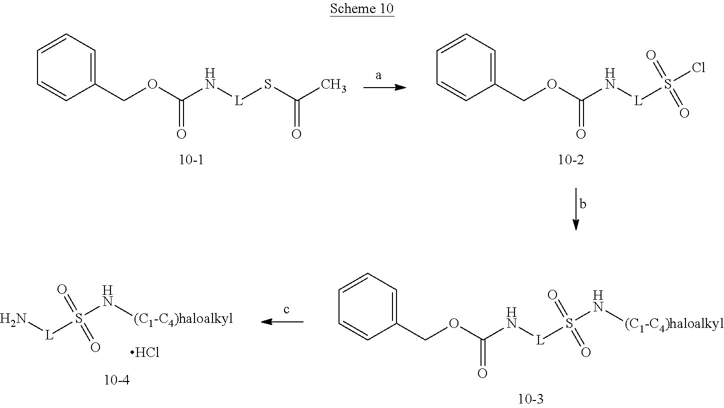

Sulfonyl chlorides 10-2 may be treated with a (C₁-C₄)haloalkyl amine in a solvent such as dichloromethane at about ambient temperature to provide sulfonamides 10-3, wherein L is as previously disclosed (Scheme 10, step b). Sulfonamides 10-3 may be treated with a hydrogen source such as hydrogen in the presence of a palladium source such as palladium on carbon in a polar solvent such as ethanol at about room temperature to provide amines 10-4, wherein L is as previously disclosed (Scheme 10, step c). Optionally, amines 10-4 may be treated with an acid such as hydrogen chloride to provide amine salts 10-4, wherein L is as previously disclosed.

### Preparation of Amines 11-3

Nitromethylene oxetanes 11-1 may be treated with thiols 8-2 in a solvent such as dichloromethane at about ambient temperature to provide nitromethyl thio oxetanes 11-2, wherein R¹³ is as previously disclosed (Scheme 11, step a). Reduction of nitromethyl thio oxetanes 11-2 may be effected by treatment with a transition metal such as zinc in the presence of a hydrogen source such as ammonium chloride in a polar solvent such as ethanol at temperatures from about 15° C. to about 50° C. to provide amines 11-3, wherein R¹³ is as previously disclosed (Scheme 11, step b).

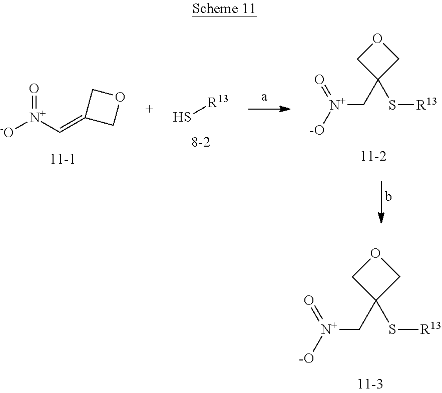

### EXAMPLES

These examples are for illustration purposes and are not to be construed as limiting this disclosure to only the embodiments disclosed in these examples.

Starting materials, reagents, and solvents that were obtained from commercial sources were used without further purification. Anhydrous solvents were purchased as Sure/Seal™ from Aldrich and were used as received. Melting points were obtained on a Thomas Hoover Unimelt capillary melting point apparatus or an OptiMelt Automated Melting Point System from Stanford Research Systems and are uncorrected. Examples using “room temperature” were conducted in climate controlled laboratories with temperatures ranging from about 20° C. to about 24° C. Molecules are given their known names, named according to naming programs within ISIS Draw, ChemDraw, or ACD Name Pro. If such programs are unable to name a molecule, such molecule is named using conventional naming rules. ¹H NMR spectral data are in ppm (δ) and were recorded at 300, 400, 500, or 600 MHz; ¹³C NMR spectral data are in ppm (δ) and were recorded at 75, 100, or 150 MHz, and 19F NMR spectral data are in ppm (δ) and were recorded at 376 MHz, unless otherwise stated.

### Example 1: Preparation of (E)-2-bromo-4-(4,4,4-trifluoro-3-(3,4,5-trichlorophenyl)but-1-en-1-yl)-N-(2-((2,2,2-trifluoroethyl)thio)ethyl)benzamide (F1)

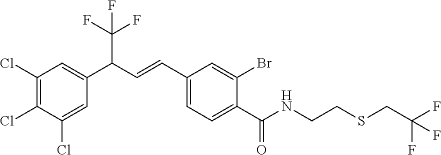 [SMILES: O=C(NCCSCC(F)(F)F)c1ccc(/C=C/C(c2cc(Cl)c(Cl)c(Cl)c2)C(F)(F)F)cc1Br]

A 20 mL vial was charged with 2-(3H-[1,2,3]triazolo[4, 5-b]pyridin-3-yl)-1,1,3,3-tetramethylisouronium hexafluorophosphate (V) (0.599 g, 1.58 mmol), (E)-2-bromo-4-(4,4,4-trifluoro-3-(3,4,5-trichlorophenyl)but-1-en-1-yl)benzoic acid (0.700 g, 1.43 mmol), 4-methylmorpholine (0.472 mL, 4.30 mmol), and 2-((2,2,2-trifluoroethyl)thio)ethanamine (C1) (0.912 g, 5.73 mmol), and N,N-dimethylformamide (3 mL) to give a brown solution. The reaction was left to stir for 3 hours. The reaction was diluted with ethyl acetate and water. The organic phase was washed with water (2×), aqueous citric acid (5%), and saturated sodium bicarbonate. The organic layer was dried over sodium sulfate, filtered, and concentrated. Purification by flash column chromatography using 0-50% ethyl acetate/hexanes provided the title compound as an orange tacky solid (0.360 g, 36%).

The following compounds were prepared according to the procedures disclosed in Example 1:

### (E)-N-(2-(Benzylsulfinyl)ethyl)-2-bromo-4-(4,4,4-trifluoro-3-(3,4,5-trichlorophenyl)but-1-en-1-yl)benzamide (F2)

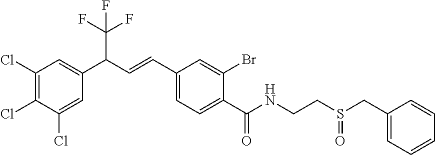 [SMILES: O=C(NCCS(=O)Cc1ccccc1)c1ccc(/C=C/C(c2cc(Cl)c(Cl)c(Cl)c2)C(F)(F)F)cc1Br]

Isolated as an orange foam (0.256 g, 32%).

### (E)-2-Bromo-N-(2-((4-chlorophenyl)sulfonyl)ethyl)-4-(4,4,4-trifluoro-3-(3,4,5-trichlorophenyl)but-1-en-1-yl)benzamide (F4)

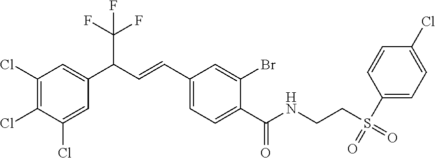 [SMILES: O=C(NCCS(=O)(=O)c1ccc(Cl)cc1)c1ccc(/C=C/C(c2cc(Cl)c(Cl)c(Cl)c2)C(F)(F)F)cc1Br]

Isolated as an orange foam (0.17 g, 57%).

### Example 2: Preparation of N-((2R)-3-methyl-1-((2,2,2-trifluoroethyl)sulfinyl)butan-2-yl)-4-((E)-4,4,4-trifluoro-3-(3,4,5-trichlorophenyl)but-1-en-1-yl)-2-(trifluoromethyl)benzamide (F15)

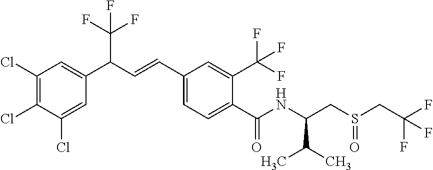 [SMILES: CC(C)[C@H](CS(=O)CC(F)(F)F)NC(=O)c1ccc(/C=C/C(c2cc(Cl)c(Cl)c(Cl)c2)C(F)(F)F)cc1C(F)(F)F]

A 20 mL round-bottomed flask was charged with N—((R)-3-methyl-1-((2,2,2-trifluoroethyl)thio)butan-2-yl)-4-((E)-4,4,4-trifluoro-3-(3,4,5-trichlorophenyl)but-1-en-1-yl)-2-(trifluoromethyl)benzamide (F14) (0.400 g, 0.605 mmol) and methanol (4 mL). Hydrogen peroxide (0.400 mL, 3.53 mmol, 30%) was added to the orange solution and the reaction vial was vortexed and then left to stir at room temperature with a loosely fitting cap for 24 hours. The reaction was then heated with stirring at 55° C. for 48 hours. The reaction mixture was cooled and stirred at room temperature for an additional 4 days. The reaction mixture was diluted with ethyl acetate and aqueous sodium thiosulfate. The layers were mixed and separated. The aqueous layer was extracted with ethyl acetate. The organic layers were combined, dried over magnesium sulfate, filtered, and concentrated. Purification by flash column chromatography using 0-100% ethylacetate/hexanes as eluent provided the title compound as a white solid (0.312 g, 73%).

### Example 2a: Preparation of 4-((Z)-1-ethoxy-4,4,4-trifluoro-3-(3,4,5-trichlorophenyl)but-1-en-1-yl)-N-((2R)-1-((2,2,2-trifluoroethyl)sulfinyl)propan-2-yl)-2-(trifluoromethyl)benzamide (F30)

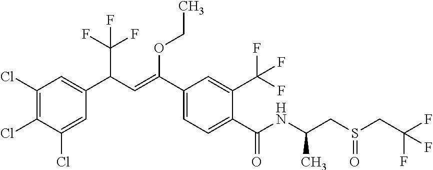 [SMILES: CCO/C(=C\C(c1cc(Cl)c(Cl)c(Cl)c1)C(F)(F)F)c1ccc(C(=O)N[C@H](C)CS(=O)CC(F)(F)F)c(C(F)(F)F)c1]

To a 25 mL vial was added 4-((Z)-1-ethoxy-4,4,4-trifluoro-3-(3,4,5-trichlorophenyl)but-1-en-1-yl)-N—((R)-1-((2,2, 2-trifluoroethyl)thio)propan-2-yl)-2-(trifluoromethyl)benzamide (F29) (0.030 g, 0.044 mmol) and hydrogen peroxide (0.0060 mg, 0.053 mmol) in hexafluoroisopropanol (0.246 mL) to give a colorless solution. The reaction was allowed to stir for 48 hours at room temperature and then quenched with sodium thiosulfate. The reaction mixture was extracted with dichloromethane, dried over magnesium sulfate, filtered, and concentrated. Purification by flash column chromatography provided the title compound as a white solid (0.032 g, 94%).

The following compounds were prepared according to the procedures disclosed in Example 2a:

### 4-((E)-4,4,4-Trifluoro-3-(3,4,5-trichlorophenyl)but-1-en-1-yl)-N-((2R)-1-((2,2,2-trifluoroethyl)sulfinyl)propan-2-yl)-2-(trifluoromethyl)benzamide (F23)

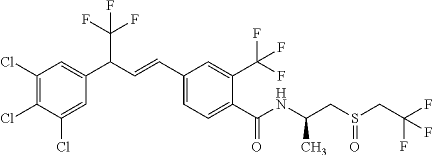 [SMILES: C[C@H](CS(=O)CC(F)(F)F)NC(=O)c1ccc(/C=C/C(c2cc(Cl)c(Cl)c(Cl)c2)C(F)(F)F)cc1C(F)(F)F]

Isolated as a white solid (0.220 g, 97%).

### 4-((E)-3-(3,5-Dichloro-4-fluorophenyl)-4,4,4-trifluorobut-1-en-1-yl)-N-((2R)-1-(2,2,2-trifluoroethyl)sulfinyl)propan-2-yl)-2-(trifluoromethyl)benzamide (F37)

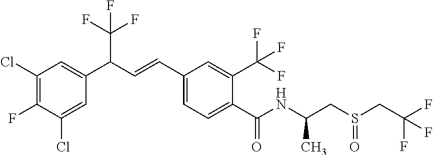 [SMILES: C[C@H](CS(=O)CC(F)(F)F)NC(=O)c1ccc(/C=C/C(c2cc(Cl)c(F)c(Cl)c2)C(F)(F)F)cc1C(F)(F)F]

Isolated as a pale-green solid (0.11 g, 68%).

### 2-Bromo-4-((E)-4,4,4-trifluoro-3-(3,4,5-trichlorophenyl)but-1-en-1-yl)-N-((2R)-1-((2,2,2-trifluoroethyl)sulfinyl)propan-2-yl)benzamide (F39)

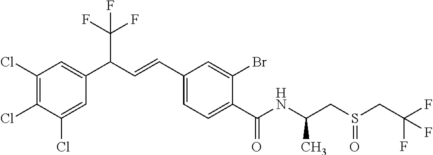 [SMILES: C[C@H](CS(=O)CC(F)(F)F)NC(=O)c1ccc(/C=C/C(c2cc(Cl)c(Cl)c(Cl)c2)C(F)(F)F)cc1Br]

Isolated as an off-white solid (0.10 g, 59%).

### 4-((E)-3-(3,5-Dibromo-4-chlorophenyl)-4,4,4-trifluorobut-1-en-1-yl)-N-((2R)-1-((2,2,2-trifluoroethyl)sulfinyl)propan-2-yl)-2-(trifluoromethyl)benzamide (F42)

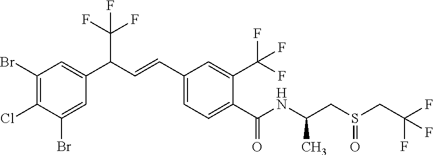 [SMILES: C[C@H](CS(=O)CC(F)(F)F)NC(=O)c1ccc(/C=C/C(c2cc(Br)c(Cl)c(Br)c2)C(F)(F)F)cc1C(F)(F)F]

Isolated as an off-white solid (0.12 g, 76%).

### 2-Methyl-4-((E)-4,4,4-trifluoro-3-(3,4,5-trichlorophenyl)but-1-en-1-yl)-N-((2R)-1-((2,2,2-trifluoroethyl)sulfinyl)propan-2-yl)benzamide (F46)

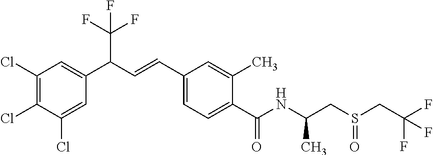 [SMILES: Cc1cc(/C=C/C(c2cc(Cl)c(Cl)c(Cl)c2)C(F)(F)F)ccc1C(=O)N[C@H](C)CS(=O)CC(F)(F)F]

Isolated as a pale-yellow solid (0.10 g, 94%).

### 2-Bromo-4-((E)-3-(3,5-dichloro-4-fluorophenyl)-4,4,4-trifluorobut-1-en-1-yl)-N-((2R)-1-((2,2,2-trifluoroethyl)sulfinyl)propan-2-yl)benzamide (F49)

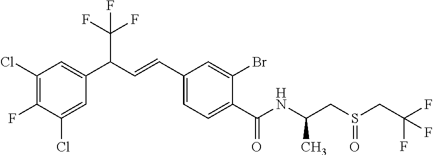 [SMILES: C[C@H](CS(=O)CC(F)(F)F)NC(=O)c1ccc(/C=C/C(c2cc(Cl)c(F)c(Cl)c2)C(F)(F)F)cc1Br]

Isolated as a pale-yellow solid (0.105 g, 61%).

### 2-Chloro-4-((E)-4,4,4-trifluoro-3-(3,4,5-trichlorophenyl)but-1-en-1-yl)-N-((2R)-1-((2,2,2-trifluoroethyl)sulfinyl)propan-2-yl)benzamide (F50)

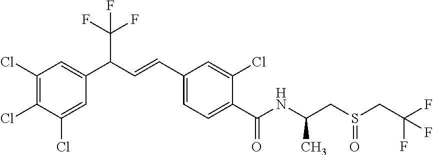 [SMILES: C[C@H](CS(=O)CC(F)(F)F)NC(=O)c1ccc(/C=C/C(c2cc(Cl)c(Cl)c(Cl)c2)C(F)(F)F)cc1Cl]

Isolated as an off-white solid (0.14 g, 86%).

### 2-Ethyl-4-((E)-4,4,4-trifluoro-3-(3,4,5-trichlorophenyl)but-1-en-1-yl)-N-((2R)-1-((2,2,2-trifluoroethyl)sulfinyl)propan-2-yl)benzamide (F55)

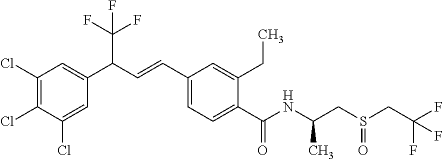 [SMILES: CCc1cc(/C=C/C(c2cc(Cl)c(Cl)c(Cl)c2)C(F)(F)F)ccc1C(=O)N[C@H](C)CS(=O)CC(F)(F)F]

Isolated as an off-white solid (0.07 g, 59%).

### 4-((E)-3-(3,5-Dibromophenyl)-4,4,4-trifluorobut-1-en-1-yl)-N-((2R)-1-((2,2,2-trifluoroethyl)sulfinyl)propan-2-yl)-2-(trifluoromethyl)benzamide (F60)

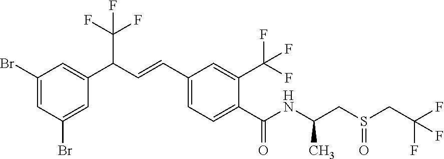 [SMILES: C[C@H](CS(=O)CC(F)(F)F)NC(=O)c1ccc(/C=C/C(c2cc(Br)cc(Br)c2)C(F)(F)F)cc1C(F)(F)F]

Isolated as an off-white solid (0.130 g, 76%).

### (E)-4-(4,4,4-Trifluoro-3-(3,4,5-trichlorophenyl)but-1-en-1-yl)-N-(1-(((2,2,2-trifluoroethyl)sulfinyl)methyl)cyclopropyl)-2-(trifluoromethyl)benzamide (F63)

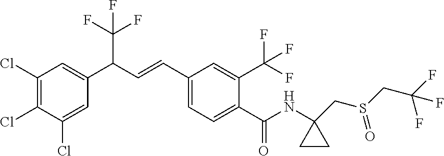 [SMILES: O=C(NC1(CS(=O)CC(F)(F)F)CC1)c1ccc(/C=C/C(c2cc(Cl)c(Cl)c(Cl)c2)C(F)(F)F)cc1C(F)(F)F]

Isolated as an off-white solid (0.11 g, 66%).

### N-((2R)-1-(Ethylsulfinyl)propan-2-yl)-4-((E)-4,4,4-trifluoro-3-(3,4,5-trichlorophenyl)but-1-en-1-yl)-2-(trifluoromethyl)benzamide (F69)

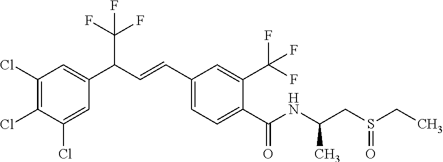 [SMILES: CCS(=O)C[C@@H](C)NC(=O)c1ccc(/C=C/C(c2cc(Cl)c(Cl)c(Cl)c2)C(F)(F)F)cc1C(F)(F)F]

Isolated as a pale-yellow solid (0.12 g, 69%).

### 2-Chloro-4-((E)-3-(3,5-dibromophenyl)-4,4,4-trifluorobut-1-en-1-yl)-N-((2R)-1-((2,2,2-trifluoroethyl)sulfinyl)propan-2-yl)benzamide (F72)

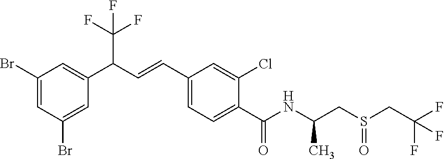 [SMILES: C[C@H](CS(=O)CC(F)(F)F)NC(=O)c1ccc(/C=C/C(c2cc(Br)cc(Br)c2)C(F)(F)F)cc1Cl]

Isolated as an off-white solid (0.12 g, 72%).

### 4-((E)-3-(3,5-Dibromophenyl)-4,4,4-trifluorobut-1-en-1-yl)-2-methyl-N-((2R)-1-((2,2,2-trifluoroethyl)sulfinyl)propan-2-yl)benzamide (F73)

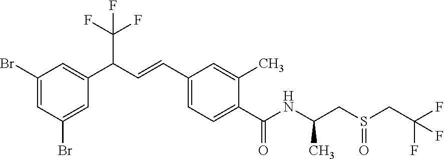 [SMILES: Cc1cc(/C=C/C(c2cc(Br)cc(Br)c2)C(F)(F)F)ccc1C(=O)N[C@H](C)CS(=O)CC(F)(F)F]

Isolated as an off-white solid (0.16 g, 81%).

### N-((2R)-1-(Isopropylsulfinyl)propan-2-yl)-4-((E)-4,4,4-trifluoro-3-(3,4,5-trichlorophenyl)but-1-en-1-yl)-2-(trifluoromethyl)benzamide (F82)

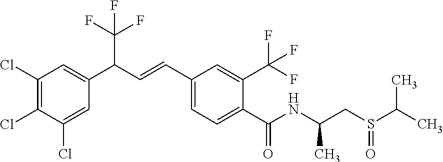 [SMILES: CC(C)S(=O)C[C@@H](C)NC(=O)c1ccc(/C=C/C(c2cc(Cl)c(Cl)c(Cl)c2)C(F)(F)F)cc1C(F)(F)F]

Isolated as a pale-yellow solid (0.14 g, 83%).

### N-((2R)-1-(Isobutylsulfinyl)propan-2-yl)-4-((E)-4,4,4-trifluoro-3-(3,4,5-trichlorophenyl)but-1-en-1-yl)-2-(trifluoromethyl)benzamide (F87)

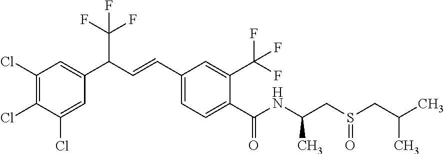 [SMILES: CC(C)CS(=O)C[C@@H](C)NC(=O)c1ccc(/C=C/C(c2cc(Cl)c(Cl)c(Cl)c2)C(F)(F)F)cc1C(F)(F)F]

Isolated as a pale-yellow solid (0.11 g, 65%).

### 4-((E)-3-(3,5-Dichlorophenyl)-4,4,4-trifluorobut-1-en-1-yl)-N-((2R)-1-((2,2,2-trifluoroethyl)sulfinyl)propan-2-yl)-2-(trifluoromethyl)benzamide (F90)

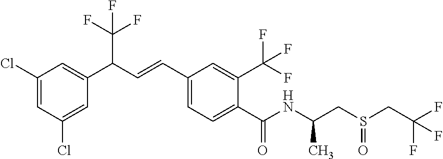 [SMILES: C[C@H](CS(=O)CC(F)(F)F)NC(=O)c1ccc(/C=C/C(c2cc(Cl)cc(Cl)c2)C(F)(F)F)cc1C(F)(F)F]

Isolated as a pale-yellow solid (0.10 g, 61%).

### 4-((E)-3-(3,5-Dichlorophenyl)-4,4,4-trifluorobut-1-en-1-yl)-2-methyl-N-((2R)-1-((2,2,2-trifluoroethyl)sulfinyl)propan-2-yl)benzamide (F91)

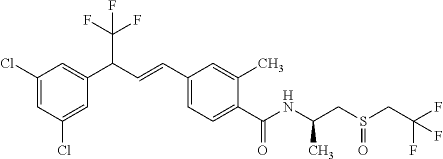 [SMILES: Cc1cc(/C=C/C(c2cc(Cl)cc(Cl)c2)C(F)(F)F)ccc1C(=O)N[C@H](C)CS(=O)CC(F)(F)F]

Isolated as an off-white solid (0.24 g, 78%).

### 2-Chloro-4-((E)-3-(3,5-dichlorophenyl)-4,4,4-trifluorobut-1-en-1-yl)-N-((2R)-1-((2,2,2-trifluoroethyl)sulfinyl)propan-2-yl)benzamide (F94)

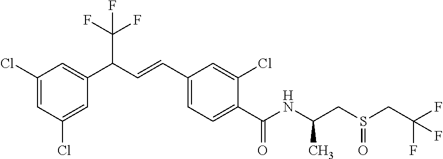 [SMILES: C[C@H](CS(=O)CC(F)(F)F)NC(=O)c1ccc(/C=C/C(c2cc(Cl)cc(Cl)c2)C(F)(F)F)cc1Cl]

Isolated as a pale-yellow solid (0.13 g, 79%).

### 2-Fluoro-6-methyl-4-((E)-4,4,4-trifluoro-3-(3,4,5-trichlorophenyl)but-1-en-1-yl)-N-((2R)-1-((2,2,2-trifluoroethyl)sulfinyl)propan-2-yl)benzamide (F99)

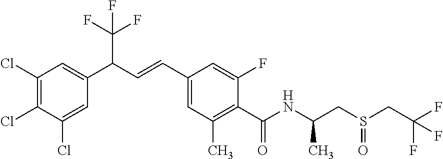 [SMILES: Cc1cc(/C=C/C(c2cc(Cl)c(Cl)c(Cl)c2)C(F)(F)F)cc(F)c1C(=O)N[C@H](C)CS(=O)CC(F)(F)F]

Isolated as a pale-yellow solid (0.18 g, 77%).

### N-((2R)-1-(Allylsulfinyl)propan-2-yl)-4-((E)-4,4,4-trifluoro-3-(3,4,5-trichlorophenyl)but-1-en-1-yl)-2-(trifluoromethyl)benzamide (F103)

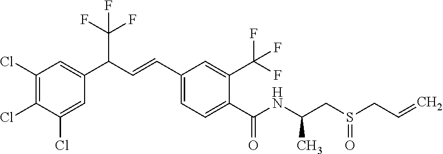 [SMILES: C=CCS(=O)C[C@@H](C)NC(=O)c1ccc(/C=C/C(c2cc(Cl)c(Cl)c(Cl)c2)C(F)(F)F)cc1C(F)(F)F]

Isolated as a pale-yellow sticky solid (0.18 g, 77%).

### 4-((E)-4,4,4-Trifluoro-3-(3,4,5-trichlorophenyl)but-1-en-1-yl)-N-((2R)-4-((2,2,2-trifluoroethyl)sulfinyl)butan-2-yl)-2-(trifluoromethyl)benzamide (F105)

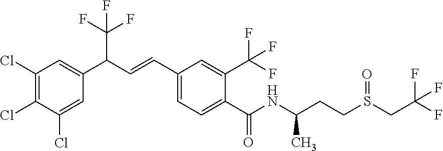 [SMILES: C[C@H](CCS(=O)CC(F)(F)F)NC(=O)c1ccc(/C=C/C(c2cc(Cl)c(Cl)c(Cl)c2)C(F)(F)F)cc1C(F)(F)F]

Isolated as a pale-yellow solid (0.10 g, 62%).

### (E)-4-(4,4,4-Trifluoro-3-(3,4,5-trichlorophenyl)but-1-en-1-yl)-N-((3-((2,2,2-trifluoroethyl)sulfinyl)oxetan-3-yl)methyl)-2-(trifluoromethyl)benzamide (F107)

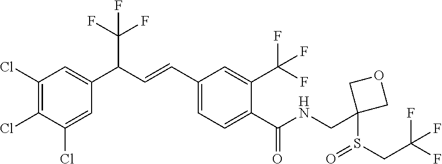 [SMILES: O=C(NCC1(S(=O)CC(F)(F)F)COC1)c1ccc(/C=C/C(c2cc(Cl)c(Cl)c(Cl)c2)C(F)(F)F)cc1C(F)(F)F]

Isolated as a brown semi-solid (0.02 g, 21%).

### N-((2R)-1-(Propylsulfinyl)propan-2-yl)-4-((E)-4,4,4-trifluoro-3-(3,4,5-trichlorophenyl)but-1-en-1-yl)-2-(trifluoromethyl)benzamide (F111)

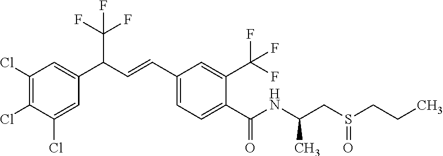 [SMILES: CCCS(=O)C[C@@H](C)NC(=O)c1ccc(/C=C/C(c2cc(Cl)c(Cl)c(Cl)c2)C(F)(F)F)cc1C(F)(F)F]

Isolated as an off-white solid (0.11 g, 65%).

### Example 3: Preparation of (E)-2-bromo-4-(4,4,4-trifluoro-3-(3,4,5-trichlorophenyl)but-1-en-1-yl)-N-(2-((2,2,2-trifluoroethyl)sulfinyl)ethyl)benzamide (F3)

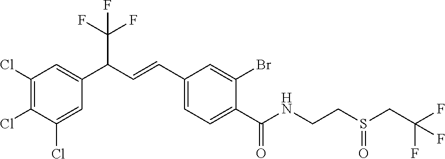 [SMILES: O=C(NCCS(=O)CC(F)(F)F)c1ccc(/C=C/C(c2cc(Cl)c(Cl)c(Cl)c2)C(F)(F)F)cc1Br]

To a 20 mL vial was added (E)-2-bromo-4-(4,4,4-trifluoro-3-(3,4,5-trichlorophenyl)but-1-en-1-yl)-N-(2-((2,2,2-trifluoroethyl)sulfinyl)ethyl)benzamide (F1) (0.169 g, 0.262 mmol) and acetic acid (1.5 mL). Sodium perborate (0.0443 g, 0.288 mmol) was added and the vial was capped and stirred at 50° C. for 3 hours. The reaction was diluted with ethyl acetate and saturated sodium bicarbonate and the biphasic system was stirred for 15 minutes. The layers were separated and the organic layer was dried over sodium sulfate, filtered, and concentrated. Purification by flash column chromatography using 30-100% ethyl acetate/hexanes provided the title compound as a colorless oil (0.296 g, 77%).

The following compounds were prepared according to the procedures disclosed in Example 3:

### (E)-2-Bromo-4-(4,4,4-trifluoro-3-(3,4,5-trichlorophenyl)but-1-en-1-yl)-N-(2-((2,2,2-trifluoroethyl)sulfonyl)ethyl)benzamide (F10)

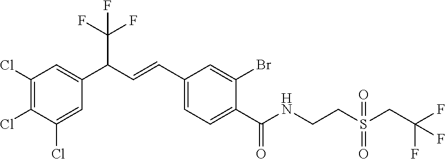 [SMILES: O=C(NCCS(=O)(=O)CC(F)(F)F)c1ccc(/C=C/C(c2cc(Cl)c(Cl)c(Cl)c2)C(F)(F)F)cc1Br]

Isolated as a colorless oil (0.075 g, 41%).

### N—((R)-3-Methyl-1-((2,2,2-trifluoroethyl)sulfonyl)butan-2-yl)-4-((E)-4,4,4-trifluoro-3-(3,4,5-trichlorophenyl)but-1-en-1-yl)-2-(trifluoromethyl)benzamide (F16)

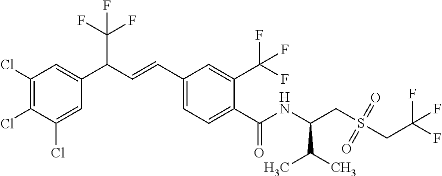 [SMILES: CC(C)[C@H](CS(=O)(=O)CC(F)(F)F)NC(=O)c1ccc(/C=C/C(c2cc(Cl)c(Cl)c(Cl)c2)C(F)(F)F)cc1C(F)(F)F]

Isolated as a white solid using 3 equivalents of sodium perborate (0.114 g, 54%).

### 4-((E)-4,4,4-Trifluoro-3-(3,4,5-trichlorophenyl)but-1-en-1-yl)-N—((R)-1-((2,2,2-trifluoroethyl)sulfonyl)butan-2-yl)-2-(trifluoromethyl)benzamide (F17)

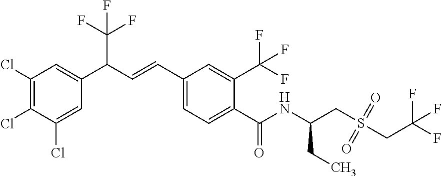 [SMILES: CC[C@H](CS(=O)(=O)CC(F)(F)F)NC(=O)c1ccc(/C=C/C(c2cc(Cl)c(Cl)c(Cl)c2)C(F)(F)F)cc1C(F)(F)F]

Isolated as a white-yellow solid using 1.47 equivalents of sodium perborate (0.22 g, 65%).

### 4-((E)-4,4,4-Trifluoro-3-(3,4,5-trichlorophenyl)but-1-en-1-yl)-N-((2R)-1-((2,2,2-trifluoroethyl)sulfinyl)butan-2-yl)-2-(trifluoromethyl)benzamide (F18)

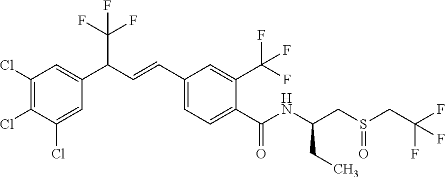 [SMILES: CC[C@H](CS(=O)CC(F)(F)F)NC(=O)c1ccc(/C=C/C(c2cc(Cl)c(Cl)c(Cl)c2)C(F)(F)F)cc1C(F)(F)F]

Isolated as a colorless oil using 1.47 equivalents of sodium perborate (0.43 g, 12%).

### 2-Bromo-N-((2R)-1-(((2,2-difluorocyclopropyl)methyl)sulfonyl)butan-2-yl)-4-((E)-4,4,4-trifluoro-3-(3,4,5-trichlorophenyl)but-1-en-1-yl)benzamide (F19)

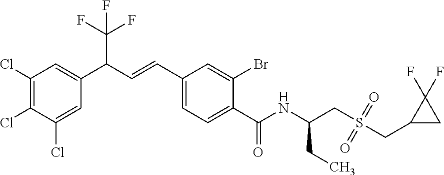 [SMILES: CC[C@H](CS(=O)(=O)CC1CC1(F)F)NC(=O)c1ccc(/C=C/C(c2cc(Cl)c(Cl)c(Cl)c2)C(F)(F)F)cc1Br]

Isolated as a yellow oil using 1.5 equivalents of sodium perborate (0.104 g, 24%).

### 2-Bromo-N-((2R)-1-(((2,2-difluorocyclopropyl)methyl)sulfinyl)butan-2-yl)-4-((E)-4,4,4-trifluoro-3-(3,4,5-trichlorophenyl)but-1-en-1-yl)benzamide (F20)

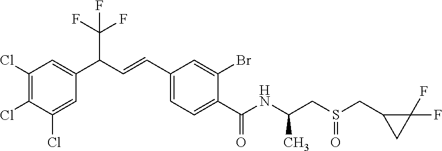 [SMILES: C[C@H](CS(=O)CC1CC1(F)F)NC(=O)c1ccc(/C=C/C(c2cc(Cl)c(Cl)c(Cl)c2)C(F)(F)F)cc1Br]

Isolated as a white solid using 1.5 equivalents of sodium perborate (0.228 g, 54%).

### 4-((E)-4,4,4-Trifluoro-3-(3,4,5-trichlorophenyl)but-1-en-1-yl)-2-(trifluoromethyl)-N—((R)-1-((3,3,3-trifluoropropyl)sulfonyl)butan-2-yl)benzamide (F21)

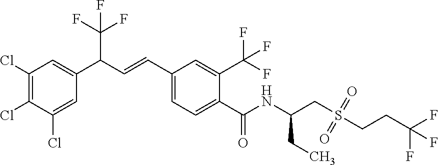 [SMILES: CC[C@H](CS(=O)(=O)CCC(F)(F)F)NC(=O)c1ccc(/C=C/C(c2cc(Cl)c(Cl)c(Cl)c2)C(F)(F)F)cc1C(F)(F)F]

Isolated as an iridescent foam using 1.7 equivalents of sodium perborate (0.107 g, 63%).

### 4-((E)-4,4,4-Trifluoro-3-(3,4,5-trichlorophenyl)but-1-en-1-yl)-N—((R)-1-((2,2,2-trifluoroethyl)sulfonyl)propan-2-yl)-2-(trifluoromethyl)benzamide (F24)

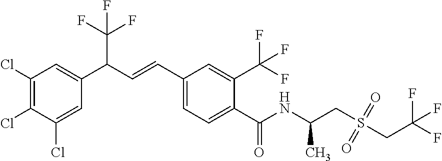 [SMILES: C[C@H](CS(=O)(=O)CC(F)(F)F)NC(=O)c1ccc(/C=C/C(c2cc(Cl)c(Cl)c(Cl)c2)C(F)(F)F)cc1C(F)(F)F]

Isolated as a white solid using 3 equivalents of sodium perborate (0.055 g, 35%).

### 4-((E)-3-(3,5-Dichloro-4-fluorophenyl)-4,4,4-trifluorobut-1-en-1-yl)-N—((R)-1-((2,2,2-trifluoroethyl)sulfonyl)propan-2-yl)-2-(trifluoromethyl)benzamide (F36)

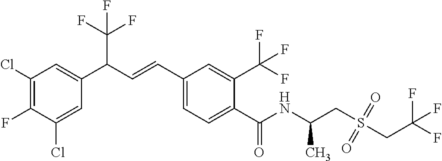 [SMILES: C[C@H](CS(=O)(=O)CC(F)(F)F)NC(=O)c1ccc(/C=C/C(c2cc(Cl)c(F)c(Cl)c2)C(F)(F)F)cc1C(F)(F)F]

Isolated as an off-white solid (0.12 g, 68%).

### 2-Methyl-4-((E)-4,4,4-trifluoro-3-(3,4,5-trichlorophenyl)but-1-en-1-yl)-N—((R)-1-((2,2,2-trifluoroethyl)sulfonyl)propan-2-yl)benzamide (F40)

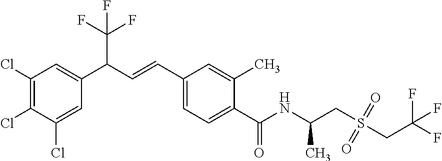 [SMILES: Cc1cc(/C=C/C(c2cc(Cl)c(Cl)c(Cl)c2)C(F)(F)F)ccc1C(=O)N[C@H](C)CS(=O)(=O)CC(F)(F)F]

Isolated as an off-white solid using 2 equivalents of sodium perborate (0.11 g, 91%).

### 2-(Difluoromethyl)-4-((E)-4,4,4-trifluoro-3-(3,4,5-trichlorophenyl)but-1-en-1-yl)-N—((R)-1-((2,2,2-trifluoroethyl)sulfonyl)propan-2-yl)benzamide (F43)

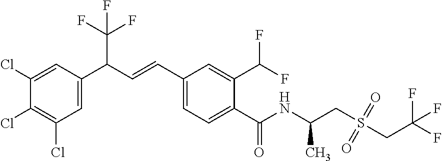 [SMILES: C[C@H](CS(=O)(=O)CC(F)(F)F)NC(=O)c1ccc(/C=C/C(c2cc(Cl)c(Cl)c(Cl)c2)C(F)(F)F)cc1C(F)F]

Isolated as a white gum using 1.8 equivalents of sodium perborate (0.108 g, 89%).

### (E)-N-(2-Methyl-1-((2,2,2-trifluoroethyl)sulfinyl)propan-2-yl)-4-(4,4,4-trifluoro-3-(3,4,5-trichlorophenyl)but-1-en-1-yl)-2-(trifluoromethyl)benzamide (F47)

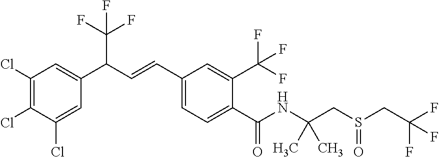 [SMILES: CC(C)(CS(=O)CC(F)(F)F)NC(=O)c1ccc(/C=C/C(c2cc(Cl)c(Cl)c(Cl)c2)C(F)(F)F)cc1C(F)(F)F]

Isolated as a white foam using 1.5 equivalents of sodium perborate (0.201 g, 87%).

### (E)-N-(2-Methyl-1-((2,2,2-trifluoroethyl)sulfonyl)propan-2-yl)-4-(4,4,4-trifluoro-3-(3,4,5-trichlorophenyl)but-1-en-1-yl)-2-(trifluoromethyl)benzamide (F48)

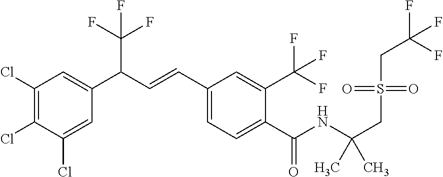 [SMILES: CC(C)(CS(=O)(=O)CC(F)(F)F)NC(=O)c1ccc(/C=C/C(c2cc(Cl)c(Cl)c(Cl)c2)C(F)(F)F)cc1C(F)(F)F]

Isolated as a yellow foam using 1.5 equivalents of sodium perborate (0.312 g, 65%).

### 2-Bromo-4-((E)-3-(3,5-dichloro-4-fluorophenyl)-4,4,4-trifluorobut-1-en-1-yl)-N—((R)-1-((2,2,2-trifluoroethyl)sulfonyl)propan-2-yl)benzamide (F52)

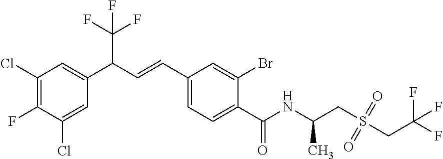 [SMILES: C[C@H](CS(=O)(=O)CC(F)(F)F)NC(=O)c1ccc(/C=C/C(c2cc(Cl)c(F)c(Cl)c2)C(F)(F)F)cc1Br]

Isolated as an off-white solid using 3 equivalents of sodium perborate (0.115 g, 68%).

### 4-((E)-3-(3,5-Dichloro-4-cyanophenyl)-4,4,4-trifluorobut-1-en-1-yl)-N-((2R)-1-((2,2,2-trifluoroethyl)sulfinyl)propan-2-yl)-2-(trifluoromethyl)benzamide (F53)

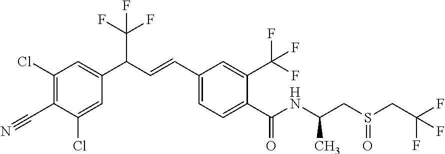 [SMILES: C[C@H](CS(=O)CC(F)(F)F)NC(=O)c1ccc(/C=C/C(c2cc(Cl)c(C#N)c(Cl)c2)C(F)(F)F)cc1C(F)(F)F]

Isolated as a pale-brown solid using 1.7 equivalents of sodium perborate (0.091 g, 45%).

### 2-Chloro-4-((E)-4,4,4-trifluoro-3-(3,4,5-trichlorophenyl)but-1-en-1-yl)-N—((R)-1-((2,2,2-trifluoroethyl)sulfonyl)propan-2-yl)benzamide (F58)

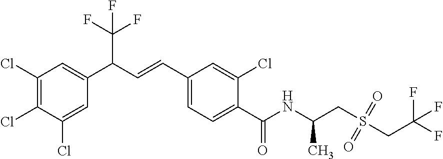 [SMILES: C[C@H](CS(=O)(=O)CC(F)(F)F)NC(=O)c1ccc(/C=C/C(c2cc(Cl)c(Cl)c(Cl)c2)C(F)(F)F)cc1Cl]

Isolated as an off-white solid using 3 equivalents of sodium perborate (0.14 g, 77%).

### 4-((E)-3-(3,5-Dibromo-4-chlorophenyl)-4,4,4-trifluorobut-1-en-1-yl)-N—((R)-1-((2,2,2-trifluoroethyl)sulfonyl)propan-2-yl)-2-(trifluoromethyl)benzamide (F59)

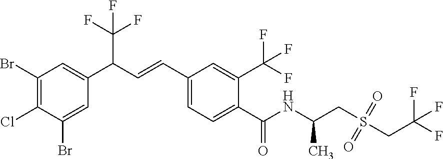 [SMILES: C[C@H](CS(=O)(=O)CC(F)(F)F)NC(=O)c1ccc(/C=C/C(c2cc(Br)c(Cl)c(Br)c2)C(F)(F)F)cc1C(F)(F)F]

Isolated as a brown solid using 3 equivalents of sodium perborate (0.115 g, 78%).

### 4-((E)-3-(3,5-Dibromophenyl)-4,4,4-trifluorobut-1-en-1-yl)-N—((R)-1-((2,2,2-trifluoroethyl)sulfonyl)propan-2-yl)-2-(trifluoromethyl)benzamide (F61)

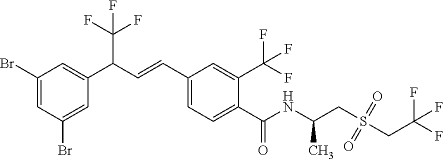 [SMILES: C[C@H](CS(=O)(=O)CC(F)(F)F)NC(=O)c1ccc(/C=C/C(c2cc(Br)cc(Br)c2)C(F)(F)F)cc1C(F)(F)F]

Isolated as a pale-yellow solid using 3 equivalents of sodium perborate (0.165 g, 67%).

### 2-Chloro-4-((E)-3-(3,5-dibromophenyl)-4,4,4-trifluorobut-1-en-1-yl)-N—((R)-1-((2,2,2-trifluoroethyl)sulfonyl)propan-2-yl)benzamide (F65)

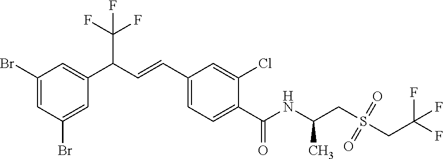 [SMILES: C[C@H](CS(=O)(=O)CC(F)(F)F)NC(=O)c1ccc(/C=C/C(c2cc(Br)cc(Br)c2)C(F)(F)F)cc1Cl]

Isolated as an off-white solid using 3 equivalents of sodium perborate (0.168 g, 80%).

### 4-((E)-3-(3,5-Dibromophenyl)-4,4,4-trifluorobut-1-en-1-yl)-2-methyl-N—((R)-1-((2,2,2-trifluoroethyl)sulfonyl)propan-2-yl)benzamide (F66)

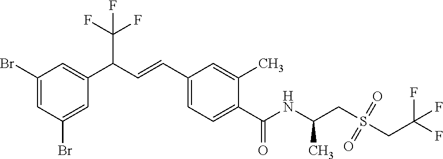 [SMILES: Cc1cc(/C=C/C(c2cc(Br)cc(Br)c2)C(F)(F)F)ccc1C(=O)N[C@H](C)CS(=O)(=O)CC(F)(F)F]

Isolated as a white solid using 3 equivalents of sodium perborate (0.19 g, 77%).

### N—((R)-1-(Ethylsulfonyl)propan-2-yl)-4-((E)-4,4,4-trifluoro-3-(3,4,5-trichlorophenyl)but-1-en-1-yl)-2-(trifluoromethyl)benzamide (F68)

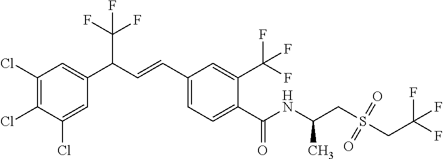 [SMILES: C[C@H](CS(=O)(=O)CC(F)(F)F)NC(=O)c1ccc(/C=C/C(c2cc(Cl)c(Cl)c(Cl)c2)C(F)(F)F)cc1C(F)(F)F]

Isolated as a pale-yellow solid using 3 equivalents of sodium perborate (0.110 g, 60%).

### 2-Ethyl-4-((E)-4,4,4-trifluoro-3-(3,4,5-trichlorophenyl)but-1-en-1-yl)-N—((R)-1-((2,2,2-trifluoroethyl)sulfonyl)propan-2-yl)benzamide (F71)

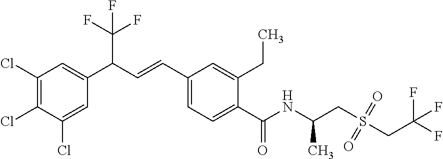 [SMILES: CCc1cc(/C=C/C(c2cc(Cl)c(Cl)c(Cl)c2)C(F)(F)F)ccc1C(=O)N[C@H](C)CS(=O)(=O)CC(F)(F)F]

Isolated as an off-white solid using 3.5 equivalents of sodium perborate (0.070 g, 64%).

### 4-((E)-3-(7-Chlorobenzo[d][1,3]dioxol-5-yl)-4,4,4-trifluorobut-1-en-1-yl)-N—((R)-1-((2,2,2-trifluoroethyl)sulfonyl)propan-2-yl)-2-(trifluoromethyl)benzamide (F76)

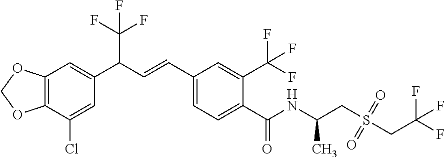 [SMILES: C[C@H](CS(=O)(=O)CC(F)(F)F)NC(=O)c1ccc(/C=C/C(c2cc(Cl)c3c(c2)OCO3)C(F)(F)F)cc1C(F)(F)F]

Isolated as an off-white solid using 3 equivalents of sodium perborate (0.08 g, 65%).

### 4-((E)-4,4,4-Trifluoro-3-(3,4,5-trichlorophenyl)but-1-en-1-yl)-N-((2R)-1-((2,2,2-trifluoroethyl)sulfinyl)propan-2-yl)benzamide (FC77)

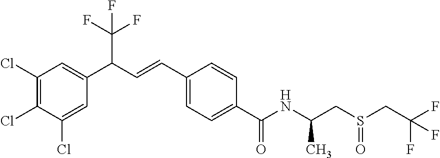 [SMILES: C[C@H](CS(=O)CC(F)(F)F)NC(=O)c1ccc(/C=C/C(c2cc(Cl)c(Cl)c(Cl)c2)C(F)(F)F)cc1]

Isolated as a colorless foam/glass using 1.5 equivalents of sodium perborate (0.055 g, 67%).

### 4-((E)-4,4,4-Trifluoro-3-(3,4,5-trichlorophenyl)but-1-en-1-yl)-N—((R)-1-((2,2,2-trifluoroethyl)sulfonyl)propan-2-yl)benzamide (FC78)

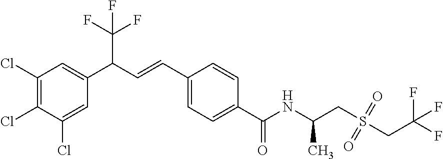 [SMILES: C[C@H](CS(=O)(=O)CC(F)(F)F)NC(=O)c1ccc(/C=C/C(c2cc(Cl)c(Cl)c(Cl)c2)C(F)(F)F)cc1]

Isolated as a colorless foam/glass using 1.5 equivalents of sodium perborate (0.058 g, 69%).

### N—((R)-1-(Propylsulfonyl)propan-2-yl)-4-((E)-4,4,4-trifluoro-3-(3,4,5-trichlorophenyl)but-1-en-1-yl)-2-(trifluoromethyl)benzamide (F79)

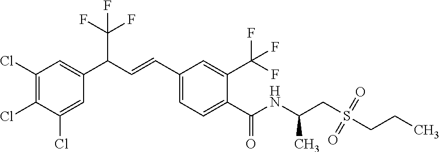 [SMILES: CCCS(=O)(=O)C[C@@H](C)NC(=O)c1ccc(/C=C/C(c2cc(Cl)c(Cl)c(Cl)c2)C(F)(F)F)cc1C(F)(F)F]

Isolated as a pale-yellow solid using 3 equivalents of sodium perborate (0.14 g, 85%).

### N—((R)-1-(Isopropylsulfonyl)propan-2-yl)-4-((E)-4,4,4-trifluoro-3-(3,4,5-trichlorophenyl)but-1-en-1-yl)-2-(trifluoromethyl)benzamide (F80)

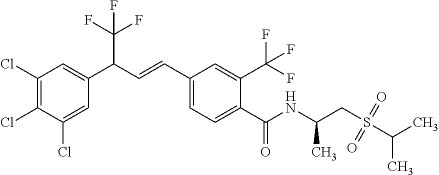 [SMILES: CC(C)S(=O)(=O)C[C@@H](C)NC(=O)c1ccc(/C=C/C(c2cc(Cl)c(Cl)c(Cl)c2)C(F)(F)F)cc1C(F)(F)F]

Isolated as a pale-yellow solid using 3 equivalents of sodium perborate (0.110 g, 62%).

### N—((R)-1-(Isobutylsulfonyl)propan-2-yl)-4-((E)-4,4,4-trifluoro-3-(3,4,5-trichlorophenyl)but-1-en-1-yl)-2-(trifluoromethyl)benzamide (F81)

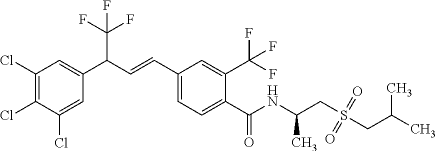 [SMILES: CC(C)CS(=O)(=O)C[C@@H](C)NC(=O)c1ccc(/C=C/C(c2cc(Cl)c(Cl)c(Cl)c2)C(F)(F)F)cc1C(F)(F)F]

Isolated as an off-white solid using 3 equivalents of sodium perborate (0.110 g, 59%).

### 4-((E)-4,4-Difluoro-3-(3,4,5-trichlorophenyl)pent-1-en-1-yl)-N—((R)-1-((2,2,2-trifluoroethyl)sulfonyl)propan-2-yl)-2-(trifluoromethyl)benzamide (F84)

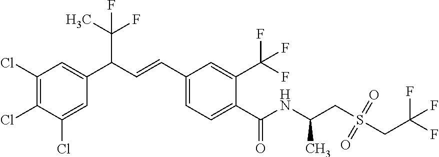 [SMILES: C[C@H](CS(=O)(=O)CC(F)(F)F)NC(=O)c1ccc(/C=C/C(c2cc(Cl)c(Cl)c(Cl)c2)C(C)(F)F)cc1C(F)(F)F]

Isolated using 1.5 equivalents of sodium perborate (0.03 g, 48%).

### 4-((E)-3-(3,5-Dichlorophenyl)-4,4,4-trifluorobut-1-en-1-yl)-2-methyl-N—((R)-1-((2,2,2-trifluoroethyl)sulfonyl)propan-2-yl)benzamide (F92)

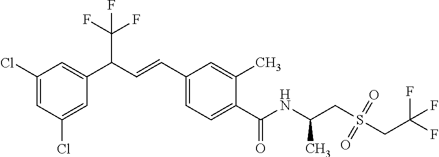 [SMILES: Cc1cc(/C=C/C(c2cc(Cl)cc(Cl)c2)C(F)(F)F)ccc1C(=O)N[C@H](C)CS(=O)(=O)CC(F)(F)F]

Isolated as an off-white solid using 3 equivalents of sodium perborate (0.15 g, 63%).

### 4-((E)-3-(3,5-Dichlorophenyl)-4,4,4-trifluorobut-1-en-1-yl)-N—((R)-1-((2,2,2-trifluoroethyl)sulfonyl)propan-2-yl)-2-(trifluoromethyl)benzamide (F93)

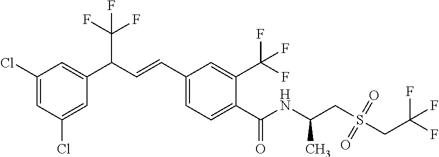 [SMILES: C[C@H](CS(=O)(=O)CC(F)(F)F)NC(=O)c1ccc(/C=C/C(c2cc(Cl)cc(Cl)c2)C(F)(F)F)cc1C(F)(F)F]

Isolated as a pale-yellow solid using 3 equivalents of sodium perborate (0.12 g, 71%).

### 2-Chloro-4-((E)-3-(3,5-dichlorophenyl)-4,4,4-trifluorobut-1-en-1-yl)-N—((R)-1-((2,2,2-trifluoroethyl)sulfonyl)propan-2-yl)benzamide (F95)

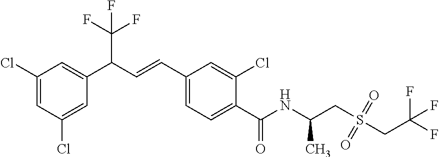 [SMILES: C[C@H](CS(=O)(=O)CC(F)(F)F)NC(=O)c1ccc(/C=C/C(c2cc(Cl)cc(Cl)c2)C(F)(F)F)cc1Cl]

Isolated as a pale-yellow solid using 3 equivalents of sodium perborate (0.11 g, 62%).

### 2-Fluoro-6-methyl-4-((E)-4,4,4-trifluoro-3-(3,4,5-trichlorophenyl)but-1-en-1-yl)-N—((R)-1-((2,2,2-trifluoroethyl)sulfonyl)propan-2-yl)benzamide (F98)

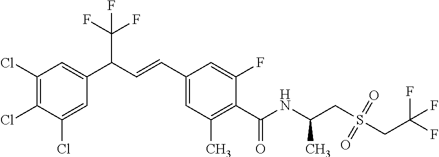 [SMILES: Cc1cc(/C=C/C(c2cc(Cl)c(Cl)c(Cl)c2)C(F)(F)F)cc(F)c1C(=O)N[C@H](C)CS(=O)(=O)CC(F)(F)F]

Isolated as a pale-yellow solid using 3 equivalents of sodium perborate (0.14 g, 60%).

### N—((R)-1-(Allylsulfonyl)propan-2-yl)-4-((E)-4,4,4-trifluoro-3-(3,4,5-trichlorophenyl)but-1-en-1-yl)-2-(trifluoromethyl)benzamide (F102)

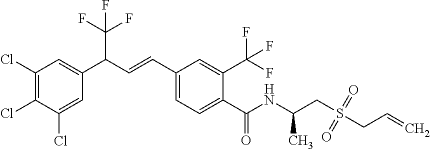 [SMILES: C=CCS(=O)(=O)C[C@@H](C)NC(=O)c1ccc(/C=C/C(c2cc(Cl)c(Cl)c(Cl)c2)C(F)(F)F)cc1C(F)(F)F]

Isolated as a pale-yellow gum using 3 equivalents of sodium perborate (0.21 g, 85%).

### 4-((E)-4,4,4-Trifluoro-3-(3,4,5-trichlorophenyl)but-1-en-1-yl)-N—((R)-4-((2,2,2-trifluoroethyl)sulfonyl)butan-2-yl)-2-(trifluoromethyl)benzamide (F106)

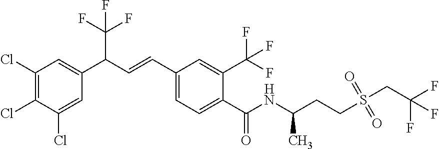 [SMILES: C[C@H](CCS(=O)(=O)CC(F)(F)F)NC(=O)c1ccc(/C=C/C(c2cc(Cl)c(Cl)c(Cl)c2)C(F)(F)F)cc1C(F)(F)F]

Isolated as a pale-yellow solid using 3 equivalents of sodium perborate (0.18 g, 79%).

### (E)-4-(4,4,4-Trifluoro-3-(3,4,5-trichlorophenyl)but-1-en-1-yl)-N-(1-(((2,2,2-trifluoroethyl)sulfonyl)methyl)cyclopropyl)-2-(trifluoromethyl)benzamide (F112)

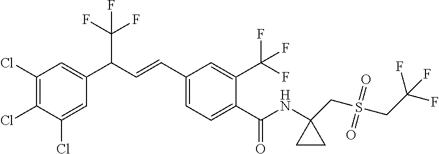 [SMILES: O=C(NC1(CS(=O)(=O)CC(F)(F)F)CC1)c1ccc(/C=C/C(c2cc(Cl)c(Cl)c(Cl)c2)C(F)(F)F)cc1C(F)(F)F]

Isolated as a yellow solid using 3 equivalents of sodium perborate (0.13 g, 67%).

### Example 4: Preparation of 4-((E)-4,4,4-trifluoro-3-(3,4,5-trichlorophenyl)but-1-en-1-yl)-2-(trifluoromethyl)-N—((R)-1-((3,3,3-trifluoropropyl)thio)butan-2-yl)benzamide (F1)

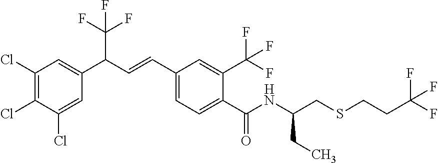 [SMILES: CC[C@H](CSCCC(F)(F)F)NC(=O)c1ccc(/C=C/C(c2cc(Cl)c(Cl)c(Cl)c2)C(F)(F)F)cc1C(F)(F)F]

To a 20 mL vial was added (R)-tert-butyl (1-((3,3,3-trifluoropropyl)thio)butan-2-yl)carbamate (C7) (0.148 g, 0.491 mmol) (as a 2:1 mixture with (R)-tert-butyl (1-mercaptobutan-2-yl)carbamate (0.0560 g, 0.273 mmol)). Hydrogen chloride (3.00 mL, 12.0 mmol, 4 M in dioxane) was added and the reaction was left to stir for 2.5 hours at room temperature. The reaction mixture was concentrated and dried in a 40° C. vacuum oven for 1 hour. To the residue was added (E)-4-(4,4,4-trifluoro-3-(3,4,5-trichlorophenyl)but-1-en-1-yl)-2-(trifluoromethyl)benzoyl chloride (0.379 g, 0.764 mmol) in dichloromethane (3 mL) to give a brown solution. 4-Methylmorpholine (0.400 mL, 3.64 mmol) was then added to the reaction mixture causing an exotherm and a precipitate formation. The resulting brown mixture was capped and stirred overnight. The suspension was diluted with water and dichloromethane. The layers were separated and the aqueous layer was extracted with dichloromethane (2×). The organic layers were combined, dried over magnesium sulfate, filtered and concentrated. Purification by flash column chromatography using 0-30% ethyl acetate/hexanes provided the title compound as a yellow oil (0.216 g, 63%).

The following compounds were prepared according to the procedures disclosed in Example 4:

### 4-((E)-4,4,4-Trifluoro-3-(3,4,5-trichlorophenyl)but-1-en-1-yl)-N—((R)-1-((2,2,2-trifluoroethyl)thio)butan-2-yl)-2-(trifluoromethyl)benzamide (F12)

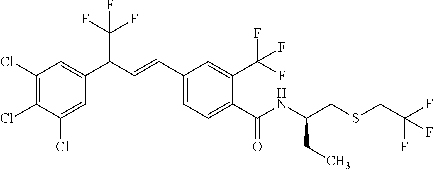 [SMILES: CC[C@H](CSCC(F)(F)F)NC(=O)c1ccc(/C=C/C(c2cc(Cl)c(Cl)c(Cl)c2)C(F)(F)F)cc1C(F)(F)F]

Isolated as an orange oil (0.378 g, 69%).

### N-((2R)-1-(((2,2-Difluorocyclopropyl)methyl)thio)butan-2-yl)-4-((E)-4,4,4-trifluoro-3-(3,4,5-trichlorophenyl)but-1-en-1-yl)-2-(trifluoromethyl)benzamide (F13)

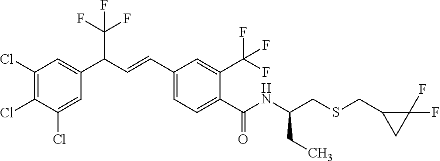 [SMILES: CC[C@H](CSCC1CC1(F)F)NC(=O)c1ccc(/C=C/C(c2cc(Cl)c(Cl)c(Cl)c2)C(F)(F)F)cc1C(F)(F)F]

Isolated as an orange oil (0.444 g, 70%).

### N—((R)-3-Methyl-1-((2,2,2-trifluoroethyl)thio)butan-2-yl)-4-((E)-4,4,4-trifluoro-3-(3,4,5-trichlorophenyl)but-1-en-1-yl)-2-(trifluoromethyl)benzamide (F14)

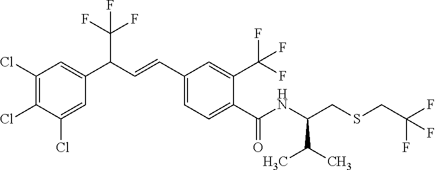 [SMILES: CC(C)[C@H](CSCC(F)(F)F)NC(=O)c1ccc(/C=C/C(c2cc(Cl)c(Cl)c(Cl)c2)C(F)(F)F)cc1C(F)(F)F]

Isolated as an orange solid (0.68 g, 57%).

### Example 5: Preparation of 4-((E)-4,4,4-trifluoro-3-(3,4,5-trichlorophenyl)but-1-en-1-yl)-N—((R)-1-((2,2,2-trifluoroethyl)thio)propan-2-yl)-2-(trifluoromethyl)benzamide (F22)

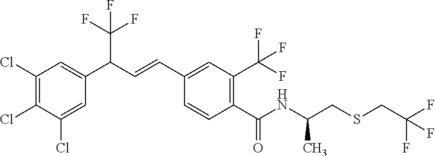 [SMILES: C[C@H](CSCC(F)(F)F)NC(=O)c1ccc(/C=C/C(c2cc(Cl)c(Cl)c(Cl)c2)C(F)(F)F)cc1C(F)(F)F]

A 20 mL vial was charged with (1-cyano-2-ethoxy-2-oxoethylidenaminooxy)dimethylamino-morpholino-carbenium hexafluorophosphate (0.986 g, 2.30 mmol), (E)-4-(4,4,4-trifluoro-3-(3,4,5-trichlorophenyl)but-1-en-1-yl)-2-(trifluoromethyl)benzoic acid (1.00 g, 2.09 mmol), (R)-1-((2,2,2-trifluoroethyl)thio)propan-2-amine hydrochloride (0.483 g, 2.30 mmol), and 1, 2-dichloroethane (7 mL). To the red suspension was added 4-methylmorpholine (0.691 mL, 6.28 mmol) and the reaction was left to stir overnight. The reaction mixture was diluted with ethyl acetate and hydrochloric acid (1 M). The layers were separated and the organic layer was washed with aqueous sodium bicarbonate and brine. The resulting organic layer was dried over magnesium sulfate, filtered, and concentrated. Purification by flash column chromatography using 0-30% ethyl acetate/hexanes provided the title compound as an orange glass (0.900 g, 65%).

The following compounds were prepared according to the procedures disclosed in Example 5:

### (E)-N-(2,2-Bis(ethylthio)ethyl)-4-(4,4,4-trifluoro-3-(3,4,5-trichlorophenyl)but-1-en-1-yl)-2-(trifluoromethyl)benzamide (F113)

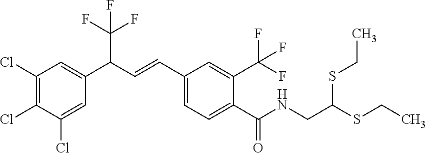 [SMILES: CCSC(CNC(=O)c1ccc(/C=C/C(c2cc(Cl)c(Cl)c(Cl)c2)C(F)(F)F)cc1C(F)(F)F)SCC]

Isolated as a brown oil (0.05 g, 9%).

### Example 6: Preparation of 2-bromo-N—((R)-4-(methylsulfonyl)-1-oxo-1-((2,2,2-trifluoroethyl)amino)butan-2-yl)-4-((E)-4,4,4-trifluoro-3-(3,4,5-trichlorophenyl)but-1-en-1-yl)benzamide (F25)

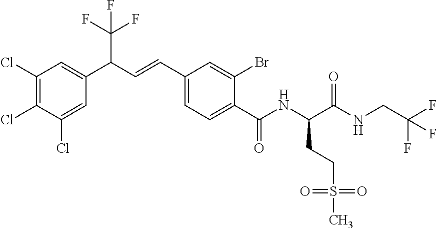 [SMILES: CS(=O)(=O)CC[C@@H](NC(=O)c1ccc(/C=C/C(c2cc(Cl)c(Cl)c(Cl)c2)C(F)(F)F)cc1Br)C(=O)NCC(F)(F)F]

To 2-bromo-4-[(E)-4,4,4-trifluoro-3-(3,4,5-trichlorophenyl)but-1-enyl]benzoic acid (0.200 g, 0.410 mmol), (2R)-2-amino-4-methylsulfonyl-N-(2,2,2-trifluoroethyl)butanamide hydrochloride (C15) (0.135 g, 0.450 mmol) in dichloromethane (3 mL) were added diisopropylethylamine (0.0700 mL, 0.499 mol) and benzotriazol-1-yl-oxytripyrrolidinophosphonium hexafluorophosphate (0.213 g, 0.410 mmol). The reaction mixture was stirred at room temperature for 12 hours. The reaction was poured into ice water and extracted with ethyl acetate, dried over sodium sulfate, filtered, and concentrated. Purification by flash column chromatography using 20% ethyl acetate/petroleum ether provided the title compound as an off-white solid (0.10 g, 33%).

The following compounds were prepared according to the procedures disclosed in Example 6:

### N—((R)-4-(Methylsulfonyl)-1-oxo-1-((2,2,2-trifluoroethyl)amino)butan-2-yl)-4-((E)-4,4,4-trifluoro-3-(3,4,5-trichlorophenyl)but-1-en-1-yl)-2-(trifluoromethyl)benzamide (F26)

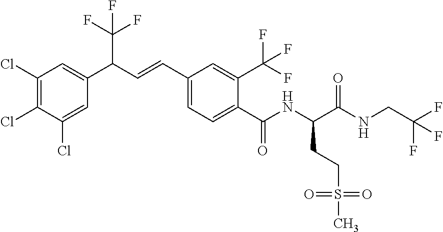 [SMILES: CS(=O)(=O)CC[C@@H](NC(=O)c1ccc(/C=C/C(c2cc(Cl)c(Cl)c(Cl)c2)C(F)(F)F)cc1C(F)(F)F)C(=O)NCC(F)(F)F]

Isolated as an off-white solid (0.14 g, 46%).

### N—((R)-4-(Methylsulfonyl)-1-oxo-1-((2,2,2-trifluoroethyl)amino)butan-2-yl)-4-((E)-4,4,4-trifluoro-3-(3,4,5-trichlorophenyl)but-1-en-1-yl)-2-(trifluoromethyl)benzamide (F27)

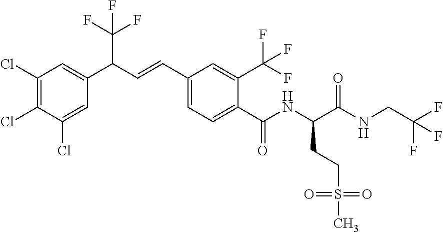 [SMILES: CS(=O)(=O)CC[C@@H](NC(=O)c1ccc(/C=C/C(c2cc(Cl)c(Cl)c(Cl)c2)C(F)(F)F)cc1C(F)(F)F)C(=O)NCC(F)(F)F]

Isolated as a brown solid (0.14 g, 55%).

### N—((R)-4-(Methylthio)-1-oxo-1-((2,2,2-trifluoroethyl)amino)butan-2-yl)-4-((E)-4,4,4-trifluoro-3-(3,4,5-trichlorophenyl)but-1-en-1-yl)-2-(trifluoromethyl)benzamide (F28)

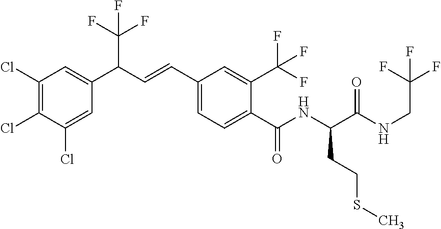 [SMILES: CSCC[C@@H](NC(=O)c1ccc(/C=C/C(c2cc(Cl)c(Cl)c(Cl)c2)C(F)(F)F)cc1C(F)(F)F)C(=O)NCC(F)(F)F]

Isolated as a brown solid (0.12 g, 52%/).

### 2-Bromo-4-((E)-3-(3,5-dichloro-4-fluorophenyl)-4,4,4-trifluorobut-1-en-1-yl)-N—((R)-1-((2,2,2-trifluoroethyl)thio)propan-2-yl)benzamide (F34)

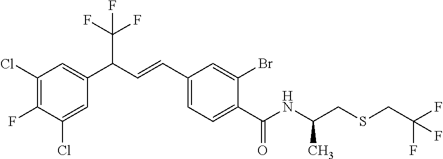 [SMILES: C[C@H](CSCC(F)(F)F)NC(=O)c1ccc(/C=C/C(c2cc(Cl)c(F)c(Cl)c2)C(F)(F)F)cc1Br]

Isolated as a brown gum (0.40 g, 66%).

### 4-((E)-3-(3,5-Dichloro-4-fluorophenyl)-4,4,4-trifluorobut-1-en-1-yl)-N—((R)-1-((2,2,2-trifluoroethyl)thio)propan-2-yl)-2-(trifluoromethyl)benzamide (F35)

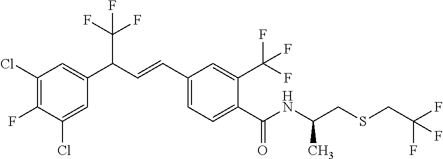 [SMILES: C[C@H](CSCC(F)(F)F)NC(=O)c1ccc(/C=C/C(c2cc(Cl)c(F)c(Cl)c2)C(F)(F)F)cc1C(F)(F)F]

Isolated as a brown gum (0.40 g, 69%). 2-Bromo-4-((E)-4,4,4-trifluoro-3-(3,4,5-trichlorophenyl)but-1-en-1-yl)-N-((R)-1-((2,2,2-trifluoroethyl) thio)propan-2-yl)benzamide (F38)

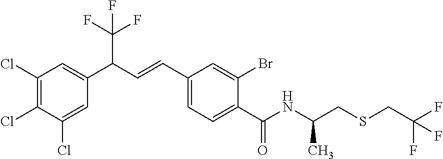 [SMILES: C[C@H](CSCC(F)(F)F)NC(=O)c1ccc(/C=C/C(c2cc(Cl)c(Cl)c(Cl)c2)C(F)(F)F)cc1Br]

Isolated as an off-white solid (0.40 g, 55%).

### 4-((E)-3-(3,5-Dibromo-4-chlorophenyl)-4,4,4-trifluorobut-1-en-1-yl)-N—((R)-1-((2,2,2-trifluoroethyl)thio)propan-2-yl)-2-(trifluoromethyl)benzamide (F41)

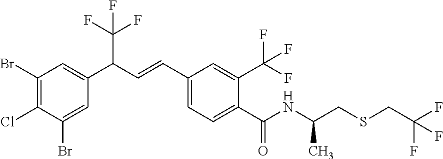 [SMILES: C[C@H](CSCC(F)(F)F)NC(=O)c1ccc(/C=C/C(c2cc(Br)c(Cl)c(Br)c2)C(F)(F)F)cc1C(F)(F)F]

Isolated as a brown gum (0.40 g, 54%).

### 4-((E)-4,4,4-Trifluoro-3-(3,4,5-trichorophenyl)but-1-en-1-yl)-N—((R)-1-(N-(2,2,2-trifluoroethyl)sulfamoyl)propan-2-yl)-2-(trifluoromethyl)benzamide (F45)

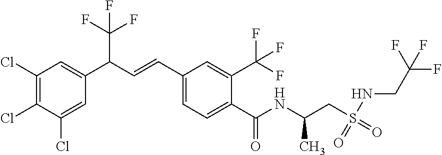 [SMILES: C[C@H](CS(=O)(=O)NCC(F)(F)F)NC(=O)c1ccc(/C=C/C(c2cc(Cl)c(Cl)c(Cl)c2)C(F)(F)F)cc1C(F)(F)F]

Isolated as an off-white solid (0.20 g, 63%).

### 2-Methyl-4-((E)-4,4,4-trifluoro-3-(3,4,5-trichlorophenyl)but-1-en-1-yl)-N—((R)-1-((2,2,2-trifluoroethyl)thio)propan-2-yl)benzamide (F51)

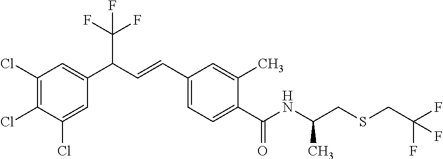 [SMILES: Cc1cc(/C=C/C(c2cc(Cl)c(Cl)c(Cl)c2)C(F)(F)F)ccc1C(=O)N[C@H](C)CSCC(F)(F)F]

Isolated as a yellow liquid (0.40 g, 64%).

### 2-Ethyl-4-((E)-4,4,4-trifluoro-3-(3,4,5-trichlorophenyl)but-1-en-1-yl)-N—((R)-1-((2,2,2-trifluoroethyl)thio)propan-2-yl)benzamide (F54

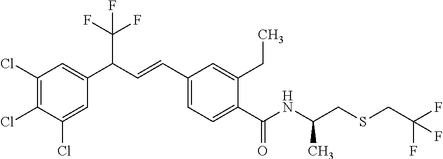 [SMILES: CCc1cc(/C=C/C(c2cc(Cl)c(Cl)c(Cl)c2)C(F)(F)F)ccc1C(=O)N[C@H](C)CSCC(F)(F)F]

Isolated as a yellow liquid (0.45 g, 71%).

### 4-((E)-3-(3,5-Dibromophenyl)-4,4,4-trifluorobut-1-en-1-yl)-N—((R)-1-((2,2,2-trifluoroethyl)thio)propan-2-yl)-2-(trifluoromethyl)benzamide (F56)

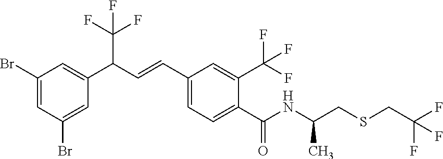 [SMILES: C[C@H](CSCC(F)(F)F)NC(=O)c1ccc(/C=C/C(c2cc(Br)cc(Br)c2)C(F)(F)F)cc1C(F)(F)F]

Isolated as a brown gum (0.185 g, 65%).

### 4-((E)-3-(3,5-Dibromophenyl)-4,4,4-trifluorobut-1-en-1-yl)-2-methyl-N—((R)-1-((2,2,2-trifluoroethyl)thio)propan-2-yl)benzamide (F57)

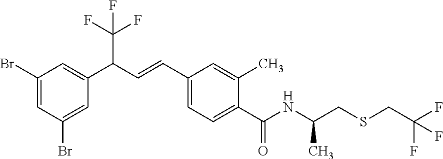 [SMILES: Cc1cc(/C=C/C(c2cc(Br)cc(Br)c2)C(F)(F)F)ccc1C(=O)N[C@H](C)CSCC(F)(F)F]

Isolated as a brown gum (0.310 g, 76%).

### N—((R)-1-(Ethylthio)propan-2-yl)-4-((E)-4,4,4-trifluoro-3-(3,4,5-trichlorophenyl)but-1-en-1-yl)-2-(trifluoromethyl)benzamide (F62)

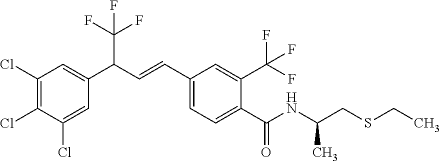 [SMILES: CCSC[C@@H](C)NC(=O)c1ccc(/C=C/C(c2cc(Cl)c(Cl)c(Cl)c2)C(F)(F)F)cc1C(F)(F)F]

Isolated as a pale-yellow gum (0.48 g, 76%).

### 2-Chloro-4-((E)-3-(3,5-dibromophenyl)-4,4,4-trifluorobut-1-en-1-yl)-N—((R)-1-((2,2,2-trifluoroethyl)thio)propan-2-yl)benzamide (F64)

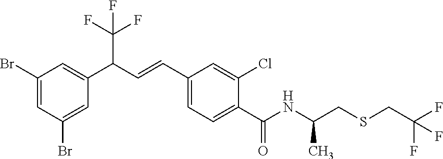 [SMILES: C[C@H](CSCC(F)(F)F)NC(=O)c1ccc(/C=C/C(c2cc(Br)cc(Br)c2)C(F)(F)F)cc1Cl]

Isolated as a brown gum (0.5 g, 68%).

### 2-Chloro-4-((E)-4,4,4-trifluoro-3-(3,4,5-trichlorophenyl)but-1-en-1-yl)-N—((R)-1-((2,2,2-trifluoroethyl)thio)propan-2-yl)benzamide (F67)

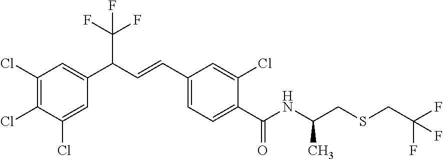 [SMILES: C[C@H](CSCC(F)(F)F)NC(=O)c1ccc(/C=C/C(c2cc(Cl)c(Cl)c(Cl)c2)C(F)(F)F)cc1Cl]

Isolated as a pale-yellow gum (0.4 g, 66%).

### N—((R)-1-(Propylthio)propan-2-yl)-4-((E)-4,4,4-trifluoro-3-(3,4,5-trichlorophenyl)but-1-en-1-yl)-2-(trifluoromethyl)benzamide (F70)

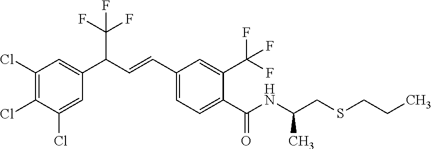 [SMILES: CCCSC[C@@H](C)NC(=O)c1ccc(/C=C/C(c2cc(Cl)c(Cl)c(Cl)c2)C(F)(F)F)cc1C(F)(F)F]

Isolated as a pale-brown gum (0.40 g, 69%).

### 4-((E)-3-(7-Chlorobenzo[d][1,3]dioxol-5-yl)-4,4,4-trifluorobut-1-en-1-yl)-N—((R)-1-((2,2,2-trifluoroethyl)thio)propan-2-yl)-2-(trifluoromethyl)benzamide (F75)

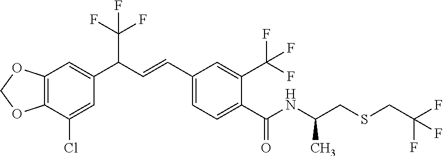 [SMILES: C[C@H](CSCC(F)(F)F)NC(=O)c1ccc(/C=C/C(c2cc(Cl)c3c(c2)OCO3)C(F)(F)F)cc1C(F)(F)F]

Isolated as a brown gum (0.166 g, 65%).

### (E)-4-(4,4,4-Trifluoro-3-(3,4,5-trichlorophenyl)but-1-en-1-yl)-N-((3-((2,2,2-trifluoroethyl)thio)oxetan-3-yl)methyl)-2-(trifluoromethyl)benzamide (F83)

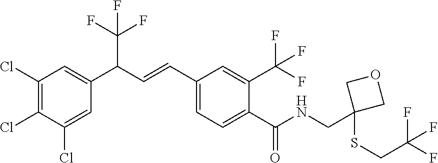 [SMILES: O=C(NCC1(SCC(F)(F)F)COC1)c1ccc(/C=C/C(c2cc(Cl)c(Cl)c(Cl)c2)C(F)(F)F)cc1C(F)(F)F]

Isolated as a brown semi-solid (0.05 g, 11%).

### N—((R)-1-(Isobutylthio)propan-2-yl)-4-((E)-4,4,4-trifluoro-3-(3,4,5-trichlorophenyl)but-1-en-1-yl)-2-(trifluoromethyl)benzamide (F85)

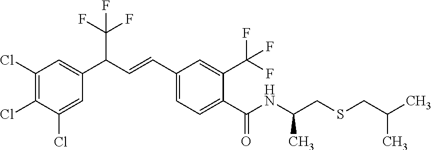 [SMILES: CC(C)CSC[C@@H](C)NC(=O)c1ccc(/C=C/C(c2cc(Cl)c(Cl)c(Cl)c2)C(F)(F)F)cc1C(F)(F)F]

Isolated as a pale-brown gum (0.40 g, 65%).

### N-((R)-1-(Isopropylthio)propan-2-yl)-4-((E)-4,4,4-trifluoro-3-(3,4,5-trichlorophenyl) but-1-en-1-yl)-2-(trifluoromethyl) benzamide (F86)

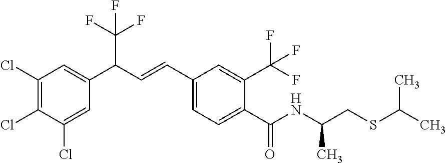 [SMILES: CC(C)SC[C@@H](C)NC(=O)c1ccc(/C=C/C(c2cc(Cl)c(Cl)c(Cl)c2)C(F)(F)F)cc1C(F)(F)F]

Isolated as a pale-brown gum (0.40 g, 68%).

### 4-((E)-3-(3,5-Dichlorophenyl)-4,4,4-trifluorobut-1-en-1-yl)-N—((R)-1-((2,2,2-trifluoroethyl)thio)propan-2-yl)-2-(trifluoromethyl)benzamide (F88)

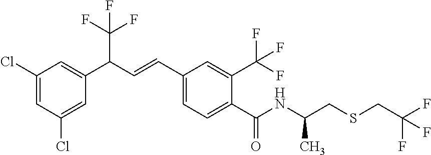 [SMILES: C[C@H](CSCC(F)(F)F)NC(=O)c1ccc(/C=C/C(c2cc(Cl)cc(Cl)c2)C(F)(F)F)cc1C(F)(F)F]

Isolated as a brown sticky solid (0.45 g, 62%).

### 4-((E)-3-(3,5-Dichlorophenyl)-4,4,4-trifluorobut-1-en-1-yl)-2-methyl-N—((R)-1-((2,2,2-trifluoroethyl)thio)propan-2-yl)benzamide (F89)

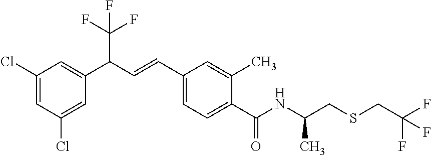 [SMILES: Cc1cc(/C=C/C(c2cc(Cl)cc(Cl)c2)C(F)(F)F)ccc1C(=O)N[C@H](C)CSCC(F)(F)F]

Isolated as a yellow sticky solid (0.50 g, 76%).

### 4-((E)-4,4,4-Trifluoro-3-(3,4,5-trichlorophenyl)but-1-en-1-yl)-N—((R)-4-((2,2,2-trifluoroethyl)thio)butan-2-yl)-2-(trifluoromethyl)benzamide (F96)

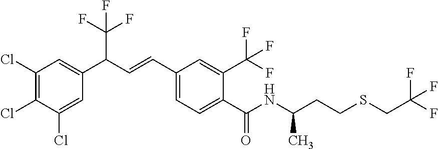 [SMILES: C[C@H](CCSCC(F)(F)F)NC(=O)c1ccc(/C=C/C(c2cc(Cl)c(Cl)c(Cl)c2)C(F)(F)F)cc1C(F)(F)F]

Isolated as a pale-yellow sticky solid (0.50 g, 77%).

### 2-Fluoro-6-methyl-4-((E)-4,4,4-trifluoro-3-(3,4,5-trichlorophenyl)but-1-en-1-yl)-N—((R)-1-((2,2,2-trifluoroethyl)thio)propan-2-yl)benzamide (F97)

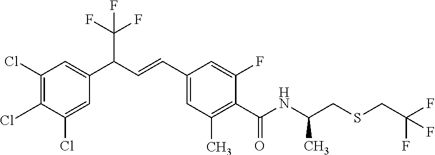 [SMILES: Cc1cc(/C=C/C(c2cc(Cl)c(Cl)c(Cl)c2)C(F)(F)F)cc(F)c1C(=O)N[C@H](C)CSCC(F)(F)F]

Isolated as a pale-yellow gum (0.50 g, 73%).

### 4-((E)-3-(3,4-Dichloro-5-formylphenyl)-4,4,4-trifluorobut-1-en-1-yl)-N—((R)-1-((2,2,2-trifluoroethyl)thio)propan-2-yl)-2-(trifluoromethyl)benzamide (F100)

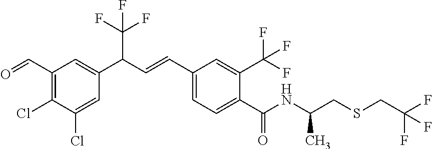 [SMILES: C[C@H](CSCC(F)(F)F)NC(=O)c1ccc(/C=C/C(c2cc(Cl)c(Cl)c(C=O)c2)C(F)(F)F)cc1C(F)(F)F]

Isolated as a yellow gum (0.15 g, 34%).

### N—((R)-1-(Allylthio)propan-2-yl)-4-((E)-4,4,4-trifluoro-3-(3,4,5-trichlorophenyl)but-1-en-1-yl)-2-(trifluoromethyl)benzamide (F101)

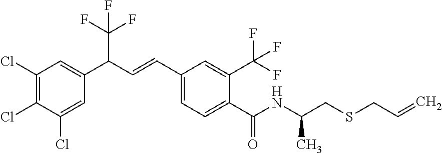 [SMILES: C=CCSC[C@@H](C)NC(=O)c1ccc(/C=C/C(c2cc(Cl)c(Cl)c(Cl)c2)C(F)(F)F)cc1C(F)(F)F]

Isolated as a pale-yellow gum (0.40 g, 58%).

### 2-Chloro-4-((E)-3-(3,5-dichlorophenyl)-4,4,4-trifluorobut-1-en-1-yl)-N—((R)-1-((2,2,2-trifluoroethyl)thio)propan-2-yl)benzamide (F104)

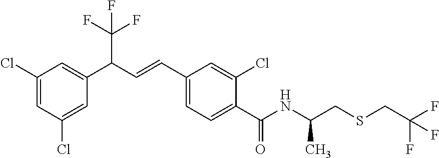 [SMILES: C[C@H](CSCC(F)(F)F)NC(=O)c1ccc(/C=C/C(c2cc(Cl)cc(Cl)c2)C(F)(F)F)cc1Cl]

Isolated as a yellow sticky solid (0.50 g, 73%).

### 4-((E)-3-(3,5-Dichloro-4-cyanophenyl)-4,4,4-trifluorobut-1-en-1-yl)-N—((R)-1-((2,2,2-trifluoroethyl)thio)propan-2-yl)-2-(trifluoromethyl)benzamide (F109)

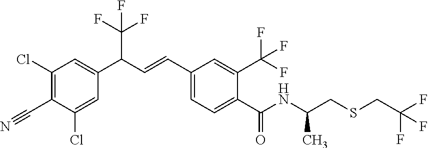 [SMILES: C[C@H](CSCC(F)(F)F)NC(=O)c1ccc(/C=C/C(c2cc(Cl)c(C#N)c(Cl)c2)C(F)(F)F)cc1C(F)(F)F]

Isolated (0.40 g, 55%).

### (E)-4-(4,4,4-Trifluoro-3-(3,4,5-trichlorophenyl)but-1-en-1-yl)-N-(1-(((2,2,2-trifluoroethyl)thio)methyl)cyclopropyl)-2-(trifluoromethyl)benzamide (F110)

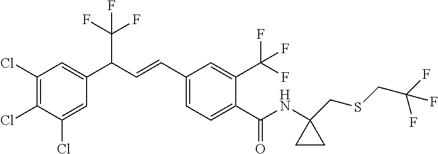 [SMILES: O=C(NC1(CSCC(F)(F)F)CC1)c1ccc(/C=C/C(c2cc(Cl)c(Cl)c(Cl)c2)C(F)(F)F)cc1C(F)(F)F]

Isolated as a brown solid (0.40 g, 61%).

### Example 7: Preparation of 4-((Z)-1-ethoxy-4,4,4-trifluoro-3-(3,4,5-trichlorophenyl)but-1-en-1-yl)-N—((R)-1-((2,2,2-trifluoroethyl)thio)propan-2-yl)-2-(trifluoromethyl)benzamide (F29)

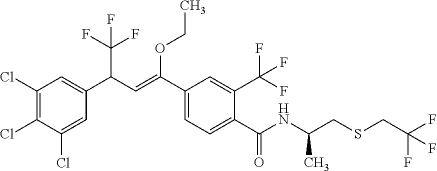 [SMILES: CCO/C(=C\C(c1cc(Cl)c(Cl)c(Cl)c1)C(F)(F)F)c1ccc(C(=O)N[C@H](C)CSCC(F)(F)F)c(C(F)(F)F)c1]

To a 25 mL vial was added (Z)-4-(1-ethoxy-4,4,4-trifluoro-3-(3,4,5-trichlorophenyl)but-1-en-1-yl)-2-(trifluoromethyl)benzoic acid (C18) (0.070 g, 0.13 mmol) and dichloromethane (1.3 mL) to give a colorless solution. (R)-1-((2,2,2-Trifluoroethyl)thio)propan-2-amine hydrochloride (C13) (0.042 g, 0.20 mmol) and ((1H-benzo[d][1,2,3]triazol-1-yl)oxy)tri(pyrrolidin-1-yl)phosphonium hexafluorophosphate (V) (0.11 g, 0.2 mmol) were then added. Triethylamine (0.075 mL, 0.54 mmol) was added and the reaction turned homogeneous. The reaction was stirred at room temperature overnight and then concentrated. Purification by flash column chromatography provided the title compound as a colorless oil (0.080 g, 81%).

The following compounds were prepared according to the procedures disclosed in Example 7:

### 4-((Z)-4,4,4-Trifluoro-1-methoxy-3-(3,4,5-trichlorophenyl)but-1-en-1-yl)-N—((R)-1-((2,2,2-trifluoroethyl)thio)propan-2-yl)-2-(trifluoromethyl)benzamide (F31)

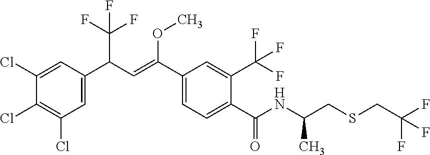 [SMILES: CO/C(=C\C(c1cc(Cl)c(Cl)c(Cl)c1)C(F)(F)F)c1ccc(C(=O)N[C@H](C)CSCC(F)(F)F)c(C(F)(F)F)c1]

Isolated as a white gum (0.017 g, 73%).

### 2-(Difluoromethyl)-4-((E)-4,4,4-trifluoro-3-(3,4,5-trichlorophenyl)but-1-en-1-yl)-N—((R)-1-((2,2,2-trifluoroethyl)thio)propan-2-yl)benzamide (F32)

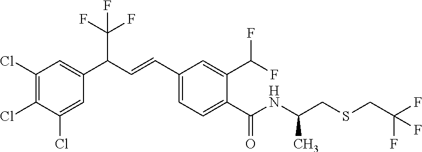 [SMILES: C[C@H](CSCC(F)(F)F)NC(=O)c1ccc(/C=C/C(c2cc(Cl)c(Cl)c(Cl)c2)C(F)(F)F)cc1C(F)F]

Isolated as a yellow oil (0.07 g, 59%).

### 4-((E)-4,4-Difluoro-3-(3,4,5-trichlorophenyl)pent-1-en-1-yl)-N—((R)-1-((2,2,2-trifluoroethyl)thio)propan-2-yl)-2-(trifluoromethyl)benzamide (F108)

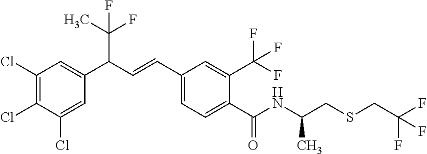 [SMILES: C[C@H](CSCC(F)(F)F)NC(=O)c1ccc(/C=C/C(c2cc(Cl)c(Cl)c(Cl)c2)C(C)(F)F)cc1C(F)(F)F]

Isolated as a yellow foam (0.094 g, 85%).

### Example 8: Preparation of 4-((E)-1-ethoxy-4,4,4-trifluoro-3-(3,4,5-trichlorophenyl)but-1-en-1-yl)-N—((R)-1-((2,2,2-trifluoroethyl)thio)propan-2-yl)-2-(trifluoromethyl)benzamide (F33)

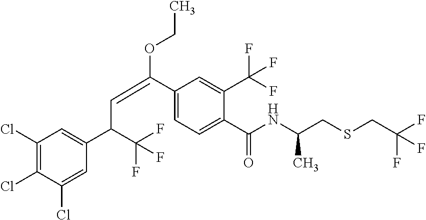 [SMILES: CCO/C(=C/C(c1cc(Cl)c(Cl)c(Cl)c1)C(F)(F)F)c1ccc(C(=O)N[C@H](C)CSCC(F)(F)F)c(C(F)(F)F)c1]

In an NMR tube, 4-((Z)-1-ethoxy-4,4,4-trifluoro-3-(3,4,5-trichlorophenyl)but-1-en-1-yl)-N—((R)-1-((2,2,2-trifluoroethyl)thio)propan-2-yl)-2-(trifluoromethyl)benzamide (F29) (0.02 g, 0.03 mmol) was dissolved in acetone-d₆ (1 mL). The reaction was set up in a UV chamber and irradiated for 7 days. The reaction mixture was then concentrated and purified by flash column chromatography to give the title compound as a colorless oil (0.04 g, 19%).

### Example 9: Preparation of (E)-N-(2-methyl-1-((2,2,2-trifluoroethyl)thio)propan-2-yl)-4-(4,4,4-trifluoro-3-(3,4,5-trichlorophenyl)but-1-en-1-yl)-2-(trifluoromethyl)benzamide (F44)

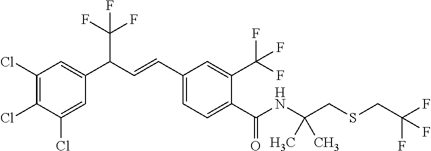 [SMILES: CC(C)(CSCC(F)(F)F)NC(=O)c1ccc(/C=C/C(c2cc(Cl)c(Cl)c(Cl)c2)C(F)(F)F)cc1C(F)(F)F]

To a 20 mL vial were added 2-methyl-1-((2,2,2-trifluoroethyl)thio)propan-2-amine hydrochloride (C30) (0.361 g, 1.61 mmol), (E)-4-(4,4,4-trifluoro-3-(3,4,5-trichlorophenyl)but-1-en-1-yl)-2-(trifluoromethyl)benzoyl chloride (0.800 g, 1.61 mmol), and 1,2-dichloroethane (4.5 mL) to give a brown suspension. 4-Methylmorpholine (0.355 mL, 3.23 mmol) was added and the reaction was stirred at 40° C. in a closed container overnight. The reaction was diluted with ethyl acetate and washed with citric acid (5%). The organic phase was concentrated followed by purification by flash column chromatography using 0-30% ethyl acetate/hexanes to provide the title compound as a red gum (0.73 g, 67%).

### Example 10: Preparation of 4-((E)-4,4,4-Trifluoro-3-(3,4,5-trichlorophenyl)but-1-en-1-yl)-N—((R)-1-((2,2,2-trifluoroethyl)thio)propan-2-yl)benzamide (FC74)

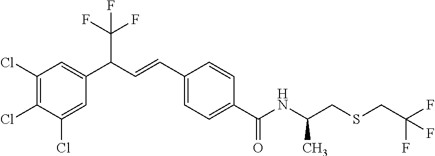 [SMILES: C[C@H](CSCC(F)(F)F)NC(=O)c1ccc(/C=C/C(c2cc(Cl)c(Cl)c(Cl)c2)C(F)(F)F)cc1]

To a 20 mL vial was added (E)-4-(4,4,4-trifluoro-3-(3,4,5-trichlorophenyl)but-1-en-1-yl)benzoic acid (0.200 g, 0.488 mmol) and dichloromethane (2.5 mL). To this was added oxalyl chloride (0.0510 mL, 0.586 mmol) followed by N,N-dimethylformamide (0.00378 mL, 0.0490 mmol). The solution was left to stir overnight at room temperature under a loose cap. To the vial was added an additional amount of dichloromethane (1.5 mL) followed by (R)-1-((2,2,2-trifluoroethyl)thio)propan-2-amine hydrochloride (C13) (0.123 g, 0.586 mmol). To this was added 4-methylmorpholine (0.161 mL, 1.47 mmol) and the mixture was stirred for 30 minutes at room temperature. Purification by flash column chromatography using 0-100% ethyl acetate/hexanes as eluent provided the title compound as a yellow gum (0.196 g, 60%).

### Example 11: Preparation of 2-((2,2,2-trifluoroethyl)thio)ethanamine (C1)

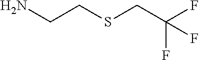

To a 100 mL round-bottomed flask charged with sodium hydride (60% oil immersion, 2.32 g, 58.1 mmol) and N,N-dimethylformamide (26.4 mL) was added 2-aminoethanethiol hydrochloride (3.00 g, 26.4 mmol) portionwise over 3 minutes. 2,2,2-Trifluoroethyl trifluoromethanesulfonate (6.13 g, 26.4 mmol) was added via a syringe over 1 minute. After cooling to room temperature, the reaction mixture was poured into water and extracted with diethyl ether. The organics were pooled and were washed with aqueous sodium hydroxide, dried over sodium sulfate, filtered, and concentrated providing the title compound (2.96 g, 71%, 71% pure). The crude material was taken on to the next step without purification.

### Example 12: Preparation of (R)-tert-butyl(1-((3,3,3-trifluoropropyl)thio)butan-2-yl)carbamate (C7)

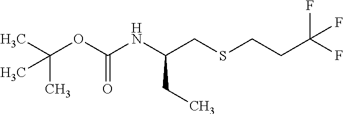 [SMILES: CC[C@H](CSCCC(F)(F)F)NC(=O)OC(C)(C)C]

To a 20 mL vial was added (R)—S-(2-((tert-butoxycarbonyl)amino)butyl) ethanethioate (0.400 g, 1.39 mmol) and methanol (7.5 mL). The solution was cooled to 0° C. under nitrogen. A freshly prepared solution of potassium hydroxide (4.34 M in methanol, 0.640 mL, 2.78 mmol) was added and the solution was left to stir for 15 minutes. 3-Bromo-1,1,1-trifluoropropane (0.246 g, 1.39 mmol) was added in one portion and the reaction was left to warm to room temperature and stirred for 36 hours. Methanol was removed under a stream of nitrogen and the residue was partioned between dichloromethane and water and hydrochloric acid (5.56 mmol). The aqueous layer was extracted with dichloromethane (2×) and the organics were combined, dried over magnesium sulfate, filtered, and concentrated. Purification by flash column chromatography using 0-20% ethyl acetate/hexanes as eluent provided the title compound as a colorless oil (0.218 g, 42%, material was 75% pure.): ¹⁹F NMR (376 MHz, CDCl₃) δ −66.35; ESIMS m/z 302 ([M+H]⁺).

The following compounds were prepared according to the procedures disclosed in Example 12:

### (R)-tert-Butyl (1-((2,2,2-trifluoroethyl)thio)butan-2-yl)carbamate (C8)

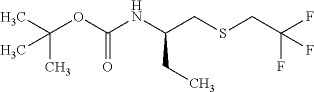 [SMILES: CC[C@H](CSCC(F)(F)F)NC(=O)OC(C)(C)C]

Isolated as a colorless solid using 2,2,2-trifluoroethyl trifluoromethanesulfonate (0.266 g, 57%): ¹H NMR (400 MHz, CDCl₃) δ 4.49 (s, 1H), 3.75-3.60 (m, 1H), 3.26-3.01 (m, 2H), 2.90-2.68 (m, 2H), 1.63 (dtd, J=12.8, 7.5, 5.4 Hz, 1H), 1.50-1.39 (m, 10H), 0.95 (t, J=7.4 Hz, 3H); ¹⁹F NMR (376 MHz, CDCl₃) δ −66.50; EIMS m/z 232 ([M-tBu]⁺).

### tert-Butyl ((2R)-1-(((2,2-difluorocyclopropyl)methyl)thio)butan-2-yl)carbamate (C9)

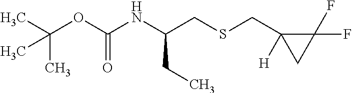 [SMILES: CC[C@H](CSCC1CC1(F)F)NC(=O)OC(C)(C)C]

Isolated as a white solid using 2,2 difluorocyclopropylmethyl bromide (0.283 g, 65%): ¹H NMR (400 MHz, CDCl₃) mixture of diastereomers δ 4.54 (s, 1H), 3.75-3.62 (m, 1H), 2.78-2.54 (m, 4H), 1.78 (ddq, J=13.3, 11.3, 7.2 Hz, 1H), 1.64 (dtdd, J=17.5, 7.6, 5.3, 2.7 Hz, 1H), 1.56-1.37 (m, 11H), 1.09 (dqd, J=13.2, 7.5, 3.7 Hz, 1H), 0.94 (dd, J=7.7, 7.1 Hz, 3H); ¹⁹F NMR (376 MHz, CDCl₃) mixture of diastereomers δ −127.78 (d, J=38.1 Hz), −128.19 (d, J=37.9 Hz), −142.60 (d, J=31.4 Hz), −143.02 (d, J=31.6 Hz); EIMS m/z 195 ([M-Boc]⁻).

### (R)-tert-Butyl (3-methyl-1-((2,2,2-trifluoroethyl)thio)butan-2-yl)carbamate (C10)

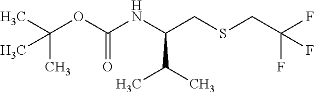 [SMILES: CC(C)[C@H](CSCC(F)(F)F)NC(=O)OC(C)(C)C]

Isolated as a white solid using 2,2,2-trifluoroethyl trifluoromethanesulfonate (0.54 g, 55%): ¹H NMR (400 MHz, CDCl₃) δ 4.49 (d, J=9.7 Hz, 1H), 3.62 (p, J=6.4 Hz, 1H), 3.28-2.99 (m, 2H), 2.90-2.51 (m, 2H), 1.99-1.73 (m, 1H), 1.45 (s, 9H), 1.06-0.74 (m, 6H); ¹⁹F NMR (376 MHz, CDCl₃) δ −66.34; EIMS m/z 301 ([M]⁺).

### (R)-tert-Butyl (1-((2,2,2-trifluoroethyl)thio)propan-2-yl)carbamate (C14)

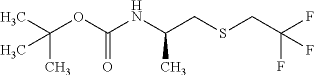 [SMILES: C[C@H](CSCC(F)(F)F)NC(=O)OC(C)(C)C]

Isolated as a golden liquid using 2,2,2-trifluoroethyl trifluoromethanesulfonate (46.5 g, 59%): ¹H NMR (400 MHz, CDCl₃) δ 4.55 (br s, 1H), 3.86-3.71 (m, 1H), 3.13 (m, 2H), 2.77 (d, J=8.0 Hz, 2H), 1.45 (s, 9H), 1.22 (d, J=8.0 Hz, 3H); ¹⁹F NMR (376 MHz, CDCl₃) δ −66.57; EIMS m/z 273 ([M]⁺).

### (R)-tert-Butyl (1-(ethylthio)propan-2-yl)carbamate (C39)

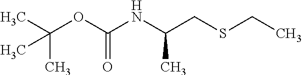

Isolated as a brown gum using ethyl trifluoromethanesulfonate (0.65 g, 52%): ¹H NMR (400 MHz, CDCl₃) δ 4.55 (br s, 1H), 3.84 (br s, 1H), 2.71-2.70 (m, 2H), 2.59-2.54 (m, 2H), 1.44 (s, 9H), 1.30-1.19 (m, 6H); IR (thin film) 3350, 2973, 2928, 1694, 1172 cm⁻¹; EIMS m/z 219 ([M]⁺).

### (R)-tert-Butyl (1-(propylthio)propan-2-yl)carba mate (C41)

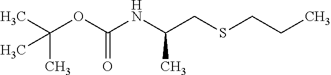

Isolated as a brown gum using iodopropane (0.6 g, 57%): ¹H NMR (300 MHz, CDCl₃) δ 4.61 (br s, 1H), 3.96-3.83 (m, 1H), 2.70-2.69 (m, 2H), 2.63-2.50 (m, 2H), 1.70-1.54 (m, 2H), 1.44 (s, 9H), 1.28-1.19 (m, 3H), 1.02-0.98 (m, 3H); IR (thin film) 3350, 2973, 2928, 1694, 1172 cm⁻¹; EIMS m/z 233 ([M]⁺).

### (R)-tert-Butyl (1-(isobutylthio)propan-2-yl)carbamate (C49)

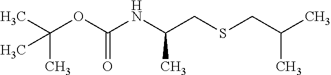 [SMILES: CC(C)CSC[C@@H](C)NC(=O)OC(C)(C)C]

Isolated as a pale-yellow liquid using 1-iodo-2-methylpropane (0.6 g, 55%): ¹H NMR (300 MHz, DMSO-d₆) δ 6.78 (br s, 1H), 3.51-3.56 (m, 1H), 2.45-2.37 (m, 3H), 1.78-1.70 (m, 1H), 1.51-1.49 (m, 1H), 1.37 (s, 9H), 1.08 (d, J=6.6 Hz, 3H), 0.96 (d, J=1.8 Hz, 3H), 0.91 (d, J=6.6 Hz, 3H); IR (thin film) 3356, 2964, 2931, 1699, 1171 cm⁻¹; EIMS m/z 247 ([M]⁺).

### (R)-tert-Butyl (1-(isopropylthio)propan-2-yl)carbamate (C51)

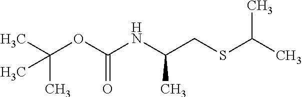

Isolated as a pale-yellow liquid using isopropyliodide (0.6 g, 54%): ¹H NMR (300 MHz, DMSO-d₆) δ 6.80 (br s, 1H), 3.56-3.51 (m, 1H), 2.96-2.91 (m, 1H), 2.60-2.54 (m, 1H), 2.46-2.40 (m, 1H), 1.37 (s, 9H), 1.24 (d, J=6.9 Hz, 3H), 1.21 (d, J=6.9 Hz, 3H), 1.09 (d, J=3.0 Hz, 3H); IR (thin film) 3348, 2973, 2929, 1698, 1172 cm⁻¹; EIMS m/z 233 ([M]⁺).

### (R)-tert-Butyl (1-((2,2,2-trifluoroethyl)thio)propan-2-yl)carbamate (C53)

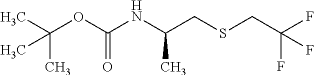 [SMILES: C[C@H](CSCC(F)(F)F)NC(=O)OC(C)(C)C]

Isolated as a white solid using 2,2,2-trifluoroethyl trifluoromethanesulfonate (0.9 g, 77%): ¹H NMR (400 MHz, CDCl₃) δ 4.40 (br s, 1H), 4.32-4.26 (m, 2H), 3.80 (br s, 1H), 1.96-1.59 (m, 2H), 1.28 (s, 9H), 1.19 (d, J=6.2 Hz, 3H).

### (R)-tert-Butyl (1-(allylthio)propan-2-yl)carbamate (C67)

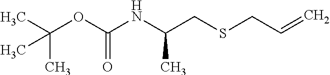

Isolated as a brown liquid using allyl bromide (1.40 g, 62%): ¹H NMR (300 MHz, DMSO-d₆) δ 6.79 (d, J=8.1 Hz, 1H), 5.82-5.68 (m, 1H), 5.14-5.04 (m, 2H), 3.60-3.50 (m, 1H), 3.14 (d, J=6.9 Hz, 2H), 2.47-2.40 (m, 2H), 1.27 (s, 9H), 1.08 (d, J=6.3 Hz, 3H); IR (thin film) 3351, 2977, 1698, 1171 cm⁻¹; EIMS m/z 231 ([M]⁺).

### Example 13: Preparation of (R)—S-(2-((tert-butoxycarbonyl)amino)-3-methylbutyl) ethanethioate (C11)

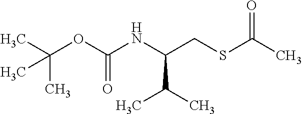 [SMILES: CC(=O)SC[C@H](NC(=O)OC(C)(C)C)C(C)C]

To a 100 mL round-bottomed flask charged with thioacetic acid (1.60 mL, 22.8 mmol) in N,N-dimethylformamide (15 mL) under an atmosphere of nitrogen, sodium hydride (60% oil immersion, 0.819 g, 20.5 mmol) was added portionwise and the suspension was stirred for 15 minutes. (R)-2-((tert-Butoxycarbonyl)amino)-3-methylbutyl methanesulfonate (C12) (3.20 g, 11.4 mmol) was added as a solid and the reaction mixture was stirred for 4 hours. The reaction mixture was diluted with ethyl acetate and water. The layers were separated and the aqueous layer was extracted with ethyl acetate (2×). The combined organic layers were washed with water (2×) and brine, dried over magnesium sulfate, filtered and concentrated. Purification by flash column chromatography using 0-50% ethyl acetate/hexanes as eluent provided the title compound as a beige solid (1.20 g, 34%): ¹H NMR (400 MHz, CDCl₃) δ 4.54 (d, J=10.2 Hz, 1H), 3.57 (tq, J=9.7, 4.5 Hz, 1H), 3.20-2.88 (m, 2H), 2.34 (s, 3H), 1.79 (dq, J=13.3, 6.7 Hz, 1H), 1.43 (s, 9H), 0.94 (dd, J=8.4, 6.7 Hz, 6H); ¹³C NMR (101 MHz, CDCl₃) δ 195.92, 155.82, 79.06, 55.66, 31.98, 31.89, 30.52, 28.34, 19.24, 17.91; ESIMS m/z 284 ([M+Na]⁺).

The following compounds were prepared according to the procedures disclosed in Example 13:

### (R)—S-(3-((tert-Butoxycarbonyl)amino)butyl) ethanethioate (C54)

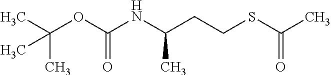 [SMILES: CC(=O)SCC[C@@H](C)NC(=O)OC(C)(C)C]

Isolated as a brown solid (1.4 g, 50%): ¹H NMR (300 MHz, CDCl₃) δ 4.36 (br s, 1H), 3.71-3.69 (m, 1H), 2.98-2.78 (m, 2H), 2.32 (s, 3H), 1.72-1.67 (m, 2H), 1.44 (s, 9H), 1.15 (d, J=6.6 Hz, 3H).

### Example 14: Preparation of (R)-2-((tert-butoxycarbonyl)amino)-3-methylbutyl methanesulfonate (C12)

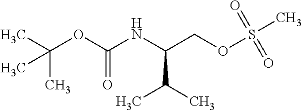 [SMILES: CC(C)[C@H](COS(C)(=O)=O)NC(=O)OC(C)(C)C]

To a 250 mL round-bottomed flask was added (R)-tert-butyl (1-hydroxy-3-methylbutan-2-yl)carbamate (7.64 g, 37.6 mmol), dichloromethane (100 mL) and triethylamine (15.7 mL, 113 mmol). The solution was cooled in an acetone/aqueous ice bath. Methanesulfonyl chloride (4.36 mL, 56.4 mmol) was added in portions over 30 minutes. The reaction was removed from the ice bath and allowed to warm to room temperature over 1.5 hours. The reaction was diluted with water and saturated sodium bicarbonate and the layers were separated. The organic layer was washed with aqueous citric acid (5%, 2×). The aqueous layers were combined and extracted with dichloromethane. The combined organic layers were dried over magnesium sulfate, filtered and concentrated to provide the title compound as a pale yellow solid (8.05 g, 69%): ¹H NMR (400 MHz, CDCl₃) δ 4.63 (d, J=9.4 Hz, 1H), 4.27 (d, J=4.5 Hz, 2H), 3.64 (tt, J=9.2, 4.5 Hz, 1H), 3.03 (s, 3H), 1.95-1.78 (m, 1H), 1.45 (s, 9H), 0.99 (d, J=6.8 Hz, 3H), 0.97 (d, J=6.8 Hz, 3H); ¹³C NMR (101 MHz, CDCl₃) δ 155.61, 79.78, 69.71, 54.90, 37.39, 29.08, 28.34, 19.39, 18.45; IR (thin film) 2967, 1734, 1636, 1529 cm⁻¹.

The following compounds were prepared according to the procedures disclosed in Example 14:

### (R)-3-((tert-Butoxycarbonyl)amino)butyl methanesulfonate (C55)

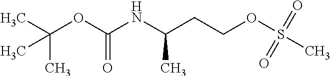 [SMILES: C[C@H](CCOS(C)(=O)=O)NC(=O)OC(C)(C)C]

Isolated as a white solid using methanesulfonic anhydride (1.47 g, 61%): ¹H NMR (300 MHz, CDCl₃) δ 4.45 (br s, 1H), 4.30-4.26 (m, 1H), 3.86-3.71 (m, 1H), 3.63-3.61 (m, 1H), 3.03 (s, 3H), 1.85-1.74 (m, 2H), 1.44 (s, 9H), 1.21 (d, J=6.0 Hz, 3H).

### Example 15: Preparation of (R)-1-((2,2,2-trifluoroethyl)thio)propan-2-amine hydrochloride (C13)

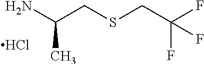

To (R)-tert-butyl (1-((2,2,2-trifluoroethyl)thio)propan-2-yl)carbamate (C14) (1.0 g, 3.7 mmol) in dichloromethane at 0° C. was added hydrogen chloride (4 N in dioxane, 5 mL). The solution was warmed to room temperature over 2 hours. The reaction mixture was concentrated in vacuo and the residue was washed with petroleum ether and dried under vacuum to provide the title compound as an off-white solid (0.70 g, 91%): ¹H NMR (300 MHz, DMSO-d₆) δ 8.09-7.85 (br s, 3H), 3.64-3.53 (m, 2H), 3.36 (br s, 1H), 2.95-2.88 (m, 1H), 2.81-2.74 (m, 1H), 1.26 (d, J=6.6 Hz, 3H); ESIMS m/z 174 ([M+H]⁺) (free base).

The following compounds were prepared according to the procedures disclosed in Example 15:

### (R)-2-Amino-4-(methylthio)-N-(2,2,2-trifluoroethyl)butanamide hydrochloride (C16)

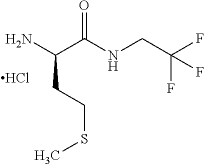

Isolated as an off-white solid (0.6 g, 73%): ¹H NMR (300 MHz, DMSO-d₆) δ 9.35 (s, 1H), 8.37 (br s, 3H), 4.10-3.94 (m, 3H), 3.56 (s, 3H), 2.50-2.40 (m, 2H), 2.05-1.98 (s, 2H); IR (thin film) 3359, 2924, 1678, 1150 cm⁻¹; EIMS m/z 230 ([M]⁺) (free base).

### (R)-1-(Ethylthio)propan-2-amine hydrochloride (C38)

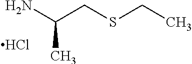

Isolated as a brown solid (0.35 g, 66%): mp 70-72° C.; ¹H NMR (400 MHz, DMSO-d₆) δ 8.06 (br s, 3H), 3.29-3.26 (m, 1H), 2.79-2.74 (m, 1H), 2.65-2.49 (m, 3H), 1.26 (d, J=6.4 Hz, 3H), 1.21 (t, J=5.4 Hz, 3H); EIMS m/z 119 ([M]⁺) (free base).

### (R)-1-(Isobutylthio)propan-2-amine hydrochloride (C48)

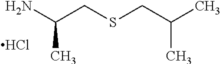

Isolated as a brown gum (0.30 g, 73%): ¹H NMR (300 MHz, DMSO-d₆) δ 8.01 (br s, 3H), 3.32-3.30 (m, 1H), 2.73-2.64 (m, 2H), 2.45 (d, J=6.6 Hz, 2H), 1.77-1.73 (m, 1H), 1.24 (d, J=6.6 Hz, 3H), 0.98-0.94 (m, 6H); IR (thin film) 3436, 2988, 1275, 750 cm⁻¹; ESIMS m/z 148 ([M+H]⁺) (free base).

### (R)-1-(Isopropylthio)propan-2-amine hydrochloride (C50)

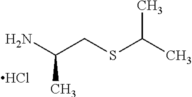

Isolated as a brown solid (0.27 g, 68%): mp 139-142° C.; ¹H NMR (300 MHz, DMSO-d₆) δ 8.05 (br s, 3H), 3.28-3.20 (m, 1H), 3.01-2.92 (m, 1H), 2.80-2.74 (m, 1H), 2.65-2.58 (m, 1H), 1.28-1.19 (m, 9H); ESIMS m/z 134 ([M+H]⁺) (free base).

### (R)-4-((2,2,2-Trifluoroethyl)thio)butan-2-amine hydrochloride (C52)

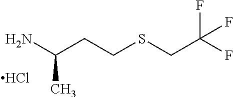

Isolated as a brown solid (0.4 g, 57%): mp 139-142° C.; ¹H NMR (400 MHz, DMSO-d₆) δ 8.18 (br s, 3H), 3.58-3.51 (m, 2H), 3.36-3.27 (m, 1H), 2.81-2.72 (m, 2H), 1.96-1.92 (m, 1H), 1.90-1.76 (m, 1H), 1.21 (d, J=6.8 Hz, 3H).

### (R)-1-(Allylthio)propan-2-amine hydrochloride (C66)

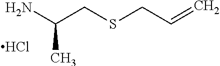

Isolated as a black gum (0.9 g, 96%): ¹H NMR (300 MHz, DMSO-d₆) δ 8.05 (br s, 3H), 5.81-5.70 (m, 1H), 5.24-5.04 (m, m2H), 3.20 (d, J=4.8 Hz, 2H), 2.73-2.66 (m, 1H), 2.58-2.54 (m, 2H), 1.25 (d, J=6.6 Hz, 3H); IR (thin film) 3435, 2923, 1633, 750 cm⁻¹; ESIMS m/z 132 ([M+H]⁺) (free base).

### (R)-2-Amino-4-(methylsulfonyl)-N-(2,2,2-trifluoroethyl)butanamide hydrochloride (C15)

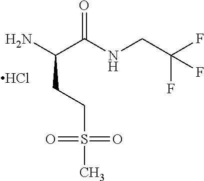 [SMILES: Cl ; CS(=O)(=O)CC[C@@H](N)C(=O)NCC(F)(F)F]

Isolated as an off-white solid (0.3 g, 91%): ¹H NMR (300 MHz, DMSO-d₆) δ 9.32 (t, 1=6.0 Hz, 1H), 8.42 (br s, 3H), 4.10-3.97 (m, 3H), 3.21-3.14 (m, 2H), 3.03 (s, 3H), 2.24-2.17 (m, 2H); EIMS m/z 262 ([M]⁺) (free base).

### (R)-1-(Propylthio)propan-2-amine hydrochloride (C40)

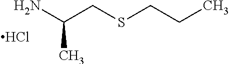

Isolated as a brown gum (0.3 g, 75%): ¹H NMR (400 MHz, DMSO-d₆) δ 8.0-7.8 (br s, 3H), 2.76-2.70 (m, 2H), 2.64-2.54 (m, 1H), 2.50-2.49 (m, 2H), 1.60-1.51 (m, 2H), 1.25 (d, J=5.4 Hz, 3H), 0.96 (t, J=7.2 Hz, 3H); IR (thin film) 3439, 2962, 2930, 1604, 750 cm⁻¹; ESIMS m/z 134 ([M+H]⁺) (free base).

### Example 16: Preparation of (R)-tert-butyl (4-(methylthio)-1-oxo-1-((2,2,2-trifluoroethyl)amino)butan-2-yl)carbamate (C17)

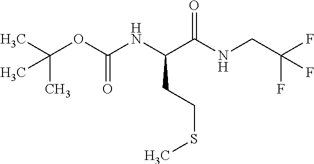 [SMILES: CSCC[C@@H](NC(=O)OC(C)(C)C)C(=O)NCC(F)(F)F]

Isobutyl chloroformate (0.71 g, 5.2 mmol) and 4-methylmorpholine (0.61 g, 6.0 mmol) were added to a stirred solution of (R)-2-((tert-butoxycarbonyl)amino)-4-(methylthio)butanoic acid (1.0 g, 4.0 mmol) in tetrahydrofuran (10 mL) cooled to −78° C. and stirring was continued for another 20 minutes. 2,2,2-Trifluoroethanamine (0.44 g, 4.4 mmol) was then added, the reaction mixture was warmed to room temperature and stirred for 2 hours. The reaction mixture was poured into ice water and extracted with ethyl acetate, dried over sodium sulfate, filtered, and concentrated to provide the title compound as an off-white solid (1.1 g, 83%): mp 91-94° C.; ¹H NMR (400 MHz, DMSO-d₆) δ 8.49 (t, J=6.0 Hz, 1H), 7.06 (d, J=8.0 Hz, 1H), 4.06-4.01 (m, 1H), 3.96-3.80 (m, 2H), 2.50-2.40 (m, 2H), 2.32 (s, 3H), 1.81-1.76 (m, 2H), 1.32 (s, 9H); IR (thin film) 3312, 3229, 1685, 1120 cm⁻¹; EIMS m/z 330 ([M]⁺).

The following compounds were prepared according to the procedures disclosed in Example 16:

### Example 17: Preparation of (Z)-4-(1-ethoxy-4,4,4-trifluoro-3-(3,4,5-trichlorophenyl)but-1-en-1-yl)-2-(trifluoromethyl)benzoic acid (C18)

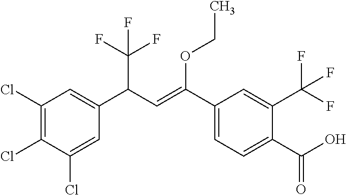 [SMILES: CCO/C(=C\C(c1cc(Cl)c(Cl)c(Cl)c1)C(F)(F)F)c1ccc(C(=O)O)c(C(F)(F)F)c1]

To a 50 mL round-bottomed flask were added (Z)-methyl 4-(1-ethoxy-4,4,4-trifluoro-3-(3,4,5-trichlorophenyl)but-1-en-1-yl)-2-(trifluoromethyl)benzoate (C19) (0.500 g, 0.933 mmol), 1,2-dichloroethane (9.3 mL) and trimethylstannanol (0.844 g, 4.67 mmol). The reaction mixture was stirred at 80° C. Upon completion of the reaction, the mixture was concentrated and dissolved in ethyl acetate. The solution was washed with pH 4 buffer (3×) and volatiles were removed under vacuum. Purification by flash column chromatography using 25% methanol/dichloromethane as eluent provided the title compound as a yellow gum (0.25 g, 47%): ¹H NMR (400 MHz, CDCl₃) δ 8.00 (d, J=8.1 Hz, 1H), 7.91-7.81 (m, 1H), 7.72 (dd, J=8.1, 1.7 Hz, 1H), 7.42 (s, 2H), 5.55 (d, J=9.5 Hz, 1H), 4.67 (p, J=9.2 Hz, 1H), 3.69 (qd, J=7.0, 2.1 Hz, 2H), 1.26 (t, J=7.0 Hz, 3H); ¹⁹F NMR (376 MHz, CDCl₃) δ −59.48, −69.08; ESIMS m/z 521 ([M+H]⁺).

The following compounds were prepared according to the procedures disclosed in Example 17:

### (Z)-4-(4,4,4-Trifluoro-1-methoxy-3-(3,4,5-trichlorophenyl)but-1-en-1-yl)-2-(trifluoromethyl)benzoic acid (C21)

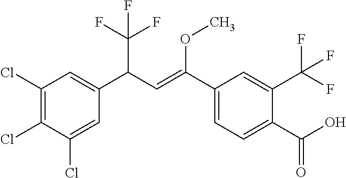 [SMILES: CO/C(=C\C(c1cc(Cl)c(Cl)c(Cl)c1)C(F)(F)F)c1ccc(C(=O)O)c(C(F)(F)F)c1]

Isolated as a brown oil (0.15 g, 95%): ¹H NMR (400 MHz, Methanol-d₄) δ 7.85 (d, J=1.3 Hz, 2H), 7.84 (d, J=1.9 Hz, 1H), 7.70 (s, 2H), 5.91 (d, J=9.4 Hz, 1H), 4.97-4.86 (m, 1H), 3.50 (s, 3H); ¹⁹F NMR (376 MHz, Methanol-d₄) δ −60.87, −70.92; ESIMS m/z 506 ([M+H]⁺).

### Example 18: Preparation of (Z)-methyl 4-(1-ethoxy-4,4,4-trifluoro-3-(3,4,5-trichlorophenyl)but-1-en-1-yl)-2-(trifluoromethyl)benzoate (C19)

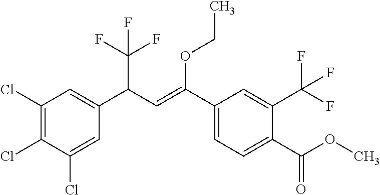 [SMILES: CCO/C(=C\C(c1cc(Cl)c(Cl)c(Cl)c1)C(F)(F)F)c1ccc(C(=O)OC)c(C(F)(F)F)c1]

To a 25 mL round-bottomed flask were added 2,2′-bipyridine (0.0338 g, 0.216 mmol), methyl 4-(1-ethoxyvinyl)-2-(trifluoromethyl)benzoate (C21) (0.889 g, 3.24 mmol), 5-(1-bromo-2,2,2-trifluoroethyl)-1,2,3-trichlorobenzene (0.370 g, 1.08 mmol), and 1,2 dichlorobenzene (4.3 mL). Copper(I) chloride (0.0107 g, 0.108 mmol) was added and the reaction mixture was purged with nitrogen for 5 minutes. The reaction was heated at 180° C. for 2.5 days. The reaction mixture was cooled to room temperature and loaded directly on silica gel. Purification by flash column chromatography using 0-10% ethyl acetate/hexanes as eluent provided the title compound as a colorless oil (0.405 g, 64%): ¹H NMR (400 MHz, CDCl₃) δ 7.85-7.80 (m, 1H), 7.79 (d, J=1.7 Hz, 1H), 7.68 (dd, J=8.0, 1.7 Hz, 1H), 7.42 (s, 2H), 5.52 (d, J=9.5 Hz, 1H), 4.67 (p, J=9.2 Hz, 1H), 3.95 (s, 3H), 3.68 (qd, J=7.1, 2.2 Hz, 2H), 1.25 (t, J=7.0 Hz, 3H); ¹⁹F NMR (376 MHz, CDCl₃) δ −59.79, −69.12; ESIMS m/z 535 ([M+H]⁺).

The following compounds were prepared according to the procedures disclosed in Example 18:

### (Z)-Methyl 4-(4,4,4-trifluoro-1-methoxy-3-(3,4,5-trichlorophenyl)but-1-en-1-yl)-2-(trifluoromethyl)benzoate (C22)

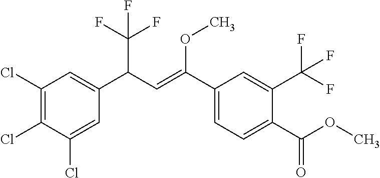 [SMILES: COC(=O)c1ccc(/C(=C/C(c2cc(Cl)c(Cl)c(Cl)c2)C(F)(F)F)OC)cc1C(F)(F)F]

Isolated as a brown oil (3.00 g, 47%): ¹H NMR (400 MHz, CDCl₃) δ 7.86-7.81 (m, 1H), 7.81-7.78 (m, 1H), 7.68 (dd, 3=8.0, 1.7 Hz, 1H), 7.42 (s, 2H), 5.53 (d, J=9.5 Hz, 1H), 4.67 (p, J=9.2 Hz, 1H), 3.95 (s, 3H), 3.50 (s, 3H); ¹⁹F NMR (376 MHz, CDCl₃) δ −59.79, −69.20; ESIMS m/z 521 ([M+H]⁺).

### (E)-2-(Difluoromethyl)-4-(4,4,4-trifluoro-3-(3,4,5-trichlorophenyl)but-1-en-1-yl)benzoic acid (C26)

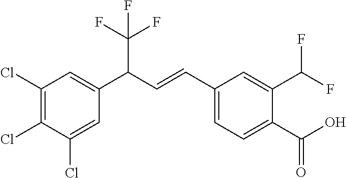 [SMILES: O=C(O)c1ccc(/C=C/C(c2cc(Cl)c(Cl)c(Cl)c2)C(F)(F)F)cc1C(F)F]

Isolated as a brown oil (1.08 g, 88%): ¹H NMR (400 MHz, Methanol-d₄) δ 8.05 (dd, J=8.2, 1.1 Hz, 1H), 7.85 (dd, J=2.0, 1.0 Hz, 1H), 7.78-7.72 (m, 1H), 7.70 (s, 2H), 7.58 (t, J=55.6 Hz, 1H), 6.86 (d, J=15.9 Hz, 1H), 6.78 (dd, 3=15.8, 8.2 Hz, 1H), 4.57 (p, J=9.0 Hz, 1H); ESIMS m/z 458 ([M−H]⁻).

### (E)-2-Ethyl-4-(4,4,4-trifluoro-3-(3,4,5-trichlorophenyl)but-1-en-1-yl)benzoic acid (C36)

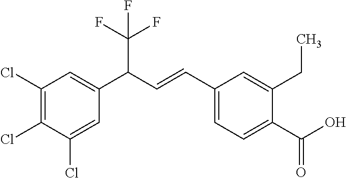 [SMILES: CCc1cc(/C=C/C(c2cc(Cl)c(Cl)c(Cl)c2)C(F)(F)F)ccc1C(=O)O]

Isolated as a brown gum using copper(I) bromide (0.90 g, 34%): ¹H NMR (400 MHz, DMSO-d₆) δ 12.92 (s, 1H), 7.98 (s, 3H), 7.79-7.71 (m, 1H), 7.49-7.45 (m, 1H), 6.94 (dd, J=15.6, 9.2 Hz, 1H), 6.78 (d, J=15.6 Hz, 1H), 4.87-4.82 (m, 1H), 2.95-2.90 (m, 2H), 1.16 (t, J=7.6 Hz, 3H); IR (thin film) 3468, 2976, 1693, 1252, 1114 cm⁻¹; ESIMS m/z 435 ([M−H]⁻).

### (E)-4-(3-(7-Chlorobenzo[d][1,3]dioxol-5-yl)-4,4,4-trifluorobut-1-en-1-yl)-2-(trifluoromethyl)benzoic acid (C42)

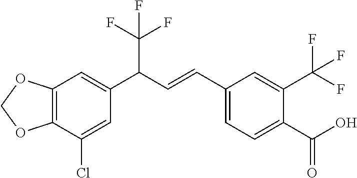 [SMILES: O=C(O)c1ccc(/C=C/C(c2cc(Cl)c3c(c2)OCO3)C(F)(F)F)cc1C(F)(F)F]

Isolated as a brown gum (0.46 g, 64%): ¹H NMR (300 MHz, DMSO-d₆) δ 13.59 (br s, 1H), 8.01 (s, 1H), 7.93 (d, J=8.1 Hz, 1H), 7.80 (d, J=8.1 Hz, 1H), 7.16 (s, 2H), 7.07 (dd, J=16.2, 9.3 Hz, 1H), 6.86 (d, J=15.9 Hz, 1H), 6.16 (s, 2H), 4.69-4.52 (m, 1H); IR (thin film) 3445, 2920, 1713, 1169, 706 cm⁻¹; ESIMS m/z 452 ([M+H]⁺).

### (E)-2-Fluoro-6-methyl-4-(4,4,4-trifluoro-3-(3,4,5-trichlorophenyl)but-1-en-1-yl)benzoic acid (C57)

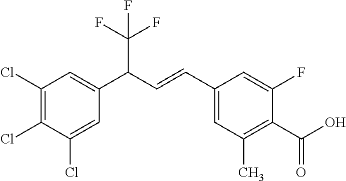 [SMILES: Cc1cc(/C=C/C(c2cc(Cl)c(Cl)c(Cl)c2)C(F)(F)F)cc(F)c1C(=O)O]

Isolated as a brown gum using copper(I) bromide (0.38 g, 60%): ¹H NMR (300 MHz, DMSO-d₆) δ 13.50 (br s, 1H), 7.89 (s, 2H), 7.42 (d, J=10.2 Hz, 1H), 7.31 (s, 1H), 6.98 (dd, J=15.6, 8.7 Hz, 1H), 6.73 (d, J=15.0 Hz, 1H), 4.87-4.81 (m, 1H), 2.34 (s, 3H); IR (thin film) 3445, 2925, 1710, 749 cm⁻¹; ESIMS m/z 439 ([M−H]⁻).

### (E)-4-(3-(3,4-Dichloro-5-(1,3-dioxolan-2-yl)phenyl)-4,4,4-trifluorobut-1-en-1-yl)-2-(trifluoromethyl)benzoic acid (C60)

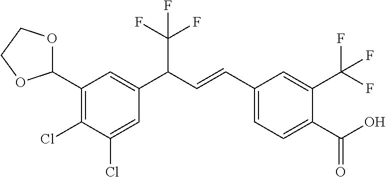 [SMILES: O=C(O)c1ccc(/C=C/C(c2cc(Cl)c(Cl)c(C3OCCO3)c2)C(F)(F)F)cc1C(F)(F)F]

Isolated using copper(I) bromide which was taken to next step without characterization (0.9 g, 100%).

### (E)-4-(4,4-Difluoro-3-(3,4,5-trichlorophenyl)pent-1-en-1-yl)-2-(trifluoromethyl)benzoic acid (C68)

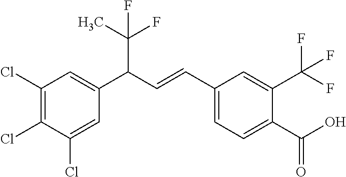 [SMILES: CC(F)(F)C(/C=C/c1ccc(C(=O)O)c(C(F)(F)F)c1)c1cc(Cl)c(Cl)c(Cl)c1]

Isolated as an orange foam using copper(I) bromide (0.150 g, 69%): ¹H NMR (400 MHz, CDCl₃) δ 7.97 (d, J=8.1 Hz, 1H), 7.81-7.73 (m, 1H), 7.64 (dd, J=8.2, 1.7 Hz, 1H), 7.40 (s, 2H), 6.67-6.51 (m, 2H), 3.81 (td, J=14.4, 7.0 Hz, 1H), 1.63 (t, J=18.4 Hz, 3H); ¹⁹F NMR (376 MHz, CDCl₃) δ −59.52, −92.51-−95.69 (m); ESIMS m/z 473 ([M+H]⁺).

### (E)-4-(3-(3,5-Dichloro-4-cyanophenyl)-4,4,4-trifluorobut-1-en-1-yl)-2-(trifluoromethyl)benzoic acid (C72)

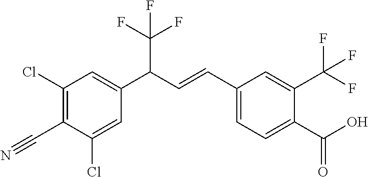 [SMILES: N#Cc1c(Cl)cc(C(/C=C/c2ccc(C(=O)O)c(C(F)(F)F)c2)C(F)(F)F)cc1Cl]

Isolated as a brown gum using copper(I) bromide (0.4 g, 59%): ¹H NMR (300 MHz, DMSO-d₆) δ 13.62 (br s, 1H), 8.04 (m, 3H), 7.94 (d, J=8.1 Hz, 1H), 7.81 (d, J=8.1 Hz, 1H), 7.11 (dd, J=15.8, 8.9 Hz, 1H), 7.01 (d, J=15.9 Hz, 1H), 4.98 (m, 1H); IR (thin film) 2166, 1653, 1166 cm⁻¹; ESIMS m/z 466 ([M−H]⁻).

### Example 19: Preparation of methyl 4-(1-ethoxyvinyl)-2-(trifluoromethyl)benzoate (C20)

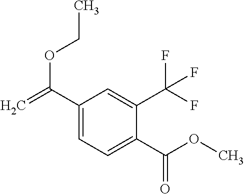 [SMILES: C=C(OCC)c1ccc(C(=O)OC)c(C(F)(F)F)c1]

To a 100 mL round-bottomed flask were added methyl 4-bromo-2-(trifluoromethyl)benzoate (4.0 g, 14 mmol) and N,N-dimethylformamide (35 mL). Bis(triphenylphosphine)palladium(II) dichloride (0.30 g, 0.42 mmol) and tributyl(1-ethoxyvinyl)stannane (5.4 mL, 16 mmol) were added and the reaction mixture was stirred at 80° C. for 3 hours under nitrogen. After cooling to room temperature, a potassium fluoride (1.0 M) solution was added to the mixture followed by dilution with diethyl ether. The mixture was stirring overnight, the layers were separated, the aqueous layer was extracted with diethyl ether (2×) and the pooled organics were dried over sodium sulfate and concentrated. Purification by flash column chromatography provided the title compound as a colorless oil (3.0 g, 70%): ¹H NMR (400 MHz, CDCl₃) δ 7.99 (dd, J=1.2, 0.4 Hz, 1H), 7.83 (dt, J=12.0, 6.0 Hz, 1H), 7.77 (d, J=8.2 Hz, 1H), 4.78 (d, J=3.1 Hz, 1H), 4.36 (d, J=3.1 Hz, 1H), 4.01-3.87 (m, 5H), 1.44 (q, 3=6.9 Hz, 3H).

### Example 20: Preparation of methyl 4-(1-methoxyvinyl)-2-(trifluoromethyl)benzoate (C23)

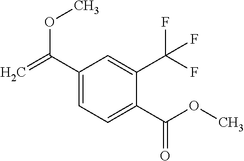 [SMILES: C=C(OC)c1ccc(C(=O)OC)c(C(F)(F)F)c1]

To a 100 mL round-bottomed flask were added methyl 4-(1,1-dimethoxyethyl)-2-(trifluoromethyl)benzoate (C24) (1.00 g, 3.42 mmol), 4-nitrobenzoic acid (0.572 g, 3.42 mmol), and pyridine (11.4 mL). Trimethylchlorosilane (2.17 mL, 17.1 mmol) was added and the reaction was stirred at 70° C. overnight. The reaction mixture was quenched with pH 7 buffer and extracted with diethyl ether. Purification by flash column chromatography provided the title compound as a colorless liquid (0.405 g, 43%): ¹H NMR (400 MHz, CDCl₃) δ 7.99 (dt, J=1.8, 0.6 Hz, 1H), 7.83 (ddd, J=8.2, 1.7, 0.6 Hz, 1H), 7.77 (dt, J=8.1, 0.6 Hz, 1H), 4.81 (d, J=3.3 Hz, 1H), 4.39 (d, J=3.4 Hz, 1H), 3.93 (s, 3H), 3.77 (s, 3H); ¹⁹F NMR (376 MHz, CDCl₃) δ −59.72; EIMS m/z 260 ([M+H]⁺).

### Example 21: Preparation of methyl 4-(1,1-dimethoxyethyl)-2-(trifluoromethyl)benzoate (C24)

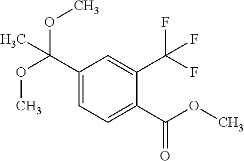 [SMILES: COC(=O)c1ccc(C(C)(OC)OC)cc1C(F)(F)F]

To a 50 mL round-bottomed flask were added methyl 4-acetyl-2-(trifluoromethyl)benzoate (C25) (3.50 g, 14.2 mmol), trimethoxymethane (15.6 mL, 143 mmol), and methanol (47.4 mL). Sulfuric acid (catalytic amount) was added and the reaction mixture was stirred at room temperature overnight. Triethylamine was added and the reaction mixture was concentrated providing the title compound as a yellow oil (4.50 g, 97%). The material was carried on to the next step without further purification or characterization.

### Example 22: Preparation of methyl 4-acetyl-2-(trifluoromethyl)benzoate (C25)

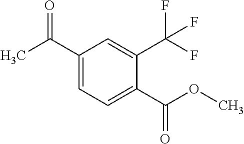 [SMILES: COC(=O)c1ccc(C(C)=O)cc1C(F)(F)F]

To a 50 mL round-bottomed flask kept under a nitrogen atmosphere were added methyl 4-(1-ethoxyvinyl)-2-(trifluoromethyl)benzoate (C20) (0.300 g, 1.09 mmol), dry tetrahydrofuran (9.1 mL), and hydrogen chloride (2 M in tetrahydrofuran, 0.547 mL, 1.09 mmol). The reaction mixture was stirred at room temperature for 30 minutes. The reaction mixture was quenched by pouring it into a saturated solution of sodium bicarbonate. The mixture was extracted with dichloromethane (3×). The combined organic layers were dried, filtered, and concentrated to provide the title compound as a yellow oil (0.216 g, 72%): ¹H NMR (400 MHz, CDCl₃) δ 8.34-8.23 (m, 1H), 8.17 (dd, J=8.0, 1.4 Hz, 1H), 7.85 (dd, J=14.6, 8.0 Hz, 1H), 3.97 (d, J=1.9 Hz, 3H), 2.67 (s, 3H).

### Example 23: Preparation of 2-(difluoromethyl)-4-vinylbenzoic acid (C27)

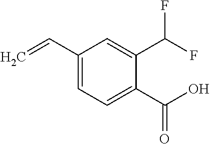

To a 250 mL round-bottomed flask were added methyl 2-(difluoromethyl)-4-vinylbenzoate (C28) (2.00 g, 9.43 mmol), lithium hydroxide hydrate (1.19 g, 28.3 mmol), methanol (9.4 mL), water (9.4 mL), and tetrahydrofuran (28 mL). The reaction mixture was stirred overnight and concentrated under vacuum. The aqueous layer was quenched with pH 4 buffer, extracted with ethyl acetate, and concentrated. Purification by flash column chromatography provided the title compound as a white solid (1.8 g, 92%): ¹H NMR (400 MHz, Methanol-d₄) δ 8.03 (dd, J=8.2, 1.3 Hz, 1H), 7.82 (s, 1H), 7.68-7.64 (m, 1H), 7.54 (t, J=56.0 Hz, 1H), 6.85 (dd, J=17.6, 11.0 Hz, 1H), 5.98 (dd, J=17.6, 0.7 Hz, 1H), 5.45 (dd, J=10.9, 0.7 Hz, 1H); ¹⁹F NMR (376 MHz, Methanol-d₄) δ −115.11.

### Example 24: Preparation of methyl 2-(difluoromethyl)-4-vinylbenzoate (C28)

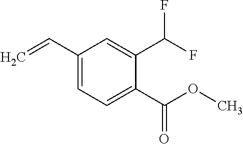

To a 250 mL round-bottomed flask were added methyl 4-bromo-2-(difluoromethyl)benzoate (C29) (2.5 g, 9.4 mmol), potassium vinyltrifluoroborate (3.8 g, 28 mmol), potassium carbonate (3.7 g, 26 mmol), and dimethylsulfoxide (47 mL). The reaction mixture was purged with nitrogen for 30 minutes followed by addition of [1,1′-bis(diphenylphosphino)ferrocene]dichloropalladium(II) with dichloromethane (0.39 g, 0.47 mmol). The reaction mixture was purged with nitrogen for another 15 minutes. The reaction was heated at 45° C. for 24 hours. After cooling to room temperature, the reaction was purified by flash column chromatography providing the title compound as a colorless oil (1.8 g, 85%): ¹H NMR (400 MHz, CDCl₃) δ 7.97 (dt, J=8.0, 1.2 Hz, 1H), 7.81 (d, J=2.0 Hz, 1H), 7.55 (t, J=55.5 Hz, 1H), 7.50 (dd, J=8.2, 2.0 Hz, 1H), 6.73 (dd, J=17.6, 10.9 Hz, 1H), 5.90 (d, J=17.6 Hz, 1H), 5.43 (d, J=10.9 Hz, 1H), 3.90 (s, 3H); ¹³C NMR (101 MHz, CDCl₃) δ 166.05, 141.89, 135.72 (t, J=22.0 Hz), 135.25, 131.37, 127.64 (t, J=1.9 Hz), 127.33 (t, J=5.1 Hz), 123.64 (t, J=8.1 Hz), 117.61, 111.86 (t, J=237.8 Hz), 52.35; ¹⁹F NMR (376 MHz, CDCl₃) δ −114.05.

The following compounds were prepared according to the procedures disclosed in Example 24:

### 2-Ethyl-4-vinylbenzoic acid (C37)

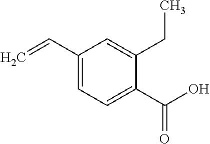

Isolated as a white solid (0.30 g, 56%): ¹H NMR (400 MHz, DMSO-d₆) δ 12.96 (s, 1H), 7.79-7.75 (m, 1H), 7.39-7.37 (m, 2H), 6.79-6.72 (m, 1H), 5.97 (d, J=17.6 Hz, 1H), 5.38 (d, J=11.2 Hz, 1H), 2.96-2.90 (m, 2H), 1.18-1.12 (m, 3H); IR (thin film) 3468, 2976, 1693, 1252, 1114 cm⁻¹; EIMS m/z 176 ([M]⁺).

### 2-(Trifluoromethyl)-4-vinylbenzoic acid (C45)

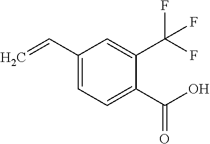

Isolated as an off-white solid (9.15 g, 67%): mp 82-83° C.; ¹H NMR (400 MHz, CDCl₃) δ 7.97 (d, J=8.0 Hz, 1H), 7.80 (s, 1H), 7.65 (d, J=8.1 Hz, 1H), 6.78 (dd, J=17.6, 10.9 Hz, 1H), 5.94 (d, J=17.6 Hz, 1H), 5.50 (d, J=10.9 Hz, 1H); ¹⁹F NMR (376 MHz, CDCl₃) δ −59.91; ESIMS m/z 215 ([M−H]⁻).

### Example 25: Preparation of methyl 4-bromo-2-(difluoromethyl)benzoate (C29)

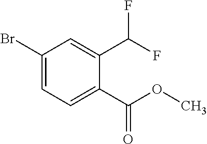

To a 100 mL round-bottomed flask were added Deoxo-Fluor® (1.14 mL, 6.17 mmol), methyl 4-bromo-2-formylbenzoate (0.500 g, 2.06 mmol), and dichloromethane (20.6 mL). Methanol (0.010 mL) was added and the reaction mixture was refluxed overnight. After cooling to room temperature, aqueous sodium bicarbonate was added to adjust the pH to 8. The organic layer was separated, dried, filtered and concentrated. Purification by flash column chromatography provided the title compound as white needles (0.42 g, 73%): ¹H NMR (400 MHz, CDCl₃) δ 7.81 (d, J=2.0 Hz, 1H), 7.76 (dt, J=8.5, 1.1 Hz, 1H), 7.57-7.49 (m, 1H), 7.39 (t, J=55.1 Hz, 1H), 3.81 (s, 3H); ¹³C NMR (101 MHz, CDCl₃) δ 165.48, 136.95 (t, J=22.5 Hz), 133.54 (t, J=1.9 Hz), 132.40, 129.31 (t, J=8.6 Hz), 127.67, 127.35 (t, J=5.0 Hz), 111.05 (t, J=239.0 Hz), 52.58; ¹⁹F NMR (376 MHz, CDCl₃) δ −114.30.

### Example 26: Preparation of 2-methyl-1-((2,2,2-trifluoroethyl)thio)propan-2-amine hydrochloride (C30)

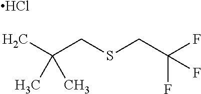

A 50 mL round-bottomed flask was charged with 4-methoxybenzyl(2-methyl-1-((2,2,2-trifluoroethyl)thio)propan-2-yl)carbamate (C31) (3.10 g, 8.82 mmol) and dichloromethane (20 mL). The reaction mixture was stirred at room temperature and trifluoroacetic acid (2.04 mL, 26.5 mmol) was added neat dropwise via a pipette. The reaction mixture was stirred for 30 minutes, concentrated under vacuum, and the residue partitioned between diethyl ether and hydrochloric acid (1 N). A gummy solid appeared and was removed by filtration. The diethyl ether layer was washed with hydrochloric acid (1 N). The combined aqueous layers were adjusted to pH >7 with sodium hydroxide (50%) and extracted with diethyl ether (3×). The diethyl ether layer was dried over magnesium sulfate and filtered. Hydrogen chloride (2 M in diethyl ether, 6 mL) was added and a white solid appeared at once. The mixture was concentrated providing the title compound as a white solid (1.86 g, 94%): mp 139-140° C.; ¹H NMR (400 MHz, DMSO-d₆) δ 8.33-5.17 (m, 3H), 3.63 (q, J=10.6 Hz, 2H), 3.00 (s, 2H), 1.29 (s, 6H); ¹⁹F NMR (376 MHz, DMSO-d₆) δ −65.39; ESIMS m/z 188 ([M+H]⁺) (free base).

### Example 27: Preparation of 4-methoxybenzyl(2-methyl-1-((2,2,2-trifluoroethyl)thio)propan-2-yl)carbamate (C31)

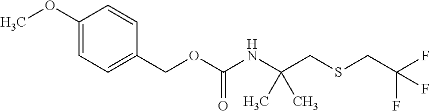 [SMILES: COc1ccc(COC(=O)NC(C)(C)CSCC(F)(F)F)cc1]

A 100 mL round-bottomed flask was charged with 2,2-dimethyl-3-((2,2,2-trifluoroethyl)thio)propanoic acid (C32) (2.45 g, 11.3 mmol), triethylamine (2.65 mL, 19.0 mmol) and 1,2-dichloroethane (30 mL). Diphenyl phosphorazidate was added neat via a syringe. The resulting mixture was stirred at room temperature for 10 minutes then at reflux for 100 minutes. (4-Methoxyphenyl)methanol (2.36 mL, 19.0 mmol) was then added neat and refluxing was continued for 18 hours. The reaction mixture was cooled and concentrated. Purification by flash column chromatography using 5-10% ethyl acetate/hexanes as eluent provided the title compound as a pale yellow oil (3.20 g, 80%): ¹H NMR (400 MHz, CDCl₃) δ 7.29 (m, 2H), 6.88 (m, 2H), 4.99 (s, 2H), 4.78 (s, 1H), 3.80 (s, 3H), 3.10 (s, 2H), 2.99 (q, J=9.9 Hz, 2H), 1.34 (s, 6H); ¹⁹F NMR (376 MHz, CDCl₃) δ −66.86; EIMS m/z 351 ([M]⁺).

### Example 28: Preparation of 2,2-dimethyl-3-((2,2,2-trifluoroethyl)thio)propanoic acid (C32)

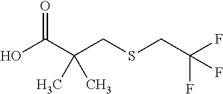

A 100 mL round-bottomed flask was charged with sodium hydride (60% oil immersion, 1.20 g, 30.0 mmol) and dry N,N-dimethylformamide (30 mL) and cooled to 0° C. A solution of 2,2,2-trifluoroethanethiol (2.61 mL, 29.3 mmol) was added dropwise. When gas evolution ceased, 3-chloro-2,2-dimethylpropanoic acid (2.00 g, 14.6 mmol) was added portionwise. The reaction was allowed to warm to room temperature and stirred overnight. The reaction mixture was added to a saturated sodium bicarbonate solution (200 mL) and extracted with diethyl ether (3×). The diethyl ether layer was separated and discarded. The aqueous layer was made acidic (pH 1) with concentrated hydrochloric acid and extracted with diethyl ether (3×). The diethyl ether layer was washed several times with brine, dried over magnesium sulfate, filtered, and concentrated providing the title compound as a pale yellow oil (2.77 g, 87%): ¹H NMR (400 MHz, CDCl₃) δ 3.13 (q, 3=9.8 Hz, 2H), 2.92 (s, 2H), 1.31 (s, 6H); ¹⁹F NMR (376 MHz, CDCl₃) δ −66.78.

### Example 29: Preparation of (R)-2-amino-N-(2,2,2-trifluoroethyl)propane-1-sulfonamide (C33)

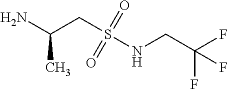

Palladium on carbon (wet, 0.120 g, 1.13 mmol) was added to a solution of (R)-benzyl(1-(N-(2,2,2-trifluoroethyl)sulfamoyl)propan-2-yl)carbamate (C34) (0.400 g, 1.13 mmol) in ethanol (10 mL). The reaction was treated with hydrogen (25 psi) at room temperature for 4 hours. The reaction mixture was filtered through Celite® and the filtrate was concentrated providing the title compound as a white solid (0.180 g, 72%): ¹H NMR (300 MHz, DMSO-d₆) δ 3.81-3.72 (m, 2H), 3.27-3.19 (m, 4H), 3.07-2.92 (m, 2H), 1.09 (d, J=6.8 Hz, 3H).

### Example 30: Preparation of (R)-benzyl(1-(N-(2,2,2-trifluoroethyl)sulfamoyl)propan-2-yl)carbamate (C34)

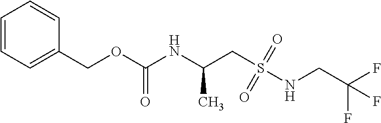 [SMILES: C[C@H](CS(=O)(=O)NCC(F)(F)F)NC(=O)OCc1ccccc1]

Triethylamine (0.10 mL, 0.69 mmol) was added to a solution of (R)-benzyl(1-(chlorosulfonyl)propan-2-yl)carbamate (C35) (0.10 g, 0.34 mmol) and 2,2,2-trifluoroethanamine (0.041 g, 0.41 mmol) in dichloromethane (3 mL). The solution was stirred at room temperature for 4 hours. The reaction mixture was poured into ice water and washed with ethyl acetate. The organic layer was separated, dried over sodium sulfate, filtered, and concentrated to provide the title compound as an off-white solid (0.10 g, 80%): ¹H NMR (300 MHz, CDCl₃) δ 7.35-7.34 (m, 5H), 5.89 (br s, 1H), 5.12-5.07 (m, 2H), 4.92-4.90 (m, 1H), 4.34-4.32 (m, 1H), 3.88-3.70 (m, 2H), 3.24-3.12 (m, 2H), 1.36 (d, J=6.9 Hz, 3H); IR (thin film) 3428, 2923, 1692, 751 cm⁻¹; ESIMS m/z 355 ([M+H]⁺).

### Example 31: Preparation of (R)-benzyl(1-(chlorosulfonyl)propan-2-yl)carbamate (C35)

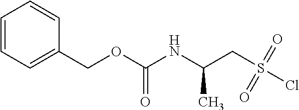 [SMILES: C[C@H](CS(=O)(=O)Cl)NC(=O)OCc1ccccc1]

Hydrogen peroxide (30% aqueous solution, 1.5 mL, 9.4 mmol) was added to a solution of (R)—S-(2-(((benzyloxy)carbonyl)amino)propyl) ethanethioate (0.50 g, 1.9 mmol) in acetic acid (2.5 mL). The reaction was stirred for 12 hours at room temperature. The precipitated solid was filtered and the volatiles were removed as an azeotrope with toluene. The residue was dissolved in dichloromethane (5 mL) and treated with oxalyl chloride (1.0 mL, 2.8 mmol). The resultant mixture was stirred for 4 hours at room temperature and concentrated under vacuum. Purification by flash column chromatography provided the title compound as an off-white solid (0.30 g, 51%): ¹H NMR (400 MHz, CDCl₃) δ 7.35-7.34 (m, 5H), 5.15 (s, 2H), 5.11-5.08 (m, 1H), 4.36 (t, J=5.4 Hz, 1H), 4.1 (br s, 1H), 3.83-3.86 (m, 1H), 1.51 (d, J=6.8 Hz, 3H); IR (thin film) 3317, 1695 cm⁻¹; ESIMS m/z 290 ([M−H]⁻).

### Example 32: Preparation of 6-(1-bromo-2,2,2-trifluoroethyl)-4-chlorobenzo[d][1,3]dioxole (C43)

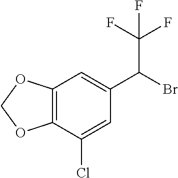 [SMILES: FC(F)(F)C(Br)c1cc(Cl)c2c(c1)OCO2]

N-Bromosuccinimide (1.76 g, 9.90 mmol) and triphenyl phosphite (3.07 g, 9.90 mmol) were added to a stirred solution of 1-(7-chlorobenzo[d][1, 3]dioxol-5-yl)-2, 2, 2-trifluoroethanol (C44) (1.68 g, 6.60 mmol) in dichloromethane (15 mL) at room temperature. The reaction mixture was heated at reflux for 6 hours and then cooled to room temperature. Purification by flash column chromatography using petroleum ether as eluent provided the title compound as a colorless liquid (0.98 g, 40%): ¹H NMR (400 MHz, CDCl₃) δ 6.96 (s, 1H), 6.92 (s, 1H), 6.09 (s, 2H), 5.02-4.97 (m, 1H); IR (thin film) 3433, 2914, 1432, 1259, 752 cm⁻¹; EIMS m/z 316 ([M]⁺).

The following compounds were prepared according to the procedures disclosed in Example 32:

### 5-(1-Bromo-2,2,2-trifluoroethyl)-2,3-dichlorobenzaldehyde (C62)

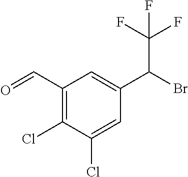 [SMILES: O=Cc1cc(C(Br)C(F)(F)F)cc(Cl)c1Cl]

Isolated as a yellow solid (3.0 g, 35%): ¹H NMR (400 MHz, DMSO-d₆) δ 10.29 (s, 1H), 8.12 (d, J=2.8 Hz, 1H), 8.02 (d, J=1.6 Hz, 1H), 6.41-6.35 (m, 1H); IR (thin film) 1698, 1115, 751 cm⁻¹; ESIMS m/z 334 ([M+H]⁺).

### 4-(1-Bromo-2, 2,2-trifluoroethyl)-2,6-dichlorobenzonitrile (C73)

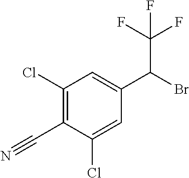 [SMILES: N#Cc1c(Cl)cc(C(Br)C(F)(F)F)cc1Cl]

Isolated as an off-white solid (2.50 g, 39%): ¹H NMR (300 MHz, DMSO-d₆) δ 7.97 (s, 2H), 6.35-6.27 (m, 1H); IR (thin film) 3423, 2997, 2245, 1114, 691 cm⁻¹; EIMS m/z 331 ([M+H]⁺).

### Example 33: Preparation of 1-(7-chlorobenzo[d][1,3]dioxol-5-yl)-2,2,2-trifluoroethanol (C44)

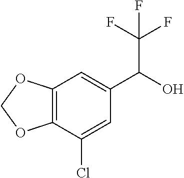 [SMILES: OC(c1cc(Cl)c2c(c1)OCO2)C(F)(F)F]

Trimethyl(trifluoromethyl)silane (1.33 g, 9.35 mmol) and tetrabutylammonium fluoride (0.393 g, 1.25 mmol) were added to a stirred solution of 7-chlorobenzo[d][1,3]dioxole-5-carbaldehyde (1.15 g, 6.23 mmol) in tetrahydrofuran (10 mL) at room temperature. The reaction was stirred for 2 hours at room temperature. The reaction mixture was quenched with hydrochloric acid (2 N) and concentrated under vacuum. The residue was diluted with dichloromethane, washed with water and brine, dried over sodium sulfate, filtered, and concentrated to provide the title compound as a brown gum (1.75 g, 106%): ¹H NMR (300 MHz, DMSO-d₆) δ 7.06 (s, 1H), 6.99 (s, 1H), 6.93 (d, J=6.0 Hz, 1H), 6.16 (s, 2H), 5.16-5.10 (m, 1H); IR (thin film) 3430, 2921, 1433, 1260, 750 cm⁻¹; ESIMS m/z 254 ([M]⁺).

The following compounds were prepared according to the procedures disclosed in Example 33:

### 1-(3,4-Dichloro-5-(1,3-dioxolan-2-yl)phenyl)-2,2,2-trifluoroethanol (C63)

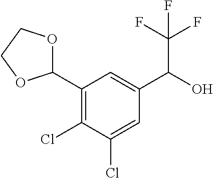 [SMILES: OC(c1cc(Cl)c(Cl)c(C2OCCO2)c1)C(F)(F)F]

Isolated as a pale-yellow solid (8.0 g, 72%): ¹H NMR (400 MHz, DMSO-d₆) δ 7.78 (d, J=2.0 Hz, 1H), 7.71 (d, J=2.3 Hz, 1H), 7.13 (d, J=5.6 Hz, 1H), 6.04 (s, 1H), 5.35-5.32 (m, 1H), 4.09-3.98 (m, 4H); IR (thin film) 3424, 1175, 750 cm⁻¹; EIMS m/z 316 ([M]⁺).

### 2,6-Dichloro-4-(2,2,2-trifluoro-1-hydroxyethyl)benzonitrile (C74)

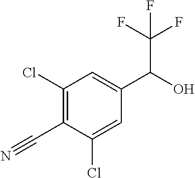 [SMILES: N#Cc1c(Cl)cc(C(O)C(F)(F)F)cc1Cl]

Isolated as an off-white solid (4.58 g, 75%): ¹H NMR (300 MHz, DMSO-d₆) δ 7.84 (s, 2H), 7.42 (d, J=6.0 Hz, 1H), 5.48-5.39 (m, 1H); IR (thin film) 3415, 2249, 1551, 820, 556 cm⁻¹; EIMS m/z 269 ([M]⁺).

### Example 34: Preparation of (3-((2,2,2-trifluoroethyl)thio)oxetan-3-yl)methanamine (C46)

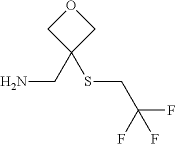 [SMILES: NCC1(SCC(F)(F)F)COC1]

Ammonium chloride (0.21 g, 3.9 mmol) was added to a mixture of 3-(nitromethyl)-3-((2,2,2-trifluoroethyl)thio)oxetane (C47) (0.30 g, 1.3 mmol) and zinc (0.25 g, 3.9 mmol) in ethanol. The reaction mixture was stirred for 18 hours at room temperature. The reaction mixture was filtered through Celite® and the filtrate was concentrated to provide the title compound which was taken to next step without further purification or characterization (0.25 g, 57%).

### Example 35: Preparation of 3-(nitromethyl)-3-((2,2,2-trifluoroethyl)thio)oxetane (C47)

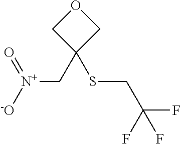

Triethylamine (0.070 mL, 0.52 mmol) was added to a mixture of 3-(nitromethylene)oxetane (0.30 g, 2.6 mmol) and 2,2,2-trifluoroethanethiol (0.30 g, 2.6 mmol) in dichloromethane (1 mL). The reaction was stirred for 1 hour at room temperature. The solvent was removed to provide the title compound as a brown gum which was taken to next step without further purification or characterization (0.38 g, 62%).

### Example 36: Preparation of (R)-tert-butyl(4-hydroxybutan-2-yl)carbamate (C56)

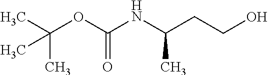

Triethylamine (2.35 mL, 16.8 mmol) and di-tert-butyl dicarbonate (2.50 g, 12.3 mmol) were added to a solution of (R)-3-aminobutan-1-ol (1.00 g, 11.2 mmol) in dichloromethane (10 mL). The reaction mixture was stirred for 4 hours at room temperature. The reaction mixture was concentrated, the residue poured into ice water, and then extracted with ethyl acetate. The organic layer was washed with water, dried over sodium sulfate, filtered, and concentrated. Purification by flash column chromatography using 20% ethyl acetate/petroleum ether as eluent provided the title compound as a white solid (1.7 g, 80%): ¹H NMR (400 MHz, DMSO-d₆) δ 6.62 (d, J=8.4 Hz, 1H), 4.43-4.31 (m, 1H), 3.55-3.52 (m, 1H), 3.40-3.38 (m, 2H), 1.55-1.48 (m, 2H), 1.43 (s, 9H), 1.01 (d, J=6.8 Hz, 3H).

### Example 37: Preparation of 2-fluoro-6-methyl-4-vinylbenzoic acid (C58)

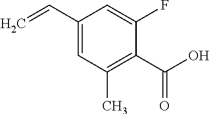

Potassium carbonate (0.237 g, 2.57 mmol) and 3-hydroxy-2,3-dimethylbutan-2-yl hydrogen vinylboronate (0.296 g, 1.71 mmol) were added to a solution of 4-bromo-2-fluoro-6-methyl-benzoic acid (0.200 g, 0.860 mmol) in dioxane (10 mL) and water (2.5 mL). The solution was degassed with nitrogen for 10 minutes. 1,1′-Bis(diphenylphosphino)ferrocene palladium(II) dichloride (0.0430 g, 0.0400 mmol) was added at room temperature and the mixture was degassed with nitrogen for 10 minutes. The reaction was heated to 100° C. for 6 hours. The reaction mixture was concentrated and the residue was washed with ethyl acetate. The aqueous layer was washed with hydrochloric acid (2 N) and extracted with ethyl acetate. The organic layer was washed with cold water, dried over sodium sulfate, filtered, and concentrated to provide the title compound as a yellow solid (0.13 g, 76%): mp 70-76° C.; ¹H NMR (300 MHz, DMSO-d₆) δ 13.45 (br s, 1H), 7.28 (s, 1H), 7.24 (d, J=5.4 Hz, 1H), 6.74 (dd, J=11.1, 17.7 Hz, 1H), 5.99 (d, J=17.4 Hz, 1H), 5.40 (d, J=10.8 Hz, 1H), 2.34 (s, 3H); ESIMS m/z 181 ([M+H]⁺).

### Example 38: Preparation of (E)-4-(3-(3,4-dichloro-5-formylphenyl)-4,4,4-trifluorobut-1-en-1-yl)-2-(trifluoromethyl)benzoic acid (C59)

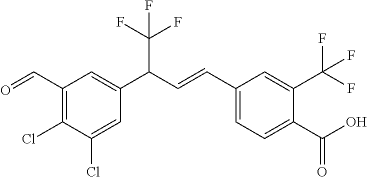 [SMILES: O=Cc1cc(C(/C=C/c2ccc(C(=O)O)c(C(F)(F)F)c2)C(F)(F)F)cc(Cl)c1Cl]

Hydrogen chloride (2 N in dioxane, 10 mL) was added to a stirred solution of (E)-4-(3-(3,4-dichloro-5-(1,3-dioxolan-2-yl)phenyl)-4,4,4-trifluorobut-1-en-1-yl)-2-(trifluoromethyl)benzoic acid (C60) (0.900 g, 1.75 mmol) in methanol (20 mL) and the reaction mixture was stirred at 85° C. for 12 hours. The reaction mixture was poured into water and washed with dichloromethane. The separated organic layer was washed with water, brine, dried over sodium sulfate, filtered, and concentrated. Purification by preparative thin layer chromatography using 30% ethyl acetate/petroleum ether as eluent provided the title compound as a clear gum (0.85 g, 43%): ¹H NMR (300 MHz, DMSO-d₆) δ 13.60 (s, 1H), 10.30 (s, 1H), 8.23 (s, 1H), 8.02-7.92 (m, 3H), 7.80 (d, J=8.4 Hz, 1H), 7.22 (dd, J=15.9, 8.7 Hz, 1H), 6.91 (d, J=15.9 Hz, 1H), 5.04-4.90 (m, 1H); IR (thin film) 3435, 1639 cm⁻¹; ESIMS m/z 469 ([M−H]⁻).

### Example 39: Preparation of 2-(5-(1-bromo-2,2,2-trifluoroethyl)-2,3-dichlorophenyl)-1,3-dioxolane (C61)

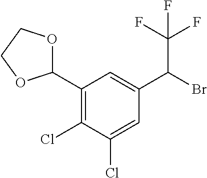 [SMILES: FC(F)(F)C(Br)c1cc(Cl)c(Cl)c(C2OCCO2)c1]

Ethylene glycol (2.2 g, 36 mmol) and para-toluenesulfonic acid (0.17 g, 0.89 mmol) were added to a stirred solution of 5-(1-bromo-2,2,2-trifluoroethyl)-2,3-dichlorobenzaldehyde (C62) (3.0 g, 8.9 mmol) in toluene (40 mL) and the reaction mixture was refluxed for 12 hours. The reaction mixture was cooled to room temperature and washed with ethyl acetate. The separated organic layer was washed with water, brine, dried over sodium sulfate, filtered, and concentrated to provide the title compound which was taken to next step without further purification or characterization (1.7 g, 43%).

The following compounds were prepared according to the procedures disclosed in Example 39:

### 2-(5-Bromo-2,3-dichlorophenyl)-1,3-dioxolane (C65)

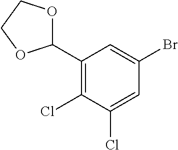 [SMILES: Clc1cc(Br)cc(C2OCCO2)c1Cl]

Isolated as a clear liquid (17.0 g, 77%): ¹H NMR (300 MHz, DMSO-d₆) δ 8.01 (d, J=2.7 Hz, 1H), 7.67 (d, J=2.1 Hz, 1H), 6.03 (s, 1H), 4.10-3.99 (m, 4H); IR (thin film) 2890, 1558, 1112, 749 cm⁻¹; ESIMS m/z 296 ([M+H]⁺).

### Example 40: Preparation of 3,4-dichloro-5-(1,3-dioxolan-2-yl)benzaldehyde (C64)

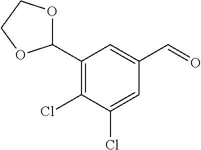 [SMILES: O=Cc1cc(Cl)c(Cl)c(C2OCCO2)c1]

Chloro(isopropyl)magnesium (100 mL, 201 mmol) was added to a stirred solution of 2-(5-bromo-2,3-dichlorophenyl)-1,3-dioxolane (C65) (15.0 g, 50.3 mmol) in tetrahydrofuran (250 mL) at 0° C. The resultant mixture was stirred at 0° C. for 30 minutes and room temperature for another 30 minutes. N,N-Dimethylformamide (11.0 mL, 151 mmol) was added at 0° C. and the reaction mixture was stirred at 0° C. for 30 minutes. After warming with stirring to room temperature over 30 minutes the reaction mixture was poured into ice water and extracted with ethyl acetate. The organic layer was washed with water, brine, dried over sodium sulfate, filtered, and concentrated. Purification by flash column chromatography using ethyl acetate/petroleum ether as eluent provided the title compound as a clear liquid (7.50 g, 59%): ¹H NMR (400 MHz, DMSO-d₆) δ 10.01 (s, 1H), 8.18 (d, J=2.0 Hz, 1H), 8.05 (d, J=2.0 Hz, 1H), 6.11 (s, 1H), 4.11-4.03 (m, 4H); IR (thin film) 3396, 2893, 1704, 1110, 748 cm⁻¹; EIMS m/z 246 ([M+H]⁺).

### Example 41: Preparation of 5-(1-bromo-2,2-difluoropropyl)-1,2,3-trichlorobenzene (C69)

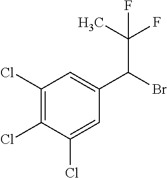

2,2-Difluoro-1-(3,4,5-trichlorophenyl)propan-1-ol (1.70 g, 6.17 mmol) was dissolved in dichloromethane (30.9 mL) at room temperature and triethylamine (1.29 mL, 9.26 mmol) was added followed by methanesulfonyl chloride (0.577 mL, 7.40 mmol). The reaction stirred for 1 hour at room temperature and then quenched by the addition of pentane (50 mL). The reaction was filtered, then concentrated to provide a white solid. The solid was re-dissolved in dichloromethane (30.9 mL) and was treated with iron(III) bromide (3.65 g, 12.3 mmol) at room temperature. The reaction was stirred overnight. The reaction was poured into water and extracted with dichloromethane. The organic layers were washed with brine, dried over sodium sulfate, filtered, and concentrated. Purification by flash column chromatography using hexanes as eluent provided a colorless oil that crystallized upon standing to provide the title compound as a white solid (1.62 g, 78%): ¹H NMR (400 MHz, CDCl₃) δ 7.52 (s, 2H), 4.85 (dd, J=12.3, 10.4 Hz, 1H), 1.77 (t, J=18.2 Hz, 3H); ¹⁹F NMR (376 MHz, CDCl₃) δ −91.56-−95.73 (m); ESIMS m/z 336 ([M+H]⁺).

### Example 42: Preparation of 2,2-difluoro-1-(3,4,5-trichlorophenyl)propan-1-ol (C70)

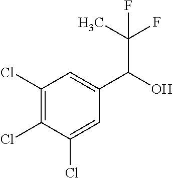

2,2-Difluoro-1-(3,4,5-trichlorophenyl)propan-1-one (C71) (1.75 g, 6.40 mmol) was dissolved in methanol (64.0 mL) at room temperature and sodium borohydride (0.290 g, 7.68 mmol) was added. The reaction stirred at room temperature for 1 hour, until gas evolution ceased. The reaction was poured into water and extracted with diethyl ether. The organic layer was washed with brine, dried over sodium sulfate, filtered, and concentrated. Purification by flash column chromatography using 0-30% acetone/hexanes as eluent provided the title compound as a clear, colorless oil (1.60 g, 91%): ¹H NMR (400 MHz, CDCl₃) δ 7.50 (d, J=0.9 Hz, 2H), 4.81 (td, J=8.7, 3.8 Hz, 1H), 1.65-1.41 (m, 3H); ¹⁹F NMR (376 MHz, CDCl₃) δ −98.54-−101.73 (m); IR (thin film) 3405, 1555, 1389 cm⁻¹.

The following compounds were prepared according to the procedures disclosed in Example 42:

### 2,6-Dichloro-4-(hydroxymethyl)benzonitrile (C76)

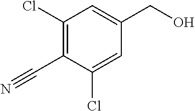 [SMILES: N#Cc1c(Cl)cc(CO)cc1Cl]

Isolated as an off-white solid (5.10 g, 81%): ¹H NMR (400 MHz, DMSO-d₆) δ 7.65 (s, 2H), 5.67 (t, J=5.6 Hz, 1H), 4.59 (d, J=5.6 Hz, 2H); IR (thin film) 3255, 2234, 1590, 1049, 569 cm⁻¹; EIMS m/z 201 ([M]⁺).

### Example 43: Preparation of 2,2-difluoro-1-(3,4,5-trichlorophenyl)propan-1-one (C71)

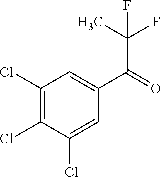

To 5-bromo-1,2,3-trichlorobenzene (2.28 g, 8.76 mmol) dissolved in diethyl ether (39.8 mL) at −78° C. under nitrogen was added n-butyllithium (3.50 mL, 8.76 mmol). The solution was stirred for 30 minutes. To this was added ethyl 2,2-difluoropropanoate (1.10 g, 7.96 mmol, as approximately 18% solution in toluene) dropwise over 10 minutes. The reaction stirred for 1 hour. The reaction mixture was quenched by addition of saturated aqueous ammonium chloride solution and was allowed to stir while warming to room temperature. The reaction was then extracted with diethyl ether, washed with water and brine, dried over sodium sulfate, filtered, and concentrated. Purification by flash column chromatography provided the title compound as a pale-yellow oil (1.76 g, 73%): ¹H NMR (400 MHz, CDCl₃) δ 8.11 (d, J=0.9 Hz, 2H), 1.89 (t, J=19.6 Hz, 3H); ¹⁹F NMR (376 MHz, CDCl₃) δ −92.66; ESIMS m/z 271 ([M−H]⁻).

### Example 44: Preparation of 2,6-dichloro-4-formylbenzonitrile (C75)

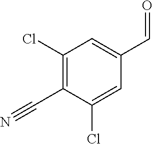 [SMILES: N#Cc1c(Cl)cc(C=O)cc1Cl]

Pyridinium chlorochromate (18.7 g, 86.6 mmol) was added to stirred solution of 2,6-dichloro-4-(hydroxymethyl)benzonitrile (C76) (5.00 g, 24.8 mmol) in dichloromethane (70 mL). The reaction mixture was stirred at room temperature for 2 hours. The reaction mixture was filtered through Celite® and concentrated. Water was added to the reaction mixture and extracted with dichloromethane. The separated organic layer was washed with brine, dried over sodium sulfate, filtered, and concentrated to provide the title compound as an off-white solid (4.95 g, 89%): ¹H NMR (300 MHz, DMSO-d₆) δ 10.04 (s, 1H), 8.18 (s, 2H); IR (thin film) 3070, 2233, 1192, 1706, 819 cm⁻¹; ESIMS m/z 199 ([M+H]⁺).

### Example 45: Preparation of methyl 3,5-dichloro-4-cyanobenzoate (C77)

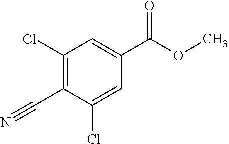

Copper(I) cyanide (7.41 g, 82.7 mmol) was added to acetonitrile and heated to 80° C. for 30 minutes. tert-Butyl nitrite (10.5 g, 102 mmol) was added followed by methyl 4-amino-3,5-dichloro-benzoate (14.0 g, 63.6 mmol) in acetonitrile (200 mL) and the reaction mixture was stirred at 80° C. for 30 minutes. The reaction mixture was poured into an ammonia solution and extracted with petroleum ether to provide the title compound as an off-white solid (7.00 g, 43%): mp 98-101° C.; ¹H NMR (400 MHz, CDCl₃) δ 8.12 (s, 2H), 3.98 (s, 3H); ESIMS m/z 229 ([M+H]⁺).

### Example 46: Preparation of (R)-tert-butyl(4-(methylsulfonyl)-1-oxo-1-((2,2,2-trifluoroethyl)amino)butan-2-yl)carbamate (C78)

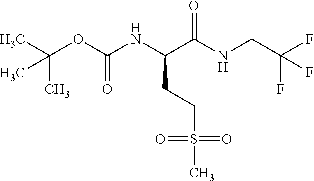 [SMILES: CC(C)(C)OC(=O)N[C@H](CCS(C)(=O)=O)C(=O)NCC(F)(F)F]

3-Chlorobenzenecarboperoxoic acid (0.783 g, 4.54 mmol) was added to a stirred solution of tert-butyl N-[(1R)-3-methylsulfanyl-1-(2,2,2-trifluoroethylcarbamoyl)propyl]carbamate (0.500 g, 1.51 mmol) in dichloromethane (10 mL) and the reaction mixture was stirred at room temperature for 3 h. The reaction mixture was poured into ice water and extracted with ethyl acetate. The organic layer was washed with aqueous sodium bicarbonate and water, dried over sodium sulfate, filtered, and concentrated to provide the title compound as an off-white solid (0.400 g, 50%): ¹H NMR (300 MHz, DMSO-d₆) δ 8.59 (t, J=6.0 Hz, 1H), 7.17 (d, J=7.8 Hz, 1H), 4.11-3.99 (m, 1H), 3.94-3.84 (m, 2H), 3.11-3.04 (m, 2H), 3.03 (s, 3H), 2.03-1.91 (m, 2H), 1.38 (s, 9H); IR (thin film) 3342, 2923, 1673, 1152, 749 cm⁻¹; ESIMS m/z 262 ([M-Boc]⁻).

### Example 47: Preparation of 2-bromo-4-((E)-4,4-difluoro-3-(3,4,5-trichlorophenyl)pent-1-en-1-yl)-N—((R)-1-((2,2,2-trifluoroethyl)sulfonyl)propan-2-yl)benzamide (F130)

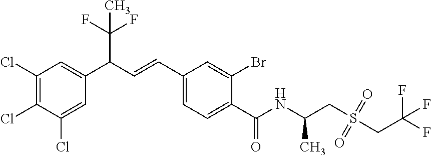 [SMILES: C[C@H](CS(=O)(=O)CC(F)(F)F)NC(=O)c1ccc(/C=C/C(c2cc(Cl)c(Cl)c(Cl)c2)C(C)(F)F)cc1Br]

Diisopropylethylamine (0.144 g, 1.11 mmol) and ((R)-1-((2,2,2-trifluoroethyl)sulfonyl)propan-2-amine hydrochloride (0.090 g, 0.37 mmol) were added to a solution of (E)-2-bromo-4-(4,4-difluoro-3-(3,4,5-trichlorophenyl)pent-1-en-1-yl)benzoic acid (C113) (0.180 g, 0.37 mmol) in dichloromethane (5 mL) at room temperature. 2-Chloro-1,3-dimethylimidazolidinium hexafluorophosphate (0.103 g, 0.37 mmol) and 1-hydroxy-7-azabenzotriazole (0.051 g, 0.37 mmol) were then added and the solution was stirred at room temperature for 2 hours. The reaction mixture was concentrated in vacuo. Purification of the crude product by column chromatography (SiO₂, 100-200 mesh, eluting with 20% ethyl acetate in hexanes) afforded the title compound as an off-white solid (0.095 g, 38%).

The following compounds were prepared according to the procedures disclosed in Example 47:

### 4-((E)-3-(3,5-Dichloro-4-fluorophenyl)-4,4-difluoropent-1-en-1-yl)-N—((R)-1-((2,2,2-trifluoroethyl)sulfonyl)propan-2-yl)-2-(trifluoromethyl)benzamide (F131)

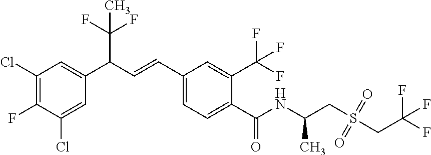 [SMILES: C[C@H](CS(=O)(=O)CC(F)(F)F)NC(=O)c1ccc(/C=C/C(c2cc(Cl)c(F)c(Cl)c2)C(C)(F)F)cc1C(F)(F)F]

Isolated as a yellow solid (0.130 g, 46%).

### 2-Chloro-4-((E)-3-(3,5-dichlorophenyl)-4,4-difluoropent-1-en-1-yl)-N—((R)-1-((2,2,2-trifluoroethyl)sulfonyl)propan-2-yl)benzamide (F132)

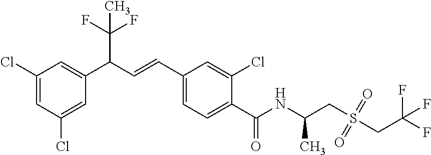 [SMILES: C[C@H](CS(=O)(=O)CC(F)(F)F)NC(=O)c1ccc(/C=C/C(c2cc(Cl)cc(Cl)c2)C(C)(F)F)cc1Cl]

Isolated as a pale yellow solid (0.100 g, 30%).

### 2-Bromo-4-((E)-3-(3,5-dichlorophenyl)-4,4-difluoropent-1-en-1-yl)-N—((R)-1-((2,2,2-trifluoroethyl)sulfonyl)propan-2-yl)benzamide (F133)

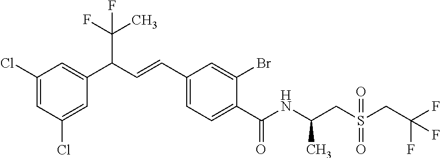 [SMILES: C[C@H](CS(=O)(=O)CC(F)(F)F)NC(=O)c1ccc(/C=C/C(c2cc(Cl)cc(Cl)c2)C(C)(F)F)cc1Br]

Isolated as a yellow solid (0.090 g, 27%).

### 4-((E)-3-(3,5-Dichlorophenyl)-4,4-difluoropent-1-en-1-yl)-N—((R)-1-((2,2,2-trifluoroethyl)sulfonyl)propan-2-yl)-2-(trifluoromethyl)benzamide (F134)

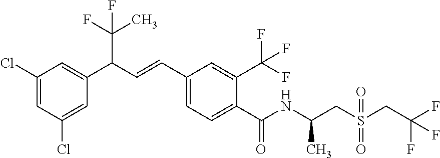 [SMILES: C[C@H](CS(=O)(=O)CC(F)(F)F)NC(=O)c1ccc(/C=C/C(c2cc(Cl)cc(Cl)c2)C(C)(F)F)cc1C(F)(F)F]

Isolated as a yellow gum (0.130 g, 46%).

### 4-((E)-4,4-Difluoro-3-(4-fluoro-3-(trifluoromethyl)phenyl)pent-1-en-1-yl)-N—((R)-1-((2,2,2-trifluoroethyl)sulfonyl)propan-2-yl)-2-(trifluoromethyl)benzamide (F135)

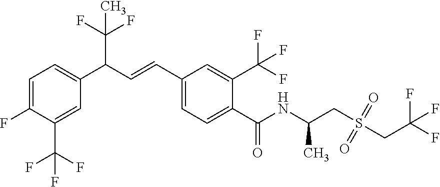 [SMILES: C[C@H](CS(=O)(=O)CC(F)(F)F)NC(=O)c1ccc(/C=C/C(c2ccc(F)c(C(F)(F)F)c2)C(C)(F)F)cc1C(F)(F)F]

Isolated as a yellow solid (0.150 g, 53%).

### 4-((E)-4,4-Difluoro-3-(3,4,5-trifluorophenyl)pent-1-en-1-yl)-N—((R)-1-((2,2,2-trifluoroethyl)sulfonyl)propan-2-yl)-2-(trifluoromethyl)benzamide (F136)

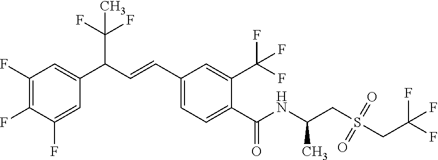 [SMILES: C[C@H](CS(=O)(=O)CC(F)(F)F)NC(=O)c1ccc(/C=C/C(c2cc(F)c(F)c(F)c2)C(C)(F)F)cc1C(F)(F)F]

Isolated as a pale yellow solid (0.090 g, 29%)

The following compound was prepared in like manner to the procedure outlined in Example 2:

### 2-Bromo-4-((E)-3-(3,4-dichlorophenyl)-4,4,4-trifluorobut-1-en-1-yl)-N-((2R)-1-((2,2,2-trifluoroethyl)sulfinyl)propan-2-yl)benzamide (F117)

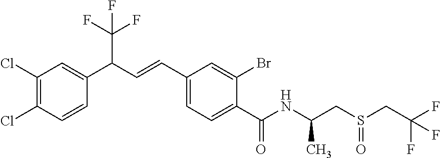 [SMILES: C[C@H](CS(=O)CC(F)(F)F)NC(=O)c1ccc(/C=C/C(c2ccc(Cl)c(Cl)c2)C(F)(F)F)cc1Br]

Isolated as a white gum (0.072 g, 95%).

The following compound was prepared in like manner to the procedure outlined in Example 3:

### (E)-4-(4,4-Difluoro-3-(3,4,5-trichlorophenyl)pent-1-en-1-yl)-N-(2-((2,2,2-trifluoroethyl)sulfonyl)ethyl)-2-(trifluoromethyl)benzamide (F126)

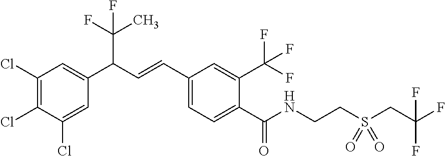 [SMILES: CC(F)(F)C(/C=C/c1ccc(C(=O)NCCS(=O)(=O)CC(F)(F)F)c(C(F)(F)F)c1)c1cc(Cl)c(Cl)c(Cl)c1]

Isolated as a white foam using two equivalents of sodium perborate (0.091 g, 52%).

### (E)-4-(4,4-Difluoro-3-(3,4,5-trichlorophenyl)pent-1-en-1-yl)-N-(2-((2-fluoroethyl)sulfinyl)ethyl)-2-(trifluoromethyl)benzamide (F129)

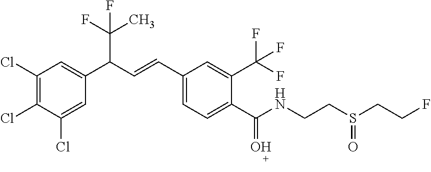 [SMILES: CC(F)(F)C(/C=C/c1ccc(C(=O)NCCS(=O)CCF)c(C(F)(F)F)c1)c1cc(Cl)c(Cl)c(Cl)c1]

Isolated as a white foam using 1 equivalent of sodium perborate (0.080 g, 90%).

### 4-((E)-4,4-Difluoro-3-(3,4,5-trichlorophenyl)pent-1-en-1-yl)-N-((2R)-1-((2-fluoroethyl)sulfinyl)propan-2-yl)-2-(trifluoromethyl)benzamide (F121) and 4-((E)-4,4-Difluoro-3-(3,4,5-trichlorophenyl)pent-1-en-1-yl)-N—((R)-1-((2-fluoroethyl)sulfonyl)propan-2-yl)-2-(trifluoromethyl)benzamide (F122)

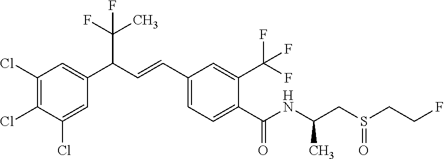 [SMILES: C[C@H](CS(=O)CCF)NC(=O)c1ccc(/C=C/C(c2cc(Cl)c(Cl)c(Cl)c2)C(C)(F)F)cc1C(F)(F)F]

Isolated as a colorless foam using 1.3 equivalents of sodium perborate (0.076 g, 48%).

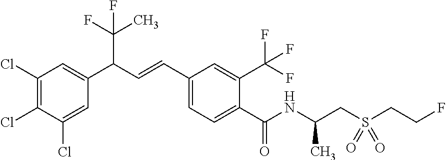 [SMILES: C[C@H](CS(=O)(=O)CCF)NC(=O)c1ccc(/C=C/C(c2cc(Cl)c(Cl)c(Cl)c2)C(C)(F)F)cc1C(F)(F)F]

Isolated as a colorless oil using 1.3 equivalents of sodium perborate (0.055 g, 36%)

The following compound was prepared in like manner to the procedure outlined in Example 6:

### 4-((E)-4,4-Difluoro-3-(3,4,5-trichlorophenyl)but-1-en-1-yl)-N—((R)-1-((2,2,2-trifluoroethyl)sulfonyl)propan-2-yl)-2-(trifluoromethyl)benzamide (F118)

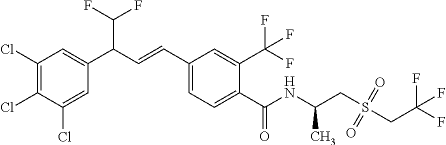 [SMILES: C[C@H](CS(=O)(=O)CC(F)(F)F)NC(=O)c1ccc(/C=C/C(c2cc(Cl)c(Cl)c(Cl)c2)C(F)F)cc1C(F)(F)F]

Isolated as an orange wax (0.114 g, 40%/o).

### 4-((E)-3-(3-Bromo-5-(2,2,2-trifluoroethoxy)phenyl)-4,4-difluoropent-1-en-1-yl)-N—((R)-1-((2,2,2-trifluoroethyl)sulfonyl)propan-2-yl)-2-(trifluoromethyl)benzamide (F120)

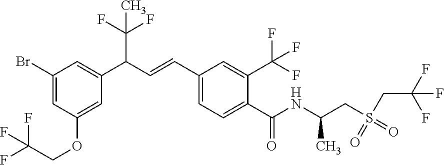 [SMILES: C[C@H](CS(=O)(=O)CC(F)(F)F)NC(=O)c1ccc(/C=C/C(c2cc(Br)cc(OCC(F)(F)F)c2)C(C)(F)F)cc1C(F)(F)F]

Isolated as an orange glass (0.074 g, 74%).

### 4-((E)-4,4-Difluoro-3-(3-fluoro-4-(trifluoromethyl)phenyl)pent-1-en-1-yl)-N—((R)-1-((2,2,2-trifluoroethyl)sulfonyl)propan-2-yl)-2-(trifluoromethyl)benzamide (F123)

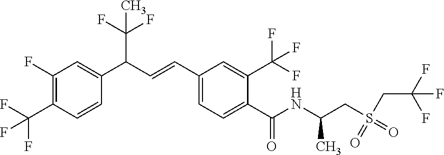 [SMILES: C[C@H](CS(=O)(=O)CC(F)(F)F)NC(=O)c1ccc(/C=C/C(c2ccc(C(F)(F)F)c(F)c2)C(C)(F)F)cc1C(F)(F)F]

Isolated as a white foamy glass (0.114 g, 81%).

The following compounds were prepared in like manner to the procedure outlined in Example 7:

### 4-((E)-3-(3-Bromo-4,5-dichlorophenyl)-4,4,4-trifluorobut-1-en-1-yl)-N—((R)-1-((2,2,2-trifluoroethyl)sulfonyl)propan-2-yl)-2-(trifluoromethyl)benzamide (F114)

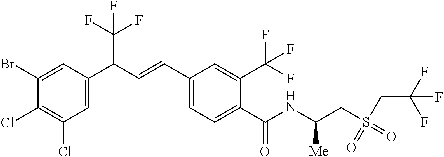 [SMILES: C[C@H](CS(=O)(=O)CC(F)(F)F)NC(=O)c1ccc(/C=C/C(c2cc(Cl)c(Cl)c(Br)c2)C(F)(F)F)cc1C(F)(F)F]

Isolated as a yellow gum (0.084 g, 69%).

### 4-((E)-3-(3,4-Dichloro-5-vinylphenyl)-4,4,4-trifluorobut-1-en-1-yl)-N—((R)-1-((2,2,2-trifluoroethyl)sulfonyl)propan-2-yl)-2-(trifluoromethyl)benzamide (F116)

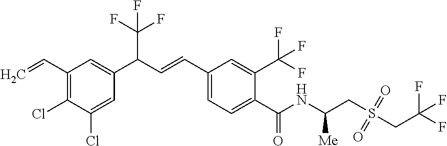 [SMILES: C=Cc1cc(C(/C=C/c2ccc(C(=O)N[C@H](C)CS(=O)(=O)CC(F)(F)F)c(C(F)(F)F)c2)C(F)(F)F)cc(Cl)c1Cl]

Isolated as a yellow wax (0.036 g, 27%).

### 4-((E)-3-(3-Bromo-4,5-dichlorophenyl)-4,4,4-trifluorobut-1-en-1-yl)-N—((R)-1-((2,2,2-trifluoroethyl)thio)propan-2-yl)-2-(trifluoromethyl)benzamide (F116)

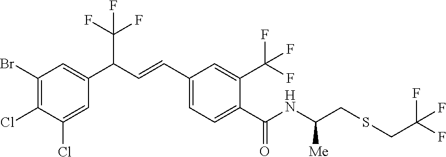 [SMILES: C[C@H](CSCC(F)(F)F)NC(=O)c1ccc(/C=C/C(c2cc(Cl)c(Cl)c(Br)c2)C(F)(F)F)cc1C(F)(F)F]

Isolated as a yellow gum (0.047 g, 27%).

### 2-Bromo-4-((E)-3-(3,4-dichlorophenyl)-4,4,4-trifluorobut-1-en-1-yl)-N—((R)-1-((2,2,2-trifluoroethyl)sulfonyl)propan-2-yl)benzamide (F124)

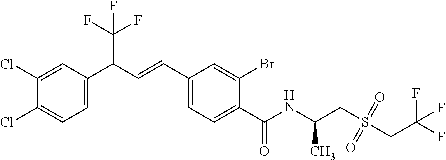 [SMILES: C[C@H](CS(=O)(=O)CC(F)(F)F)NC(=O)c1ccc(/C=C/C(c2ccc(Cl)c(Cl)c2)C(F)(F)F)cc1Br]

Isolated as a yellow gum (0.045 g, 58%).

### 2-Bromo-4-((E)-3-(3,4-dichlorophenyl)-4,4,4-trifluorobut-1-en-1-yl)-N—((R)-1-((2,2,2-trifluoroethyl)thio)propan-2-yl)benzamide (F137)

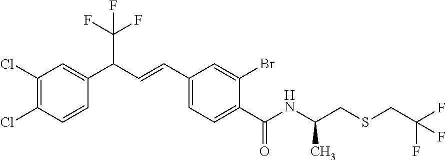 [SMILES: C[C@H](CSCC(F)(F)F)NC(=O)c1ccc(/C=C/C(c2ccc(Cl)c(Cl)c2)C(F)(F)F)cc1Br]

Isolated as a yellow gum (0.156 g, 85%).

The following compound was prepared in like manner to the procedure outlined in Example 9:

### 4-((E)-4,4-Difluoro-3-(3,4,5-trichlorophenyl)pent-1-en-1-yl)-N—((R)-1-((2-fluoroethyl)thio)propan-2-yl)-2-(trifluoromethyl)benzamide (F119)

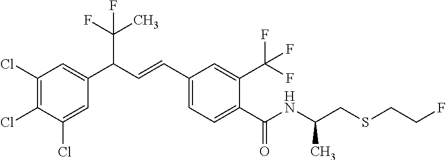 [SMILES: C[C@H](CSCCF)NC(=O)c1ccc(/C=C/C(c2cc(Cl)c(Cl)c(Cl)c2)C(C)(F)F)cc1C(F)(F)F]

Isolated as a white foam (0.248 g, 33%).

### 4-((E)-4,4-Difluoro-3-(3,4,5-trichlorophenyl)pent-1-en-1-yl)-N—((R)-1-((2-fluoroethyl)thio)ethyl)-2-(trifluoromethyl)benzamide (F128)

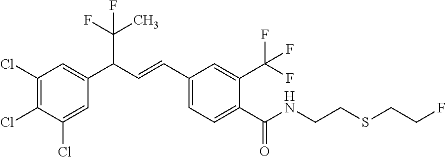 [SMILES: CC(F)(F)C(/C=C/c1ccc(C(=O)NCCSCCF)c(C(F)(F)F)c1)c1cc(Cl)c(Cl)c(Cl)c1]

Isolated as a colorless oil (0.170 g, 25%).

### ((E)-4-(4,4-Difluoro-3-(3,4,5-trichlorophenyl)pent-1-en-1-yl)-N-(2-((2, 2, 2-trifluoroethyl)thio)ethyl)-2-(trifluoromethyl)benzamide (C79)

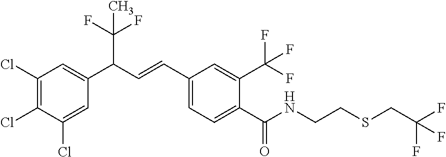 [SMILES: CC(F)(F)C(/C=C/c1ccc(C(=O)NCCSCC(F)(F)F)c(C(F)(F)F)c1)c1cc(Cl)c(Cl)c(Cl)c1]

Isolated as a colorless oil (0.150 g, 37%).

The following compound was prepared in like manner to the procedure outlined in Example 10:

### N—((R)-1-Amino-1-oxo-3-((2,2,2-trifluoroethyl)thio)propan-2-yl)-4-((E)-4,4,4-trifluoro-3-(3,4,5-trichlorophenyl)but-1-en-1-yl)-2-(trifluoromethyl)benzamide (F125)

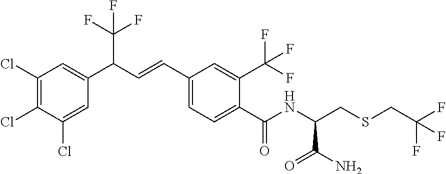 [SMILES: NC(=O)[C@H](CSCC(F)(F)F)NC(=O)c1ccc(/C=C/C(c2cc(Cl)c(Cl)c(Cl)c2)C(F)(F)F)cc1C(F)(F)F]

Isolated as an amber oil (0.515 g, 59%).

The following compounds were prepared in like manner to the procedure outlined in Example 15:

### (R)-2-Amino-3-((2,2,2-trifluoroethyl)thio)propanamide hydrochloride (C80)

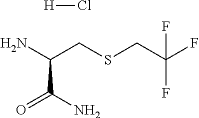

Isolated as a white amorphous solid (0.045 g, 68%): ¹H NMR (400 MHz, DMSO-d₆) δ 8.47 (s, 3H), 8.17 (s, 1H), 7.69 (s, 1H), 4.01 (dd, J=6.8, 5.7 Hz, 1H), 3.78-3.53 (m, 2H), 3.11 (dq, J=14.1, 7.6, 6.8 Hz, 2H); ¹⁹F NMR (376 MHz, DMSO-d₆) δ −60.43; ¹³C NMR (101 MHz, DMSO-d₆) δ 173.77, 131.62 (q, J=276.6, 275.8 Hz), 56.84, 38.10, 38.05 (q, J=31.3 Hz).

### (R)-1-((2-Fluoroethyl)thio)propan-2-amine hydrochloride (C81)

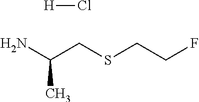

Isolated as a yellow oil (0.400 g, 87%): ¹H NMR (400 MHz, DMSO-d₆) δ 8.15 (s, 3H), 4.64 (t, J=6.1 Hz, 1H), 4.52 (t, J=6.1 Hz, 1H), 3.30 (dq, J=12.4, 6.2 Hz, 1H), 2.92 (t, J=6.1 Hz, 1H), 2.90-2.80 (m, 2H), 2.71 (dd, J=13.8, 7.4 Hz, 1H), 1.26 (d, J=6.6 Hz, 3H); IR (thin film) 3386, 2938, 1617, 1509 cm⁻¹.

### 2-((2-Fluoroethyl)thio)ethan-1-amine hydrochloride (C82)

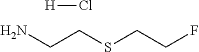

Isolated as a colorless liquid (0.357 g, 95%): ¹H NMR (500 MHz, DMSO-d₆) δ 8.13 (s, 3H), 4.62 (t, J=6.0 Hz, 1H), 4.52 (t, J=6.1 Hz, 1H), 2.97 (t, J=7.3 Hz, 2H), 2.90 (t, J=6.1 Hz, 1H), 2.86 (t, J=6.0 Hz, 1H), 2.80 (t, J=7.2 Hz, 2H); ¹⁹F NMR (471 MHz, DMSO-d₆) δ −212.81 (tt, J=46.9, 22.8 Hz); IR (thin film) 3385, 2959, 2898 cm⁻¹; HRMS-ESI (m/z) [M+H]⁺ calcd for C₄H₁₀FNS, 124.0591; found, 124.0594.

### (R)-1-((2,2,2-Trifluoroethyl)sulfonyl)propan-2-amine hydrochloride (C83)

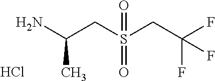

Isolated as a white solid (0.79 g, 99%): mp 129-134° C.; ¹H NMR (400 MHz, DMSO-d₆) δ 4.95 (m, 2H), 3.82 (m, 1H), 3.79 (m, 1H), 3.63 (m, 1H), 1.43 (t, J=4.1 Hz, 3H); ¹³C NMR (101 MHz, DMSO-d₆) δ 122.13 (q, J=277.1 Hz), 57.08, 54.66 (q, J=29.7 Hz), 41.39, 18.51; ¹⁹F NMR (376 MHz, DMSO-d₆) δ −59.43.

The following compounds were prepared in like manner to the procedure outlined in Example 18:

### (E)-4-(3-(3-Bromo-4,5-dichlorophenyl)-4,4,4-trifluorobut-1-en-1-yl)-2-(trifluoromethyl)benzoic acid (C84)

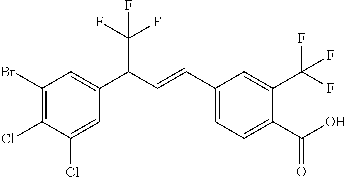 [SMILES: O=C(O)c1ccc(/C=C/C(c2cc(Cl)c(Cl)c(Br)c2)C(F)(F)F)cc1C(F)(F)F]

Isolated as a yellow gum (2.0 g, 62%): ¹H NMR (400 MHz, CDCl₃) δ 11.42 (br s, 1H), 7.80 (dt, J=8.4, 2.6 Hz, 1H), 7.62 (s, 1H), 7.49 (d, J=8.1 Hz, 1H), 7.43 (d, J=2.0 Hz, 1H), 7.32 (d, J=2.1 Hz, 1H), 6.53 (d, J=15.9 Hz, 1H), 6.38 (dd, J=15.9, 7.9 Hz, 1H), 4.02 (p, J=8.7 Hz, 1H); ¹⁹F NMR (376 MHz, CDCl₃) δ −57.88, −68.56; ESIMS m/z 522 ([M−H]⁻).

### (E)-4-(4,4-Difluoro-3-(3-fluoro-4-(trifluoromethyl)phenyl)pent-1-en-1-yl)-2-(trifluoromethyl)benzoic acid (C85)

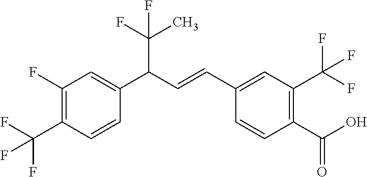 [SMILES: CC(F)(F)C(/C=C/c1ccc(C(=O)O)c(C(F)(F)F)c1)c1ccc(C(F)(F)F)c(F)c1]

Isolated as a brown oil (0.364 g, 69%): ¹H NMR (400 MHz, CDCl₃) δ 7.96 (d, J=8.1 Hz, 1H), 7.77-7.74 (m, 1H), 7.63 (ddd, J=7.9, 4.6, 2.9 Hz, 2H), 7.26 (d, J=11.2 Hz, 2H), 6.67 (dd, J=15.9, 7.9 Hz, 1H), 6.58 (d, J=15.9 Hz, 1H), 3.93 (td, J=14.2, 7.9 Hz, 1H), 1.63 (t, J=18.5 Hz, 3H); ¹⁹F NMR (376 MHz, CDCl₃) δ −59.55, −61.41 (d, J=12.5 Hz), −92.34-−95.75 (m), −113.09 (q, J=12.5 Hz); IR (thin film) 3267, 1708, 1630 cm⁻¹; ESIMS m/z 455.0 ([M−H]⁻).

### (E)-4-(4,4-Difluoro-3-(3,4,5-trichlorophenyl)but-1-en-1-yl)-2-(trifluoromethyl)benzoic acid (C86)

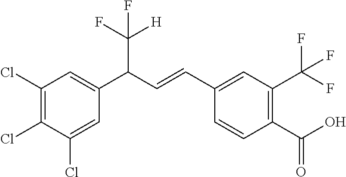 [SMILES: O=C(O)c1ccc(/C=C/C(c2cc(Cl)c(Cl)c(Cl)c2)C(F)F)cc1C(F)(F)F]

Isolated as a brown foam (2.347 g, 73%): ¹H NMR (300 MHz, CDCl₃) δ 7.98 (d, J=8.1 Hz, 1H), 7.78 (s, 1H), 7.64 (d, J=8.1 Hz, 1H), 7.38 (s, 2H), 6.69-6.44 (m, 2H), 6.03 (td, J=55.7, 3.4 Hz, 1H), 3.91 (tdd, J=15.4, 7.2, 3.5 Hz, 1H); ¹⁹F NMR (471 MHz, CDCl₃) δ −59.50, −118.25 −121.40 (m); IR (thin film) 2979, 1706 cm⁻¹; ESIMS m/z 456.9 ([M+H]⁺).

### (E)-4-(3-(3-Bromo-5-(2,2,2-trifluoroethoxy)phenyl)-4,4-difluoropent-1-en-1-yl)-2-(trifluoromethyl)benzoic acid (C87)

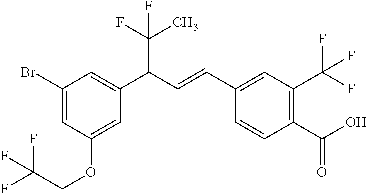 [SMILES: CC(F)(F)C(/C=C/c1ccc(C(=O)O)c(C(F)(F)F)c1)c1cc(Br)cc(OCC(F)(F)F)c1]

Isolated as an orange glass (0.415 g, 47%): ¹H NMR (400 MHz, CDCl₃) δ 7.96 (d, J=8.1 Hz, 1H), 7.76 (s, 1H), 7.63 (d, J=8.1 Hz, 1H), 7.21 (s, 1H), 7.10-7.04 (m, 1H), 6.92 (s, 1H), 6.65 (dd, J=15.9, 8.0 Hz, 1H), 6.55 (d, J=15.9 Hz, 1H), 4.36 (q, J=8.0 Hz, 2H), 3.81 (ddd, J=16.6, 12.4, 8.0 Hz, 1H), 1.60 (t, J=18.5 Hz, 3H); ¹⁹F NMR (376 MHz, CDCl₃) δ −59.50, −73.81, −89.34 −98.76 (m); IR (thin film) 3006, 1706, 1604 cm⁻¹; ESIMS m/z 544.9 ([M−H]⁻).

The following compound was prepared in like manner to the procedure outlined in Example 32:

### 1-Bromo-3-(1-bromo-2,2-difluoropropyl)-5-(2,2,2-trifluoroethoxy)benzene (C88)

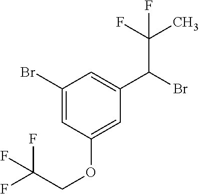 [SMILES: CC(F)(F)C(Br)c1cc(Br)cc(OCC(F)(F)F)c1]

Isolated as a pale yellow oil (1.51 g, 81%); ¹H NMR (400 MHz, CDCl₃) δ 7.31 (s, 1H), 7.10 (t, J=1.9 Hz, 1H), 7.04 (s, 1H), 4.87 (t, J=11.5 Hz, 1H), 4.35 (q, J=7.9 Hz, 2H), 1.73 (t, J=18.1 Hz, 3H); ¹⁹F NMR (376 MHz, CDCl₃) δ −73.82, −92.26-94.23 (m); IR (thin film) 1575, 1158 cm⁻¹; ESIMS m/z 412.0 ([M+H]⁺).

### 5-(1-Bromo-2,2-difluoroethyl)-1,2,3-trichlorobenzene (C89)

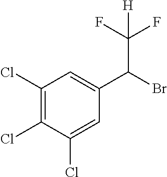 [SMILES: FC(F)C(Br)c1cc(Cl)c(Cl)c(Cl)c1]

Isolated as a clear oil (8.3 g, 67%): ¹H NMR (500 MHz, CDCl₃) δ 7.49 (s, 2H), 6.00 (td, J=55.4, 3.8 Hz, 1H), 4.85 (ddd, J=13.7, 10.4, 3.8 Hz, 1H); ¹⁹F NMR (471 MHz, CDCl₃) δ −116.16 (ddd, J=278.0, 55.2, 10.4 Hz), −119.84 (ddd, J=278.1, 55.6, 13.4 Hz); IR (thin film) 1552, 1431 cm⁻¹; ESIMS m/z 323.9 ([M+H]⁺).

### 4-(1-Bromo-2,2-difluoropropyl)-2-fluoro-1-(trifluoromethyl)benzene (C90)

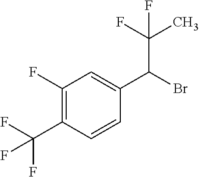 [SMILES: CC(F)(F)C(Br)c1ccc(C(F)(F)F)c(F)c1]

Isolated as a pale yellow oil (1.07 g, 78%): ¹H NMR (400 MHz, CDCl₃) δ 7.61 (t, J=7.7 Hz, 1H), 7.38 (dd, 3=14.0, 9.7 Hz, 2H), 5.05 4.85 (m, 1H), 1.77 (t, J=18.2 Hz, 3H); ¹⁹F NMR (376 MHz, CDCl₃) δ −61.60 (d, J=12.5 Hz), −91.74-−96.37 (m), −112.98 (q, J=12.5 Hz); IR (thin film) 1434, 1318 cm⁻¹; ESIMS m/z 320.0 ([M+H]⁺).

### 5-(1-Bromo-2,2-difluoropropyl)-1,3-dichloro-2-fluorobenzene (C91)

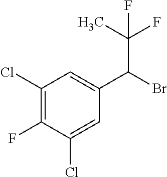

Isolated as a colorless oil (8.5 g, 73%): ¹H NMR (300 MHz, CDCl₃) δ 7.46 (d, J=6.3 Hz, 2H), 4.88 (dd, J=10.8, 12.3 Hz, 1H), 1.76 (t, J=18.6 Hz, 3H); IR (thin film) 3449, 2927, 1579, 1488 cm⁻¹; ESIMS m/z 320.00 ([M]⁺).

### 1-(1-Bromo-2,2-difluoropropyl)-3,5-dichlorobenzene (C92)

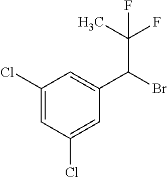

Isolated as a colorless oil (0.80 g, 78%): ¹H NMR (300 MHz, CDCl₃) δ 7.37 (s, 3H), 4.90-4.87 (m, 1H), 1.80 (t, J=18.3 Hz, 3H); IR (thin film) 3081, 1749 cm⁻¹; ESIMS m/z 303.90 ([M]⁺).

### 4-(1-Bromo-2,2-difluoropropyl)-1-fluoro-2-(trifluoromethyl)benzene (C93)

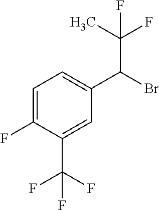 [SMILES: CC(F)(F)C(Br)c1ccc(F)c(C(F)(F)F)c1]

Isolated as a colorless oil (5.0 g, 57%): ¹H NMR (400 MHz, CDCl₃) δ 7.73-7.62 (m, 2H), 7.25 (t, J=9.6 Hz, 1H), 4.93-4.86 (m, 1H), 1.57 (t, J=11.4 Hz, 3H); IR (thin film) 1625, 935 cm⁻¹; ESIMS m/z 320.1 ([M+H]⁺).

### 5-(1-Bromo-2,2-difluoropropyl)-1,2,3-trifluorobenzene (C94)

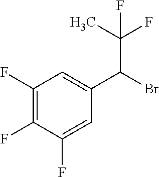

Isolated as a yellow oil (2.7 g, 48%): ¹H NMR (400 MHz, DMSO-d₆) δ 7.32 (t, J=8.0 Hz, 2H), 5.73 (d, J=12.0 Hz, 1H), 1.67 (t, J=18.8 Hz, 3H); IR (thin film) 1620, 1532, 1049 cm⁻¹; ESIMS m/z 289.2 ([M+H]⁺).

The following compound was prepared in like manner to the procedure outlined in Example 33:

### 1-(3-Bromo-4,5-dichlorophenyl)-2,2,2-trifluoroethan-1-ol (C95)

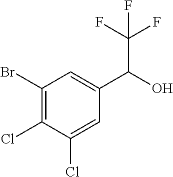 [SMILES: OC(c1cc(Cl)c(Cl)c(Br)c1)C(F)(F)F]

Isolated as a brown liquid that was used without further purification (5.5 g, 86%): ¹H NMR (400 MHz, CDCl₃) δ 7.68 (s, 1H), 7.57 (s, 1H), 5.00 (d, J=11.5 Hz, 1H), 4.75 (s, 1H); ¹⁹F NMR (376 MHz, CDCl₃) δ −78.32; ESIMS m/z 323 ([M−H]⁻).

The following compound was prepared in like manner to the procedure outlined in Example 42:

### 2,2-Difluoro-1-(3-fluoro-4-(trifluoromethyl)phenyl)propan-1-ol (C96)

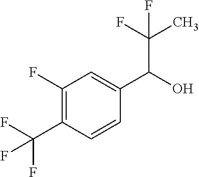 [SMILES: CC(F)(F)C(O)c1ccc(C(F)(F)F)c(F)c1]

Isolated as a clear, colorless oil (1.102 g, 91%); ¹H NMR (400 MHz, CDCl₃) δ 7.62 (t, J=7.7 Hz, 1H), 7.35 (t, J=8.3 Hz, 2H), 4.92 (td, J=8.7, 3.6 Hz, 1H), 2.65 (d, J=3.7 Hz, 1H), 1.51 (t, J=18.9 Hz, 3H); ¹⁹F NMR (376 MHz, CDCl₃) δ −61.44 (d, J=12.5 Hz), −98.37-−101.65 (m), −113.81 (q, J=12.5 Hz); IR (thin film) 3422 cm⁻¹; EIMS m/z 258.1.

### 1-(3-Bromo-5-(2,2,2-trifluoroethoxy)phenyl)-2,2-difluoropropan-1-ol (C97)

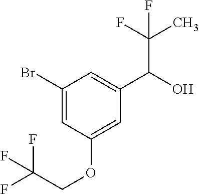 [SMILES: CC(F)(F)C(O)c1cc(Br)cc(OCC(F)(F)F)c1]

Isolated as a clear oil (1.589 g, 97%): ¹H NMR (400 MHz, CDCl₃) δ 7.30 (s, 1H), 7.10 (t, J=2.0 Hz, 1H), 7.01 (s, 1H), 4.81 (td, J=9.0, 3.7 Hz, 1H), 4.35 (q, J=8.0 Hz, 2H), 2.55 (d, J=3.6 Hz, 1H), 1.51 (t, J=18.9 Hz, 3H); ¹⁹F NMR (376 MHz, CDCl₃) δ −73.87, −98.82-−101.39 (m); IR (thin film) 3424, 1577 cm⁻¹; ESIMS m/z 350.0 ([M+H]⁺).

### 2,2-Difluoro-1-(3,4,5-trichlorophenyl)ethan-1-ol (C98)

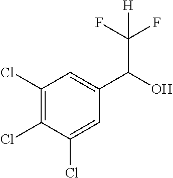 [SMILES: OC(c1cc(Cl)c(Cl)c(Cl)c1)C(F)F]

Isolated as a pale yellow solid: ¹H NMR (500 MHz, CDCl₃) δ 7.48 (s, 2H), 5.72 (td, J=55.7, 4.7 Hz, 1H), 4.80 (tt, J=9.3, 4.2 Hz, 1H), 2.65 (s, 1H); ¹⁹F NMR (471 MHz, CDCl₃) δ −127.41 (m); IR (thin film) 3381 cm⁻¹; ESIMS m/z 260.0 ([M+H]⁺).

### 1-(3,5-Dichloro-4-fluorophenyl)-2,2-difluoropropan-1-ol (C99)

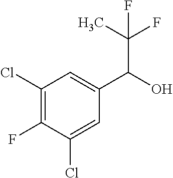

Isolated as a yellow oil (9.0 g, 75%): ¹H NMR (400 MHz, CDCl₃) δ 7.43 (d, J=6.4 Hz, 2H), 4.87-4.82 (m, 1H), 1.56-1.46 (m, 3H); IR (thin film) 3447, 2925, 1486, 1274 cm⁻¹; ESIMS m/z 258.00 ([M]⁺).

### 1-(3,5-Dichlorophenyl)-2,2-difluoropropan-1-ol (C100)

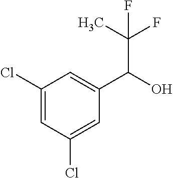

Isolated as a colorless oil (0.80 g, 81%): ¹H NMR (400 MHz, CDCl₃) δ 7.35 (s, 3H), 4.87-4.76 (m, 1H), 2.52 (br s, 1H), 1.56 (t, J=18.4 Hz, 3H); IR (thin film) 3434, 764 cm⁻¹; ESIMS m/z 240.00 ([M]⁺).

### 2,2-Difluoro-1-(4-fluoro-3-(trifluoromethyl)phenyl)propan-1-ol (C101)

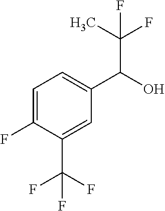 [SMILES: CC(F)(F)C(O)c1ccc(F)c(C(F)(F)F)c1]

Isolated as a yellow oil (7.0 g, 87%): ¹H NMR (300 MHz, CDCl₃) δ 7.73-7.62 (m, 2H), 7.25 (t, J=9.6 Hz, 1H), 4.93-4.86 (m, 1H), 1.57 (t, J=11.4 Hz, 3H); ¹⁹F NMR (376 MHz, CDCl₃) δ −61.44 (d, J=12.3 Hz), −99.26-−101.84 (m), −114.60 (q, J=12.8 Hz); IR (thin film) 3434, 764 cm⁻¹; ESIMS m/z 258 ([M]⁺).

### 2,2-Difluoro-1-(3,4,5-trifluorophenyl)propan-1-ol (C102)

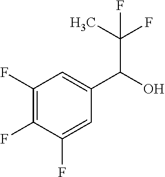

Isolated as a colorless oil (4.0 g, 76%): ¹H NMR (400 MHz, DMSO-d₆) δ 7.32 (t, J=8.0 Hz, 2H), 6.58 (d, J=6.0 Hz, 1H), 4.86-4.79 (m, 1H), 1.53 (t, J=19.2 Hz, 3H); IR (thin film) 3436, 1536, 1043 cm⁻¹; ESIMS m/z 226 ([M+H]⁺).

The following compounds were prepared in like manner to the procedure outlined in Example 43:

### 2,2-Difluoro-1-(3,4,5-trichlorophenyl)ethan-1-one (C103)

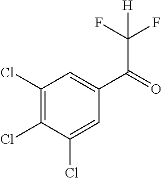 [SMILES: O=C(c1cc(Cl)c(Cl)c(Cl)c1)C(F)F]

Isolated as an off-white solid (9.25 g, 88%): mp 45-48° C.; ¹H NMR (500 MHz, CDCl₃) δ 7.71 (s, 2H), 6.21 (t, J=53.5 Hz, 1H); ¹⁹F NMR (471 MHz, CDCl₃) δ −126.71 (d, J=53.4 Hz); IR (thin film) 1743, 1559 cm⁻¹; ESIMS m/z 260.0 ([M+H]⁺).

### 1-(3-Bromo-5-(2,2,2-trifluoroethoxy)phenyl)-2,2-difluoropropan-1-one (C104)

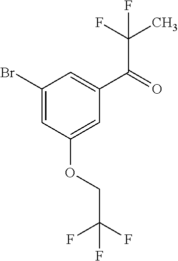 [SMILES: CC(F)(F)C(=O)c1cc(Br)cc(OCC(F)(F)F)c1]

Isolated as a clear, colorless oil (1.637 g, 79%): ¹H NMR (400 MHz, CDCl₃) δ 7.94 (s, 1H), 7.59 (s, 1H), 7.40 (t, J=2.0 Hz, 1H), 4.41 (q, J=7.9 Hz, 2H), 1.89 (t, J=19.5 Hz, 3H); ¹⁹F NMR (376 MHz, CDCl₃) δ −73.79, −92.68; IR (thin film) 1710, 1571 cm⁻¹; EIMS m/z 346.0.

### 2,2-Difluoro-1-(3-fluoro-4-(trifluoromethyl)phenyl)propan-1-one (C105)

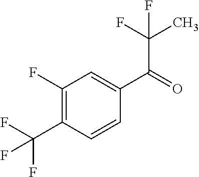 [SMILES: CC(F)(F)C(=O)c1ccc(C(F)(F)F)c(F)c1]

Isolated as a pale yellow oil (1.20 g, 57%): ¹H NMR (400 MHz, CDCl₃) δ 8.01 (d, J=8.2 Hz, 1H), 7.93 (d, J=10.8 Hz, 1H), 7.77 (t, J=7.5 Hz, 1H), 1.91 (t, J=19.6 Hz, 3H); ¹⁹F NMR (376 MHz, CDCl₃) δ −62.07 (d, J=12.8 Hz), −93.04, −112.14 (q, J=12.8 Hz); EIMS m/z 256.0.

### 1-(3,5-Dichloro-4-fluorophenyl)-2,2-difluoropropan-1-one (C106)

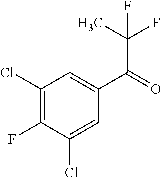

Isolated as a colorless oil (9.5 g, 78%): ¹H NMR (300 MHz, CDCl₃) δ 8.11-8.09 (m, 2H), 1.85 (t, J=19.2 Hz, 3H); IR (thin film) 3412, 2926, 1714, 1184, 815 cm⁻¹; ESIMS m/z 256.00 ([M]⁺).

### 1-(3,5-Dichlorophenyl)-2,2-difluoropropan-1-one (C107)

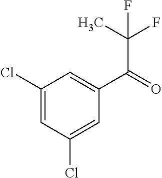

Isolated as a yellow gum (0.95 g, 85%): ¹H NMR (400 MHz, CDCl₃) δ 7.97 (d, J=1.2 Hz, 2H), 7.62 (d, J=2.0 Hz, 1H), 1.94 (t, J=19.6 Hz, 3H); IR (thin film) 3431, 1714 cm⁻¹; ESIMS m/z 238.00 ([M]⁺).

### 2,2-Difluoro-1-(4-fluoro-3-(trifluoromethyl)phenyl)propan-1-one (C108)

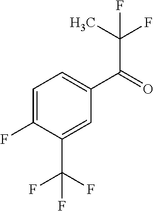 [SMILES: CC(F)(F)C(=O)c1ccc(F)c(C(F)(F)F)c1]

Isolated as a yellow oil (9.2 g, 73%): ¹H NMR (400 MHz, CDCl₃) δ 8.42-8.41 (m, 1H), 8.37-8.35 (m, 1H), 7.36 (t, J=9.2 Hz, 1H), 1.68 (t, J=19.2 Hz, 3H); IR (thin film) 1715, 1169, 765 cm⁻¹; ESIMS m/z 237.2 ([M−F]⁻).

### Example 48: Preparation of 1,3-dibromo-5-(2,2,2-trifluoroethoxy)benzene (C109)

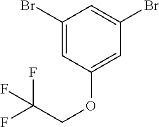 [SMILES: FC(F)(F)COc1cc(Br)cc(Br)c1]

3,5-Dibromophenol (4 g, 15.88 mmol) was dissolved in acetone (79 mL), to which were added potassium carbonate (4.39 g, 31.8 mmol), followed by 2,2,2-trifluoroethyl trifluoromethanesulfonate (3.43 mL, 23.82 mmol). The reaction mixture was stirred overnight at 23° C. The mixture was filtered through diatomaceous earth and concentrated under vacuum. The resulting material, which was spectroscopically pure and used without further purification, was isolated as a clear, colorless oil (3.60 g, 68%): ¹H NMR (400 MHz, CDCl₃) δ 7.37 (t, J=1.5 Hz, 1H), 7.26 (s, OH), 4.33 (q, J=7.9 Hz, 1H); ¹⁹F NMR (376 MHz, CDCl₃) δ −73.86; IR (thin film) 1582, 1562 cm⁻¹; EIMS m/z 333.9.

### Example 49: Preparation of (E)-4-(3-(3,4-dichloro-5-vinylphenyl)-4,4,4-trifluorobut-1-en-1-yl)-2-(trifluoromethyl)benzoic acid (C110)

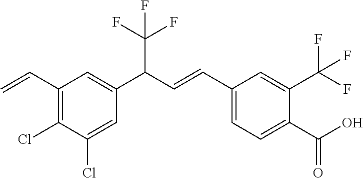 [SMILES: C=Cc1cc(C(/C=C/c2ccc(C(=O)O)c(C(F)(F)F)c2)C(F)(F)F)cc(Cl)c1Cl]

Tetrakis(triphenylphosphine)palladium(O) (0.073 g, 0.063 mmol) was added to a solution of (E)-4-(3-(3-bromo-4,5-dichlorophenyl)-4,4,4-trifluorobut-1-en-1-yl)-2-(trifluoromethyl)benzoic acid (C84) (0.3 g, 0.628 mmol) in toluene (3 mL) at room temperature. The reaction mixture was degassed by purging with nitrogen (3×10 minutes). Tributyl vinyl stannane (0.40 g, 1.26 mmol) was added to the reaction mixture. The reaction mixture was again degassed by purging with nitrogen (3×10 minutes) and was stirred at 120° C. for 3 hours. The reaction mixture was quenched with water and then extracted with ethyl acetate. The organic layer was dried over sodium sulfate, filtered, and concentrated. Purification by flash column chromatography using 60% ethyl acetate/hexanes provided the title compound as a yellow wax (0.19 g, 65%): ¹H NMR (400 MHz, CDCl₃) δ 9.23 (s, 1H), 7.96 (d, J=8.1 Hz, 1H), 7.76 (s, 1H), 7.63 (dd, J=8.2, 1.7 Hz, 1H), 7.51-7.38 (m, 2H), 7.15-7.05 (m, 1H), 6.73-6.63 (m, 1H), 6.60-6.47 (m, 1H), 5.85-5.66 (m, 1H), 5.53-5.41 (m, 1H), 4.33-4.07 (m, 1H); ¹⁹F NMR (376 MHz, CDCl₃) δ −59.46, −68.53; ESIMS m/z 469 ([M−H]⁻).

The following compound was prepared in like manner to the procedure outlined in Example 49:

### (E)-4-(3-(3,4-Dichloro-5-cyclopropylphenyl)-4,4,4-trifluorobut-1-en-1-yl)-2-(trifluoromethyl)benzoic acid (C111)

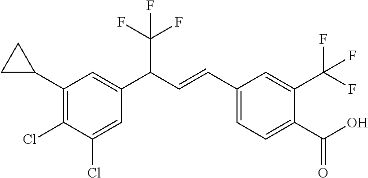 [SMILES: O=C(O)c1ccc(/C=C/C(c2cc(Cl)c(Cl)c(C3CC3)c2)C(F)(F)F)cc1C(F)(F)F]

Isolated as a yellow wax (0.301 g, 73%): ¹H NMR (400 MHz, CDCl₃) δ 9.77 (br s, 1H), 7.97 (s, 1H), 7.76 (s, 1H), 7.63 (d, J=8.1 Hz, 1H), 7.33 (d, J=2.1 Hz, 1H), 6.86 (d, J=2.1 Hz, 1H), 6.71-6.37 (m, 2H), 4.25-4.00 (m, 1H), 2.30-2.17 (m, 1H), 1.41-1.30 (m, 2H), 0.92 (td, J=7.3, 1.3 Hz, 2H); ¹⁹F NMR (376 MHz, CDCl₃) δ −59.53, −68.59; ESIMS m/z 483 ([M−H]⁻).

### Example 50: Preparation of i-bromo-5-(1-bromo-2,2,2-trifluoroethyl)-2,3-dichlorobenzene (C112)

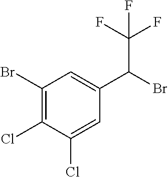 [SMILES: FC(F)(F)C(Br)c1cc(Cl)c(Cl)c(Br)c1]

N-Bromosuccinimide (12.0 g, 67.5 mmol) was added to a solution of 1-(3-bromo-4,5-dichlorophenyl)-2,2,2-trifluoroethanol (5.7 g, 17.6 mmol) in dichloromethane (176 mL). To this stirred solution was added triphenyl phosphite (17.1 mL, 65.3 mmol) slowly, dropwise, and the reaction mixture became dark brown. The reaction mixture was then heated at reflux for 3 hours. The solvent was concentrated, and the residue was triturated with diethyl ether. The solid was filtered, the filtrate was concentrated and the resultant oil was purified by flash column chromatography using hexanes as eluent to provide the title compound as a yellow oil (4.5 g, 46%): ¹H NMR (400 MHz, CDCl₃) δ 7.58 (d, J=2.1 Hz, 1H), 7.46 (d, J=2.1 Hz, 1H), 4.35 (s, 1H); ¹⁹F NMR (376 MHz, CDCl₃) δ −70.40; ESIMS m/z 386 ([M−H]⁻).

### Example 51: Preparation of (E)-2-Bromo-4-(4,4-difluoro-3-(3,4,5-trichlorophenyl)pent-1-en-1-yl)benzoic acid (C113)

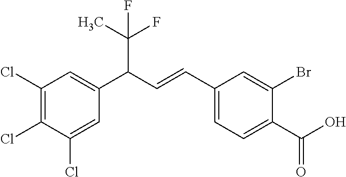 [SMILES: CC(F)(F)C(/C=C/c1ccc(C(=O)O)c(Br)c1)c1cc(Cl)c(Cl)c(Cl)c1]

2,2′-Bipyridine (0.210 g, 1.32 mmol) and copper(I) bromide (0.090 g, 0.66 mmol) were added to a solution of 2-bromo-4-vinyl benzoic acid (1.50 g, 6.61 mmol) in N-methylpyrrolidone (20 mL). The reaction mixture was purged with nitrogen for 5 minutes, then 5-(1-bromo-2,2-difluoropropyl)-1,2,3-trichlorobenzene (C69) (4.47 g, 13.2 mmol) was added and the reaction mixture was heated to 140° C. for 2 hours. The reaction mixture was poured into ice water and the mixture extracted with ethyl acetate. The organic layer was washed with brine, dried over sodium sulfate and concentrated under reduced pressure to afford the crude compound. Purification by column chromatography (SiO₂, 100-200 mesh, eluting with 10% ethyl acetate in hexanes) afforded the title compound as a brown solid (1.60 g, 50%): ¹H NMR (300 MHz, DMSO-d₆) δ 13.38 (br s, 1H), 7.90 (m, 1H), 7.80 (s, 2H), 7.72 (d, J=9.7 Hz, 1H), 7.60 (d, J=9.7 Hz, 1H), 6.96 (dd, J=9.3, 15.6 Hz, 1H), 6.65 (d, J=15.6 Hz, 1H), 4.35-4.23 (m, 1H), 1.60 (t, J=19.3 Hz, 3H); ESIMS m/z 483.1 ([M−H]⁻).

The following compounds were prepared in like manner to the procedure outlined in Example 51:

### (E)-4-(3-(3,5-Dichloro-4-fluorophenyl)-4,4-difluoropent-1-en-1-yl)-2-(trifluoromethyl)benzoic acid (C114)

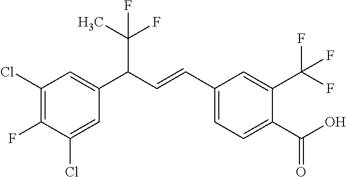 [SMILES: CC(F)(F)C(/C=C/c1ccc(C(=O)O)c(C(F)(F)F)c1)c1cc(Cl)c(F)c(Cl)c1]

Isolated as a yellow gum (1.7 g, 86%): ¹H NMR (400 MHz, CDCl₃) δ 7.96 (d, J=8.4 Hz, 1H), 7.75 (s, 1H), 7.64 (d, J=8.0 Hz, 1H), 7.33 (d, J=6.4 Hz, 2H), 6.62 (dd, J=8.4, 16.0 Hz, 1H), 6.49 (d, J=15.6 Hz, 1H), 3.84-3.74 (m, 1H), 1.65 (t, J=18.8 Hz, 3H); IR (thin film) 3502, 2927, 1712, 809 cm⁻¹; ESIMS m/z 455.2 ([M−H]⁻).

### (E)-2-Chloro-4-(3-(3,5-dichlorophenyl)-4,4-difluoropent-1-en-1-yl)benzoic acid (C115)

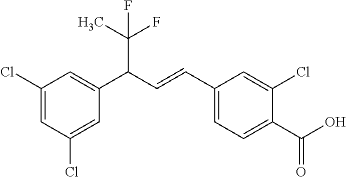 [SMILES: CC(F)(F)C(/C=C/c1ccc(C(=O)O)c(Cl)c1)c1cc(Cl)cc(Cl)c1]

Isolated as a brown gum (3.0 g, 46%): ¹H NMR (400 MHz, CDCl₃) δ 7.99 (d, J=8.0 Hz, 1H), 7.49 (s, 1H), 7.35-7.26 (m, 4H), 6.62 (dd, J=8.4, 16.0 Hz, 1H), 6.49 (d, J=15.6 Hz, 1H), 3.84-3.74 (m, 1H), 1.65 (t, J=18.0 Hz, 3H); ESIMS m/z 403.3 ([M−H]⁻).

### (E)-2-Bromo-4-(3-(3,5-dichlorophenyl)-4,4-difluoropent-1-en-1-yl)benzoic acid (C116)

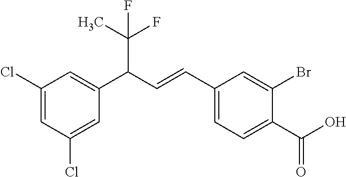 [SMILES: CC(F)(F)C(/C=C/c1ccc(C(=O)O)c(Br)c1)c1cc(Cl)cc(Cl)c1]

Isolated as a brown gum (1.9 g, 55%): ¹H NMR (300 MHz, DMSO-d₆) δ 13.36 (s, 1H), 7.94 (d, J=1.2 Hz, 1H), 7.73 (d, J=8.1 Hz, 1H), 7.61 (d, =9.3 Hz, 2H), 7.57 (s, 2H), 7.01 (dd, =9.3, 15.6 Hz, 1H), 6.67 (d, J=15.9 Hz, 1H), 4.27-4.23 (m, 1H), 1.66 (t, J=18.9 Hz, 3H); IR (thin film) 3455, 1700, 797 cm⁻¹; ESIMS m/z 472.9 ([M+Na]⁺).

### (E)-4-(3-(3,5-Dichlorophenyl)-4,4-difluoropent-1-en-1-yl)-2-(trifluoromethyl)benzoic acid (C117)

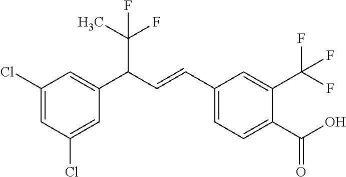 [SMILES: CC(F)(F)C(/C=C/c1ccc(C(=O)O)c(C(F)(F)F)c1)c1cc(Cl)cc(Cl)c1]

Isolated as a brown gum (1.2 g, 35%): ¹H NMR (300 MHz, DMSO-d₆) δ 13.40 (s, 1H), 7.99 (s, 1H), 7.92 (d, J=8.1 Hz, 1H), 7.79 (d, =7.8 Hz, 1H), 7.58 (s, 3H), 7.10 (dd, 3=16.2, 9.6 Hz, 1H), 6.79 (d, J=15.6 Hz, 1H), 4.34-4.12 (m, 1H), 1.67 (t, J=19.2 Hz, 3H); IR (thin film) 3028, 2928, 1710 cm⁻¹; ESIMS m/z 473.35 ([M+H]⁺).

### (E)-4-(4,4-Difluoro-3-(4-fluoro-3-(trifluoromethyl)phenyl)pent-1-en-1-yl)-2-(trifluoromethyl)benzoic acid (C118)

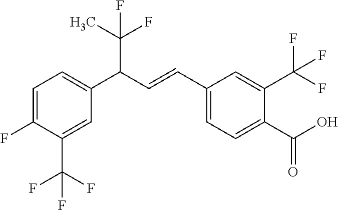 [SMILES: CC(F)(F)C(/C=C/c1ccc(C(=O)O)c(C(F)(F)F)c1)c1ccc(F)c(C(F)(F)F)c1]

Isolated as a brown gum (1.0 g, 43%): ¹H NMR (400 MHz, DMSO-d₆) b 13.50 (s, 1H), 7.97 (s, 1H), 7.92-7.86 (m, 3H), 7.79 (d, J=8.0 Hz, 1H), 7.57 (t, J=10.4 Hz, 1H), 7.01 (dd, J=16.4, 9.2 Hz, 1H), 6.78 (d, J=16.0 Hz, 1H), 4.43-4.34 (m, 1H), 1.68 (t, J=19.2 Hz, 3H); IR (thin film) 3445, 1711, 750 cm⁻¹; ESIMS m/z 454.81 ([M−H]⁻).

### (E)-4-(4,4-Difluoro-3-(3,4,5-trifluorophenyl)pent-1-en-1-yl)-2-(trifluoromethyl)benzoic acid (C119)

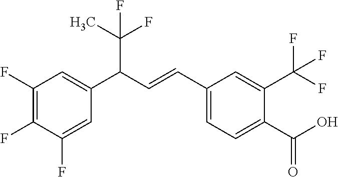 [SMILES: CC(F)(F)C(/C=C/c1ccc(C(=O)O)c(C(F)(F)F)c1)c1cc(F)c(F)c(F)c1]

Isolated as a yellow gum (0.25 g, 37%): ¹H NMR (300 MHz, DMSO-d₆) δ 13.80 (br s, 1H), 7.98 (br s, 1H), 7.90 (d, J=9.0 Hz, 1H), 7.78 (d, J=8.1 Hz, 1H), 7.52-7.47 (m, 2H), 7.00 (d, J=9.0 Hz, 1H), 6.75 (d, J=16.9 Hz, 1H), 4.26-4.21 (m, 1H), 1.61 (t, J=18.9 Hz, 3H); IR (thin film) 2924, 1712, 1533, 1139 cm⁻¹; ESIMS m/z 422.77 ([M+H]⁺).

### Example 52: Preparation of tert-butyl (R)-(1-((2,2,2-trifluoroethyl)sulfonyl)propan-2-yl)carbamate (C120)

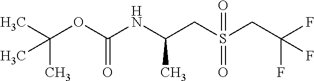 [SMILES: C[C@H](CS(=O)(=O)CC(F)(F)F)NC(=O)OC(C)(C)C]

To (R)-tert-butyl (1-((2,2,2-trifluoroethyl)thio)propan-2-yl)carbamate (C53) (5.00 g, 18.3 mmol) and acetic acid (183 mL). was added sodium perborate monohydrate (5.48 g, 54.9 mmol). The reaction mixture was heated at 55° C., then after 2 hours the mixture was concentrated in vacuo. The reaction mixture was taken up in ethyl acetate. The heterogeneous mixture was filtered and the solids washed with ethyl acetate. The combined filtrate was concentrated in vacuo. The title compound was isolated as a white solid (4.6 g, 78%): ¹H NMR (400 MHz, CDCl₃) δ 4.81 (s, 1H), 4.25-4.18 (m, 1H), 4.10-3.90 (m, 1H), 3.48 (dd, J=8.0, 14.0 Hz, 1H), 3.22 (dd, J=4.0, 16.0 Hz, 1H), 1.45 (s, 1H), 1.41 (d, J=8.0 Hz, 3H); ¹⁹F NMR (376 MHz, CDCl₃) δ −61.14; ESIMS m/z 304.0 ([M−H]⁻).

### Example 53: Preparation of tert-butyl (R)-(1-((2-fluoroethyl)thio)propan-2-yl)carbamate (C121)

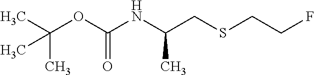

Aqueous sodium hydroxide (2.0 M, 8.5 mL, 17 mmol) was added to a solution of (R)—S-(2-((tert-butoxycarbonyl)amino)propyl)ethanethioate (2.0 g, 8.5 mmol) in isopropanol at 0° C. After stirring 2 hours, 1-fluoro-2-iodoethane (1.76 g, 10.1 mmol) was added and the reaction mixture was left to stir overnight. The reaction mixture was partioned between ethyl acetate and water. The organics were separated, washed with saturated sodium bicarbonate and brine, dried with magnesium sulfate, and concentrated. The resulting residue was purified by flash silica chromatography. The title compound was isolated as a pale yellow liquid (1.55 g 72%): ¹H NMR (500 MHz, DMSO-d₆) δ 6.80 (d, J=8.2 Hz, 1H), 4.53 (dt, J=47.3, 6.4 Hz, 2H), 3.54 (h, J=6.9 Hz, 1H), 2.81 (dt, J=21.2, 6.4 Hz, 2H), 2.63-2.51 (m, 2H), 1.38 (s, 9H), 1.07 (d, J=6.7 Hz, 3H); ¹⁹F NMR (471 MHz, DMSO-d₆) δ −211.55 (tt, J=46.9, 21.3 Hz); ¹³C NMR (126 MHz, DMSO-d₆) δ 155.41, 83.20 (d, J=167.3 Hz), 78.02, 46.51, 38.34, 31.84 (d, J=20.5 Hz), 28.72, 20.19; IR (thin film) 3340, 2975, 2931, 1687, 1504 cm⁻¹.

### Example 54: Preparation of tert-butyl (R)-(1-((2-fluoroethyl)thio)propan-2-yl)carbamate (C122)

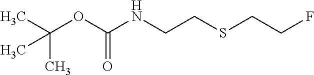

Sodium hydride (60% dispersion in paraffin, 1.0 g, 25 mmol) was added to tert-butyl (2-mercaptoethyl)carbamate (4 g, 22.57 mmol) In N,N-dimethylformamide (59.8 mL) at 0° C. After stirring for 20 minutes 1-fluoro-2-iodoethane (2.6 g, 14.95 mmol) was added and the reaction mixture was left to stir overnight. The reaction mixture was diluted with water and extracted with diethyl ether. The organic layer was washed with water and brine, dried with magnesium sulfate, filtered, and concentrated. The resulting residue was purified by flash silica chromatography. The title compound was isolated as a colorless liquid (1.5 g, 43%): ¹H NMR (500 MHz, DMSO-d₆) δ 6.98-6.86 (m, 1H), 4.53 (dt, J=47.3, 6.3 Hz, 2H), 3.08 (dt, J=7.6, 6.1 Hz, 2H), 2.81 (dt, J=21.7, 6.3 Hz, 2H), 2.57 (dd, J=7.9, 6.4 Hz, 2H), 1.38 (s, 9H); ¹³C NMR (126 MHz, DMSO-d₆) δ 155.96, 83.26 (d, J=167.6 Hz), 78.17, 40.40, 31.66, 31.27 (d, J=20.7 Hz), 28.69; ¹⁹F NMR (471 MHz, DMSO-d₆) δ −211.77 (tt, J=47.3, 21.7 Hz); IR (thin film) 1689 cm⁻¹; HRMS-ESI (m/z) [M+Na]⁺ calcd for C₉H₁₈FNO₂S, 246.0934; found, 246.0937.

### Example 55: Preparation of tert-butyl (R)-(1-amino-1-oxo-3-((2,2,2-trifluoroethyl)thio)propan-2-yl)carbamate (C123)

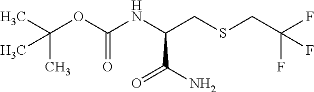 [SMILES: CC(C)(C)OC(=O)N[C@@H](CSCC(F)(F)F)C(N)=O]

Concentrated ammonium hydroxide (10 mL, 180 mmol) was added to a solution of (R)-methyl 2-((tert-butoxycarbonyl)amino)-3-((2,2,2-trifluoroethyl)thio)propanoate (1.5 g, 4.73 mmol) in toluene (10 mL) to give a biphasic solution, which was stirred rapidly for 34 hours. The biphasic solution was concentrated in vacuo at 60° C. to yield a white amorphous solid (1.3 g, 86%): ¹H NMR (400 MHz, CD₃CN) δ 6.50 (s, 1H), 5.94 (s, 1H), 5.72 (s, 1H), 3.31 (qd, J=10.4, 4.7 Hz, 2H), 3.11 (dd, J=13.8, 5.1 Hz, 1H), 2.90 (dd, J=13.7, 8.2 Hz, 1H), 1.45 (s, 9H); ¹⁹F NMR (376 MHz, CD₃CN) 6-67.29; ESIMS m/z 301 ([M−H]⁻).

### Example 56: Preparation of methyl N-(tert-butoxycarbonyl)-S-(2,2,2-trifluoroethyl)-L-cysteinate (C124)

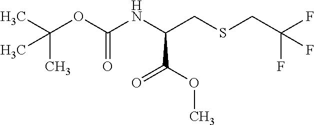 [SMILES: COC(=O)[C@H](CSCC(F)(F)F)NC(=O)OC(C)(C)C]

Cesium carbonate (15.23 g, 46.7 mmol) was added portionwise to a solution of 2,2,2-trifluoroethyl trifluoromethanesulfonate (10.85 g, 46.7 mmol) and (R)-methyl 2-((tert-butoxycarbonyl)amino)-3-mercaptopropanoate (11 g, 46.7 mmol) in degassed N,N-dimethylformamide (35 mL). The reaction suspension was stirred for three days. The reaction mixture was diluted with ethyl acetate and water. The layers were separated and the organic layer was washed with water. The organic layer was dried with magnesium sulfate, filtered, and concentrated at 50° C. The residue was purified by flash silica chromatography. The title compound was isolated as an orange solid (5.7 g, 36.5%): ¹H NMR (400 MHz, CDCl₃) δ 5.33 (d, J=7.5 Hz, 1H), 4.76-4.33 (m, 1H), 3.79 (s, 3H), 3.33-2.89 (m, 4H), 1.46 (s, 9H); ¹³C NMR (101 MHz, CDCl₃) δ 171.05, 155.08, 125.64 (q, J=276.4 Hz), 80.48, 53.21, 52.77, 35.30, 34.57 (q, J=32.9 Hz), 28.26; ¹⁹F NMR (376 MHz, CDCl₃) δ −66.60.

### Biological Assays

The following bioassays against Beet Armyworm (Spodoptera exigua), Cabbage Looper (Trichoplusia ni), Corn Earworm (Helicoverpa zea), Green Peach Aphid (Myzus persicae), and Yellow Fever Mosquito (Aedes aegypti), are included herein due to the damage they inflict. Furthermore, the Beet Armyworm, Corn Earworm, and Cabbage Looper are three good indicator species for a broad range of chewing pests. Additionally, the Green Peach Aphid is a good indicator species for a broad range of sap-feeding pests. The results with these four indicator species along with the Yellow Fever Mosquito show the broad usefulness of the molecules of Formula One in controlling pests in Phyla Arthropoda, Mollusca, and Nematoda (For further information see Methods for the Design and Optimization of New Active Ingredients, Modern Methods in Crop Protection Research, Edited by Jeschke, P., Kramer, W., Schirmer, U., and Matthias W., p. 1-20, 2012).

### Example A: Bioassays On Beet Armyworm (Spodoptera exigua, LAPHEG) (“BAW”), Corn Earworm (Helicoverpa zea, HELIZE) (“CEW”), And Cabbage Looper (Trichoplusia ni, TRIPNI) (“CL”)

Beet armyworm is a serious pest of economic concern for alfalfa, asparagus, beets, citrus, corn, cotton, onions, peas, peppers, potatoes, soybeans, sugar beets, sunflowers, tobacco, tomatoes, among other crops. It is native to Southeast Asia but is now found in Africa, Australia, Japan, North America, and Southern Europe. The larvae may feed in large swarms causing devastating crop losses. It is known to be resistant to several pesticides.

Cabbage looper is a serious pest found throughout the world. It attacks alfalfa, beans, beets, broccoli, Brussel sprouts, cabbage, cantaloupe, cauliflower, celery, collards, cotton, cucumbers, eggplant, kale, lettuce, melons, mustard, parsley, peas, peppers, potatoes, soybeans, spinach, squash, tomatoes, turnips, and watermelons, among other crops. This species is very destructive to plants due to its voracious appetite. The larvae consume three times their weight in food daily. The feeding sites are marked by large accumulations of sticky, wet, fecal material. It is known to be resistant to several pesticides.

Corn earworm is considered by some to be the most costly crop pest in North America. It often attacks valuable crops, and the harvested portion of the crop. This pest damages alfalfa, artichoke, asparagus, cabbage, cantaloupe, collard, corn, cotton, cowpea, cucumber, eggplant, lettuce, lima bean, melon, okra, pea, pepper, potato, pumpkin, snap bean, soybean, spinach, squash, sugarcane, sweet potato, tomato, and watermelon, among other crops. Furthermore, this pest is also known to be resistant to certain insecticides.

Consequently, because of the above factors control of these pests is important. Furthermore, molecules that control these pests (BAW, CEW, and CL), which are known as chewing pests, are useful in controlling other pests that chew on plants.

Certain molecules disclosed in this document were tested against BAW, CEW, and CL using procedures described in the following examples. In the reporting of the results, the “BAW, CEW, & CL Rating Table” was used (See Table Section).

### Bioassays on BAW

Bioassays on BAW were conducted using a 128-well diet tray assay. One to five second instar BAW larvae were placed in each well (3 mL) of the diet tray that had been previously filled with 1 mL of artificial diet to which 50 μg/cm² of the test molecule (dissolved in 50 μL of 90:10 acetone-water mixture) had been applied (to each of eight wells) and then allowed to dry. Trays were covered with a clear self-adhesive cover and held at 25° C., 14:10 light-dark for five to seven days. Percent mortality was recorded for the larvae in each well; activity in the eight wells was then averaged. The results are indicated in the table entitled “Table ABC: Biological Results” (See Table Section).

### Bioassays On CL

Bioassays on CL were conducted using a 128-well diet tray assay.

One to five second instar CL larvae were placed in each well (3 mL) of the diet tray that had been previously filled with 1 mL of artificial diet to which 50 μg/cm² of the test molecule (dissolved in 50 μL of 90:10 acetone-water mixture) had been applied (to each of eight wells) and then allowed to dry. Trays were covered with a clear self-adhesive cover and held at 25° C., 14:10 light-dark for five to seven days. Percent mortality was recorded for the larvae in each well; activity in the eight wells was then averaged. The results are indicated in the table entitled “Table ABC: Biological Results” (See Table Section).

### Example B: Bioassays On Green Peach Aphid (Myus persicae, MYZUPE) (“GPA”)

GPA is the most significant aphid pest of peach trees, causing decreased growth, shriveling of the leaves, and the death of various tissues. It is also hazardous because it acts as a vector for the transport of plant viruses, such as potato virus Y and potato leafroll virus to members of the nightshade/potato family Solanaceae, and various mosaic viruses to many other food crops. GPA attacks such plants as broccoli, burdock, cabbage, carrot, cauliflower, daikon, eggplant, green beans, lettuce, macadamia, papaya, peppers, sweet potatoes, tomatoes, watercress, and zucchini, among other crops. GPA also attacks many ornamental crops such as carnation, chrysanthemum, flowering white cabbage, poinsettia, and roses. GPA has developed resistance to many pesticides. Consequently, because of the above factors control of this pest is important. Furthermore, molecules that control this pest (GPA), which is known as a sap-feeding pest, are useful in controlling other pests that feed on the sap from plants.

Certain molecules disclosed in this document were tested against GPA using procedures described in the following example. In the reporting of the results, the “GPA & YFM Rating Table” was used (See Table Section).

Cabbage seedlings grown in 3-inch pots, with 2-3 small (3-5 cm) true leaves, were used as test substrate. The seedlings were infested with 20-50 GPA (wingless adult and nymph stages) one day prior to chemical application. Four pots with individual seedlings were used for each treatment. Test molecules (2 mg) were dissolved in 2 mL of acetone/methanol (1:1) solvent, forming stock solutions of 1000 ppm test molecule. The stock solutions were diluted 5× with 0.025% Tween 20 in water to obtain the solution at 200 ppm test molecule. A hand-held aspirator-type sprayer was used for spraying a solution to both sides of cabbage leaves until runoff. Reference plants (solvent check) were sprayed with the diluent only containing 20% by volume of acetone/methanol (1:1) solvent. Treated plants were held in a holding room for three days at approximately 25° C. and ambient relative humidity (RH) prior to grading. Evaluation was conducted by counting the number of live aphids per plant under a microscope. Percent Control was measured by using Abbott's correction formula (W. S. Abbott, “A Method of Computing the Effectiveness of an Insecticide” J. Econ. Entomol. 18 (1925), pp. 265-267) as follows.

Corrected % Control=100*(X−Y)/X
where
X=No. of live aphids on solvent check plants and
Y=No. of live aphids on treated plants

The results are indicated in the table entitled “Table ABC: Biological Results” (See Table Section).

### Example C: Bioassays On Yellow Fever Mosquito (Aedes aegypti, AEDSAE) (“YFM”)

YFM prefers to feed on humans during the daytime and is most frequently found in or near human habitations. YFM is a vector for transmitting several diseases. It is a mosquito that can spread the dengue fever and yellow fever viruses. Yellow fever is the second most dangerous mosquito-borne disease after malaria. Yellow fever is an acute viral hemorrhagic disease and up to 50% of severely affected persons without treatment will die from yellow fever. There are an estimated 200,000 cases of yellow fever, causing 30,000 deaths, worldwide each year. Dengue fever is a nasty, viral disease; it is sometimes called “breakbone fever” or “break-heart fever” because of the intense pain it can produce. Dengue fever kills about 20,000 people annually. Consequently, because of the above factors control of this pest is important. Furthermore, molecules that control this pest (YFM), which is known as a sucking pest, are useful in controlling other pests that cause human and animal suffering.

Certain molecules disclosed in this document were tested against YFM using procedures described in the following paragraph. In the reporting of the results, the “GPA & YFM Rating Table” was used (See Table Section).

Master plates containing 400 μg of a molecule dissolved in 100 μL of dimethyl sulfoxide (DMSO) (equivalent to a 4000 ppm solution) are used. A master plate of assembled molecules contains 15 μL per well. To this plate, 135 μL of a 90:10 water:acetone mixture is added to each well. A robot (Biomek® NXP Laboratory Automation Workstation) is programmed to dispense 15 μL aspirations from the master plate into an empty 96-well shallow plate (“daughter” plate). There are 6 reps (“daughter” plates) created per master. The created daughter plates are then immediately infested with YFM larvae.

The day before plates are to be treated, mosquito eggs are placed in Millipore water containing liver powder to begin hatching (4 g. into 400 mL). After the daughter plates are created using the robot, they are infested with 220 μL of the liver powder/larval mosquito mixture (about 1 day-old larvae). After plates are infested with mosquito larvae, a non-evaporative lid is used to cover the plate to reduce drying. Plates are held at room temperature for 3 days prior to grading. After 3 days, each well is observed and scored based on mortality. The results are indicated in the table entitled “Table ABC: Biological Results” (See Table Section). AGRICULTURALLY ACCEPTABLE ACID ADDITION SALTS, SALT DERIVATIVES, SOLVATES, ESTER DERIVATIVES, POLYMORPHS, ISOTOPES, AND RADIONUCLIDES

Molecules of Formula One may be formulated into agriculturally acceptable acid addition salts. By way of a non-limiting example, an amine function can form salts with hydrochloric, hydrobromic, sulfuric, phosphoric, acetic, benzoic, citric, malonic, salicylic, malic, fumaric, oxalic, succinic, tartaric, lactic, gluconic, ascorbic, maleic, aspartic, benzenesulfonic, methanesulfonic, ethanesulfonic, hydroxyl-methanesulfonic, and hydroxyethanesulfonic acids. Additionally, by way of a non-limiting example, an acid function can form salts including those derived from alkali or alkaline earth metals and those derived from ammonia and amines. Examples of preferred cations include sodium, potassium, and magnesium.

Molecules of Formula One may be formulated into salt derivatives. By way of a non-limiting example, a salt derivative may be prepared by contacting a free base with a sufficient amount of the desired acid to produce a salt. A free base may be regenerated by treating the salt with a suitable dilute aqueous base solution such as dilute aqueous sodium hydroxide, potassium carbonate, ammonia, and sodium bicarbonate. As an example, in many cases, a pesticide, such as 2,4-D, is made more water-soluble by converting it to its dimethylamine salt.

Molecules of Formula One may be formulated into stable complexes with a solvent, such that the complex remains intact after the non-complexed solvent is removed. These complexes are often referred to as “solvates.” However, it is particularly desirable to form stable hydrates with water as the solvent.

Molecules of Formula One may be made into ester derivatives. These ester derivatives can then be applied in the same manner as the molecules disclosed in this document is applied.

Molecules of Formula One may be made as various crystal polymorphs. Polymorphism is important in the development of agrochemicals since different crystal polymorphs or structures of the same molecule can have vastly different physical properties and biological performances.

Molecules of Formula One may be made with different isotopes. Of particular importance are molecules having ²H (also known as deuterium) or ³H (also known as tritium) in place of 1H. Molecules of Formula One may be made with different radionuclides. Of particular importance are molecules having ¹⁴C. Molecules of Formula One having deuterium, tritium, or ¹⁴C may be used in biological studies allowing tracing in chemical and physiological processes and half-life studies, as well as, MoA studies.

### Stereoisomers

Molecules of Formula One may exist as one or more stereoisomers. Thus, certain molecules may be produced as racemic mixtures. It will be appreciated by those skilled in the art that one stereoisomer may be more active than the other stereoisomers. Individual stereoisomers may be obtained by known selective synthetic procedures, by conventional synthetic procedures using resolved starting materials, or by conventional resolution procedures. Certain molecules disclosed in this document can exist as two or more isomers. The various isomers include geometric isomers, diastereomers, and enantiomers. Thus, the molecules disclosed in this document include geometric isomers, racemic mixtures, individual stereoisomers, and optically active mixtures. It will be appreciated by those skilled in the art that one isomer may be more active than the others. The structures disclosed in the present disclosure are drawn in only one geometric form for clarity, but are intended to represent all geometric forms of the molecule.

### Combinations

In another embodiment of this invention, molecules of Formula One may be used in combination (such as, in a compositional mixture, or a simultaneous or sequential application) with one or more active ingredients.

In another embodiment of this invention, molecules of Formula One may be used in combination (such as, in a compositional mixture, or a simultaneous or sequential application) with one or more active ingredients each having a MoA that is the same as, similar to, but more likely—different from, the MoA of the molecules of Formula One.

In another embodiment, molecules of Formula One may be used in combination (such as, in a compositional mixture, or a simultaneous or sequential application) with one or more molecules having acaricidal, algicidal, avicidal, bactericidal, fungicidal, herbicidal, insecticidal, molluscicidal, nematicidal, rodenticidal, and/or virucidal properties.

In another embodiment, the molecules of Formula One may be used in combination (such as, in a compositional mixture, or a simultaneous or sequential application) with one or more molecules that are antifeedants, bird repellents, chemosterilants, herbicide safeners, insect attractants, insect repellents, mammal repellents, mating disrupters, plant activators, plant growth regulators, and/or synergists.

In another embodiment, molecules of Formula One may also be used in combination (such as in a compositional mixture, or a simultaneous or sequential application) with one or more biopesticides.

In another embodiment, in a pesticidal composition combinations of a molecule of Formula One and an active ingredient may be used in a wide variety of weight ratios. For example, in a two component mixture, the weight ratio of a molecule of Formula One to an active ingredient, may be from about 100:1 to about 1:100; in another example the weight ratio may be about 50:1 to about 1:50; in another example the weight ratio may be about 20:1 to about 1:20; in another example the weight ratio may be about 10:1 to about 1:10; in another example the weight ratio may be about 5:1 to 1:5; in another example the weight ratio may be about 3:1 to about 1:3; in another example the weight ratio may be about 2:1 to about 1:2; and in a final example the weight ratio may be about 1:1 (See Table B). However, in general, weight ratios less than about 10:1 to about 1:10 are preferred. It is also preferred sometimes to use a three or four component mixture comprising a molecule of Formula One and one or more active ingredients.

TABLE B
Weight Ratios
Molecule of the Formula One:active ingredient
100:1 to 1:100
50:1 to 1:50
20:1 to 1:20
10:1 to 1:10
5:1 to 1:5
3:1 to 1:3
2:1 to 1:2
1:1

Weight ratios of a molecule of Formula One to an active ingredient may also be depicted as X:Y; wherein X is the parts by weight of a molecule of Formula One and Y is the parts by weight of active ingredient. The numerical range of the parts by weight for X is 0<X≦100 and the parts by weight for Y is 0<Y≦100 and is shown graphically in TABLE C. By way of non-limiting example, the weight ratio of a molecule of Formula One to an active ingredient may be 20:1.

TABLE C

| | | | | | | | | | | |
|---|---|---|---|---|---|---|---|---|---|---|
| active | 100 | X, Y | | X, Y | | | X, Y | | | |
| ingredient | 50 | X, Y | X, Y | X, Y | | | X, Y | X, Y | | |
| (Y) | 20 | X, Y | | X, Y | X, Y | | X, Y | | X, Y | |
| Parts by | 15 | X, Y | X, Y | | | | | X, Y | X, Y | X, Y |
| weight | 10 | X, Y | | X, Y | | | | | | |
| | 5 | X, Y | X, Y | X, Y | | | | X, Y | | |
| | 3 | X, Y | X, Y | | X, Y | X, Y | | X, Y | X, Y | X, Y |
| | 2 | X, Y | | X, Y | X, Y | | X, Y | | X, Y | |
| | 1 | X, Y | X, Y | X, Y | X, Y | X, Y | X, Y | X, Y | X, Y | X, Y |
| | | 1 | 2 | 3 | 5 | 10 | 15 | 20 | 50 | 100 |

molecule of Formula One (X) Parts by weight

Ranges of weight ratios of a molecule of Formula One to an active ingredient may be depicted as X₁:Y₁ to X₂:Y₂, wherein X and Y are defined as above.

In one embodiment, the range of weight ratios may be X₁:Y₁ to X₂:Y₂, wherein X₁>Y₁ and X₂<Y₂. By way of non-limiting example, the range of a weight ratio of a molecule of Formula One to an active ingredient may be between 3:1 and 1:3, inclusive of the endpoints.

In another embodiment, the range of weight ratios may be X₁:Y₁ to X₂:Y₂, wherein X₁>Y₁ and X₂>Y₂. By way of non-limiting example, the range of weight ratio of a molecule of Formula One to an active ingredient may be between 15:1 and 3:1, inclusive of the endpoints.

In another embodiment, the range of weight ratios may be X₁:Y₁ to X₂:Y₂, wherein X₁<Y₁ and X₂<Y₂. By way of non-limiting example, the range of weight ratios of a molecule of Formula One to an active ingredient may be between about 1:3 and about 1:20, inclusive of the endpoints.

### Formulations

A pesticide is rarely suitable for application in its pure form. It is usually necessary to add other substances so that the pesticide may be used at the required concentration and in an appropriate form, permitting ease of application, handling, transportation, storage, and maximum pesticide activity. Thus, pesticides are formulated into, for example, baits, concentrated emulsions, dusts, emulsifiable concentrates, fumigants, gels, granules, microencapsulations, seed treatments, suspension concentrates, suspoemulsions, tablets, water soluble liquids, water dispersible granules or dry flowables, wettable powders, and ultra-low volume solutions.

Pesticides are applied most often as aqueous suspensions or emulsions prepared from concentrated formulations of such pesticides. Such water-soluble, water-suspendable, or emulsifiable formulations are either solids, usually known as wettable powders, or water dispersible granules, or liquids usually known as emulsifiable concentrates, or aqueous suspensions. Wettable powders, which may be compacted to form water dispersible granules, comprise an intimate mixture of the pesticide, a carrier, and surfactants. The concentration of the pesticide is usually from about 10% to about 90% by weight. The carrier is usually selected from among the attapulgite clays, the montmorillonite clays, the diatomaceous earths, or the purified silicates. Effective surfactants, comprising from about 0.5% to about 10% of the wettable powder, are found among sulfonated lignins, condensed naphthalenesulfonates, naphthalenesulfonates, alkylbenzenesulfonates, alkyl sulfates, and non-ionic surfactants such as ethylene oxide adducts of alkyl phenols.

Emulsifiable concentrates of pesticides comprise a convenient concentration of a pesticide, such as from about 50 to about 500 grams per liter of liquid dissolved in a carrier that is either a water miscible solvent or a mixture of water-immiscible organic solvent and emulsifiers. Useful organic solvents include aromatics, especially xylenes and petroleum fractions, especially the high-boiling naphthalenic and olefinic portions of petroleum such as heavy aromatic naphtha. Other organic solvents may also be used, such as the terpenic solvents including rosin derivatives, aliphatic ketones such as cyclohexanone, and complex alcohols such as 2-ethoxyethanol. Suitable emulsifiers for emulsifiable concentrates are selected from conventional anionic and non-ionic surfactants.

Aqueous suspensions comprise suspensions of water-insoluble pesticides dispersed in an aqueous carrier at a concentration in the range from about 5% to about 50% by weight. Suspensions are prepared by finely grinding the pesticide and vigorously mixing it into a carrier comprised of water and surfactants. Ingredients, such as inorganic salts and synthetic or natural gums may also be added, to increase the density and viscosity of the aqueous carrier. It is often most effective to grind and mix the pesticide at the same time by preparing the aqueous mixture and homogenizing it in an implement such as a sand mill, ball mill, or piston-type homogenizer.

Pesticides may also be applied as granular compositions that are particularly useful for applications to the soil. Granular compositions usually contain from about 0.5% to about 10% by weight of the pesticide, dispersed in a carrier that comprises clay or a similar substance. Such compositions are usually prepared by dissolving the pesticide in a suitable solvent and applying it to a granular carrier which has been pre-formed to the appropriate particle size, in the range of from about 0.5 to about 3 mm. Such compositions may also be formulated by making a dough or paste of the carrier and molecule and crushing and drying to obtain the desired granular particle size.

Dusts containing a pesticide are prepared by intimately mixing the pesticide in powdered form with a suitable dusty agricultural carrier, such as kaolin clay, ground volcanic rock, and the like. Dusts can suitably contain from about 1% to about 10% of the pesticide. Dusts may be applied as a seed dressing or as a foliage application with a dust blower machine.

It is equally practical to apply a pesticide in the form of a solution in an appropriate organic solvent, usually petroleum oil, such as the spray oils, which are widely used in agricultural chemistry.

Pesticides can also be applied in the form of an aerosol composition. In such compositions the pesticide is dissolved or dispersed in a carrier, which is a pressure-generating propellant mixture. The aerosol composition is packaged in a container from which the mixture is dispensed through an atomizing valve.

Pesticide baits are formed when the pesticide is mixed with food or an attractant or both. When the pests eat the bait they also consume the pesticide. Baits may take the form of granules, gels, flowable powders, liquids, or solids. Baits may be used in pest harborages.

Fumigants are pesticides that have a relatively high vapor pressure and hence can exist as a gas in sufficient concentrations to kill pests in soil or enclosed spaces. The toxicity of the fumigant is proportional to its concentration and the exposure time. They are characterized by a good capacity for diffusion and act by penetrating the pest's respiratory system or being absorbed through the pest's cuticle. Fumigants are applied to control stored product pests under gas proof sheets, in gas sealed rooms or buildings or in special chambers.

Pesticides may be microencapsulated by suspending the pesticide particles or droplets in plastic polymers of various types. By altering the chemistry of the polymer or by changing factors in the processing, microcapsules may be formed of various sizes, solubility, wall thicknesses, and degrees of penetrability. These factors govern the speed with which the active ingredient within is released, which in turn, affects the residual performance, speed of action, and odor of the product.

Oil solution concentrates are made by dissolving pesticide in a solvent that will hold the pesticide in solution. Oil solutions of a pesticide usually provide faster knockdown and kill of pests than other formulations due to the solvents themselves having pesticidal action and the dissolution of the waxy covering of the integument increasing the speed of uptake of the pesticide. Other advantages of oil solutions include better storage stability, better penetration of crevices, and better adhesion to greasy surfaces.

Another embodiment is an oil-in-water emulsion, wherein the emulsion comprises oily globules which are each provided with a lamellar liquid crystal coating and are dispersed in an aqueous phase, wherein each oily globule comprises at least one molecule which is agriculturally active, and is individually coated with a monolamellar or oligolamellar layer comprising: (1) at least one non-ionic lipophilic surface-active agent, (2) at least one non-ionic hydrophilic surface-active agent and (3) at least one ionic surface-active agent, wherein the globules having a mean particle diameter of less than 800 nanometers.

### Other Formulation Components

Generally, when the molecules disclosed in Formula One are used in a formulation, such formulation can also contain other components. These components include, but are not limited to, (this is a non-exhaustive and non-mutually exclusive list) wetters, spreaders, stickers, penetrants, buffers, sequestering agents, drift reduction agents, compatibility agents, anti-foam agents, cleaning agents, and emulsifiers. A few components are described forthwith.

A wetting agent is a substance that when added to a liquid increases the spreading or penetration power of the liquid by reducing the interfacial tension between the liquid and the surface on which it is spreading. Wetting agents are used for two main functions in agrochemical formulations: during processing and manufacture to increase the rate of wetting of powders in water to make concentrates for soluble liquids or suspension concentrates; and during mixing of a product with water in a spray tank to reduce the wetting time of wettable powders and to improve the penetration of water into water-dispersible granules. Examples of wetting agents used in wettable powder, suspension concentrate, and water-dispersible granule formulations are: sodium lauryl sulfate; sodium dioctyl sulfosuccinate; alkyl phenol ethoxylates; and aliphatic alcohol ethoxylates.

A dispersing agent is a substance which adsorbs onto the surface of particles and helps to preserve the state of dispersion of the particles and prevents them from reaggregating. Dispersing agents are added to agrochemical formulations to facilitate dispersion and suspension during manufacture, and to ensure the particles redisperse into water in a spray tank. They are widely used in wettable powders, suspension concentrates and water-dispersible granules. Surfactants that are used as dispersing agents have the ability to adsorb strongly onto a particle surface and provide a charged or steric barrier to reaggregation of particles. The most commonly used surfactants are anionic, non-ionic, or mixtures of the two types. For wettable powder formulations, the most common dispersing agents are sodium lignosulfonates. For suspension concentrates, very good adsorption and stabilization are obtained using polyelectrolytes, such as sodium naphthalene sulfonate formaldehyde condensates. Tristyrylphenol ethoxylate phosphate esters are also used. Non-ionics such as alkylarylethylene oxide condensates and EO-PO block copolymers are sometimes combined with anionics as dispersing agents for suspension concentrates. In recent years, new types of very high molecular weight polymeric surfactants have been developed as dispersing agents. These have very long hydrophobic ‘backbones’ and a large number of ethylene oxide chains forming the ‘teeth’ of a ‘comb’ surfactant. These high molecular weight polymers can give very good long-term stability to suspension concentrates because the hydrophobic backbones have many anchoring points onto the particle surfaces. Examples of dispersing agents used in agrochemical formulations are: sodium lignosulfonates; sodium naphthalene sulfonate formaldehyde condensates; tristyrylphenol ethoxylate phosphate esters; aliphatic alcohol ethoxylates; alkyl ethoxylates; EO-PO block copolymers; and graft copolymers.

An emulsifying agent is a substance which stabilizes a suspension of droplets of one liquid phase in another liquid phase. Without the emulsifying agent the two liquids would separate into two immiscible liquid phases. The most commonly used emulsifier blends contain alkylphenol or aliphatic alcohol with twelve or more ethylene oxide units and the oil-soluble calcium salt of dodecylbenzenesulfonic acid. A range of hydrophile-lipophile balance (“HLB”) values from 8 to 18 will normally provide good stable emulsions. Emulsion stability can sometimes be improved by the addition of a small amount of an EO-PO block copolymer surfactant.

A solubilizing agent is a surfactant which will form micelles in water at concentrations above the critical micelle concentration. The micelles are then able to dissolve or solubilize water-insoluble materials inside the hydrophobic part of the micelle. The types of surfactants usually used for solubilization are non-ionics, sorbitan monooleates, sorbitan monooleate ethoxylates, and methyl oleate esters.

Surfactants are sometimes used, either alone or with other additives such as mineral or vegetable oils as adjuvants to spray-tank mixes to improve the biological performance of the pesticide on the target. The types of surfactants used for bioenhancement depend generally on the nature and mode of action of the pesticide. However, they are often non-ionics such as: alkyl ethoxylates; linear aliphatic alcohol ethoxylates; aliphatic amine ethoxylates.

A carrier or diluent in an agricultural formulation is a material added to the pesticide to give a product of the required strength. Carriers are usually materials with high absorptive capacities, while diluents are usually materials with low absorptive capacities. Carriers and diluents are used in the formulation of dusts, wettable powders, granules and water-dispersible granules.

Organic solvents are used mainly in the formulation of emulsifiable concentrates, oil-in-water emulsions, suspoemulsions, and ultra-low volume formulations, and to a lesser extent, granular formulations. Sometimes mixtures of solvents are used. The first main groups of solvents are aliphatic paraffinic oils such as kerosene or refined paraffins. The second main group (and the most common) comprises the aromatic solvents such as xylene and higher molecular weight fractions of C9 and C10 aromatic solvents. Chlorinated hydrocarbons are useful as cosolvents to prevent crystallization of pesticides when the formulation is emulsified into water. Alcohols are sometimes used as cosolvents to increase solvent power. Other solvents may include vegetable oils, seed oils, and esters of vegetable and seed oils.

Thickeners or gelling agents are used mainly in the formulation of suspension concentrates, emulsions and suspoemulsions to modify the rheology or flow properties of the liquid and to prevent separation and settling of the dispersed particles or droplets. Thickening, gelling, and anti-settling agents generally fall into two categories, namely water-insoluble particulates and water-soluble polymers. It is possible to produce suspension concentrate formulations using clays and silicas. Examples of these types of materials, include, but are not limited to, montmorillonite, bentonite, magnesium aluminum silicate, and attapulgite. Water-soluble polysaccharides have been used as thickening-gelling agents for many years. The types of polysaccharides most commonly used are natural extracts of seeds and seaweeds or are synthetic derivatives of cellulose. Examples of these types of materials include, but are not limited to, guar gum; locust bean gum; carrageenam; alginates; methyl cellulose; sodium carboxymethyl cellulose (SCMC); hydroxyethyl cellulose (HEC). Other types of anti-settling agents are based on modified starches, polyacrylates, polyvinyl alcohol and polyethylene oxide. Another good anti-settling agent is xanthan gum.

Microorganisms can cause spoilage of formulated products. Therefore preservation agents are used to eliminate or reduce their effect. Examples of such agents include, but are not limited to: propionic acid and its sodium salt; sorbic acid and its sodium or potassium salts; benzoic acid and its sodium salt; p-hydroxybenzoic acid sodium salt; methyl p-hydroxybenzoate; and 1,2-benzisothiazolin-3-one (BIT).

The presence of surfactants often causes water-based formulations to foam during mixing operations in production and in application through a spray tank. In order to reduce the tendency to foam, anti-foam agents are often added either during the production stage or before filling into bottles. Generally, there are two types of anti-foam agents, namely silicones and non-silicones. Silicones are usually aqueous emulsions of dimethyl polysiloxane, while the non-silicone anti-foam agents are water-insoluble oils, such as octanol and nonanol, or silica. In both cases, the function of the anti-foam agent is to displace the surfactant from the air-water interface.

“Green” agents (e.g., adjuvants, surfactants, solvents) can reduce the overall environmental footprint of crop protection formulations. Green agents are biodegradable and generally derived from natural and/or sustainable sources, e.g. plant and animal sources. Specific examples are: vegetable oils, seed oils, and esters thereof, also alkoxylated alkyl polyglucosides.

### Applications

Molecules of Formula One may be applied to any locus. Particular crop loci to apply such molecules include loci where alfalfa, almonds, apples, barley, beans, canola, corn, cotton, crucifers, lettuce, oats, oranges, pears, peppers, potatoes, rice, sorghum, soybeans, strawberries, sugarcane, sugar beets, sunflowers, tobacco, tomatoes, wheat, and other valuable crops are growing or the seeds thereof are going to be planted.

Molecules of Formula One may also be applied where plants, such as crops, are growing and where there are low levels (even no actual presence) of pests that can commercially damage such plants. Applying such molecules in such locus is to benefit the plants being grown in such locus. Such benefits, may include, but are not limited to: helping the plant grow a better root system; helping the plant better withstand stressful growing conditions; improving the health of a plant; improving the yield of a plant (e.g. increased biomass and/or increased content of valuable ingredients); improving the vigor of a plant (e.g. improved plant growth and/or greener leaves); improving the quality of a plant (e.g. improved content or composition of certain ingredients); and improving the tolerance to abiotic and/or biotic stress of the plant.

Molecules of Formula One may be applied with ammonium sulfate when growing various plants as this may provide additional benefits.

Molecules of Formula One may be applied on, in, or around plants genetically modified to express specialized traits, such as Bacillus thuringiensis or other insecticidal toxins, or those expressing herbicide resistance, or those with “stacked” foreign genes expressing insecticidal toxins, herbicide resistance, nutrition-enhancement, or any other beneficial traits.

Molecule of Formula One may be applied to the foliar and/or fruiting portions of plants to control pests. Such molecules will either come in direct contact with the pest, or the pest will consume such molecules when eating the plant or while extracting sap from the plant.

Molecule of Formula One may also be applied to the soil, and when applied in this manner, root and stem feeding pests may be controlled. The roots may absorb such molecules thereby taking it up into the foliar portions of the plant to control above ground chewing and sap feeding pests.

Systemic movement of pesticides in plants may be utilized to control pests on one portion of the plant by applying (for example by spraying a locus) a molecule of Formula One to a different portion of the plant. For example, control of foliar-feeding insects may be achieved by drip irrigation or furrow application, by treating the soil with for example pre- or post-planting soil drench, or by treating the seeds of a plant before planting.

Molecules of Formula One may be used with baits. Generally, with baits, the baits are placed in the ground where, for example, termites can come into contact with, and/or be attracted to, the bait. Baits can also be applied to a surface of a building, (horizontal, vertical, or slant surface) where, for example, ants, termites, cockroaches, and flies, can come into contact with, and/or be attracted to, the bait.

Molecules of Formula One may be encapsulated inside, or placed on the surface of a capsule. The size of the capsules can range from nanometer size (about 100-900 nanometers in diameter) to micrometer size (about 10-900 microns in diameter).

Molecules of Formula One may be applied to eggs of pests. Because of the unique ability of the eggs of some pests to resist certain pesticides, repeated applications of such molecules may be desirable to control newly emerged larvae.

Molecules of Formula One may be applied as seed treatments. Seed treatment may be applied to all types of seeds, including those from which plants genetically modified to express specialized traits will germinate. Representative examples include those expressing proteins toxic to invertebrate pests, such as Bacillus thuringiensis or other insecticidal toxins, those expressing herbicide resistance, such as “Roundup Ready” seed, or those with “stacked” foreign genes expressing insecticidal toxins, herbicide resistance, nutrition-enhancement, drought resistance, or any other beneficial traits. Furthermore, such seed treatments with molecules of Formula One may further enhance the ability of a plant to better withstand stressful growing conditions. This results in a healthier, more vigorous plant, which can lead to higher yields at harvest time. Generally, about 1 gram of such molecules to about 500 grams per 100,000 seeds is expected to provide good benefits, amounts from about 10 grams to about 100 grams per 100,000 seeds is expected to provide better benefits, and amounts from about 25 grams to about 75 grams per 100,000 seeds is expected to provide even better benefits.

Molecules of Formula One may be applied with one or more active ingredients in a soil amendment.

Molecules of Formula One may be used for controlling endoparasites and ectoparasites in the veterinary medicine sector or in the field of non-human-animal keeping. Such molecules may be applied by oral administration in the form of, for example, tablets, capsules, drinks, granules, by dermal application in the form of, for example, dipping, spraying, pouring on, spotting on, and dusting, and by parenteral administration in the form of, for example, an injection.

Molecules of Formula One may also be employed advantageously in livestock keeping, for example, cattle, sheep, pigs, chickens, salmon, and geese. They may also be employed advantageously in pets such as, horses, dogs, and cats. Particular pests to control would be fleas and ticks that are bothersome to such animals. Suitable formulations are administered orally to the animals with the drinking water or feed. The dosages and formulations that are suitable depend on the species.

Molecules of Formula One may also be used for controlling parasitic worms, especially of the intestine, in the animals listed above.

Molecules of Formula One may also be employed in therapeutic methods for human health care. Such methods include, but are limited to, oral administration in the form of, for example, tablets, capsules, drinks, granules, and by dermal application.

Molecules of Formula One may also be applied to invasive pests. Pests around the world have been migrating to new environments (for such pest) and thereafter becoming a new invasive species in such new environment. Such molecules may also be used on such new invasive species to control them in such new environments.

Consequently, in light of the above and the Tables in the Table Section, the following items are provided.

1. A molecule having the following formula

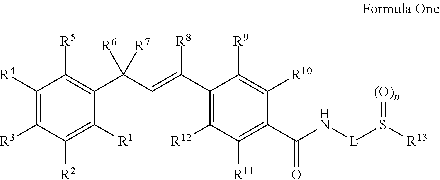

wherein:

(A) R¹, R⁵, R⁶, R⁹, and R¹² are each independently selected from H, F, Cl, Br, I, CN, (C₁-C₄)alkyl, (C₁-C₄)haloalkyl, (C₁-C₄)alkoxy, and (C₁-C₄)haloalkoxy

preferably, R¹, R⁵, R⁶, R⁹, and R¹² are H;

(B) R² is selected from H, F, Cl, Br, I, CN, (C₁-C₄)alkyl, (C₁-C₄)haloalkyl, (C₁-C₄)alkoxy, and (C₁-C₄)haloalkoxy preferably, R² are Cl or Br;

(C) R³ and R⁴ are each independently selected from (D), H, F, Cl, Br, I, CN, C(O)H, (C₁-C₄)alkyl, (C₂-C₄)alkenyl, (C₂-C₄)alkynyl, (C₁-C₄)haloalkyl, (C₁-C₄)alkoxy, and (C₁-C₄)haloalkoxy

preferably R³ and R⁴ are H, F, Cl, Br, I, CN, or C(O)H;

(D) R³ and R⁴ together can optionally form a 3- to 5-membered saturated or unsaturated, heterohydrocarbyl link, which may contain one or more heteroatoms selected from nitrogen, sulfur, and oxygen,

wherein said heterohydrocarbyl link may optionally be substituted with one or more substituents independently selected from H, F, Cl, Br, I, CN, and OH

preferably R³ and R⁴ together are —OCH₂O—;

(E) R⁷ is (C₁-C₆)haloalkyl

preferably R⁷ is CF₃ or CF₂CH₃;

(F) R⁸ is selected from H, (C₁-C₄)alkyl, (C₁-C₄)haloalkyl, and (C₁-C₄)alkoxy preferably R⁸ is H, OCH₃, or OCH₂CH₃;

(G) R¹⁰ is selected from F, Cl, Br, I, (C₁-C₄)alkyl, (C₂-C₄)alkenyl, (C₂-C₄)alkenyl, (C₁-C₄)haloalkyl, (C₁-C₄)alkoxy, and (C₁-C₄)haloalkoxy preferably R¹⁰ is F, Cl, Br, CH₃, CH₂CH₃, CHF₂, or CF₃;

(H) R¹¹ is selected from H, F, Cl, Br, I, (C₁-C₄)alkyl, or (C₁-C₄)haloalkyl preferably R¹¹ is H or CH₃;

(I) L is a linker that is selected from (C₁-C₅)alkyl, (C₁-C₄)alkoxy, (C₃-C₆)cycloalkyl-(C₁-C₄)alkyl, (C₁-C₄)alkyl-(C₃-C₆)cycloalkoxy, (C₁-C₄)alkyl-S—(C₁-C₄)alkyl, (C₁-C₄)alkyl-S(O)—(C₁-C₄)alkyl, and (C₁-C₄)alkyl-S(O)₂—(C₁-C₄)alkyl,

wherein each alkyl, alkoxy, and cycloalkyl may optionally be substituted with one or more substituents independently selected from F, Cl, Br, I, CN, OH, oxetanyl, C(═O)NH(C₁-C₄)haloalkyl, and (C₁-C₄)alkoxy

preferably L is —CH₂CH₂—, —CH(CH₃)CH₂—, —CH(CH₂CH₃)CH₂—, —CH(CH(CH₃)₂)CH₂—, —C(CH₃)₂CH₂—, —CH(CH₃)CH₂CH₂—, —CH(CH₂OCH₃)CH₂—, —C(cyclopropyl)CH₂—, —CH₂C(3,3-oxetanyl)-, or —CH₂CH(SCH₂CH₃)—;

(J) n is selected from 0, 1, and 2

(K) R¹³ is selected from (C₁-C₄)alkyl, (C₂-C₄)alkenyl, (C₁-C₄)haloalkyl, (C₁-C₄)alkoxy, (C₁-C₄)haloalkoxy, phenyl, benzyl, (C₁-C₄)alkyl-(C₃-C₆)cycloalkyl, and NH(C₁-C₄)haloalkyl, wherein each alkyl, alkenyl, haloalkyl, alkoxy, haloalkoxy, phenyl, and cycloalkyl, may optionally be substituted with one or more substituents independently selected from F, Cl, Br, I, CN, and OH

preferably R¹³ is CH₃, CH₂CH₃, CH₂CH₂CH₃, CH(CH₃)₂, CH₂CH(CH₃)₂, CH₂CH═CH₂, CH₂CF₃, CH₂CH₂CF₃, phenyl, CH₂phenyl, CH₂cyclopropyl, or NHCH₂CF₃, wherein each phenyl and cyclopropyl is optionally substituted with one or more substituents selected from F, Cl, Br, and CN; and

agriculturally acceptable acid addition salts, salt derivatives, solvates, ester derivatives, crystal polymorphs, isotopes, resolved stereoisomers, and tautomers, of the molecules of Formula One.

2. A molecule according to 1 wherein

(A) R¹, R⁵, R⁶, R, and R¹² are H;

(B) R² is selected from the group consisting of Cl and Br;

(C) R³ and R⁴ are, each independently selected from the group consisting of (D), H, F, Cl, Br, I, CN, and C(O)H;

(D) R³ and R⁴ together can optionally form a 3- to 5-membered saturated or unsaturated, heterohydrocarbyl link, which may contain one or more heteroatoms selected from the group consisting of nitrogen, sulfur, and oxygen,

wherein said heterohydrocarbyl link may optionally be substituted with one or more substituents independently selected from the group consisting of H, F, Cl, Br, I, CN, and OH;

(E) R⁷ is (C₁-C₆)haloalkyl;

(F) R⁸ is selected from the group consisting of H and (C₁-C₄)alkoxy;

(G) R¹⁰ is selected from the group consisting of F, Cl, Br, I, (C₁-C₄)alkyl, and (C₁-C₄)haloalkyl;

(H) R¹¹ is selected from the group consisting of H and (C₁-C₄)alkyl;

(I) L is a linker that is selected from the group consisting of (C₁-C₈)alkyl, (C₁-C₄)alkoxy, (C₃-C₆)cycloalkyl-(C₁-C₄)alkyl, (C₁-C₄)alkyl-(C₃-C₆)cycloalkoxy, and (C₁-C₄)alkyl-S—(C₁-C₄)alkyl, wherein each alkyl, alkoxy, and cycloalkyl may optionally be substituted with one or more (C₁-C₄)alkoxy substituents;

(J) n is selected from the group consisting of 0, 1, and 2; and

(K) R¹³ is selected from the group consisting of (C₁-C₄)alkyl, (C₂-C₄)alkenyl, (C₁-C₄)haloalkyl, phenyl, benzyl, (C₁-C₄)alkyl-(C₃-C₆)cycloalkyl, and NH(C₁-C₄)haloalkyl,

wherein each alkyl, alkenyl, haloalkyl, phenyl, and cycloalkyl may optionally be substituted with one or more substituents independently selected from the group consisting of F, Cl, Br, I, and CN.

3. A molecule according to 1 wherein

(A) R¹, R⁵, R⁶, R⁹, and R¹² are H;

(B) R² is selected from the group consisting of Cl and Br;

(C) R³ and R⁴ are, each independently selected from the group consisting of H, F, Cl, Br, I, and CN.

(E) R⁷ is (C₁-C₆)haloalkyl;

(F) R⁸ is H;

(G) R¹⁰ is selected from the group consisting of F, Cl, Br, I, (C₁-C₄)alkyl, and (C₁-C₄)haloalkyl;

(H) R¹³ is selected from the group consisting of H and (C₁-C₄)alkyl;

(I) L is a linker that is selected from the group consisting of (C₁-C₅)alkyl, (C₃-C₆)cycloalkyl-(C₁-C₄)alkyl, and (C₁-C₄)alkyl-S—(C₁-C₄)alkyl;

(J) n is selected from the group consisting of 0, 1, and 2; and

(K) R¹³ is selected from the group consisting of (C₁-C₄)alkyl, (C₂-C₄)alkenyl, (C₁-C₄)haloalkyl, benzyl, (C₁-C₄)alkyl-(C₃-C₆)cycloalkyl, and NH(C₁-C₄)haloalkyl,

wherein each alkyl, alkenyl, haloalkyl, phenyl, and cycloalkyl, may optionally be substituted with one or more substituents independently selected from the group consisting of F, Cl, Br, and I.

4. A molecule according to 1 wherein said molecule is selected from one of the molecules in Table 1.
5. A pesticidal composition comprising a molecule according to any one of 1, 2, 3, or 4, further comprising one or more active ingredients.
6. A pesticidal composition according to 5 wherein said active ingredient is from AIGA.
7. A pesticidal composition according to 5 wherein said active ingredient is selected from the group consisting of AI-1, 1,3-dichloropropene, chlorpyrifos, chlorpyrifos-methyl, hexaflumuron, methoxyfenozide, noviflumuron, spinetoram, spinosad, sulfoxaflor, and sulfuryl fluoride.
8. A pesticidal composition comprising a molecule according to any one of 1, 2, 3, or 4, further comprising a MoA Material.
9. A pesticidal composition according to 7 wherein said MoA Material is from MoAMGA.
10. A pesticidal composition according to any one of 5, 6, 7, 8, or 9, wherein the weight ratio of the molecule according to Formula One to said active ingredient is

(a) 100:1 to 1:100;

(b) 50:1 to 1:50;

(c) 20:1 to 1:20;

(d) 10:1 to 1:10;

(e) 5:1 to 1:5;

(f) 3:1 to 1:3;

(g) 2:1 to 1:2; or

(h) 1:1.

11. A process to control a pest said process comprising applying to a locus, a pesticidally effective amount of a molecule according to any one of the 1, 2, 3, or 4.
12. A process to control a pest said process comprising applying to a locus, a pesticidally effective amount of a pesticidal composition according to any one of the 5, 6, 7, 8, 9, or 10.
13. A molecule according to any one of 1, 2, 3, or 4, or a pesticidal composition according to any of 5, 6, 7, 8, 9, or 10, wherein said molecule is in the form of agriculturally acceptable acid addition salt.
14. A molecule according to any one of 1, 2, 3, or 4, or a pesticidal composition according to any of 5, 6, 7, 8, 9, or 10, wherein said molecule is in the form of a salt derivative.
15. A molecule according to any one of 1, 2, 3, or 4, or a pesticidal composition according to any of 5, 6, 7, 8, 9, or 10, wherein said molecule is in the form of solvate.
16. A molecule according to any one of 1, 2, 3, or 4, or a pesticidal composition according to any of 5, 6, 7, 8, 9, or 10, wherein said molecule is in the form of an ester derivative.
17. A molecule according to any one of 1, 2, 3, or 4, or a pesticidal composition according to any of 5, 6, 7, 8, 9, or 10, wherein said molecule is in the form of a crystal polymorph.
18. A molecule according to any one of 1, 2, 3, or 4, or a pesticidal composition according to any of 5, 6, 7, 8, 9, or 10, wherein said molecule has deuterium, tritium, and or ¹⁴C.
19. A molecule according to any one of 1, 2, 3, or 4, or a pesticidal composition according to any of 5, 6, 7, 8, 9, or 10, wherein said molecule is in the form of one or more stereoisomers.
20. A molecule according to any one of 1, 2, 3, or 4, or a pesticidal composition according to any of 5, 6, 7, 8, 9, or 10, wherein said molecule is in the form of a resolved stereoisomer.
21. A pesticidal composition according to any of 5, 6, 7, 8, 9, or 10, wherein said pesticidal composition further comprises another active ingredient.
22. A pesticidal composition according to any of 5, 6, 7, 8, 9, or 10, wherein said pesticidal composition further comprises two more active ingredients.
23. A pesticidal composition according to any of 5, 6, 7, 8, 9, or 10, wherein said active ingredient has a MOA different from the MoA of said molecule of Formula One.
24. A pesticidal composition according to any of 5, 6, 7, 8, 9, or 10, wherein said pesticidal composition comprises an active ingredient having acaricidal, algicidal, avicidal, bactericidal, fungicidal, herbicidal, insecticidal, molluscicidal, nematicidal, rodenticidal, and/or virucidal properties.
25. A pesticidal composition according to any of 5, 6, 7, 8, 9, or 10, wherein said pesticidal composition comprises an active ingredient that is an antifeedant, bird repellent, chemosterilant, herbicide safener, insect attractant, insect repellent, mammal repellent, mating disrupter, plant activator, plant growth regulator, and/or synergist.
26. A pesticidal composition according to any of 5, 6, 7, 8, 9, or 10, wherein said pesticidal composition comprises an active ingredient that is a biopesticide.
27. A pesticidal composition according to any of 5, 6, 7, 8, 9, or 10, wherein said weight ratio of a molecule of Formula One to an active ingredient is 100:1 to 1:100.
28. A pesticidal composition according to any of 5, 6, 7, 8, 9, or 10, wherein said weight ratio of a molecule of Formula One to an active ingredient is 50:1 to 1:50.
29. A pesticidal composition according to any of 5, 6, 7, 8, 9, or 10, wherein said weight ratio of a molecule of Formula One to an active ingredient is 20:1 to 1:20.
30. A pesticidal composition according to any of 5, 6, 7, 8, 9, or 10, wherein said weight ratio of a molecule of Formula One to an active ingredient is 10:1 to 1:10.
31. A pesticidal composition according to any of 5, 6, 7, 8, 9, or 10, wherein said weight ratio of a molecule of Formula One to an active ingredient is 5:1 to 1:5.
32. A pesticidal composition according to any of 5, 6, 7, 8, 9, or 10, wherein said weight ratio of a molecule of Formula One to an active ingredient is 3:1 to 1:3.
33. A pesticidal composition according to any of 5, 6, 7, 8, 9, or 10, wherein said weight ratio of a molecule of Formula One to an active ingredient is 2:1 to 1:2.
34. A pesticidal composition according to any of 5, 6, 7, 8, 9, or 10, wherein said weight ratio of a molecule of Formula One to an active ingredient is 1:1
35. A pesticidal composition according to any of 5, 6, 7, 8, 9, or 10, wherein said the weight ratio of a molecule of Formula One to an active ingredient is depicted as X:Y; wherein X is the parts by weight of a molecule of Formula One and Y is the parts by weight of active ingredient; further wherein the numerical range of the parts by weight for X is 0<X≦100 and the parts by weight for Y is 0<Y≦100; and further wherein X and Y are selected from Table C.
36. A pesticidal composition according to 35 wherein a range of weight ratios of a molecule of Formula One to an active ingredient is depicted as X₁:Y₁ to X₂:Y₂; further wherein X₁>Y₁ and X₂<Y₂.
37. A pesticidal composition according to 35 wherein a range of weight ratios of a molecule of Formula One to an active ingredient is depicted as to X₁:Y₁ to X₂:Y₂; further wherein X₁>Y₁ and X₂>Y₂.
38. A pesticidal composition according to 35 wherein a range of weight ratios of a molecule of Formula One to an active ingredient is depicted as X₁:Y₁ to X₂:Y₂; further wherein X₁<Y₁ and X₂<Y₂.
39. A pesticidal composition according to 35 wherein said composition is synergistic.
40. A process according to 12 wherein said pest is from Phylum Arthropoda.
41. A process according to 12 wherein said pest is from Phylum Mollusca.
42. A process according to 12 wherein said pest is from Phylum Nematoda.
43. A process according to 12 wherein said pests are are ants, aphids, beetles, bristletails, cockroaches, crickets, earwigs, fleas, flies, grasshoppers, leafhoppers, lice (including sea lice), locusts, mites, moths, nematodes, scales, symphylans, termites, thrips, ticks, wasps, and/or whiteflies.
44. A process according to 12 wherein said locus is where alfalfa, almonds, apples, barley, beans, canola, corn, cotton, crucifers, lettuce, oats, oranges, pears, peppers, potatoes, rice, sorghum, soybeans, strawberries, sugarcane, sugar beets, sunflowers, tobacco, tomatoes, wheat, and other valuable crops are growing or the seeds thereof are planted.
45. A pesticidal composition according to any of 5, 6, 7, 8, 9, or 10, wherein said pesticidal composition further comprises ammonium sulfate.
46. A process according to 12 wherein said locus is where plants genetically modified to express specialized traits are planted.
47. A process according to 12 wherein said applying is done to the foliar and/or fruiting portions of plants.
48. A process according to 12 wherein said applying is done to the soil.
49. A process according to 12 wherein said applying is done by drip irrigation, furrow application, or pre- or post-planting soil drench.
50. A process according to 12 wherein said applying is done to the foliar and/or fruiting portions of plants., or by treating the seeds of a plant before planting.
51. A pesticidal composition comprising a molecule according to any one of 1, 2, 3, or 4, and a seed.
52. A process comprising applying a molecule according to any one of 1, 2, 3, or 4, or a pesticidal composition according to any of 5, 6, 7, 8, 9, or 10, to a seed.
53. A process comprising applying a molecule according to 1, 2, 3, or 4, to a locus that includes a non-human animal to control endoparasites and/or ectoparasites.
54. A process to produce a pesticidal composition, said process comprising mixing a molecule according to any one of claims 1, 2, 3, or 4, with one or more active ingredients.

The headings in this document are for convenience only and must not be used to interpret any portion hereof.

### Table Section

TABLE 1
Structure and Preparation Method for F Series Molecules

| No. | Structure | Prep.* |
|---|---|---|
| F1 | 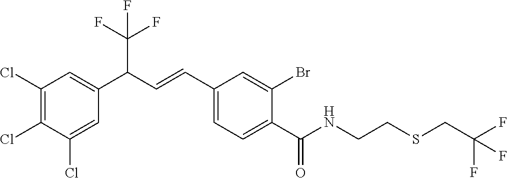 [SMILES: O=C(NCCSCC(F)(F)F)c1ccc(/C=C/C(c2cc(Cl)c(Cl)c(Cl)c2)C(F)(F)F)cc1Br] | 1 |
| F2 | 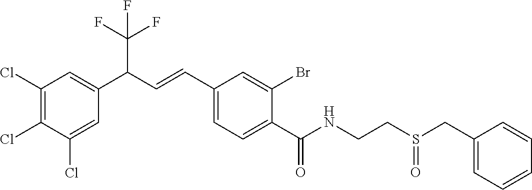 [SMILES: O=C(NCCS(=O)Cc1ccccc1)c1ccc(/C=C/C(c2cc(Cl)c(Cl)c(Cl)c2)C(F)(F)F)cc1Br] | 1 |
| F3 | 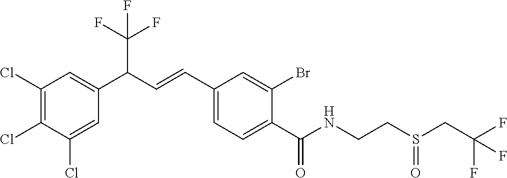 [SMILES: O=C(NCCS(=O)CC(F)(F)F)c1ccc(/C=C/C(c2cc(Cl)c(Cl)c(Cl)c2)C(F)(F)F)cc1Br] | 3 |
| F4 | 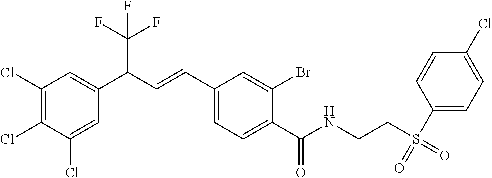 [SMILES: O=C(NCCS(=O)(=O)c1ccc(Cl)cc1)c1ccc(/C=C/C(c2cc(Cl)c(Cl)c(Cl)c2)C(F)(F)F)cc1Br] | 1 |
| F10 | 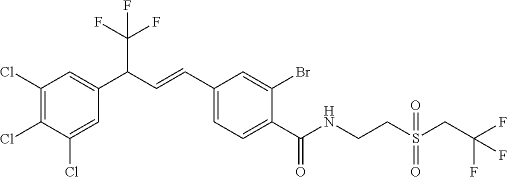 [SMILES: O=C(NCCS(=O)(=O)CC(F)(F)F)c1ccc(/C=C/C(c2cc(Cl)c(Cl)c(Cl)c2)C(F)(F)F)cc1Br] | 3 |
| F11 | 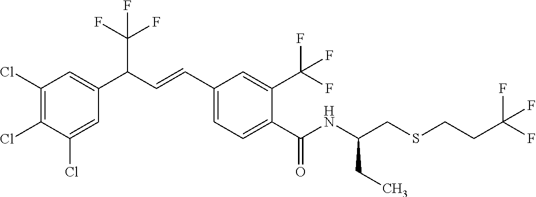 [SMILES: CC[C@H](CSCCC(F)(F)F)NC(=O)c1ccc(/C=C/C(c2cc(Cl)c(Cl)c(Cl)c2)C(F)(F)F)cc1C(F)(F)F] | 4 |
| F12 | 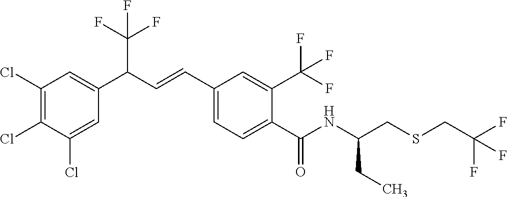 [SMILES: CC[C@H](CSCC(F)(F)F)NC(=O)c1ccc(/C=C/C(c2cc(Cl)c(Cl)c(Cl)c2)C(F)(F)F)cc1C(F)(F)F] | 4 |
| F13 | 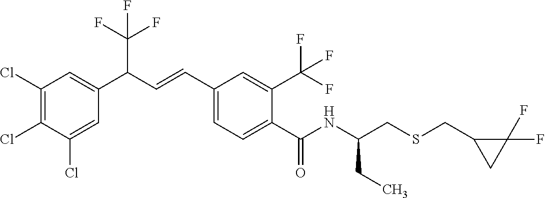 [SMILES: CC[C@H](CSCC1CC1(F)F)NC(=O)c1ccc(/C=C/C(c2cc(Cl)c(Cl)c(Cl)c2)C(F)(F)F)cc1C(F)(F)F] | 4 |
| F14 | 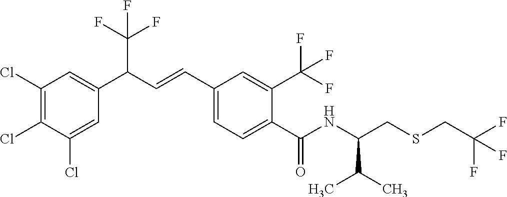 [SMILES: CC(C)[C@H](CSCC(F)(F)F)NC(=O)c1ccc(/C=C/C(c2cc(Cl)c(Cl)c(Cl)c2)C(F)(F)F)cc1C(F)(F)F] | 4 |
| F15 | 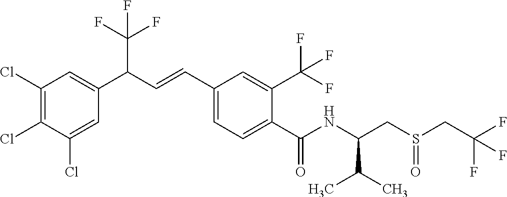 [SMILES: CC(C)[C@H](CS(=O)CC(F)(F)F)NC(=O)c1ccc(/C=C/C(c2cc(Cl)c(Cl)c(Cl)c2)C(F)(F)F)cc1C(F)(F)F] | 2 |
| F16 | 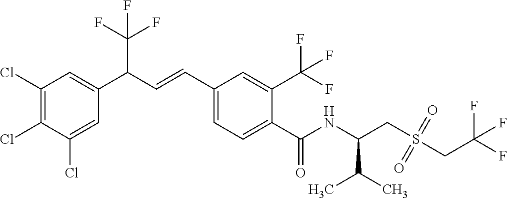 [SMILES: CC(C)[C@H](CS(=O)(=O)CC(F)(F)F)NC(=O)c1ccc(/C=C/C(c2cc(Cl)c(Cl)c(Cl)c2)C(F)(F)F)cc1C(F)(F)F] | 3 |
| F17 | 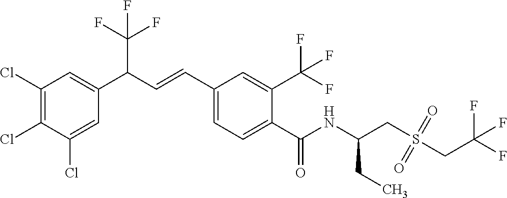 [SMILES: CC[C@H](CS(=O)(=O)CC(F)(F)F)NC(=O)c1ccc(/C=C/C(c2cc(Cl)c(Cl)c(Cl)c2)C(F)(F)F)cc1C(F)(F)F] | 3 |
| F18 | 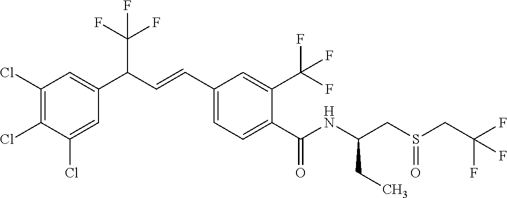 [SMILES: CC[C@H](CS(=O)CC(F)(F)F)NC(=O)c1ccc(/C=C/C(c2cc(Cl)c(Cl)c(Cl)c2)C(F)(F)F)cc1C(F)(F)F] | 3 |
| F19 | 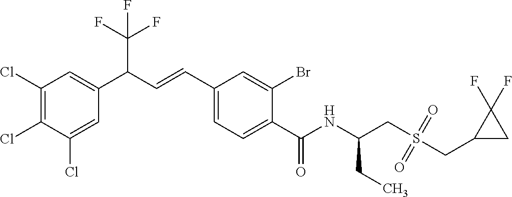 [SMILES: CC[C@H](CS(=O)(=O)CC1CC1(F)F)NC(=O)c1ccc(/C=C/C(c2cc(Cl)c(Cl)c(Cl)c2)C(F)(F)F)cc1Br] | 3 |
| F20 | 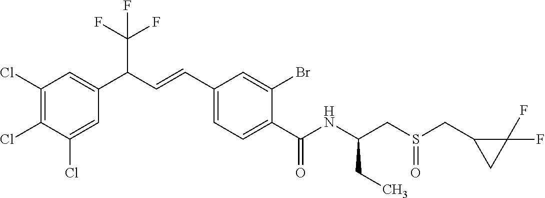 [SMILES: CC[C@H](CS(=O)CC1CC1(F)F)NC(=O)c1ccc(/C=C/C(c2cc(Cl)c(Cl)c(Cl)c2)C(F)(F)F)cc1Br] | 3 |
| F21 | 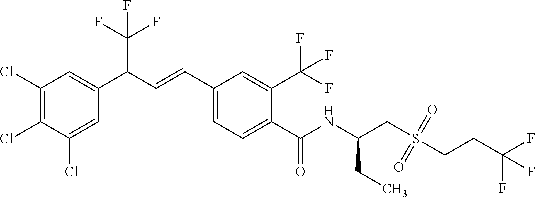 [SMILES: CC[C@H](CS(=O)(=O)CCC(F)(F)F)NC(=O)c1ccc(/C=C/C(c2cc(Cl)c(Cl)c(Cl)c2)C(F)(F)F)cc1C(F)(F)F] | 3 |
| F22 | 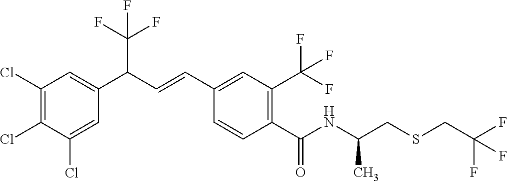 [SMILES: C[C@H](CSCC(F)(F)F)NC(=O)c1ccc(/C=C/C(c2cc(Cl)c(Cl)c(Cl)c2)C(F)(F)F)cc1C(F)(F)F] | 5 |
| F23 | 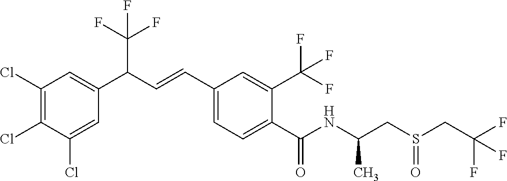 [SMILES: C[C@H](CS(=O)CC(F)(F)F)NC(=O)c1ccc(/C=C/C(c2cc(Cl)c(Cl)c(Cl)c2)C(F)(F)F)cc1C(F)(F)F] | 2a |
| F24 | 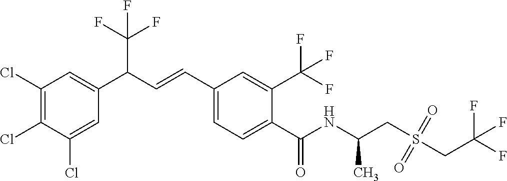 [SMILES: C[C@H](CS(=O)(=O)CC(F)(F)F)NC(=O)c1ccc(/C=C/C(c2cc(Cl)c(Cl)c(Cl)c2)C(F)(F)F)cc1C(F)(F)F] | 3 |
| F25 | 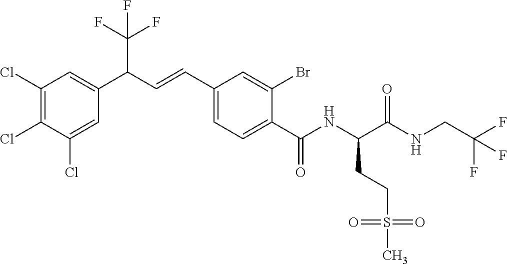 [SMILES: CS(=O)(=O)CC[C@@H](NC(=O)c1ccc(/C=C/C(c2cc(Cl)c(Cl)c(Cl)c2)C(F)(F)F)cc1Br)C(=O)NCC(F)(F)F] | 6 |
| F26 | 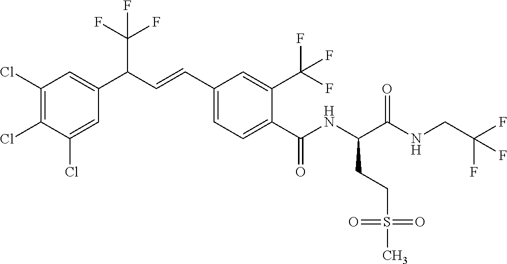 [SMILES: CS(=O)(=O)CC[C@@H](NC(=O)c1ccc(/C=C/C(c2cc(Cl)c(Cl)c(Cl)c2)C(F)(F)F)cc1C(F)(F)F)C(=O)NCC(F)(F)F] | 6 |
| F27 | 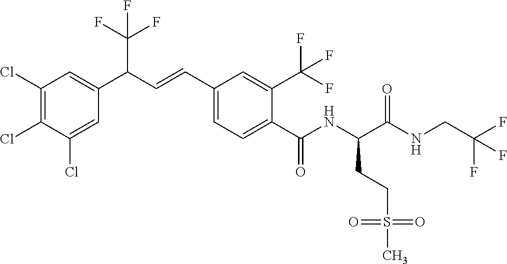 [SMILES: CS(=O)(=O)CC[C@@H](NC(=O)c1ccc(/C=C/C(c2cc(Cl)c(Cl)c(Cl)c2)C(F)(F)F)cc1C(F)(F)F)C(=O)NCC(F)(F)F] | 6 |
| F28 | 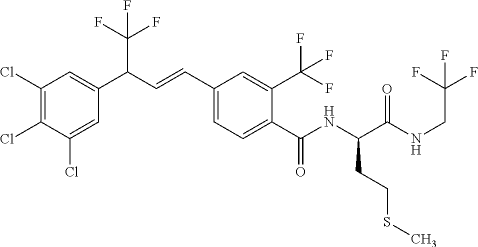 [SMILES: CSCC[C@@H](NC(=O)c1ccc(/C=C/C(c2cc(Cl)c(Cl)c(Cl)c2)C(F)(F)F)cc1C(F)(F)F)C(=O)NCC(F)(F)F] | 6 |
| F29 | 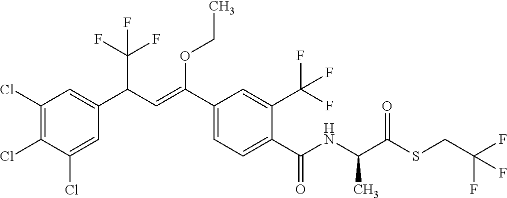 [SMILES: CCO/C(=C\C(c1cc(Cl)c(Cl)c(Cl)c1)C(F)(F)F)c1ccc(C(=O)N[C@H](C)C(=O)SCC(F)(F)F)c(C(F)(F)F)c1] | 7 |
| F30 | 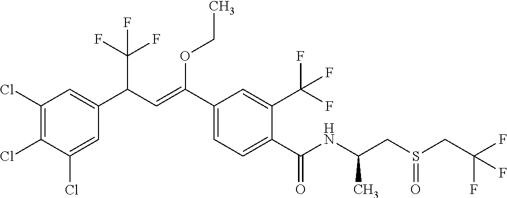 [SMILES: CCO/C(=C\C(c1cc(Cl)c(Cl)c(Cl)c1)C(F)(F)F)c1ccc(C(=O)N[C@H](C)CS(=O)CC(F)(F)F)c(C(F)(F)F)c1] | 2a |
| F31 | 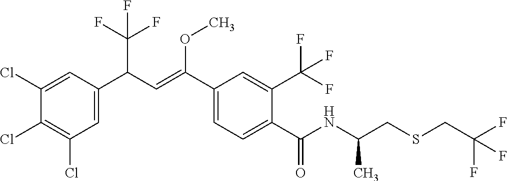 [SMILES: CO/C(=C\C(c1cc(Cl)c(Cl)c(Cl)c1)C(F)(F)F)c1ccc(C(=O)N[C@H](C)CSCC(F)(F)F)c(C(F)(F)F)c1] | 7 |
| F32 | 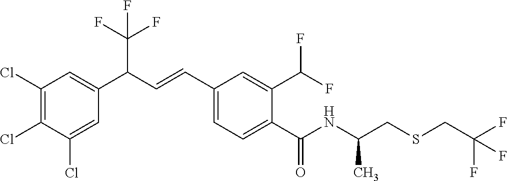 [SMILES: C[C@H](CSCC(F)(F)F)NC(=O)c1ccc(/C=C/C(c2cc(Cl)c(Cl)c(Cl)c2)C(F)(F)F)cc1C(F)F] | 7 |
| F33 | 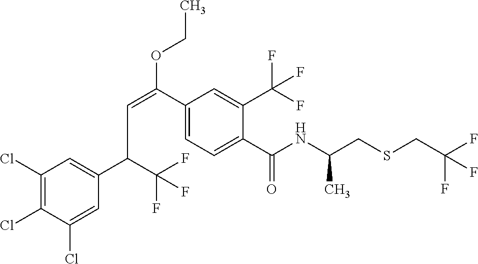 [SMILES: CCO/C(=C/C(c1cc(Cl)c(Cl)c(Cl)c1)C(F)(F)F)c1ccc(C(=O)N[C@H](C)CSCC(F)(F)F)c(C(F)(F)F)c1] | 8 |
| F34 | 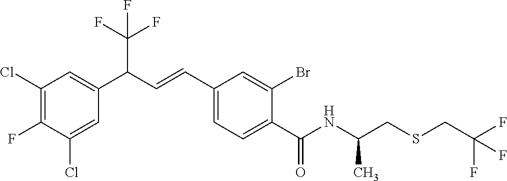 [SMILES: C[C@H](CSCC(F)(F)F)NC(=O)c1ccc(/C=C/C(c2cc(Cl)c(F)c(Cl)c2)C(F)(F)F)cc1Br] | 6 |
| F35 | 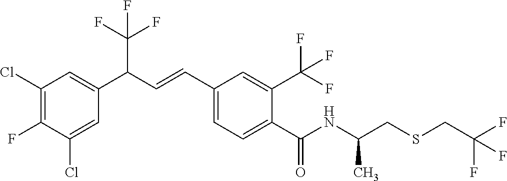 [SMILES: C[C@H](CSCC(F)(F)F)NC(=O)c1ccc(/C=C/C(c2cc(Cl)c(F)c(Cl)c2)C(F)(F)F)cc1C(F)(F)F] | 6 |
| F36 | 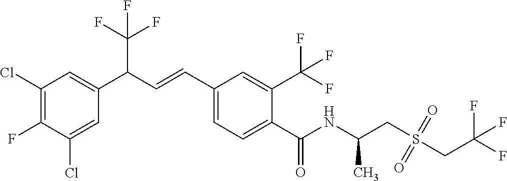 [SMILES: C[C@H](CS(=O)(=O)CC(F)(F)F)NC(=O)c1ccc(/C=C/C(c2cc(Cl)c(F)c(Cl)c2)C(F)(F)F)cc1C(F)(F)F] | 3 |
| F37 | 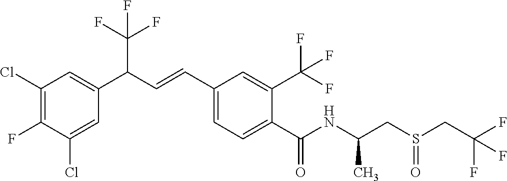 [SMILES: C[C@H](CS(=O)CC(F)(F)F)NC(=O)c1ccc(/C=C/C(c2cc(Cl)c(F)c(Cl)c2)C(F)(F)F)cc1C(F)(F)F] | 2a |
| F38 | 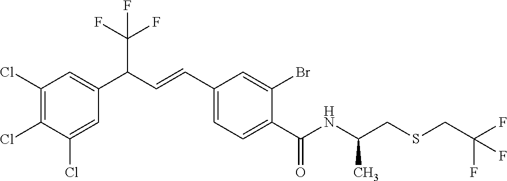 [SMILES: C[C@H](CSCC(F)(F)F)NC(=O)c1ccc(/C=C/C(c2cc(Cl)c(Cl)c(Cl)c2)C(F)(F)F)cc1Br] | 6 |
| F39 | 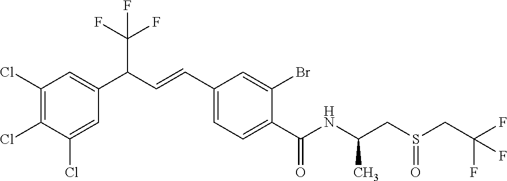 [SMILES: C[C@H](CS(=O)CC(F)(F)F)NC(=O)c1ccc(/C=C/C(c2cc(Cl)c(Cl)c(Cl)c2)C(F)(F)F)cc1Br] | 2a |
| F40 | 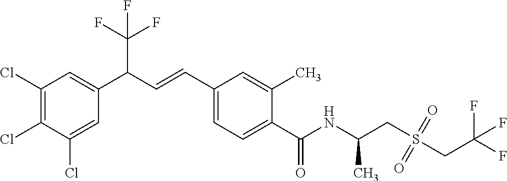 [SMILES: Cc1cc(/C=C/C(c2cc(Cl)c(Cl)c(Cl)c2)C(F)(F)F)ccc1C(=O)N[C@H](C)CS(=O)(=O)CC(F)(F)F] | 3 |
| F41 | 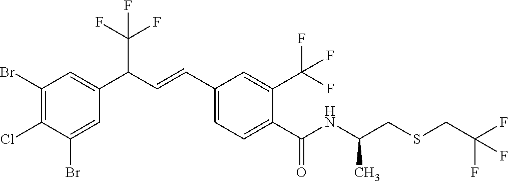 [SMILES: C[C@H](CSCC(F)(F)F)NC(=O)c1ccc(/C=C/C(c2cc(Br)c(Cl)c(Br)c2)C(F)(F)F)cc1C(F)(F)F] | 6 |
| F42 | 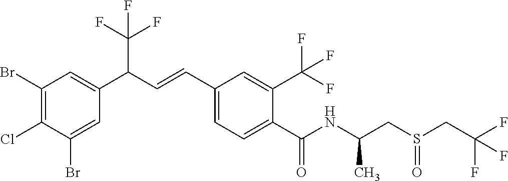 [SMILES: C[C@H](CS(=O)CC(F)(F)F)NC(=O)c1ccc(/C=C/C(c2cc(Br)c(Cl)c(Br)c2)C(F)(F)F)cc1C(F)(F)F] | 2a |
| F43 | 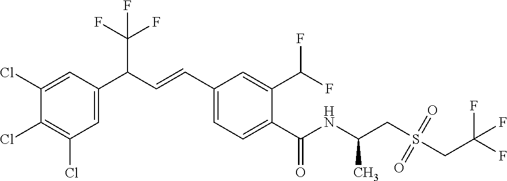 [SMILES: C[C@H](CS(=O)(=O)CC(F)(F)F)NC(=O)c1ccc(/C=C/C(c2cc(Cl)c(Cl)c(Cl)c2)C(F)(F)F)cc1C(F)F] | 3 |
| F44 | 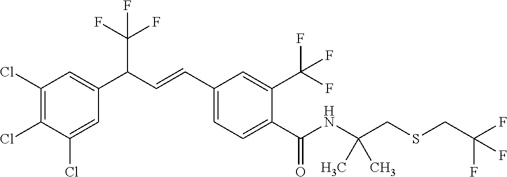 [SMILES: CC(C)(CSCC(F)(F)F)NC(=O)c1ccc(/C=C/C(c2cc(Cl)c(Cl)c(Cl)c2)C(F)(F)F)cc1C(F)(F)F] | 9 |
| F45 | 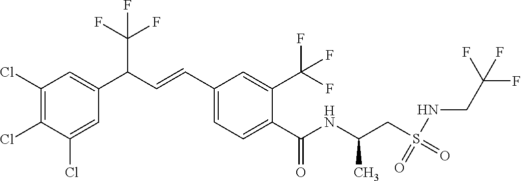 [SMILES: C[C@H](CS(=O)(=O)NCC(F)(F)F)NC(=O)c1ccc(/C=C/C(c2cc(Cl)c(Cl)c(Cl)c2)C(F)(F)F)cc1C(F)(F)F] | 6 |
| F46 | 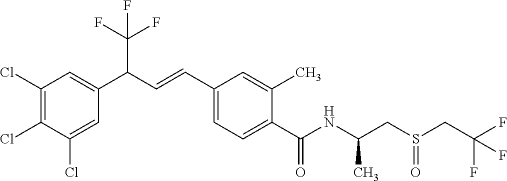 [SMILES: Cc1cc(/C=C/C(c2cc(Cl)c(Cl)c(Cl)c2)C(F)(F)F)ccc1C(=O)N[C@H](C)CS(=O)CC(F)(F)F] | 2a |
| F47 | 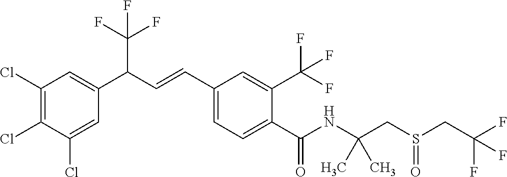 [SMILES: CC(C)(CS(=O)CC(F)(F)F)NC(=O)c1ccc(/C=C/C(c2cc(Cl)c(Cl)c(Cl)c2)C(F)(F)F)cc1C(F)(F)F] | 3 |
| F48 | 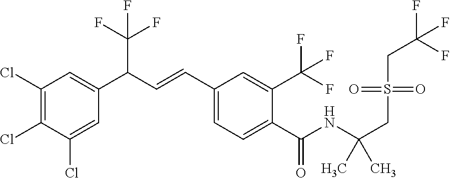 [SMILES: CC(C)(CS(=O)(=O)CC(F)(F)F)NC(=O)c1ccc(/C=C/C(c2cc(Cl)c(Cl)c(Cl)c2)C(F)(F)F)cc1C(F)(F)F] | 3 |
| F49 | 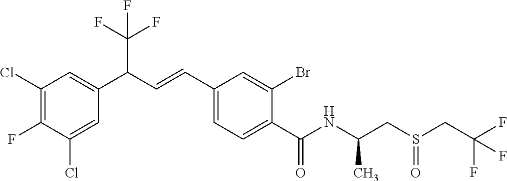 [SMILES: C[C@H](CS(=O)CC(F)(F)F)NC(=O)c1ccc(/C=C/C(c2cc(Cl)c(F)c(Cl)c2)C(F)(F)F)cc1Br] | 2a |
| F50 | 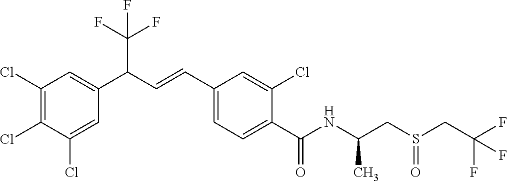 [SMILES: C[C@H](CS(=O)CC(F)(F)F)NC(=O)c1ccc(/C=C/C(c2cc(Cl)c(Cl)c(Cl)c2)C(F)(F)F)cc1Cl] | 2a |
| F51 | 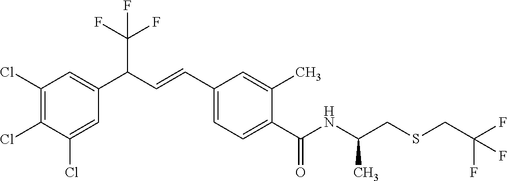 [SMILES: Cc1cc(/C=C/C(c2cc(Cl)c(Cl)c(Cl)c2)C(F)(F)F)ccc1C(=O)N[C@H](C)CSCC(F)(F)F] | 6 |
| F52 | 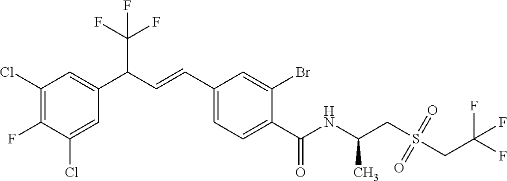 [SMILES: C[C@H](CS(=O)(=O)CC(F)(F)F)NC(=O)c1ccc(/C=C/C(c2cc(Cl)c(F)c(Cl)c2)C(F)(F)F)cc1Br] | 3 |
| F53 | 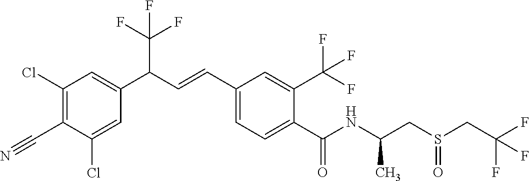 [SMILES: C[C@H](CS(=O)CC(F)(F)F)NC(=O)c1ccc(/C=C/C(c2cc(Cl)c(C#N)c(Cl)c2)C(F)(F)F)cc1C(F)(F)F] | 3 |
| F54 | 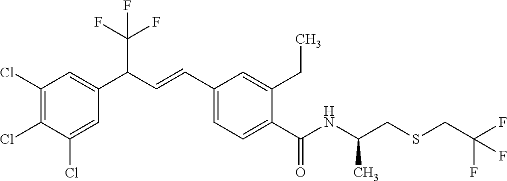 [SMILES: CCc1cc(/C=C/C(c2cc(Cl)c(Cl)c(Cl)c2)C(F)(F)F)ccc1C(=O)N[C@H](C)CSCC(F)(F)F] | 6 |
| F55 | 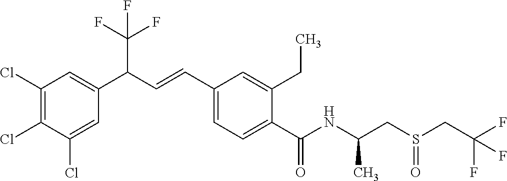 [SMILES: CCc1cc(/C=C/C(c2cc(Cl)c(Cl)c(Cl)c2)C(F)(F)F)ccc1C(=O)N[C@H](C)CS(=O)CC(F)(F)F] | 2a |
| F56 | 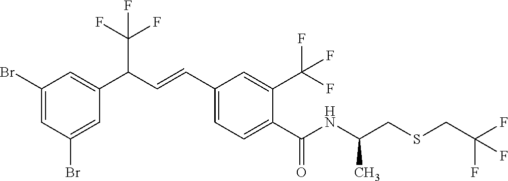 [SMILES: C[C@H](CSCC(F)(F)F)NC(=O)c1ccc(/C=C/C(c2cc(Br)cc(Br)c2)C(F)(F)F)cc1C(F)(F)F] | 6 |
| F57 | 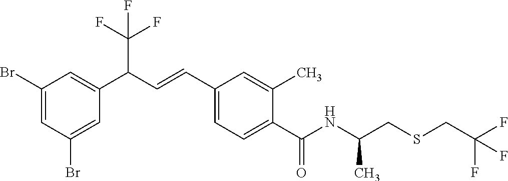 [SMILES: Cc1cc(/C=C/C(c2cc(Br)cc(Br)c2)C(F)(F)F)ccc1C(=O)N[C@H](C)CSCC(F)(F)F] | 6 |
| F58 | 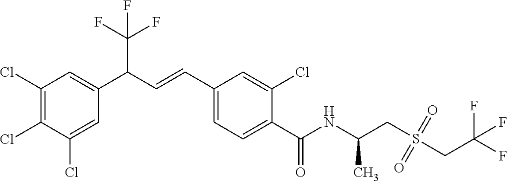 [SMILES: C[C@H](CS(=O)(=O)CC(F)(F)F)NC(=O)c1ccc(/C=C/C(c2cc(Cl)c(Cl)c(Cl)c2)C(F)(F)F)cc1Cl] | 3 |
| F59 | 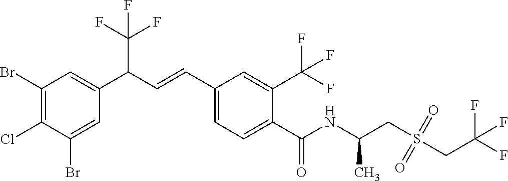 [SMILES: C[C@H](CS(=O)(=O)CC(F)(F)F)NC(=O)c1ccc(/C=C/C(c2cc(Br)c(Cl)c(Br)c2)C(F)(F)F)cc1C(F)(F)F] | 3 |
| F60 | 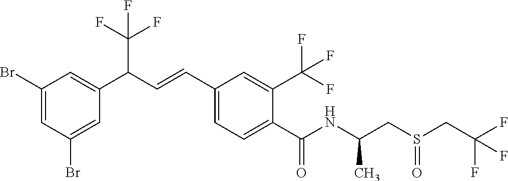 [SMILES: C[C@H](CS(=O)CC(F)(F)F)NC(=O)c1ccc(/C=C/C(c2cc(Br)cc(Br)c2)C(F)(F)F)cc1C(F)(F)F] | 2a |
| F61 | 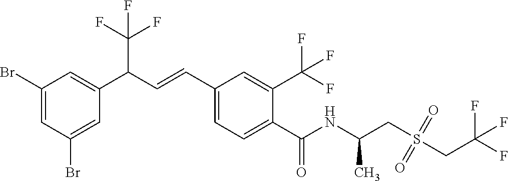 [SMILES: C[C@H](CS(=O)(=O)CC(F)(F)F)NC(=O)c1ccc(/C=C/C(c2cc(Br)cc(Br)c2)C(F)(F)F)cc1C(F)(F)F] | 3 |
| F62 | 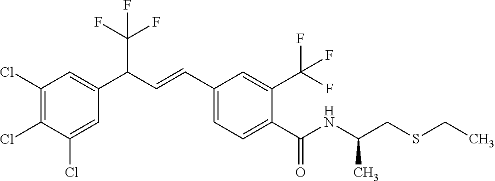 [SMILES: CCSC[C@@H](C)NC(=O)c1ccc(/C=C/C(c2cc(Cl)c(Cl)c(Cl)c2)C(F)(F)F)cc1C(F)(F)F] | 6 |
| F63 | 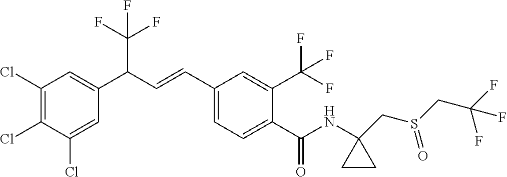 [SMILES: O=C(NC1(CS(=O)CC(F)(F)F)CC1)c1ccc(/C=C/C(c2cc(Cl)c(Cl)c(Cl)c2)C(F)(F)F)cc1C(F)(F)F] | 2a |
| F64 | 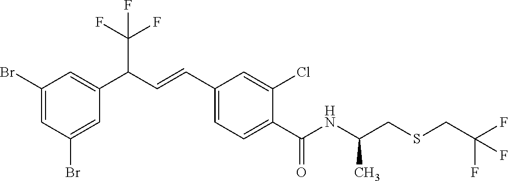 [SMILES: C[C@H](CSCC(F)(F)F)NC(=O)c1ccc(/C=C/C(c2cc(Br)cc(Br)c2)C(F)(F)F)cc1Cl] | 6 |
| F65 | 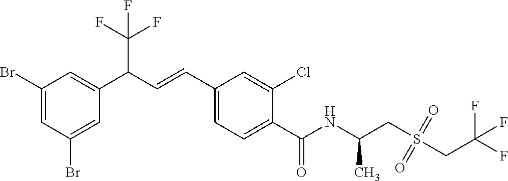 [SMILES: C[C@H](CS(=O)(=O)CC(F)(F)F)NC(=O)c1ccc(/C=C/C(c2cc(Br)cc(Br)c2)C(F)(F)F)cc1Cl] | 3 |
| F66 | 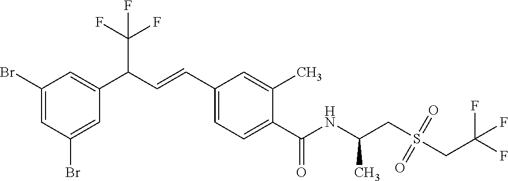 [SMILES: Cc1cc(/C=C/C(c2cc(Br)cc(Br)c2)C(F)(F)F)ccc1C(=O)N[C@H](C)CS(=O)(=O)CC(F)(F)F] | 3 |
| F67 | 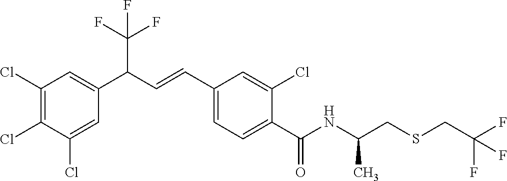 [SMILES: C[C@H](CSCC(F)(F)F)NC(=O)c1ccc(/C=C/C(c2cc(Cl)c(Cl)c(Cl)c2)C(F)(F)F)cc1Cl] | 6 |
| F68 | 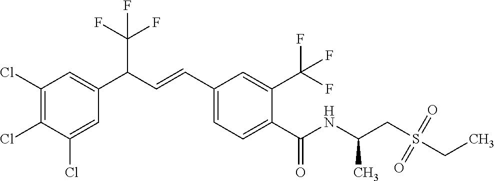 [SMILES: CCS(=O)(=O)C[C@@H](C)NC(=O)c1ccc(/C=C/C(c2cc(Cl)c(Cl)c(Cl)c2)C(F)(F)F)cc1C(F)(F)F] | 3 |
| F69 | 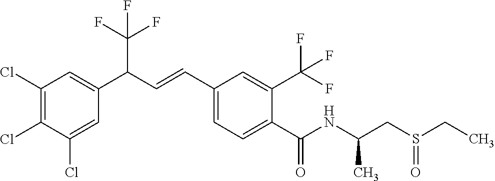 [SMILES: CCS(=O)C[C@@H](C)NC(=O)c1ccc(/C=C/C(c2cc(Cl)c(Cl)c(Cl)c2)C(F)(F)F)cc1C(F)(F)F] | 2a |
| F70 | 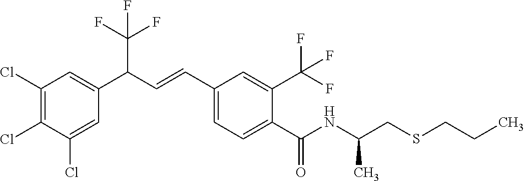 [SMILES: CCCSC[C@@H](C)NC(=O)c1ccc(/C=C/C(c2cc(Cl)c(Cl)c(Cl)c2)C(F)(F)F)cc1C(F)(F)F] | 6 |
| F71 | 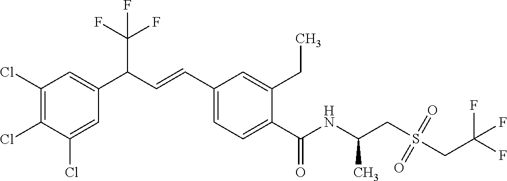 [SMILES: CCc1cc(/C=C/C(c2cc(Cl)c(Cl)c(Cl)c2)C(F)(F)F)ccc1C(=O)N[C@H](C)CS(=O)(=O)CC(F)(F)F] | 3 |
| F72 | 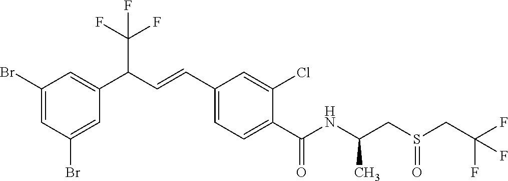 [SMILES: C[C@H](CS(=O)CC(F)(F)F)NC(=O)c1ccc(/C=C/C(c2cc(Br)cc(Br)c2)C(F)(F)F)cc1Cl] | 2a |
| F73 | 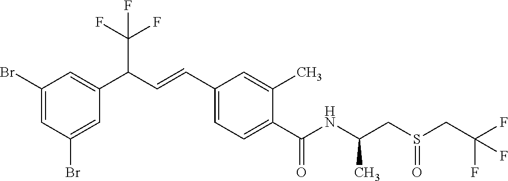 [SMILES: Cc1cc(/C=C/C(c2cc(Br)cc(Br)c2)C(F)(F)F)ccc1C(=O)N[C@H](C)CS(=O)CC(F)(F)F] | 2a |
| F75 | 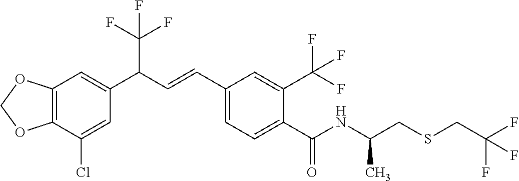 [SMILES: C[C@H](CSCC(F)(F)F)NC(=O)c1ccc(/C=C/C(c2cc(Cl)c3c(c2)OCO3)C(F)(F)F)cc1C(F)(F)F] | 6 |
| F76 | 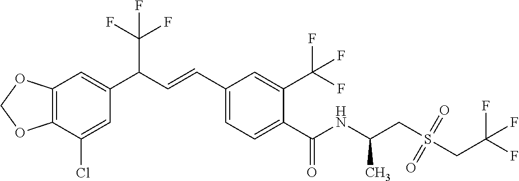 [SMILES: C[C@H](CS(=O)(=O)CC(F)(F)F)NC(=O)c1ccc(/C=C/C(c2cc(Cl)c3c(c2)OCO3)C(F)(F)F)cc1C(F)(F)F] | 3 |
| F79 | 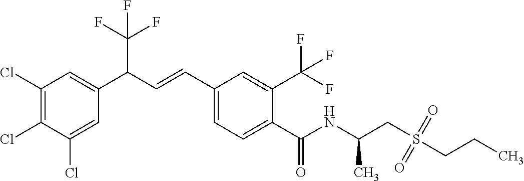 [SMILES: CCCS(=O)(=O)C[C@@H](C)NC(=O)c1ccc(/C=C/C(c2cc(Cl)c(Cl)c(Cl)c2)C(F)(F)F)cc1C(F)(F)F] | 3 |
| F80 | 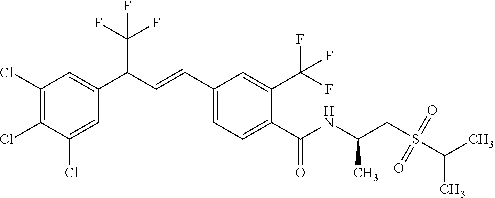 [SMILES: CC(C)S(=O)(=O)C[C@@H](C)NC(=O)c1ccc(/C=C/C(c2cc(Cl)c(Cl)c(Cl)c2)C(F)(F)F)cc1C(F)(F)F] | 3 |
| F81 | 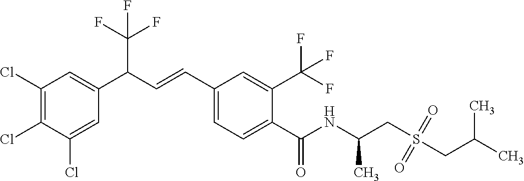 [SMILES: CC(C)CS(=O)(=O)C[C@@H](C)NC(=O)c1ccc(/C=C/C(c2cc(Cl)c(Cl)c(Cl)c2)C(F)(F)F)cc1C(F)(F)F] | 3 |
| F82 | 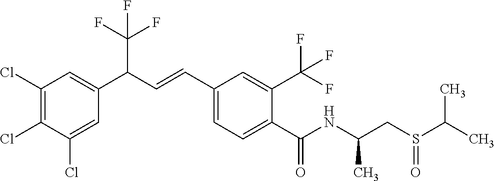 [SMILES: CC(C)S(=O)C[C@@H](C)NC(=O)c1ccc(/C=C/C(c2cc(Cl)c(Cl)c(Cl)c2)C(F)(F)F)cc1C(F)(F)F] | 2a |
| F83 | 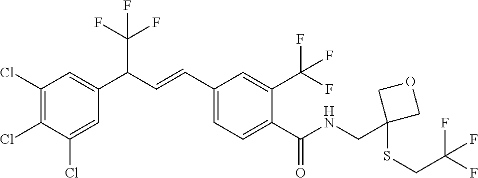 [SMILES: O=C(NCC1(SCC(F)(F)F)COC1)c1ccc(/C=C/C(c2cc(Cl)c(Cl)c(Cl)c2)C(F)(F)F)cc1C(F)(F)F] | 6 |
| F84 | 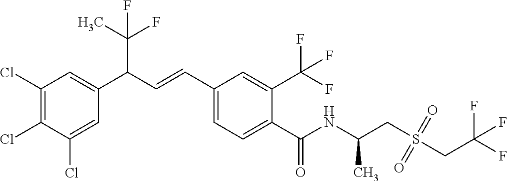 [SMILES: C[C@H](CS(=O)(=O)CC(F)(F)F)NC(=O)c1ccc(/C=C/C(c2cc(Cl)c(Cl)c(Cl)c2)C(C)(F)F)cc1C(F)(F)F] | 3 |
| F85 | 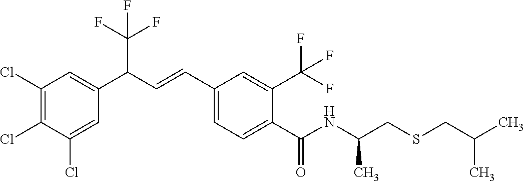 [SMILES: CC(C)CSC[C@@H](C)NC(=O)c1ccc(/C=C/C(c2cc(Cl)c(Cl)c(Cl)c2)C(F)(F)F)cc1C(F)(F)F] | 6 |
| F86 | 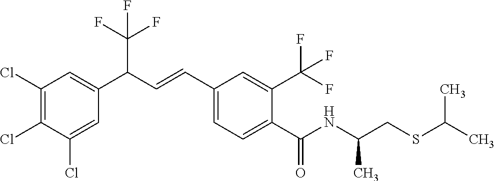 [SMILES: CC(C)SC[C@@H](C)NC(=O)c1ccc(/C=C/C(c2cc(Cl)c(Cl)c(Cl)c2)C(F)(F)F)cc1C(F)(F)F] | 6 |
| F87 | 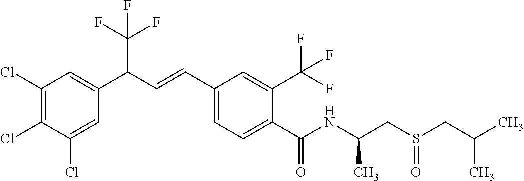 [SMILES: CC(C)CS(=O)C[C@@H](C)NC(=O)c1ccc(/C=C/C(c2cc(Cl)c(Cl)c(Cl)c2)C(F)(F)F)cc1C(F)(F)F] | 2a |
| F88 | 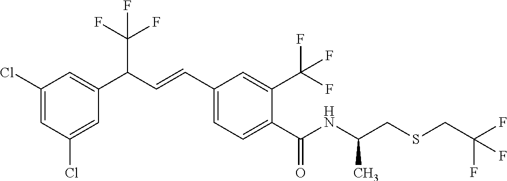 [SMILES: C[C@H](CSCC(F)(F)F)NC(=O)c1ccc(/C=C/C(c2cc(Cl)cc(Cl)c2)C(F)(F)F)cc1C(F)(F)F] | 6 |
| F89 | 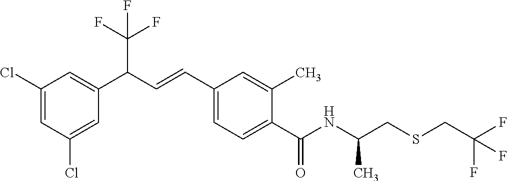 [SMILES: Cc1cc(/C=C/C(c2cc(Cl)cc(Cl)c2)C(F)(F)F)ccc1C(=O)N[C@H](C)CSCC(F)(F)F] | 6 |
| F90 | 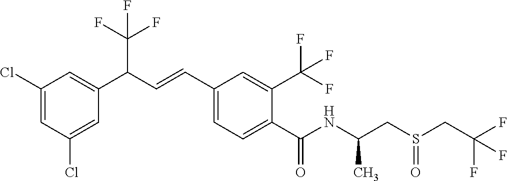 [SMILES: C[C@H](CS(=O)CC(F)(F)F)NC(=O)c1ccc(/C=C/C(c2cc(Cl)cc(Cl)c2)C(F)(F)F)cc1C(F)(F)F] | 2a |
| F91 | 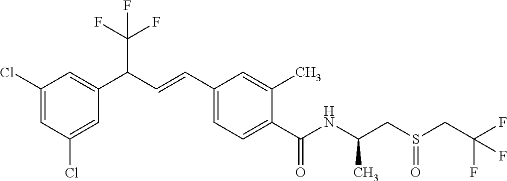 [SMILES: Cc1cc(/C=C/C(c2cc(Cl)cc(Cl)c2)C(F)(F)F)ccc1C(=O)N[C@H](C)CS(=O)CC(F)(F)F] | 2a |
| F92 | 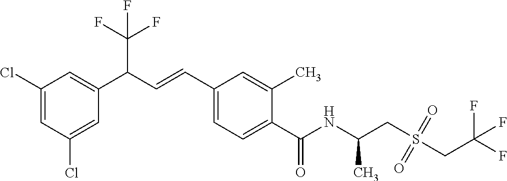 [SMILES: Cc1cc(/C=C/C(c2cc(Cl)cc(Cl)c2)C(F)(F)F)ccc1C(=O)N[C@H](C)CS(=O)(=O)CC(F)(F)F] | 3 |
| F93 | 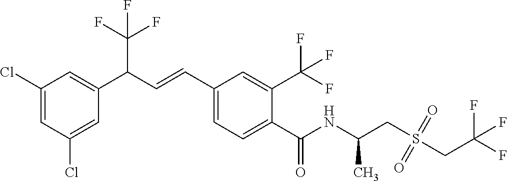 [SMILES: C[C@H](CS(=O)(=O)CC(F)(F)F)NC(=O)c1ccc(/C=C/C(c2cc(Cl)cc(Cl)c2)C(F)(F)F)cc1C(F)(F)F] | 3 |
| F94 | 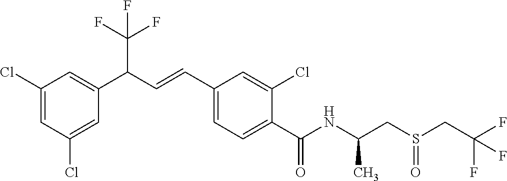 [SMILES: C[C@H](CS(=O)CC(F)(F)F)NC(=O)c1ccc(/C=C/C(c2cc(Cl)cc(Cl)c2)C(F)(F)F)cc1Cl] | 2a |
| F95 | 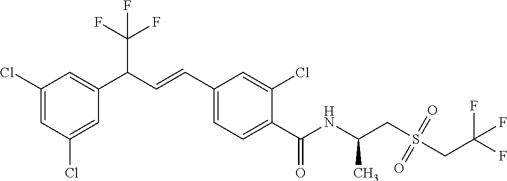 [SMILES: C[C@H](CS(=O)(=O)CC(F)(F)F)NC(=O)c1ccc(/C=C/C(c2cc(Cl)cc(Cl)c2)C(F)(F)F)cc1Cl] | 3 |
| F96 | 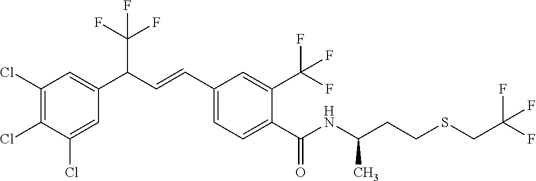 [SMILES: C[C@H](CCSCC(F)(F)F)NC(=O)c1ccc(/C=C/C(c2cc(Cl)c(Cl)c(Cl)c2)C(F)(F)F)cc1C(F)(F)F] | 6 |
| F97 | 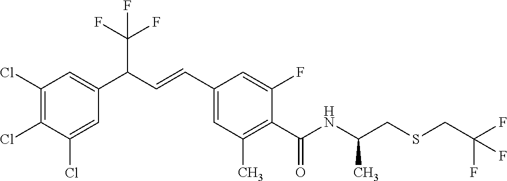 [SMILES: Cc1cc(/C=C/C(c2cc(Cl)c(Cl)c(Cl)c2)C(F)(F)F)cc(F)c1C(=O)N[C@H](C)CSCC(F)(F)F] | 6 |
| F98 | 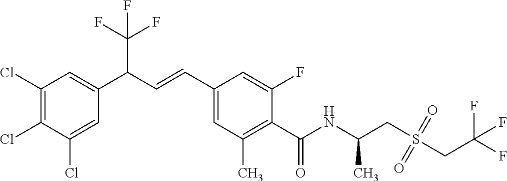 [SMILES: Cc1cc(/C=C/C(c2cc(Cl)c(Cl)c(Cl)c2)C(F)(F)F)cc(F)c1C(=O)N[C@H](C)CS(=O)(=O)CC(F)(F)F] | 3 |
| F99 | 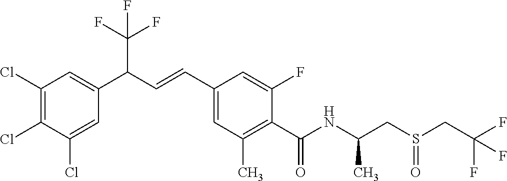 [SMILES: Cc1cc(/C=C/C(c2cc(Cl)c(Cl)c(Cl)c2)C(F)(F)F)cc(F)c1C(=O)N[C@H](C)CS(=O)CC(F)(F)F] | 2a |
| F100 | 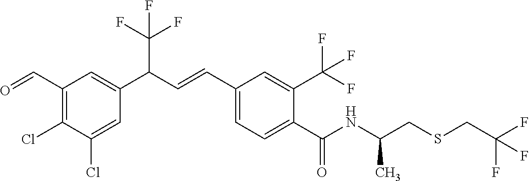 [SMILES: C[C@H](CSCC(F)(F)F)NC(=O)c1ccc(/C=C/C(c2cc(Cl)c(Cl)c(C=O)c2)C(F)(F)F)cc1C(F)(F)F] | 6 |
| F101 | 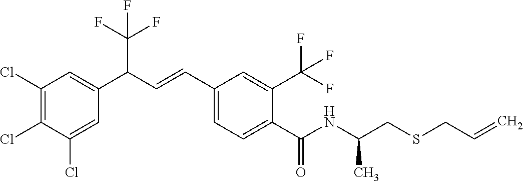 [SMILES: C=CCSC[C@@H](C)NC(=O)c1ccc(/C=C/C(c2cc(Cl)c(Cl)c(Cl)c2)C(F)(F)F)cc1C(F)(F)F] | 6 |
| F102 | 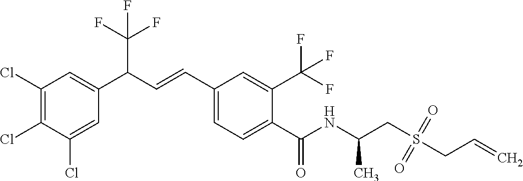 [SMILES: C=CCS(=O)(=O)C[C@@H](C)NC(=O)c1ccc(/C=C/C(c2cc(Cl)c(Cl)c(Cl)c2)C(F)(F)F)cc1C(F)(F)F] | 3 |
| F103 | 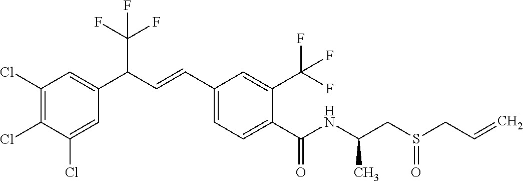 [SMILES: C=CCS(=O)C[C@@H](C)NC(=O)c1ccc(/C=C/C(c2cc(Cl)c(Cl)c(Cl)c2)C(F)(F)F)cc1C(F)(F)F] | 2a |
| F104 | 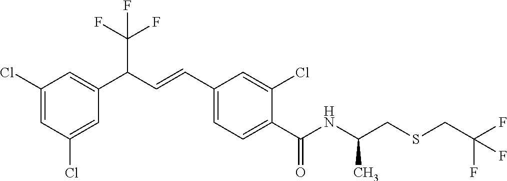 [SMILES: C[C@H](CSCC(F)(F)F)NC(=O)c1ccc(/C=C/C(c2cc(Cl)cc(Cl)c2)C(F)(F)F)cc1Cl] | 6 |
| F105 | 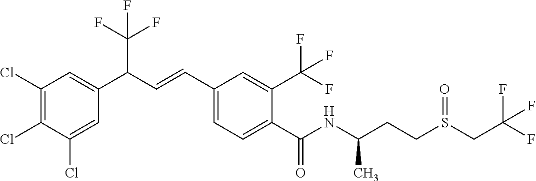 [SMILES: C[C@H](CCS(=O)CC(F)(F)F)NC(=O)c1ccc(/C=C/C(c2cc(Cl)c(Cl)c(Cl)c2)C(F)(F)F)cc1C(F)(F)F] | 2a |
| F106 | 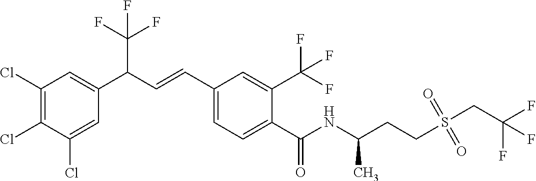 [SMILES: C[C@H](CCS(=O)(=O)CC(F)(F)F)NC(=O)c1ccc(/C=C/C(c2cc(Cl)c(Cl)c(Cl)c2)C(F)(F)F)cc1C(F)(F)F] | 3 |
| F107 | 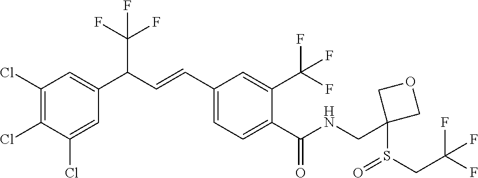 [SMILES: O=C(NCC1(S(=O)CC(F)(F)F)COC1)c1ccc(/C=C/C(c2cc(Cl)c(Cl)c(Cl)c2)C(F)(F)F)cc1C(F)(F)F] | 2a |
| F108 | 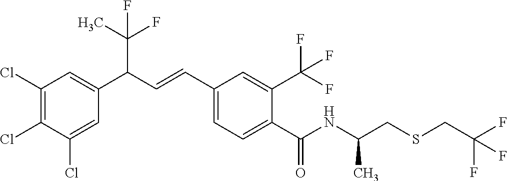 [SMILES: C[C@H](CSCC(F)(F)F)NC(=O)c1ccc(/C=C/C(c2cc(Cl)c(Cl)c(Cl)c2)C(C)(F)F)cc1C(F)(F)F] | 7 |
| F109 | 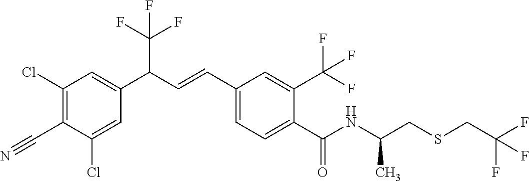 [SMILES: C[C@H](CSCC(F)(F)F)NC(=O)c1ccc(/C=C/C(c2cc(Cl)c(C#N)c(Cl)c2)C(F)(F)F)cc1C(F)(F)F] | 6 |
| F110 | 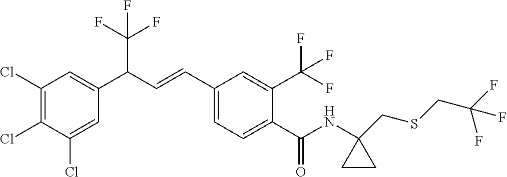 [SMILES: O=C(NC1(CSCC(F)(F)F)CC1)c1ccc(/C=C/C(c2cc(Cl)c(Cl)c(Cl)c2)C(F)(F)F)cc1C(F)(F)F] | 6 |
| F111 | 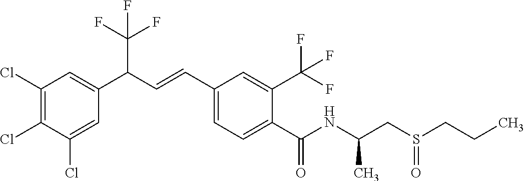 [SMILES: CCCS(=O)C[C@@H](C)NC(=O)c1ccc(/C=C/C(c2cc(Cl)c(Cl)c(Cl)c2)C(F)(F)F)cc1C(F)(F)F] | 2a |
| F112 | 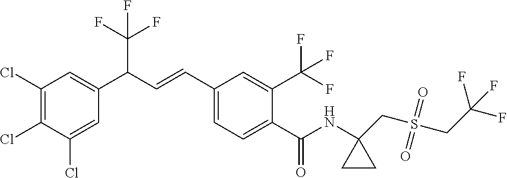 [SMILES: O=C(NC1(CS(=O)(=O)CC(F)(F)F)CC1)c1ccc(/C=C/C(c2cc(Cl)c(Cl)c(Cl)c2)C(F)(F)F)cc1C(F)(F)F] | 3 |
| F113 | 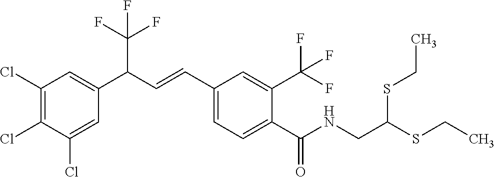 [SMILES: CCSC(CNC(=O)c1ccc(/C=C/C(c2cc(Cl)c(Cl)c(Cl)c2)C(F)(F)F)cc1C(F)(F)F)SCC] | 5 |
| F114 | 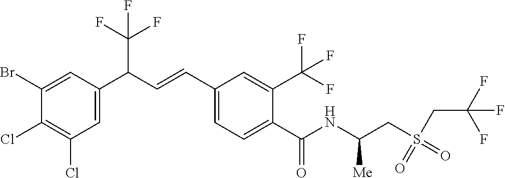 [SMILES: C[C@H](CS(=O)(=O)CC(F)(F)F)NC(=O)c1ccc(/C=C/C(c2cc(Cl)c(Cl)c(Br)c2)C(F)(F)F)cc1C(F)(F)F] | 7 |
| F115 | 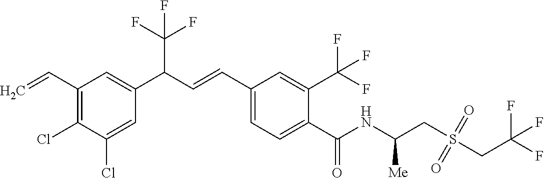 [SMILES: C=Cc1cc(C(/C=C/c2ccc(C(=O)N[C@H](C)CS(=O)(=O)CC(F)(F)F)c(C(F)(F)F)c2)C(F)(F)F)cc(Cl)c1Cl] | 7 |
| F116 | 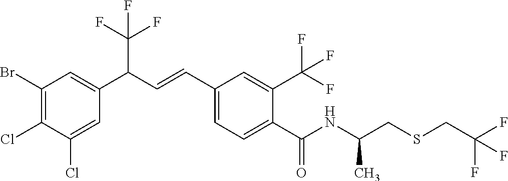 [SMILES: C[C@H](CSCC(F)(F)F)NC(=O)c1ccc(/C=C/C(c2cc(Cl)c(Cl)c(Br)c2)C(F)(F)F)cc1C(F)(F)F] | 7 |
| F117 | 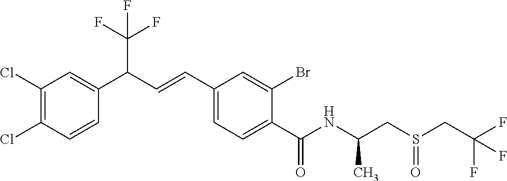 [SMILES: C[C@H](CS(=O)CC(F)(F)F)NC(=O)c1ccc(/C=C/C(c2ccc(Cl)c(Cl)c2)C(F)(F)F)cc1Br] | 2 |
| F118 | 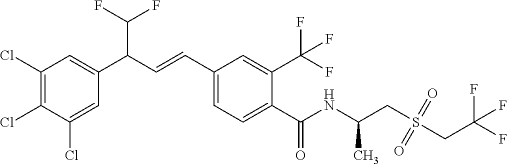 [SMILES: C[C@H](CS(=O)(=O)CC(F)(F)F)NC(=O)c1ccc(/C=C/C(c2cc(Cl)c(Cl)c(Cl)c2)C(F)F)cc1C(F)(F)F] | 6 |
| F119 | 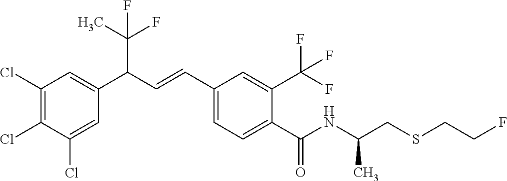 [SMILES: C[C@H](CSCCF)NC(=O)c1ccc(/C=C/C(c2cc(Cl)c(Cl)c(Cl)c2)C(C)(F)F)cc1C(F)(F)F] | 9 |
| F120 | 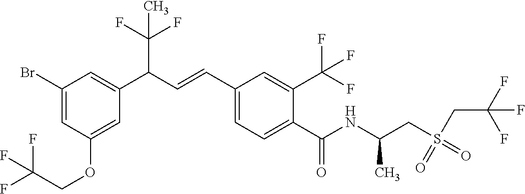 [SMILES: C[C@H](CS(=O)(=O)CC(F)(F)F)NC(=O)c1ccc(/C=C/C(c2cc(Br)cc(OCC(F)(F)F)c2)C(C)(F)F)cc1C(F)(F)F] | 6 |
| F121 | 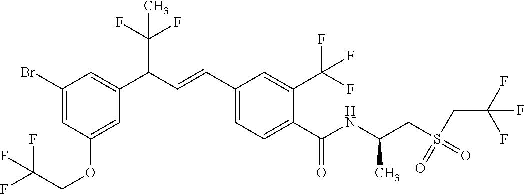 [SMILES: C[C@H](CS(=O)(=O)CC(F)(F)F)NC(=O)c1ccc(/C=C/C(c2cc(Br)cc(OCC(F)(F)F)c2)C(C)(F)F)cc1C(F)(F)F] | 3 |
| F122 | 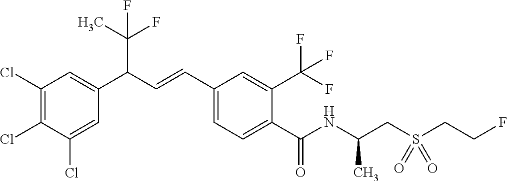 [SMILES: C[C@H](CS(=O)(=O)CCF)NC(=O)c1ccc(/C=C/C(c2cc(Cl)c(Cl)c(Cl)c2)C(C)(F)F)cc1C(F)(F)F] | 3 |
| F123 | 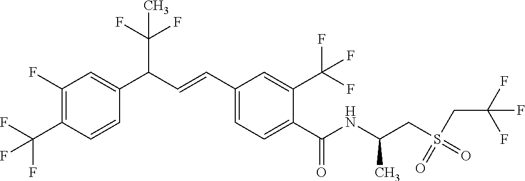 [SMILES: C[C@H](CS(=O)(=O)CC(F)(F)F)NC(=O)c1ccc(/C=C/C(c2ccc(C(F)(F)F)c(F)c2)C(C)(F)F)cc1C(F)(F)F] | 6 |
| F124 | 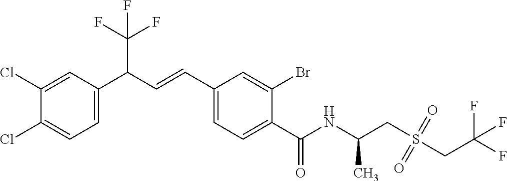 [SMILES: C[C@H](CS(=O)(=O)CC(F)(F)F)NC(=O)c1ccc(/C=C/C(c2ccc(Cl)c(Cl)c2)C(F)(F)F)cc1Br] | 7 |
| F125 | 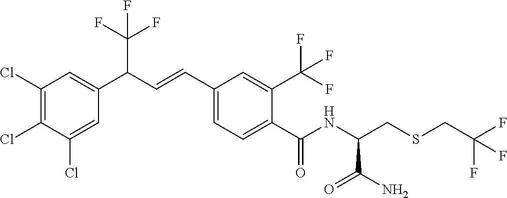 [SMILES: NC(=O)[C@H](CSCC(F)(F)F)NC(=O)c1ccc(/C=C/C(c2cc(Cl)c(Cl)c(Cl)c2)C(F)(F)F)cc1C(F)(F)F] | 10 |
| F126 | 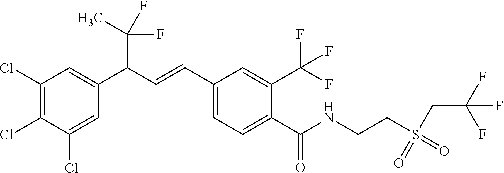 [SMILES: CC(F)(F)C(/C=C/c1ccc(C(=O)NCCS(=O)(=O)CC(F)(F)F)c(C(F)(F)F)c1)c1cc(Cl)c(Cl)c(Cl)c1] | 3 |
| F128 | 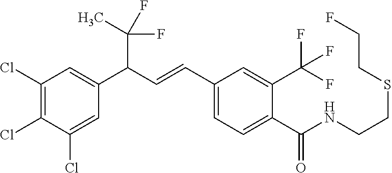 [SMILES: CC(F)(F)C(/C=C/c1ccc(C(=O)NCCSCCF)c(C(F)(F)F)c1)c1cc(Cl)c(Cl)c(Cl)c1] | 9 |
| F129 | 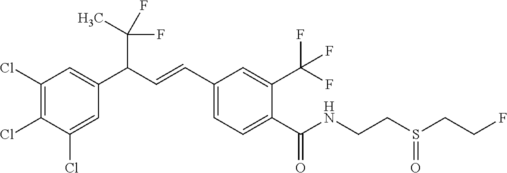 [SMILES: CC(F)(F)C(/C=C/c1ccc(C(=O)NCCS(=O)CCF)c(C(F)(F)F)c1)c1cc(Cl)c(Cl)c(Cl)c1] | 3 |
| F130 | 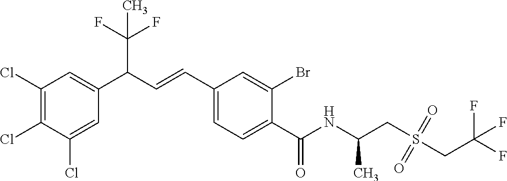 [SMILES: C[C@H](CS(=O)(=O)CC(F)(F)F)NC(=O)c1ccc(/C=C/C(c2cc(Cl)c(Cl)c(Cl)c2)C(C)(F)F)cc1Br] | 47 |
| F131 | 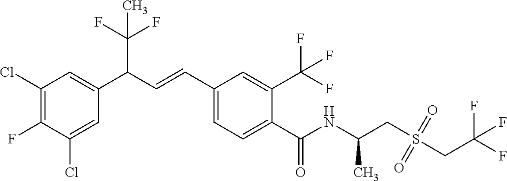 [SMILES: C[C@H](CS(=O)(=O)CC(F)(F)F)NC(=O)c1ccc(/C=C/C(c2cc(Cl)c(F)c(Cl)c2)C(C)(F)F)cc1C(F)(F)F] | 47 |
| F132 | 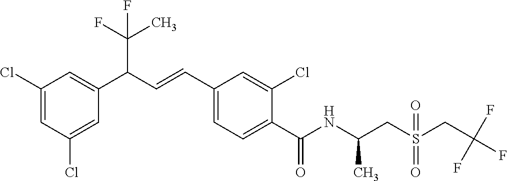 [SMILES: C[C@H](CS(=O)(=O)CC(F)(F)F)NC(=O)c1ccc(/C=C/C(c2cc(Cl)cc(Cl)c2)C(C)(F)F)cc1Cl] | 47 |
| F133 | 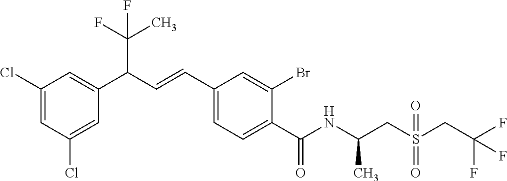 [SMILES: C[C@H](CS(=O)(=O)CC(F)(F)F)NC(=O)c1ccc(/C=C/C(c2cc(Cl)cc(Cl)c2)C(C)(F)F)cc1Br] | 47 |
| F134 | 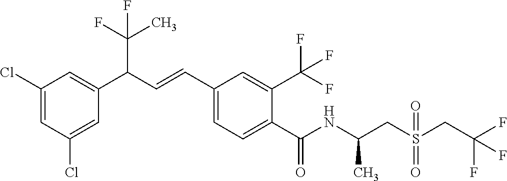 [SMILES: C[C@H](CS(=O)(=O)CC(F)(F)F)NC(=O)c1ccc(/C=C/C(c2cc(Cl)cc(Cl)c2)C(C)(F)F)cc1C(F)(F)F] | 47 |
| F135 | 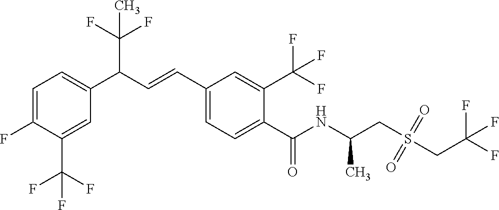 [SMILES: C[C@H](CS(=O)(=O)CC(F)(F)F)NC(=O)c1ccc(/C=C/C(c2ccc(F)c(C(F)(F)F)c2)C(C)(F)F)cc1C(F)(F)F] | 47 |
| F136 | 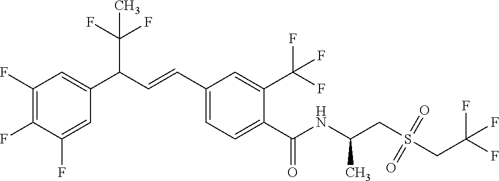 [SMILES: C[C@H](CS(=O)(=O)CC(F)(F)F)NC(=O)c1ccc(/C=C/C(c2cc(F)c(F)c(F)c2)C(C)(F)F)cc1C(F)(F)F] | 47 |
| F137 | 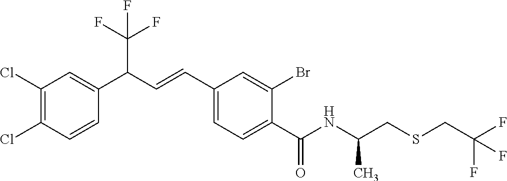 [SMILES: C[C@H](CSCC(F)(F)F)NC(=O)c1ccc(/C=C/C(c2ccc(Cl)c(Cl)c2)C(F)(F)F)cc1Br] | 7 |

*prepared according to example number

TABLE 2
Structure and Preparation Method for C Series Molecules

| No. | Structure | Prep.* |
|---|---|---|
| C1 | 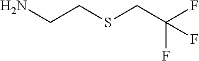 | 11 |
| C7 | 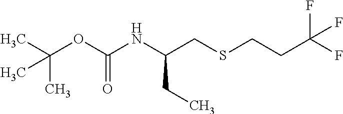 [SMILES: CC[C@H](CSCCC(F)(F)F)NC(=O)OC(C)(C)C] | 12 |
| C8 | 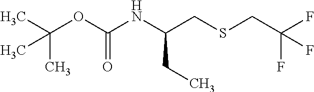 [SMILES: CC[C@H](CSCC(F)(F)F)NC(=O)OC(C)(C)C] | 12 |
| C9 | 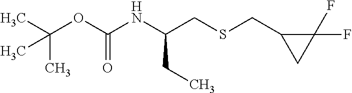 [SMILES: CC[C@H](CSCC1CC1(F)F)NC(=O)OC(C)(C)C] | 12 |
| C10 | 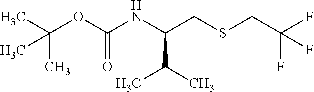 [SMILES: CC(C)[C@H](CSCC(F)(F)F)NC(=O)OC(C)(C)C] | 12 |
| C11 | 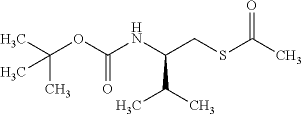 [SMILES: CC(=O)SC[C@H](NC(=O)OC(C)(C)C)C(C)C] | 13 |
| C12 | 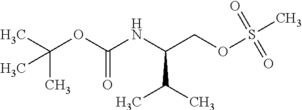 [SMILES: CC(C)[C@H](COS(C)(=O)=O)NC(=O)OC(C)(C)C] | 14 |
| C13 | 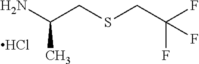 | 15 |
| C14 | 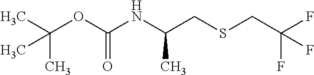 [SMILES: C[C@H](CSCC(F)(F)F)NC(=O)OC(C)(C)C] | 12 |
| C15 | 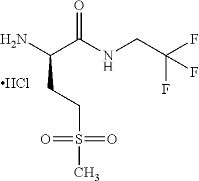 [SMILES: Cl ; CS(=O)(=O)CC[C@@H](N)C(=O)NCC(F)(F)F] | 15 |
| C16 | 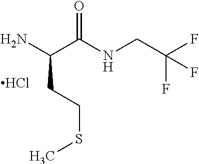 | 15 |
| C17 | 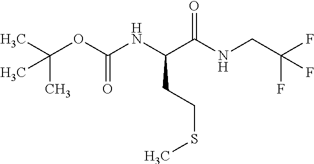 [SMILES: CSCC[C@@H](NC(=O)OC(C)(C)C)C(=O)NCC(F)(F)F] | 16 |
| C18 | 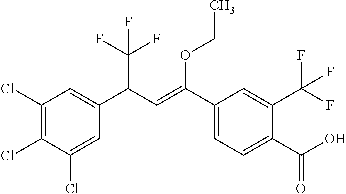 [SMILES: CCO/C(=C\C(c1cc(Cl)c(Cl)c(Cl)c1)C(F)(F)F)c1ccc(C(=O)O)c(C(F)(F)F)c1] | 17 |
| C19 | 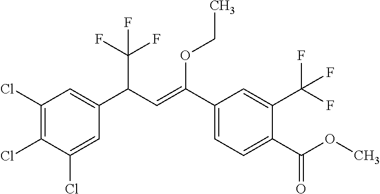 [SMILES: CCO/C(=C\C(c1cc(Cl)c(Cl)c(Cl)c1)C(F)(F)F)c1ccc(C(=O)OC)c(C(F)(F)F)c1] | 18 |
| C20 | 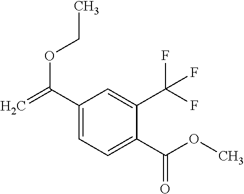 [SMILES: C=C(OCC)c1ccc(C(=O)OC)c(C(F)(F)F)c1] | 19 |
| C21 | 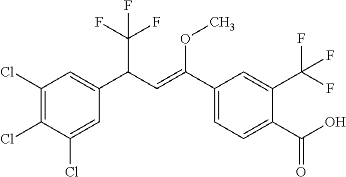 [SMILES: CO/C(=C\C(c1cc(Cl)c(Cl)c(Cl)c1)C(F)(F)F)c1ccc(C(=O)O)c(C(F)(F)F)c1] | 17 |
| C22 | 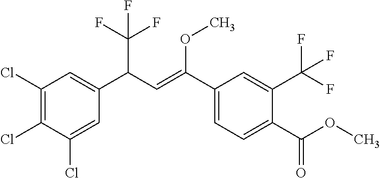 [SMILES: COC(=O)c1ccc(/C(=C/C(c2cc(Cl)c(Cl)c(Cl)c2)C(F)(F)F)OC)cc1C(F)(F)F] | 18 |
| C23 | 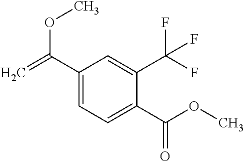 [SMILES: C=C(OC)c1ccc(C(=O)OC)c(C(F)(F)F)c1] | 20 |
| C24 | 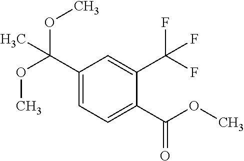 [SMILES: COC(=O)c1ccc(C(C)(OC)OC)cc1C(F)(F)F] | 21 |
| C25 | 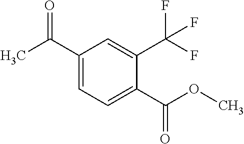 [SMILES: COC(=O)c1ccc(C(C)=O)cc1C(F)(F)F] | 22 |
| C26 | 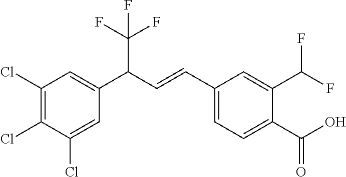 [SMILES: O=C(O)c1ccc(/C=C/C(c2cc(Cl)c(Cl)c(Cl)c2)C(F)(F)F)cc1C(F)F] | 18 |
| C27 | 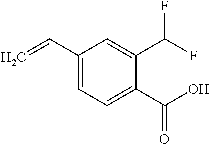 | 23 |
| C28 | 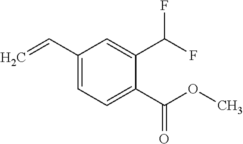 | 24 |
| C29 | 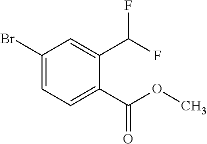 | 25 |
| C30 | 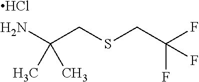 | 26 |
| C31 | 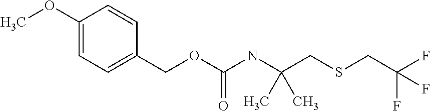 [SMILES: COc1ccc(COC(=O)NC(C)(C)CSCC(F)(F)F)cc1] | 27 |
| C32 | 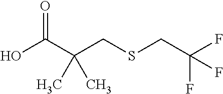 | 28 |
| C33 | 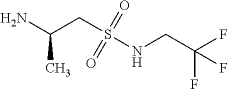 | 29 |
| C34 | 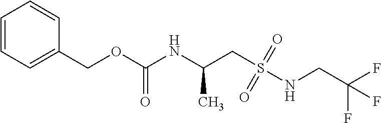 [SMILES: C[C@H](CS(=O)(=O)NCC(F)(F)F)NC(=O)OCc1ccccc1] | 30 |
| C35 | 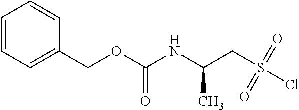 [SMILES: C[C@H](CS(=O)(=O)Cl)NC(=O)OCc1ccccc1] | 31 |
| C36 | 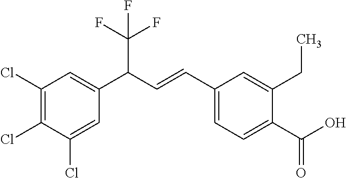 [SMILES: CCc1cc(/C=C/C(c2cc(Cl)c(Cl)c(Cl)c2)C(F)(F)F)ccc1C(=O)O] | 18 |
| C37 | 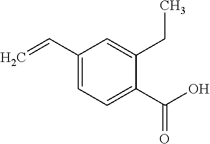 | 24 |
| C38 | 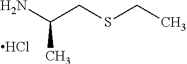 | 15 |
| C39 | 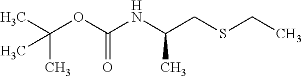 | 12 |
| C40 | 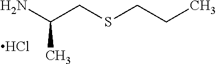 | 15 |
| C41 | 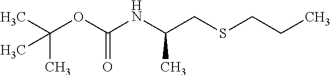 | 12 |
| C42 | 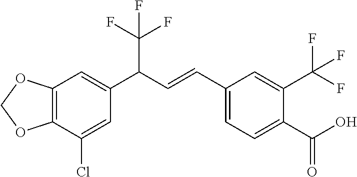 [SMILES: O=C(O)c1ccc(/C=C/C(c2cc(Cl)c3c(c2)OCO3)C(F)(F)F)cc1C(F)(F)F] | 18 |
| C43 | 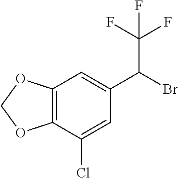 [SMILES: FC(F)(F)C(Br)c1cc(Cl)c2c(c1)OCO2] | 32 |
| C44 | 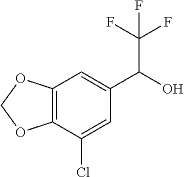 [SMILES: OC(c1cc(Cl)c2c(c1)OCO2)C(F)(F)F] | 33 |
| C45 | 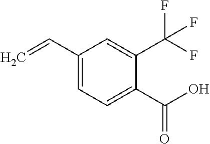 | 24 |
| C46 | 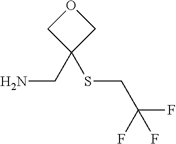 [SMILES: NCC1(SCC(F)(F)F)COC1] | 34 |
| C47 | 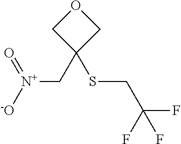 | 35 |
| C48 | 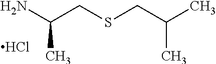 | 15 |
| C49 | 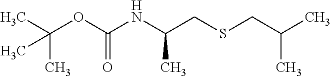 [SMILES: CC(C)CSC[C@@H](C)NC(=O)OC(C)(C)C] | 12 |
| C50 | 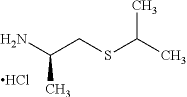 | 15 |
| C51 | 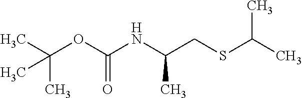 | 12 |
| C52 | 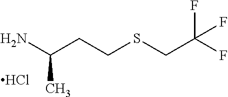 | 15 |
| C53 | 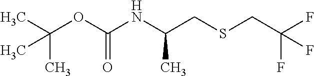 [SMILES: C[C@H](CSCC(F)(F)F)NC(=O)OC(C)(C)C] | 12 |
| C54 | 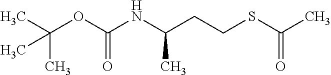 [SMILES: CC(=O)SCC[C@@H](C)NC(=O)OC(C)(C)C] | 13 |
| C55 | 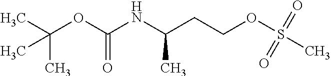 [SMILES: C[C@H](CCOS(C)(=O)=O)NC(=O)OC(C)(C)C] | 14 |
| C56 | 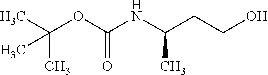 | 36 |
| C57 | 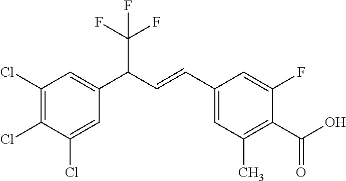 [SMILES: Cc1cc(/C=C/C(c2cc(Cl)c(Cl)c(Cl)c2)C(F)(F)F)cc(F)c1C(=O)O] | 18 |
| C58 | 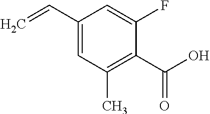 | 37 |
| C59 | 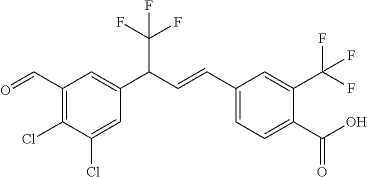 [SMILES: O=Cc1cc(C(/C=C/c2ccc(C(=O)O)c(C(F)(F)F)c2)C(F)(F)F)cc(Cl)c1Cl] | 38 |
| C60 | 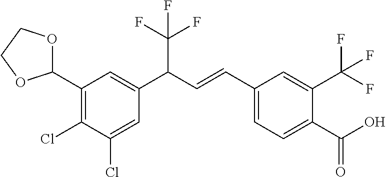 [SMILES: O=C(O)c1ccc(/C=C/C(c2cc(Cl)c(Cl)c(C3OCCO3)c2)C(F)(F)F)cc1C(F)(F)F] | 18 |
| C61 | 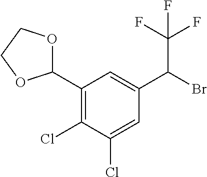 [SMILES: FC(F)(F)C(Br)c1cc(Cl)c(Cl)c(C2OCCO2)c1] | 39 |
| C62 | 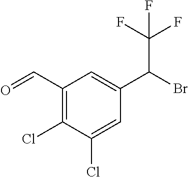 [SMILES: O=Cc1cc(C(Br)C(F)(F)F)cc(Cl)c1Cl] | 32 |
| C63 | 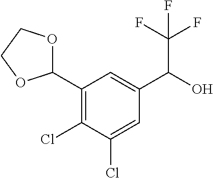 [SMILES: OC(c1cc(Cl)c(Cl)c(C2OCCO2)c1)C(F)(F)F] | 33 |
| C64 | 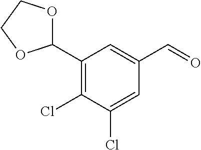 [SMILES: O=Cc1cc(Cl)c(Cl)c(C2OCCO2)c1] | 39 |
| C65 | 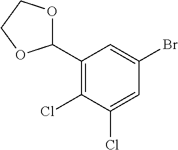 [SMILES: Clc1cc(Br)cc(C2OCCO2)c1Cl] | 39 |
| C66 | 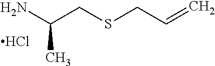 | 15 |
| C67 | 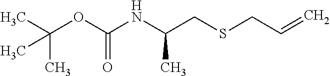 | 12 |
| C68 | 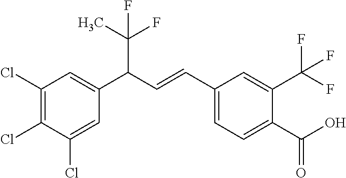 [SMILES: CC(F)(F)C(/C=C/c1ccc(C(=O)O)c(C(F)(F)F)c1)c1cc(Cl)c(Cl)c(Cl)c1] | 18 |
| C69 | 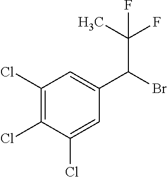 | 41 |
| C70 | 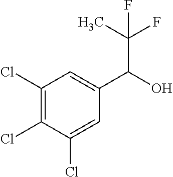 | 42 |
| C71 | 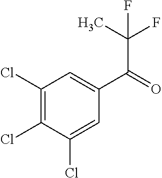 | 43 |
| C72 | 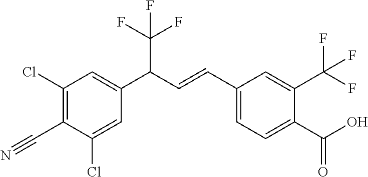 [SMILES: N#Cc1c(Cl)cc(C(/C=C/c2ccc(C(=O)O)c(C(F)(F)F)c2)C(F)(F)F)cc1Cl] | 18 |
| C73 | 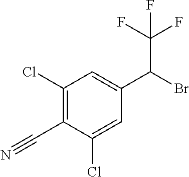 [SMILES: N#Cc1c(Cl)cc(C(Br)C(F)(F)F)cc1Cl] | 32 |
| C74 | 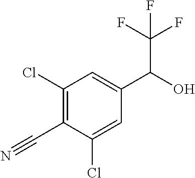 [SMILES: N#Cc1c(Cl)cc(C(O)C(F)(F)F)cc1Cl] | 33 |
| C75 | 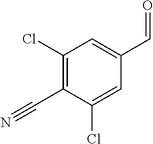 [SMILES: N#Cc1c(Cl)cc(C=O)cc1Cl] | 44 |
| C76 | 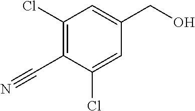 [SMILES: N#Cc1c(Cl)cc(CO)cc1Cl] | 42 |
| C77 | 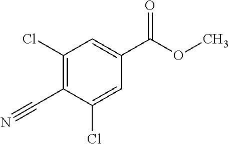 | 45 |
| C78 | 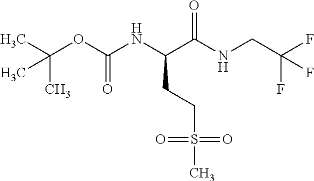 [SMILES: CC(C)(C)OC(=O)N[C@H](CCS(C)(=O)=O)C(=O)NCC(F)(F)F] | 46 |
| C79 | 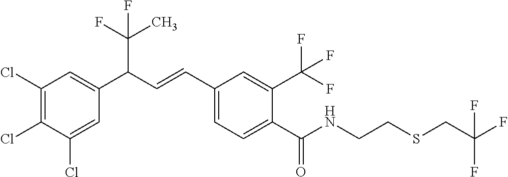 [SMILES: CC(F)(F)C(/C=C/c1ccc(C(=O)NCCSCC(F)(F)F)c(C(F)(F)F)c1)c1cc(Cl)c(Cl)c(Cl)c1] | 9 |
| C80 | 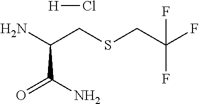 | 15 |
| C81 | 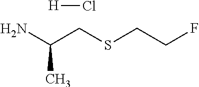 | 15 |
| C82 | 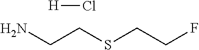 | 15 |
| C83 | 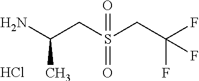 | 15 |
| C84 | 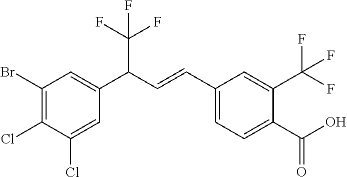 [SMILES: O=C(O)c1ccc(/C=C/C(c2cc(Cl)c(Cl)c(Br)c2)C(F)(F)F)cc1C(F)(F)F] | 18 |
| C85 | 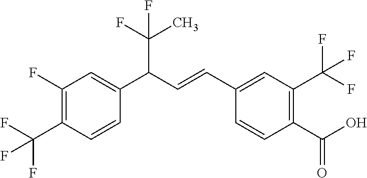 [SMILES: CC(F)(F)C(/C=C/c1ccc(C(=O)O)c(C(F)(F)F)c1)c1ccc(C(F)(F)F)c(F)c1] | 18 |
| C86 | 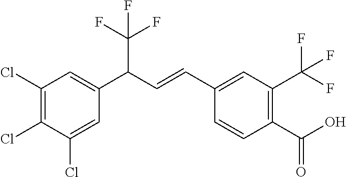 [SMILES: O=C(O)c1ccc(/C=C/C(c2cc(Cl)c(Cl)c(Cl)c2)C(F)(F)F)cc1C(F)(F)F] | 18 |
| C87 | 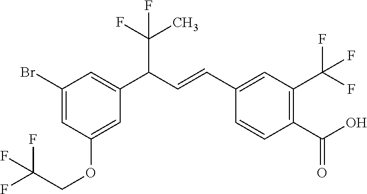 [SMILES: CC(F)(F)C(/C=C/c1ccc(C(=O)O)c(C(F)(F)F)c1)c1cc(Br)cc(OCC(F)(F)F)c1] | 18 |
| C88 | 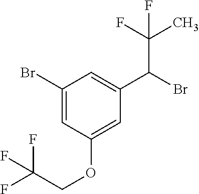 [SMILES: CC(F)(F)C(Br)c1cc(Br)cc(OCC(F)(F)F)c1] | 32 |
| C89 | 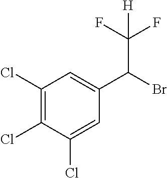 [SMILES: FC(F)C(Br)c1cc(Cl)c(Cl)c(Cl)c1] | 32 |
| C90 | 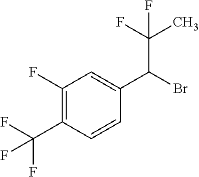 [SMILES: CC(F)(F)C(Br)c1ccc(C(F)(F)F)c(F)c1] | 32 |
| C91 | 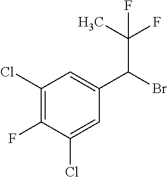 | 32 |
| C92 | 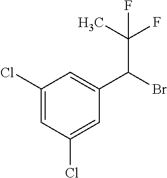 | 32 |
| C93 | 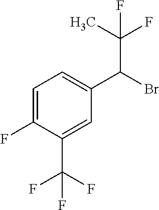 [SMILES: CC(F)(F)C(Br)c1ccc(F)c(C(F)(F)F)c1] | 32 |
| C94 | 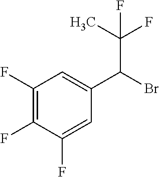 | 32 |
| C95 | 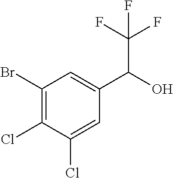 [SMILES: OC(c1cc(Cl)c(Cl)c(Br)c1)C(F)(F)F] | 33 |
| C96 | 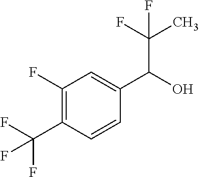 [SMILES: CC(F)(F)C(O)c1ccc(C(F)(F)F)c(F)c1] | 42 |
| C97 | 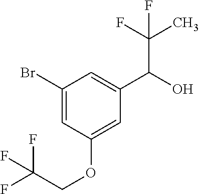 [SMILES: CC(F)(F)C(O)c1cc(Br)cc(OCC(F)(F)F)c1] | 42 |
| C98 | 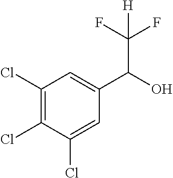 [SMILES: OC(c1cc(Cl)c(Cl)c(Cl)c1)C(F)F] | 42 |
| C99 | 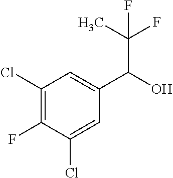 | 42 |
| C100 | 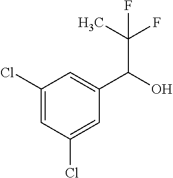 | 42 |
| C101 | 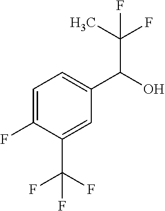 [SMILES: CC(F)(F)C(O)c1ccc(F)c(C(F)(F)F)c1] | 42 |
| C102 | 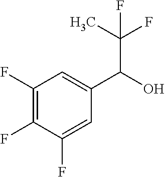 | 42 |
| C103 | 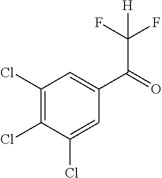 [SMILES: O=C(c1cc(Cl)c(Cl)c(Cl)c1)C(F)F] | 43 |
| C104 | 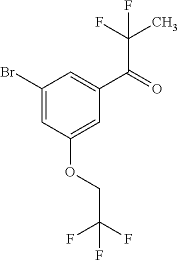 [SMILES: CC(F)(F)C(=O)c1cc(Br)cc(OCC(F)(F)F)c1] | 43 |
| C105 | 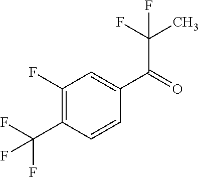 [SMILES: CC(F)(F)C(=O)c1ccc(C(F)(F)F)c(F)c1] | 43 |
| C106 | 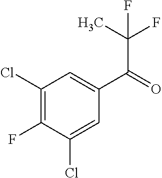 | 43 |
| C107 | 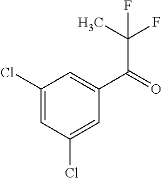 | 43 |
| C108 | 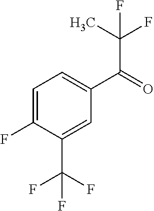 [SMILES: CC(F)(F)C(=O)c1ccc(F)c(C(F)(F)F)c1] | 43 |
| C109 | 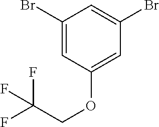 [SMILES: FC(F)(F)COc1cc(Br)cc(Br)c1] | 48 |
| C110 | 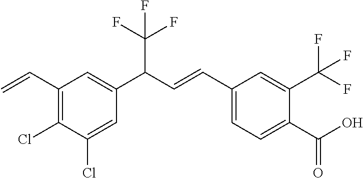 [SMILES: C=Cc1cc(C(/C=C/c2ccc(C(=O)O)c(C(F)(F)F)c2)C(F)(F)F)cc(Cl)c1Cl] | 49 |
| C111 | 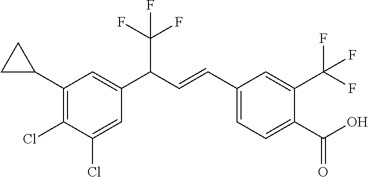 [SMILES: O=C(O)c1ccc(/C=C/C(c2cc(Cl)c(Cl)c(C3CC3)c2)C(F)(F)F)cc1C(F)(F)F] | 49 |
| C112 | 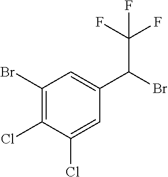 [SMILES: FC(F)(F)C(Br)c1cc(Cl)c(Cl)c(Br)c1] | 50 |
| C113 | 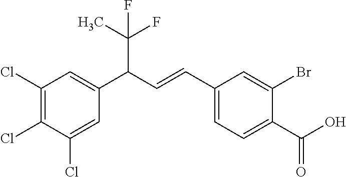 [SMILES: CC(F)(F)C(/C=C/c1ccc(C(=O)O)c(Br)c1)c1cc(Cl)c(Cl)c(Cl)c1] | 51 |
| C114 | 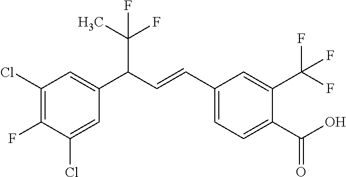 [SMILES: CC(F)(F)C(/C=C/c1ccc(C(=O)O)c(C(F)(F)F)c1)c1cc(Cl)c(F)c(Cl)c1] | 51 |
| C115 | 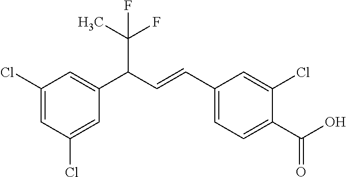 [SMILES: CC(F)(F)C(/C=C/c1ccc(C(=O)O)c(Cl)c1)c1cc(Cl)cc(Cl)c1] | 51 |
| C116 | 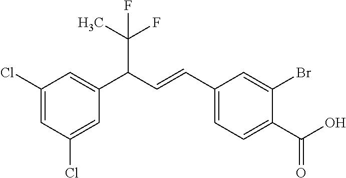 [SMILES: CC(F)(F)C(/C=C/c1ccc(C(=O)O)c(Br)c1)c1cc(Cl)cc(Cl)c1] | 51 |
| C117 | 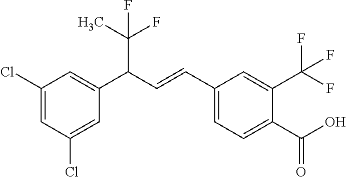 [SMILES: CC(F)(F)C(/C=C/c1ccc(C(=O)O)c(C(F)(F)F)c1)c1cc(Cl)cc(Cl)c1] | 51 |
| C118 | 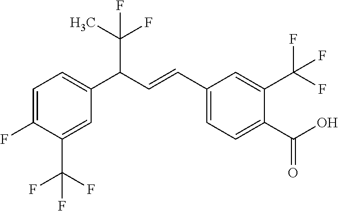 [SMILES: CC(F)(F)C(/C=C/c1ccc(C(=O)O)c(C(F)(F)F)c1)c1ccc(F)c(C(F)(F)F)c1] | 51 |
| C119 | 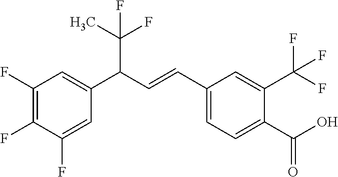 [SMILES: CC(F)(F)C(/C=C/c1ccc(C(=O)O)c(C(F)(F)F)c1)c1cc(F)c(F)c(F)c1] | 51 |
| C120 | 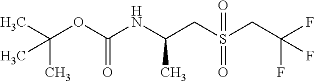 [SMILES: C[C@H](CS(=O)(=O)CC(F)(F)F)NC(=O)OC(C)(C)C] | 52 |
| C121 | 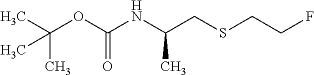 | 53 |
| C122 | 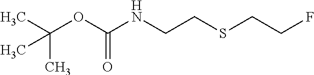 | 54 |
| C123 | 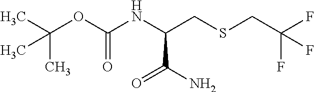 [SMILES: CC(C)(C)OC(=O)N[C@@H](CSCC(F)(F)F)C(N)=O] | 55 |
| C124 | 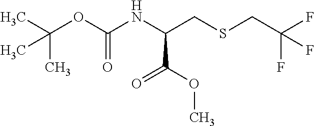 [SMILES: COC(=O)[C@H](CSCC(F)(F)F)NC(=O)OC(C)(C)C] | 56 |

*prepared according to example number

TABLE 3
Analytical Data for Molecules in Table 1

| | Mp | | | ¹³C NMR; |
|---|---|---|---|---|
| No. | (° C.) | Mass (m/z) | ¹H NMR | ¹⁹F NMR; IR |
| F1 | | ESIMS 628 | ¹H NMR (400 MHz, | ¹⁹F NMR (376 MHz, |
| | | ([M + H]⁺) | DMSO-d₆) δ 8.59 (t, J = 5.7 Hz, | DMSO-d₆) |
| | | | 1H), | δ −65.01, |
| | | | 7.94-7.92 (m, 3H), | −68.03 |
| | | | 7.60 (dd, J = 7.9, 1.6 Hz, | |
| | | | 1H), 7.37 (d, J = 7.8 Hz, | |
| | | | 1H), 6.97 (dd, J = 15.7, | |
| | | | 9.3 Hz, 1H), | |
| | | | 6.75 (d, J = 15.7 Hz, | |
| | | | 1H), 4.83 (p, J = 9.4 Hz, | |
| | | | 1H), 3.55 (q, J = 10.8 Hz, | |
| | | | 2H), | |
| | | | 3.44 (q, J = 6.6 Hz, 2H), | |
| | | | 2.84 (t, J = 6.9 Hz, | |
| | | | 2H) | |
| F2 | | ESIMS 652 | ¹H NMR (400 MHz, | ¹⁹F NMR (376 MHz, |
| | | ([M + H]⁺) | DMSO-d₆) δ 8.75 (t, J = 5.7 Hz, | DMSO-d₆) |
| | | | 1H), | δ −68.04 |
| | | | 7.95 (d, J = 1.6 Hz, 1H), | |
| | | | 7.93 (s, 2H), | |
| | | | 7.61 (dd, J = 8.1, 1.6 Hz, | |
| | | | 1H), 7.42-7.31 (m, | |
| | | | 6H), 6.99 (dd, J = 15.7, | |
| | | | 9.2 Hz, 1H), | |
| | | | 6.78 (d, J = 15.6 Hz, | |
| | | | 1H), 4.85 (p, J = 9.4 Hz, | |
| | | | 1H), 4.24 (d, J = 12.8 Hz, | |
| | | | 1H), | |
| | | | 4.07 (d, J = 12.7 Hz, 1H), | |
| | | | 3.75-3.52 (m, 2H), | |
| | | | 3.06 (dt, J = 13.1, | |
| | | | 7.2 Hz, 1H), 2.84 (dt, | |
| | | | J = 13.2, 6.0 Hz, 1H) | |
| F3 | | ESIMS 646 | ¹H NMR (400 MHz, | ¹⁹F NMR (376 MHz, |
| | | ([M + H]⁺) | CDCl₃) δ 7.57 (d, J = 1.6 Hz, | CDCl₃) δ |
| | | | 1H), 7.45 (d, | −60.78, −68.61 |
| | | | J = 8.0 Hz, 1H), | |
| | | | 7.41 (s, 2H), 7.33 (dd, J = 8.0, | |
| | | | 1.6 Hz, 1H), | |
| | | | 7.13 (t, J = 5.8 Hz, 1H), | |
| | | | 6.52 (d, J = 15.9 Hz, | |
| | | | 1H), 6.38 (dd, J = 15.9, | |
| | | | 7.9 Hz, 1H), | |
| | | | 4.11 (p, J = 8.6 Hz, | |
| | | | 1H), 4.00 (dq, J = 14.6, | |
| | | | 5.5 Hz, 1H), | |
| | | | 3.89 (dddd, J = 14.6, | |
| | | | 8.5, 6.1, 4.3 Hz, 1H), | |
| | | | 3.54 (qd, J = 9.9, 6.0 Hz, | |
| | | | 2H), 3.36 (ddd, J = 13.4, | |
| | | | 8.5, 4.9 Hz, | |
| | | | 1H), 3.12 (ddd, J = 13.3, | |
| | | | 5.8, 4.4 Hz, 1H) | |
| F4 | | ESIMS 690 | ¹H NMR (400 MHz, | ¹⁹F NMR (376 MHz, |
| | | ([M + H]⁺) | DMSO-d₆) δ 8.55 (t, J = 5.5 Hz, | DMSO-d₆) |
| | | | 1H), | δ −68.01 |
| | | | 8.02-7.94 (m, 2H), | |
| | | | 7.93 (d, J = 1.6 Hz, 1H), | |
| | | | 7.91 (s, 2H), | |
| | | | 7.80-7.74 (m, 2H), | |
| | | | 7.57 (dd, J = 8.0, 1.6 Hz, | |
| | | | 1H), 7.24 (d, J = 7.9 Hz, | |
| | | | 1H), 6.97 (dd, J = 15.7, | |
| | | | 9.2 Hz, 1H), | |
| | | | 6.75 (d, J = 15.7 Hz, | |
| | | | 1H), 4.83 (p, J = 9.4 Hz, | |
| | | | 1H), 3.60 (dd, J = 7.2, | |
| | | | 5.6 Hz, 2H), | |
| | | | 3.49 (td, J = 7.0, 3.3 Hz, | |
| | | | 2H) | |
| F10 | | ESIMS 662 | ¹H NMR (400 MHz, | ¹⁹F NMR (376 MHz, |
| | | ([M + H]⁺) | CDCl₃) δ 7.57 (d, J = 1.6 Hz, | CDCl₃) δ |
| | | | 1H), 7.46 (d, | −60.97, −68.59 |
| | | | J = 8.0 Hz, 1H), | |
| | | | 7.41 (s, 2H), 7.33 (dd, J = 8.0, | |
| | | | 1.6 Hz, 1H), | |
| | | | 6.86 (t, J = 6.1 Hz, 1H), | |
| | | | 6.38 (dd, J = 15.9, | |
| | | | 7.8 Hz, 1H), 4.11 (p, | |
| | | | J = 8.6 Hz, 1H), | |
| | | | 4.04-3.88 (m, 4H), | |
| | | | 3.58-3.49 (m, 2H) | |
| F11 | | ESIMS 660 | ¹H NMR (400 MHz, | ¹⁹F NMR (376 MHz, |
| | | ([M − H]⁻) | CDCl₃) δ | CDCl₃) δ |
| | | | 7.73-7.64 (m, 1H), 7.59 (dd, J = 8.0, | −58.92, −66.28, |
| | | | 1.7 Hz, 1H), | −68.59 |
| | | | 7.53 (d, J = 7.9 Hz, | |
| | | | 1H), 7.42 (s, 2H), | |
| | | | 6.63 (d, J = 15.9 Hz, | |
| | | | 1H), 6.43 (dd, J = 15.9, | |
| | | | 7.9 Hz, 1H), | |
| | | | 5.83 (d, J = 8.6 Hz, | |
| | | | 1H), 4.28-4.06 (m, | |
| | | | 2H), 2.84 (dd, J = 13.4, | |
| | | | 5.6 Hz, 1H), | |
| | | | 2.80-2.69 (m, 3H), | |
| | | | 2.49-2.29 (m, 2H), | |
| | | | 1.75 (dtd, J = 14.9, | |
| | | | 7.4, 5.5 Hz, 1H), | |
| | | | 1.66-1.52 (m, 1H), | |
| | | | 1.01 (t, J = 7.4 Hz, 3H) | |
| F12 | | ESIMS 646 | ¹H NMR (400 MHz, | ¹⁹F NMR (376 MHz, |
| | | ([M − H]⁻) | CDCl₃) δ | CDCl₃) δ |
| | | | 7.70-7.67 (m, 1H), 7.60 (dd, J = 8.0, | −58.96, −66.43, |
| | | | 1.7 Hz, 1H), | −68.59 |
| | | | 7.54 (d, J = 8.0 Hz, | |
| | | | 1H), 7.42 (s, 2H), | |
| | | | 6.63 (d, J = 15.8 Hz, | |
| | | | 1H), 6.44 (dd, J = 15.9, | |
| | | | 7.9 Hz, 1H), | |
| | | | 5.77 (d, J = 8.8 Hz, | |
| | | | 1H), 4.23 (tq, J = 8.5, | |
| | | | 5.7 Hz, 1H), | |
| | | | 4.13 (p, J = 8.4 Hz, 1H), | |
| | | | 3.30-3.06 (m, 2H), | |
| | | | 2.91 (qd, J = 13.3, | |
| | | | 5.9 Hz, 2H), | |
| | | | 1.74 (dtd, J = 14.8, 7.4, | |
| | | | 5.5 Hz, 1H), | |
| | | | 1.67-1.52 (m, 1H), 1.02 (t, | |
| | | | J = 7.4 Hz, 3H) | |
| F13 | | ESIMS 654 | mixture of | ¹⁹F NMR (376 MHz, |
| | | ([M − H]⁻) | diastereomers: ¹H | CDCl₃) δ |
| | | | NMR (400 MHz, | −58.94 (d, J = 11.5 Hz), |
| | | | CDCl₃) δ | −68.59, −128.12 (dd, |
| | | | 7.72-7.65 (m, 1H), 7.59 (dd, J = 8.0, | J = 157.1, |
| | | | 1.6 Hz, 1H), | 24.1 Hz), |
| | | | 7.55 (dd, J = 8.0, 4.7 Hz, | −142.75 (dd, J = 157.1, |
| | | | 1H), 7.42 (s, 2H), | 89.1 Hz) |
| | | | 6.63 (d, J = 15.9 Hz, | |
| | | | 1H), 6.43 (dd, J = 15.9, | |
| | | | 7.8 Hz, 1H), | |
| | | | 5.79 (dd, J = 16.8, | |
| | | | 8.8 Hz, 1H), 4.23 (tq, | |
| | | | J = 8.4, 5.9 Hz, 1H), | |
| | | | 4.18-4.08 (m, 1H), | |
| | | | 2.90-2.77 (m, 2H), | |
| | | | 2.76-2.63 (m, 2H), | |
| | | | 1.90-1.69 (m, 2H), | |
| | | | 1.67-1.44 (m, 2H), | |
| | | | 1.10 (dddd, J = 15.0, | |
| | | | 9.5, 7.6, 3.9 Hz, 1H), | |
| | | | 1.01 (t, J = 7.4 Hz, | |
| | | | 3H) | |
| F14 | | ESIMS 660 | ¹H NMR (400 MHz, | ¹⁹F NMR (376 MHz, |
| | | ([M − H]⁻) | CDCl₃) δ | CDCl₃) δ |
| | | | 7.72-7.66 (m, 1H), | −58.97, −66.28, |
| | | | 7.62-7.53 (m, | −68.59 |
| | | | 2H), 7.42 (s, | |
| | | | 2H), 6.63 (d, J = 15.9 Hz, | |
| | | | 1H), | |
| | | | 6.44 (dd, J = 15.9, 7.9 Hz, | |
| | | | 1H), 5.73 (d, J = 9.4 Hz, | |
| | | | 1H), 3.24 (dq, J = 15.7, | |
| | | | 9.7 Hz, 1H), | |
| | | | 3.10 (dq, J = 15.6, | |
| | | | 10.1 Hz, 1H), | |
| | | | 2.98 (dd, J = 13.4, 4.8 Hz, | |
| | | | 1H), 2.82 (dd, J = 13.3, | |
| | | | 7.9 Hz, 1H), | |
| | | | 1.96 (dp, J = 13.3, | |
| | | | 6.8 Hz, 1H), 1.03 (d, | |
| | | | J = 6.7 Hz, 3H), | |
| | | | 0.98 (d, J = 6.8 Hz, 3H) | |
| F15 | | ESIMS 676 | mixture of | ¹⁹F NMR (376 MHz, |
| | | ([M − H]⁻) | diastereomers ¹H | Acetonitrile-d₃) |
| | | | NMR (400 MHz, | δ −59.57, |
| | | | Acetonitrile-d₃) δ | −59.59, −61.54, |
| | | | 7.88 (s, 1H), | −61.54, −61.84 |
| | | | 7.80-7.74 (m, 1H), | |
| | | | 7.67 (s, 2H), | |
| | | | 7.65-7.50 (m, 1H), 7.03 (d, J = 9.2 Hz | |
| | | | 1H), 6.83 (d, | |
| | | | J = 15.9 Hz, 1H), | |
| | | | 6.73 (dd, J = 15.9, | |
| | | | 8.2 Hz, 1H), 4.49 (p, | |
| | | | J = 9.0 Hz, 1H), | |
| | | | 4.44-4.30 (m, 1H), | |
| | | | 3.92-3.55 (m, 2H), | |
| | | | 3.29-2.91 (m, 2H), | |
| | | | 2.10-1.99 (m, 1H), | |
| | | | 1.05-0.96 (m, 6H) | |
| F16 | | ESIMS 692 | ¹H NMR (400 MHz, | ¹⁹F NMR (376 MHz, |
| | | ([M − H]⁻) | Acetonitrile-d₃) δ | Acetonitrile-d₃) |
| | | | 7.91-7.84 (m, 1H), | δ −59.55, |
| | | | 7.81-7.74 (m, 1H), | −61.52, −69.67 |
| | | | 7.69-7.59 (m, 3H), | |
| | | | 7.04 (d, J = 9.4 Hz, | |
| | | | 1H), 6.83 (d, J = 15.9 Hz, | |
| | | | 1H), | |
| | | | 6.73 (dd, J = 15.9, 8.2 Hz, | |
| | | | 1H), 4.65-4.44 (m, | |
| | | | 2H), 4.36-4.13 (m, | |
| | | | 3H), 3.56-3.37 (m, | |
| | | | 2H), 2.07-1.99 (m, | |
| | | | 1H), 1.01 (d, J = 6.8 Hz, | |
| | | | 3H), 0.97 (d, J = 6.8 Hz, | |
| | | | 3H) | |
| F17 | | ESIMS 680 | ¹H NMR (400 MHz, | ¹⁹F NMR (376 MHz, |
| | | ([M + H]⁺) | Acetonitrile-d₃) δ | Acetonitrile-d₃) |
| | | | 7.93-7.86 (m, 1H), | δ −59.65, |
| | | | 7.77 (dd, J = 8.1, 1.7 Hz, | −61.55, −69.67 |
| | | | 1H), 7.66 (s, 2H), | |
| | | | 7.60 (d, J = 8.0 Hz, | |
| | | | 1H), 7.05 (d, J = 8.8 Hz, | |
| | | | 1H), 6.83 (d, J = 15.9 Hz, | |
| | | | 1H), | |
| | | | 6.73 (dd, J = 15.9, 8.2 Hz, | |
| | | | 1H), 4.60-4.43 (m, | |
| | | | 2H), 4.31-4.19 (m, | |
| | | | 2H), 3.58-3.41 (m, | |
| | | | 2H), 1.87-1.60 (m, | |
| | | | 2H), 1.00 (t, J = 7.4 Hz, | |
| | | | 3H) | |
| F18 | | ESIMS 664 | ¹H NMR (400 MHz, | ¹⁹F NMR (376 MHz, |
| | | ([M + H]⁺) | Acetonitrile-d₃) δ | Acetonitrile-d₃) |
| | | | 7.93-7.84 (m, 1H), | δ −59.61, |
| | | | 7.75 (dt, J = 8.1, 1.9 Hz, | −59.65, −61.59, |
| | | | 1H), 7.66 (s, 2H) | −61.80, −69.66, |
| | | | 7.55 (dd, J = 15.9, | −69.66 |
| | | | 8.0 Hz, 1H), | |
| | | | 7.19-7.02 (m, 1H), | |
| | | | 6.82 (d, J = 15.9 Hz, 1H), | |
| | | | 6.73 (dd, J = 15.9, | |
| | | | 8.2 Hz, 1H), | |
| | | | 4.61-4.25 (m, 2H), | |
| | | | 3.92-3.54 (m, 2H), | |
| | | | 3.24-2.98 (m, 2H), | |
| | | | 1.87-1.55 (m, 2H), | |
| | | | 1.08-0.96 (m, 3H) | |
| F19 | | | ¹H MMR (400 MHz, | ¹⁹F NMR (376 MHz, |
| | | | CDCl₃) δ 7.65 (s, 1H), | CDCl₃) δ |
| | | | 7.57 (dd, J = 3.3, 1.6 Hz, | −58.97, −58.99, |
| | | | 2H), 7.42 (s, 2H), | −68.58, |
| | | | 6.62 (d, J = 15.9 Hz, | −130.11 (d, J = 46.3 Hz), |
| | | | 1H), 6.50-6.28 (m, | −130.53 (d, J = 46.3 Hz), |
| | | | 2H), 4.46 (dddd, J = 14.6, | −140.63 (d, J = 17.1 Hz), |
| | | | 8.4, 5.6, 2.8 Hz, | −141.05 (d, J = 17.2 Hz) |
| | | | 1H), | |
| | | | 4.21-4.04 (m, 1H), | |
| | | | 3.52-3.23 (m, 3H), 3.13 (dddd, | |
| | | | J = 25.5, 14.6, 7.8, | |
| | | | 2.7 Hz, 1H), | |
| | | | 2.11-1.98 (m, 1H), | |
| | | | 1.98-1.79 (m, 2H), | |
| | | | 1.79-1.69 (m, 1H), | |
| | | | 1.49-1.35 (m, 1H), | |
| | | | 1.04 (td, J = 7.4, 1.3 Hz, | |
| | | | 3H) | |
| F20 | | | ¹H NMR (400 MHz, | ¹⁹F NMR (376 MHz, |
| | | | CDCl₃) δ | CDCl₃) δ |
| | | | 7.69-7.65 (m, 1H), 7.57 (dd, J = 8.0, | −58.94, −68.60, |
| | | | 1.7 Hz, 1H), | −129.50 (d, J = 157.5 Hz), |
| | | | 7.52 (d, J = 7.9 Hz, | −140.66 (d, J = 157.6 Hz) |
| | | | 1H), 7.41 (s, 2H), | |
| | | | 6.93 (d, J = 8.4 Hz, | |
| | | | 1H), 6.61 (d, J = 15.9 Hz, | |
| | | | 1H), | |
| | | | 6.42 (dd, J = 15.9, 7.9 Hz, | |
| | | | 1H), 4.50 (ttd, J = 8.3, | |
| | | | 6.2, 3.7 Hz, 1H), | |
| | | | 4.12 (p, J = 8.7 Hz, | |
| | | | 1H), 3.09 (dd, J = 13.3, | |
| | | | 3.7 Hz, 1H), | |
| | | | 3.00 (dd, J = 13.2, | |
| | | | 6.1 Hz, 1H), | |
| | | | 2.95-2.82 (m, 2H), | |
| | | | 2.14-1.97 (m, 2H), | |
| | | | 1.91 (ddd, J = 14.0, 7.6, | |
| | | | 6.7 Hz, 1H), | |
| | | | 1.72 (tdd, J = 11.7, 8.1, | |
| | | | 5.0 Hz, 1H), | |
| | | | 1.26 (dtd, J = 12.8, 7.8, | |
| | | | 3.4 Hz, 1H), 1.08 (t, | |
| | | | J = 7.4 Hz, 3H) | |
| F21 | | ESIMS 694 | 1H MMR (400 MHz, | ¹⁹F NMR (376 MHz, |
| | | ([M + H]⁺) | DMSO-d₆) δ 8.63 (d, | DMSO-d₆) |
| | | | J = 8.6 Hz, 1H), | δ −57.88, |
| | | | 8.02 (s, 1H), | −64.46, −68.00 |
| | | | 7.98-7.86 (m, 3H), 7.07 (dd, J = 15.8, | |
| | | | 9.1 Hz, 1H), | |
| | | | 6.89 (d, J = 15.8 Hz, | |
| | | | 1H), 4.87 (p, J = 9.4 Hz, | |
| | | | 1H), 4.39 (qt, J = 8.9, | |
| | | | 4.5 Hz, 1H), | |
| | | | 3.58-3.38 (m, 4H), | |
| | | | 2.89-2.70 (m, 2H), | |
| | | | 1.81-1.51 (m, 2H), | |
| | | | 0.93 (t, J = 7.3 Hz, 3H) | |
| F22 | | ESIMS 632 | ¹H NMR (400 MHz, | ¹⁹F NMR (376 MHz, |
| | | ([M − H]⁻) | CDCl₃) δ | CDCl₃) δ |
| | | | 7.72-7.65 (m, 1H), 7.60 (dd, J = 8.0, | −58.87, −66.50, |
| | | | 1.7 Hz, 1H), | −68.58 |
| | | | 7.54 (d, J = 7.9 Hz, | |
| | | | 1H), 7.41 (s, 2H), | |
| | | | 6.62 (d, J = 15.9 Hz, | |
| | | | 1H), 6.43 (dd, J = 15.9, | |
| | | | 7.9 Hz, 1H), | |
| | | | 5.80 (d, J = 8.2 Hz, | |
| | | | 1H), 4.39 (dq, J = 8.2, | |
| | | | 6.3 Hz, 1H), | |
| | | | 4.23-4.04 (m, 1H), | |
| | | | 3.29-3.04 (m, 2H), | |
| | | | 2.99-2.80 (m, 2H), | |
| | | | 1.34 (d, J = 6.7 Hz, 3H) | |
| F23 | | ESIMS 648 | mixture of | mixture of |
| | | ([M − H]⁻) | diastereomers: ¹H | diastereomers: |
| | | | NMR (400 MHz, | ¹⁹F NMR (376 MHz, |
| | | | DMSO-d₆) δ | DMSO-d₆) |
| | | | 8.80-8.70 (m, 1H), 8.02 (t, | δ −57.82, |
| | | | J = 1.6 Hz, 1H), | −57.82, −59.60, |
| | | | 7.96-7.86 (m, 3H), | −59.73, −67.94 |
| | | | 7.50 (dd, J = 15.3, 7.9 Hz, | |
| | | | 1H), 7.14-7.01 (m, | |
| | | | 1H), 6.88 (d, J = 15.7 Hz, | |
| | | | 1H), | |
| | | | 4.87 (p, J = 9.4 Hz, 1H), | |
| | | | 4.50-4.32 (m, 1H), | |
| | | | 4.21-3.90 (m, 2H), | |
| | | | 3.28-2.99 (m, 2H), | |
| | | | 1.30 (dd, J = 6.7, 3.6 Hz, | |
| | | | 3H) | |
| F24 | | ESIMS 664 | ¹H NMR (400 MHz, | ¹⁹F NMR (376 MHz, |
| | | ([M − H]⁻) | Acetonitrile-d₃) δ | Acetonitrile-d₃) |
| | | | 7.94-7.85 (m, 1H), | δ −59.72, |
| | | | 7.77 (dd, J = 8.0, 1.7 Hz, | −61.61, −69.68 |
| | | | 1H), 7.67 (s, 2H), | |
| | | | 7.55 (d, J = 8.0 Hz, | |
| | | | 1H), 7.08 (d, J = 8.3 Hz, | |
| | | | 1H), 6.83 (d, J = 15.9 Hz, | |
| | | | 1H), | |
| | | | 6.73 (dd, J = 15.9, 8.2 Hz, | |
| | | | 1H), 4.64 (dtd, J = 8.3, | |
| | | | 6.9, 5.5 Hz, 1H), | |
| | | | 4.49 (p, J = 9.0 Hz, | |
| | | | 1H), 4.25 (qd, J = 9.7, | |
| | | | 2.7 Hz, 2H), | |
| | | | 3.57 (dd, J = 14.5, 7.1 Hz, | |
| | | | 1H), 3.41 (dd, J = 14.4, | |
| | | | 5.5 Hz, 1H), | |
| | | | 1.41 (d, J = 6.8 Hz, | |
| | | | 3H) | |
| F25 | | ESIMS 731 | ¹H NMR (400 MHz, | IR (thin film) |
| | | ([M + H]⁺) | DMSO-d₆) δ 8.80 (d, | 3406, 2928, |
| | | | J = 8.0 Hz, 1H), | 1654, 1164, |
| | | | 8.67 (t, J = 6.4 Hz, 1H), | 846 cm⁻¹ |
| | | | 7.94 (s, 1H), 7.91 (s, | |
| | | | 2H), 7.62 (d, J = 8.0 Hz, | |
| | | | 1H), 7.45 (d, J = 7.6 Hz, | |
| | | | 1H), | |
| | | | 7.00 (dd, J = 15.6, 9.2 Hz, | |
| | | | 1H), 6.77 (d, J = 15.6 Hz, | |
| | | | 1H) | |
| | | | 4.85-4.41 (m, 1H), | |
| | | | 4.60-4.56 (m, 1H), | |
| | | | 4.03-3.93 (m, 2H), 3.19 (t, | |
| | | | J = 8.4 Hz, 2H), | |
| | | | 2.99 (s, 3H), | |
| | | | 2.18-2.05 (m, 2H) | |
| F26 | | ESIMS 723 | ¹H NMR (300 MHz, | IR (thin film) |
| | | ([M + H]⁺) | DMSO-d₆) δ 8.89 (d, | 3319, 2932, |
| | | | J = 7.8 Hz, 1H), | 1661, 1167, |
| | | | 8.75 (t, J = 6.0 Hz, 1H), | 845 cm⁻¹ |
| | | | 8.00 (s, 1H), 7.92 (s, | |
| | | | 2H), 7.89 (s, 1H), | |
| | | | 7.60 (d, J = 7.8 Hz, | |
| | | | 1H), 7.11 (dd, J = 16.2, | |
| | | | 9.3 Hz, 1H), | |
| | | | 6.90 (d, J = 16.2 Hz, | |
| | | | 1H) 4.88-4.83 (m, | |
| | | | 1H), 4.62-4.55 (m, | |
| | | | 1H), 4.04-3.93 (m, | |
| | | | 2H), 3.15-3.09 (m, | |
| | | | 2H), 2.99 (s, 3H), | |
| | | | 2.16-2.04 (m, 2H) | |
| F27 | | ESIMS 701 | ¹H NMR (400 MHz, | IR (thin film) |
| | | ([M + H]⁺) | DMSO-d₆) δ 8.69 (d, | 3284, 2921, |
| | | | J = 8.0 Hz 1H), | 1684, 1165 |
| | | | 8.63 (t, J = 6.4 Hz, 1H), | 967, 748 cm⁻¹ |
| | | | 7.93 (d, J = 8.8 Hz, | |
| | | | 3H), 7.61 (d, J = 8.0 Hz, | |
| | | | 1H), 7.40 (dd, J = 7.6 Hz, | |
| | | | 1H), | |
| | | | 6.97 (dd, J = 15.6, 9.2 Hz, | |
| | | | 1H), 6.77 (dd, J = 15.6 Hz, | |
| | | | 1H) | |
| | | | 4.85-4.81 (m, 1H), | |
| | | | 4.58-4.53 (m, 1H), | |
| | | | 4.03-3.86 (m, 2H), | |
| | | | 2.60-2.51 (m, 2H), | |
| | | | 2.03 (s, 3H), | |
| | | | 1.98-1.89 (m, 2H) | |
| F28 | | ESIMS 689 | ¹H NMR (400 MHz, | IR (thin film) |
| | | ([M − H]⁻) | DMSO-d₆) δ 8.78 (d, | 3290, 2923, |
| | | | J = 8.4 Hz, 1H), | 1652, 1165, |
| | | | 8.67 (t, J = 6.4 Hz, 1H), | 809 cm⁻¹ |
| | | | 7.99 (s, 1H), 7.92 (s, | |
| | | | 1H), 7.89 (s, 2H), | |
| | | | 7.54 (d, J = 7.6 Hz, | |
| | | | 1H), 7.06 (dd, J = 16.0, | |
| | | | 8.8 Hz, 1H), | |
| | | | 6.88 (d, J = 15.6 Hz, | |
| | | | 1H) 4.88-4.81 (m, | |
| | | | 1H), 4.58-4.53 (m, | |
| | | | 1H), 4.04-3.85 (m, | |
| | | | 2H), 2.50-2.44 (m, | |
| | | | 2H), 2.05 (s, 3H), | |
| | | | 1.98-1.85 (m, 2H) | |
| F29 | | ESIMS 676 | ¹H NMR (400 MHz, | ¹⁹F NMR (376 MHz, |
| | | ([M − H]⁻) | CDCl₃) δ 7.74 (td, J = 1.3, | CDCl₃) δ |
| | | | 0.7 Hz, 1H), | −58.83, −66.49, |
| | | | 7.66 (dt, J = 7.9, 1.5 Hz, | −69.14 |
| | | | 1H), 7.59 (d, J = 7.9 Hz, | |
| | | | 1H), 7.41 (s, 2H), | |
| | | | 5.81 (d, J = 8.3 Hz, | |
| | | | 1H), 5.47 (d, J = 9.5 Hz, | |
| | | | 1H), 4.67 (q, J = 9.2 Hz, | |
| | | | 1H), | |
| | | | 4.41 (dq, J = 8.1, 6.2 Hz, | |
| | | | 1H), 3.67 (qd, J = 7.1, | |
| | | | 2.0 Hz, 2H), | |
| | | | 3.29-3.08 (m, 2H), | |
| | | | 2.90 (t, J = 5.4 Hz, 2H), | |
| | | | 1.35 (d, J = 6.7 Hz, | |
| | | | 3H), 1.24 (t, J = 7.0 Hz, | |
| | | | 3H) | |
| F30 | | ESIMS 694 | ¹H NMR (400 MHz, | major isomer: |
| | | ([M + H]⁺) | CDCl₃) δ | ¹⁹F NMR (376 MHz, |
| | | | 7.76-7.69 (m, 1H), 7.64 (td, J = 8.2, | CDCl₃) δ |
| | | | 1.7 Hz, 1H), | −58.87, −60.78, |
| | | | 7.56 (dd, J = 13.4, 7.9 Hz, | −69.17; minor |
| | | | 1H), 7.41 (s, 2H), | isomer: ¹⁹F |
| | | | 6.92-6.28 (m, 1H), | NMR (376 MHz, |
| | | | 5.56-5.38 (m, 1H), | CDCl₃) δ |
| | | | 4.66 (dddd, J = 20.5, | −58.84, −60.60, |
| | | | 15.1, 11.8, 5.6 Hz, | −69.16 |
| | | | 2H), 3.66 (tdd, J = 9.6, | |
| | | | 5.3, 2.9 Hz, 2H), | |
| | | | 3.61-3.45 (m, 1H), | |
| | | | 3.20 (qd, J = 13.4, | |
| | | | 5.8 Hz, 2H), 1.54 (d, | |
| | | | J = 6.8 Hz, 3H), | |
| | | | 1.33-1.08 (m, 3H) | |
| F31 | | ESIMS 664 | ¹H NMR (400 MHz, | ¹⁹F NMR (376 MHz, |
| | | ([M + H]⁺) | CDCl₃) δ 7.74 (dd, J = 1.7, | CDCl₃) δ |
| | | | 0.9 Hz, 1H), | −58.83, −66.49, |
| | | | 7.67 (dt, J = 7.6, 1.4 Hz, | −69.22 |
| | | | 1H), 7.59 (d, J = 8.0 Hz, | |
| | | | 1H), 7.42 (s, | |
| | | | 2H), 5.82 (d, J = 8.2 Hz, | |
| | | | 1H), 5.47 (d, J = 9.5 Hz, | |
| | | | 1H), 4.66 (p, | |
| | | | J = 9.1 Hz, 1H), | |
| | | | 4.41 (dq, J = 8.1, 6.3 Hz, | |
| | | | 1H), 3.49 (s, 3H), | |
| | | | 3.26-3.04 (m, 2H), | |
| | | | 3.04-2.78 (m, 2H), | |
| | | | 1.35 (d, J = 6.7 Hz, | |
| | | | 3H) | |
| F32 | | ESIMS 614 | ¹H NMR (400 MHz, | ¹⁹F NMR (376 MHz, |
| | | ([M − H]⁻) | CDCl₃) δ 7.74 (s, 1H), | CDCl₃) δ |
| | | | 7.51 (d, J = 1.3 Hz, | −66.46, −68.61, |
| | | | 2H), 7.42 (s, 2H), | −113.04 (dd, J = 39.9, |
| | | | 7.24 (t, J = 55.1 Hz, | 11.1 Hz) |
| | | | 1H), 6.63 (d, J = 15.9 Hz, | |
| | | | 1H), | |
| | | | 6.44 (dd, J = 15.9, 7.9 Hz, | |
| | | | 1H), 5.95 (d, J = 8.2 Hz, | |
| | | | 1H), | |
| | | | 4.48-4.28 (m, 1H), 3.17 (ttd, J = 15.6, | |
| | | | 9.8, 5.7 Hz, | |
| | | | 2H), 2.91 (qd, J = 13.3, | |
| | | | 6.0 Hz, 2H), | |
| | | | 1.36 (d, J = 6.7 Hz, | |
| | | | 3H) | |
| F33 | | ESIMS 678 | ¹H NMR (400 MHz, | ¹⁹F NMR (376 MHz, |
| | | ([M + H]⁺) | CDCl₃) δ | CDCl₃) δ |
| | | | 7.62-7.58 (m, 1H), | −58.89, −66.47, |
| | | | 7.58-7.54 (m, 1H), | −69.86 |
| | | | 7.52-7.45 (m, 1H), 7.26 (s, | |
| | | | 2H), 5.82 (d, J = 8.2 Hz, | |
| | | | 1H), 5.05 (d, J = 10.6 Hz, | |
| | | | 1H), | |
| | | | 4.52-4.32 (m, 1H), | |
| | | | 3.98 (qd, J = 7.0, 3.1 Hz, | |
| | | | 2H), 3.77 (ddt, J = 13.5, | |
| | | | 9.8, 6.7 Hz, | |
| | | | 1H), 3.32-3.04 (m, | |
| | | | 2H), 3.00-2.83 (m, | |
| | | | 2H), 1.40 (t, J = 7.0 Hz, | |
| | | | 3H), 1.36 (dd, J = 6.8, | |
| | | | 1.3 Hz, 3H) | |
| F34 | | ESIMS 624 | ¹H NMR (400 MHz, | IR (thin film) |
| | | ([M − H]⁻) | DMSO-d₆) δ 8.43 (d, | 3431, 2924, |
| | | | J = 8.4 Hz, 1H), | 1642, 1115, |
| | | | 7.92 (s, 1H), | 749 cm⁻¹ |
| | | | 7.86-7.85 (m, 2H), 7.59 (d, J = 7.6 Hz, | |
| | | | 1H), 7.35 (d, | |
| | | | J = 8.0 Hz, 1H), | |
| | | | 6.99 (dd, J = 9.6, 16.0 Hz, | |
| | | | 1H), 6.76 (d, J = 16.4 Hz, | |
| | | | 1H), 4.80 (t, | |
| | | | J = 9.6 Hz, 1H), | |
| | | | 4.10 (m, 1H), | |
| | | | 3.57-3.51 (m, 2H), 2.80 (t, J = 7.6 Hz, | |
| | | | 2H), 1.21 (d, | |
| | | | J = 6.8 Hz, 3H) | |
| F35 | | ESIMS 616 | ¹H NMR (400 MHz, | IR (thin film) |
| | | ([M + H]⁺) | DMSO-d₆) δ 8.52 (d, | 3431, 2925, |
| | | | J = 8.4 Hz, 1H), | 1645, 1115 cm⁻¹ |
| | | | 7.99 (s, 1H), | |
| | | | 7.90-7.86 (m, 2H), 7.48 (d, J = 8.0 Hz, | |
| | | | 1H), 7.28 (d, | |
| | | | J = 8.4 Hz, 1H), | |
| | | | 7.07 (dd, J = 8.8, 15.6 Hz, | |
| | | | 1H), 6.87 (d, J = 15.6 Hz, | |
| | | | 1H), 4.85 (t, | |
| | | | J = 9.6 Hz, 1H), | |
| | | | 4.10-4.06 (m, 1H), | |
| | | | 3.56-3.51 (m, 2H), | |
| | | | 2.83-2.76 (m, 2H), | |
| | | | 1.20 (d, J = 6.8 Hz, 3H) | |
| F36 | 117-119 | ESIMS 646 | ¹H NMR (300 MHz, | |
| | | ([M − H]⁻) | DMSO-d₆) δ 8.74 (dd, | |
| | | | J = 8.1 Hz, 1H), | |
| | | | 8.00 (s, 1H), | |
| | | | 7.92-7.89 (m, 1H), 7.88 (d, J = 6.3 Hz, | |
| | | | 1H), 7.52 (d, | |
| | | | J = 7.8 Hz, 1H), | |
| | | | 7.45 (d, J = 7.2 Hz, 1H), | |
| | | | 7.09 (dd, J = 15.6, | |
| | | | 8.7 Hz, 1H), 6.88 (d, | |
| | | | J = 15.6 Hz, 1H), | |
| | | | 4.86-4.68 (m, 3H), | |
| | | | 4.55-4.51 (m, 1H), | |
| | | | 3.60-3.44 (m, 2H), | |
| | | | 1.30 (d, J = 6.6 Hz, | |
| | | | 3H) | |
| F37 | 114-118 | ESIMS 633 | ¹H NMR (400 MHz, | |
| | | ([M + H]⁺) | DMSO-d₆) δ 8.78 (dd, | |
| | | | J = 8.0, 8.1 Hz, 1H), | |
| | | | 8.00 (s, 1H), | |
| | | | 7.91-7.86 (m, 3H), | |
| | | | 7.52-7.46 (m, 1H), | |
| | | | 7.08 (dd, J = 16.0, 8.8 Hz, | |
| | | | 1H), 6.87 (d, J = 15.6 Hz, | |
| | | | 1H), 4.85 (t, | |
| | | | J = 9.2 Hz, 1H), | |
| | | | 4.38-4.36 (m, 1H), | |
| | | | 4.09-3.98 (m, 2H), | |
| | | | 3.16-3.08 (m, 2H), | |
| | | | 1.28 (d, J = 4.0 Hz, 3H) | |
| F38 | | ESIMS 642 | ¹H NMR (400 MHz, | IR (thin film) |
| | | ([M + H]⁺) | DMSO-d₆) δ 8.48 (d, | 3261, 1643, |
| | | | J = 7.8 Hz, 1H), | 1115, 744 cm⁻¹ |
| | | | 7.88 (s, 2H), | |
| | | | 7.64-7.61 (m, 2H), | |
| | | | 7.55-7.52 (m, 1H), 6.89 (dd, J = 15.9, | |
| | | | 9.0 Hz, 1H), | |
| | | | 6.78 (d, J = 15.9 Hz, | |
| | | | 1H), 4.85-4.79 (m, | |
| | | | 1H), 4.10-4.06 (m, | |
| | | | 1H), 3.59-3.37 (m, | |
| | | | 2H), 2.89-2.78 (m, | |
| | | | 1H), 2.73-2.72 (m, | |
| | | | 1H), 1.22-1.21 (m, | |
| | | | 3H) | |
| F39 | 164-166 | ESIMS 658 | ¹H NMR (300 MHz, | |
| | | ([M + H]⁺) | DMSO-d₆) δ 8.75 (d, | |
| | | | J = 8.4, 1H), 7.88 (s, | |
| | | | 2H), 7.65-7.53 (m, | |
| | | | 2H), 6.94 (dd, J = 15.6, | |
| | | | 9.0 Hz, 1H), | |
| | | | 6.78 (d, J = 15.6 Hz, | |
| | | | 1H), 4.86-4.80 (m, | |
| | | | 1H), 4.41-4.37 (m, | |
| | | | 2H), 4.10-3.98 (m, | |
| | | | 2H), 3.31-3.09 (m, | |
| | | | 2H), 1.34-1.31 (m, | |
| | | | 3H) | |
| F40 | 156-158 | ESIMS 610 | ¹H NMR (300 MHz, | |
| | | ([M + H]⁺) | DMSO-d₆) δ 8.45 (d, | |
| | | | J = 8.4 Hz, 1H), | |
| | | | 7.91-7.86 (m, 2H), | |
| | | | 7.46 (s, 1H), 7.42 (d, J = 8.1 Hz, | |
| | | | 1H), 7.32 (d, | |
| | | | J = 7.5 Hz, 1H), | |
| | | | 6.88 (dd, J = 15.6, 8.7 Hz, | |
| | | | 1H), 6.75 (d, J = 15.6 Hz, | |
| | | | 1H), | |
| | | | 4.86-4.67 (m, 3H), | |
| | | | 4.61-4.52 (m, 1H), | |
| | | | 3.62-3.31 (m, 2H), | |
| | | | 2.34 (s, 3H), 1.31 (d, J = 6.6 Hz, | |
| | | | 3H) | |
| F41 | | ESIMS 719 | ¹H NMR (400 MHz, | IR (thin film) |
| | | ([M + H]⁺) | DMSO-d₆) δ 8.51 (d, | 3270, 1645, |
| | | | J = 8.0 Hz, 1H), | 1313, 1118, |
| | | | 8.05 (s, 2H), 7.99 (s, 1H), | 750 cm⁻¹ |
| | | | 7.90 (d, J = 8.4 Hz, | |
| | | | 1H), 7.48 (d, J = 8.4 Hz, | |
| | | | 1H), 7.09 (dd, J = 15.6, | |
| | | | 8.8 Hz, 1H), | |
| | | | 6.87 (d, J = 15.6 Hz, | |
| | | | 1H), 4.86-4.81 (m, | |
| | | | 1H), 4.10-4.02 (m, | |
| | | | 1H), 3.57-3.49 (m, | |
| | | | 2H), 2.85-2.76 (m, | |
| | | | 2H), 1.19 (d, J = 6.8 Hz, | |
| | | | 3H) | |
| F42 | 123-125 | ESIMS 736 | ¹H NMR (400 MHz, | |
| | | ([M + H]⁺) | DMSO-d₆) δ 8.69 (d, | |
| | | | J = 8.4 Hz, 1H), | |
| | | | 8.05-8.02 (m, 3H), | |
| | | | 7.91 (d, J = 7.6 Hz, 1H), | |
| | | | 7.52 (dd, J = 15.2, | |
| | | | 8.0 Hz, 1H), | |
| | | | 7.08 (dd, J = 15.6, 8.8 Hz, | |
| | | | 1H), 6.88 (d, J = 15.6 Hz, | |
| | | | 1H), | |
| | | | 4.86-4.82 (m, 1H), | |
| | | | 4.38-4.37 (m, 1H), | |
| | | | 4.13-3.95 (m, 2H), | |
| | | | 3.31-3.13 (m, 2H), | |
| | | | 1.30 (d, J = 6.4 Hz, 3H) | |
| F43 | | ESIMS 648 | ¹H NMR (400 MHz, | ¹⁹F NMR (376 MHz, |
| | | ([M + H]⁺) | CDCl₃) δ 7.73 (s, 1H), | CDCl₃) δ |
| | | | 7.52 (q, J = 8.0 Hz, | −61.12, −68.59, |
| | | | 2H), 7.42 (s, 2H), | −113.27 (d, J = 83.6 Hz) |
| | | | 7.38-7.05 (m, 1H), | |
| | | | 6.63 (d, J = 15.9 Hz, | |
| | | | 1H), 6.45 (dd, J = 15.9, | |
| | | | 7.9 Hz, 1H), | |
| | | | 4.68 (dt, J = 13.0, | |
| | | | 6.6 Hz, 1H), | |
| | | | 4.19-3.88 (m, 3H), | |
| | | | 3.52 (dd, J = 14.5, 6.4 Hz, | |
| | | | 1H), 3.43 (dd, J = 14.5, | |
| | | | 5.0 Hz, 1H), | |
| | | | 1.55 (d, J = 6.9 Hz, | |
| | | | 3H) | |
| F44 | | ESIMS 646 | ¹H NMR (400 MHz, | ¹⁹F NMR (376 MHz, |
| | | ([M − H]⁻) | CDCl₃) δ 7.69 | CDCl₃) δ |
| | | | 7.65 (m, 1H), 7.59 (dd, J = 8.0, | −58.79, −66.79, |
| | | | 1.7 Hz, 1H), | −68.60 |
| | | | 7.53 (d, J = 8.0 Hz, | |
| | | | 1H), 7.41 (s, 2H), | |
| | | | 6.62 (d, J = 15.9 Hz, | |
| | | | 1H), 6.43 (dd, J = 15.9, | |
| | | | 7.9 Hz, 1H), | |
| | | | 5.67 (s, 1H), | |
| | | | 4.19-4.07 (m, 1H), | |
| | | | 3.30 (s, 2H), 3.14 (q, J = 9.8 Hz, | |
| | | | 2H), 1.48 (s, | |
| | | | 6H) | |
| F45 | | ESIMS 681 | ¹H NMR (300 MHz, | IR (thin film) |
| | | ([M + H]⁺) | DMSO-d₆) δ 8.62 (d, | 3289, 2929, |
| | | | J = 8.1 Hz, 1H), | 1656, 1125 cm⁻¹ |
| | | | 8.23 (t, J = 6.6 Hz, 1H), | |
| | | | 7.99 (s, 1H), 7.92 (s, | |
| | | | 3H), 7.55 (d, J = 7.5 Hz, | |
| | | | 1H), 7.09 (dd, J = 15.9, | |
| | | | 9.0, 1H), | |
| | | | 6.88 (d, J = 15.9 Hz, | |
| | | | 1H), 4.89-4.82 (m, | |
| | | | 1H), 4.39-4.35 (m, | |
| | | | 1H), 3.88-3.76 (m, | |
| | | | 2H), 3.40-3.33 (m, | |
| | | | 1H), 3.26-3.17 (m, | |
| | | | 1H), 1.25 (d, J = 6.6 Hz, | |
| | | | 3H) | |
| F46 | 144-148 | ESIMS 594 | ¹H NMR (300 MHz, | |
| | | ([M + H]⁺) | DMSO-d₆) δ 8.41 (d, | |
| | | | J = 8.1 Hz, 1H), | |
| | | | 7.89 (s, 2H), 7.47 (s, 1H), | |
| | | | 7.42 (d, J = 8.1 Hz, | |
| | | | 1H), 7.31-7.25 (m, | |
| | | | 1H), 6.88 (dd, J = 15.9, | |
| | | | 8.4 Hz, 1H), | |
| | | | 6.75 (d, J = 15.3 Hz, | |
| | | | 1H), 4.85-4.79 (m, | |
| | | | 1H), 4.42-4.77 (m, | |
| | | | 1H), 4.11-3.97 (m, | |
| | | | 2H), 3.20-3.08 (m, | |
| | | | 2H), 2.34 (s, 3H), | |
| | | | 1.31-1.29 (d, J = 6.6 Hz, | |
| | | | 3H) | |
| F47 | | ESIMS 662 | mixture of | mixture of |
| | | ([M − H]⁻) | diastereomers ¹H | diastereomers |
| | | | NMR (400 MHz, | ¹⁹F NMR (376 MHz, |
| | | | Acetone-d₆) δ | DMSO-d₆) |
| | | | 7.92 (d, J = 1.6 Hz, 1H), | δ −57.69, |
| | | | 7.87-7.83 (m, 1H), | 59.63, −59.63, |
| | | | 7.81 (s, 2H), 7.65 (s, | −67.96 |
| | | | 1H), 7.60 (d, J = 7.9 Hz, | |
| | | | 1H), 7.03 (dd, J = 15.8, | |
| | | | 7.9 Hz, 1H), | |
| | | | 6.96 (d, J = 15.8 Hz, | |
| | | | 1H), 4.74 (p, J = 9.0 Hz, | |
| | | | 1H), | |
| | | | 3.96-3.76 (m, 2H), | |
| | | | 3.63-3.48 (m, 2H), | |
| | | | 1.60 (d, J = 4.4 Hz, | |
| | | | 6H) | |
| F48 | | ESIMS 678 | ¹H NMR (400 MHz, | ¹⁹F NMR (376 MHz, |
| | | ([M − H]⁻) | Acetone-d₆) δ | CDCl₃) δ |
| | | | 7.93-7.90 (m, 1H), | −58.88, −60.97, |
| | | | 7.84 (dd, J = 8.0, 1.7 Hz, | −68.60 |
| | | | 1H), 7.81 (s, 2H), | |
| | | | 7.69-7.59 (m, 2H), | |
| | | | 7.03 (dd, J = 15.8, | |
| | | | 7.9 Hz, 1H), 6.96 (d, | |
| | | | J = 15.8 Hz, 1H), | |
| | | | 4.79-4.68 (m, 1H), | |
| | | | 4.41 (q, J = 9.9 Hz, | |
| | | | 2H), 4.08 (s, 2H), | |
| | | | 1.66 (s, 6H) | |
| F49 | 158-161 | ESIMS 642 | ¹H NMR (400 MHz, | |
| | | ([M + H]⁺) | DMSO-d₆) δ 8.71 (dd, | |
| | | | J = 8.4, 8.0 Hz, 1H), | |
| | | | 7.93 (s, 1H), 7.86 (d, | |
| | | | J = 6.4 Hz, 2H), | |
| | | | 7.60 (d, J = 8.0 Hz, 1H), | |
| | | | 7.37-7.33 (m, 1H), | |
| | | | 6.99 (dd, J = 15.6, | |
| | | | 9.2 Hz, 1H), 6.76 (d, | |
| | | | J = 15.6 Hz, 1H), | |
| | | | 4.82 (t, J = 9.2 Hz, | |
| | | | 1H), 4.40-4.37 (m, | |
| | | | 1H), 4.13-3.98 (m, | |
| | | | 2H), 3.13-3.08 (m, | |
| | | | 2H), 1.32 (d, J = 4.2 Hz, | |
| | | | 3H) | |
| F50 | 140-142 | ESIMS 614 | ¹H NMR (300 MHz, | |
| | | ([M + H]⁺) | DMSO-d₆) δ 8.70 (dd, | |
| | | | J = 8.4, 8.4 Hz, 1H), | |
| | | | 7.90 (s, 2H), 7.79 (s, | |
| | | | 1H), 7.57 (d, J = 8.4 Hz, | |
| | | | 1H), | |
| | | | 7.41-7.37 (m, 1H), 7.01 (dd, J = 15.9, | |
| | | | 9.3 Hz, 1H), | |
| | | | 6.78 (d, J = 15.9 Hz, | |
| | | | 1H), 4.86 (t, J = 9.3 Hz, | |
| | | | 1H), | |
| | | | 4.41-4.37 (m, 1H), | |
| | | | 4.14-3.98 (m, 2H), | |
| | | | 3.14-3.04 (m, 2H), 1.31 (d, J = 3.9 Hz, | |
| | | | 3H) | |
| F51 | | ESIMS 578 | ¹H NMR (300 MHz, | IR (thin film) |
| | | ([M + H]⁺) | DMSO-d₆) δ 8.22 (d, | 3437, 1637, |
| | | | J = 8.1 Hz, 1H), | 1114, 749 cm⁻¹ |
| | | | 7.88 (s, 2H), 7.45 (s, 1H), | |
| | | | 7.40 (d, J = 8.7 Hz, | |
| | | | 1H), 7.28 (d, J = 7.5 Hz, | |
| | | | 1H), 6.78 (dd, J = 15.6, | |
| | | | 8.7 Hz, 1H), | |
| | | | 6.74 (d, J = 15.9 Hz, | |
| | | | 1H), | |
| | | | 4.85-4.79 (m, | |
| | | | 1H), 4.14-4.09 (m, | |
| | | | 1H), 3.57-3.30 (m, | |
| | | | 2H), 2.81 (d, J = 6.9 Hz, | |
| | | | 2H), 2.34 (s, 3H), | |
| | | | 1.20 (d, J = 6.6 Hz, | |
| | | | 3H) | |
| F52 | 85-87 | ESIMS 658 | ¹H NMR (300 MHz, | |
| | | ([M + H]⁺) | DMSO-d₆) δ 8.66 (dd, | |
| | | | J = 7.5 Hz, 1H), | |
| | | | 7.94 (s, 1H), | |
| | | | 7.87-7.84 (m, 2H), 7.62 (d, J = 7.8 Hz, | |
| | | | 1H), 7.38 (d, | |
| | | | J = 8.1 Hz, 1H), | |
| | | | 7.00 (dd, J = 15.6, 9.6 Hz, | |
| | | | 1H), | |
| | | | 6.76 (d, J = 15.6 Hz, | |
| | | | 1H), 4.83-4.68 (m, 3H), | |
| | | | 4.55-4.50 (m, 1H), | |
| | | | 3.62-3.43 (m, 2H), | |
| | | | 1.33 (d, J = 6.9 Hz, 3H) | |
| F53 | 110-114 | ESIMS 639 | ¹H NMR (400 MHz, | |
| | | ([M + H]⁺) | DMSO-d₆) δ 8.69 (d, | |
| | | | J = 7.6 Hz, 1H), | |
| | | | 8.02-8.00 (m, 3H), | |
| | | | 7.91 (d, J = 8.4 Hz, 1H), | |
| | | | 7.53 (dd, J = 14.4, | |
| | | | 7.6 Hz, 1H), | |
| | | | 7.09 (dd, J = 16.4, 9.2 Hz, | |
| | | | 1H), 6.90 (d, J = 15.2 Hz, | |
| | | | 1H), | |
| | | | 4.96-4.94 (m, 1H), | |
| | | | 4.38-4.36 (m, 1H), | |
| | | | 4.09-3.97 (m, 2H), | |
| | | | 3.20-3.10 (m, 2H), | |
| | | | 1.30 (d, J = 6.8 Hz, 3H) | |
| F54 | | ESIMS 592 | ¹H NMR (400 MHz, | IR (thin film) |
| | | ([M + H]⁺) | DMSO-d₆) δ 8.26 (d, | 3263, 1637, |
| | | | J = 8.4 Hz, 1H), | 1115, 750 cm⁻¹ |
| | | | 7.89 (s, 2H), 7.46 (s, 1H), | |
| | | | 7.41-7.39 (m, 1H), | |
| | | | 7.26 (d, J = 8.0 Hz, | |
| | | | 1H), 6.87 (dd, J = 15.6, | |
| | | | 8.8 Hz, 1H), | |
| | | | 6.76 (d, J = 15.6 Hz, | |
| | | | 1H), 4.84-4.80 (m, | |
| | | | 1H), 4.13-4.10 (m, | |
| | | | 1H), 3.57-3.52 (m, | |
| | | | 2H), 2.82-2.80 (m, | |
| | | | 2H), 2.73-2.69 (m, | |
| | | | 2H) 1.31-1.29 (m, | |
| | | | 3H), 1.17-1.15 (t, J = 8.4 Hz, | |
| | | | 3H) | |
| F55 | | ESIMS 609 | ¹H NMR (400 MHz, | IR (thin film) |
| | | ([M + H]⁺) | DMSO-d₆) δ 8.55 (d, | 3290, 1642, |
| | | | J = 6.3 Hz, 1H), | 1252, 808 cm⁻¹ |
| | | | 8.44 (d, J = 8.0 Hz, 1H), | |
| | | | 7.89 (s, 1H), | |
| | | | 7.47-7.41 (m, 2H), | |
| | | | 7.28-7.24 (m, 1H), | |
| | | | 6.87 (dd, J = 15.6, 8.8 Hz, | |
| | | | 1H), 6.76 (d, J = 15.6 Hz, | |
| | | | 1H), | |
| | | | 4.84-4.80 (m, 1H), | |
| | | | 4.41-4.37 (m, 1H), | |
| | | | 4.12-3.97 (m, 2H), | |
| | | | 3.21-3.11 (m, 2H), | |
| | | | 3.05-2.50 (m, 2H) | |
| | | | 1.31-1.29 (m, 3H), | |
| | | | 1.17-1.15 (t, J = 8.4 Hz, | |
| | | | 3H) | |
| F56 | | ESIMS 686 | ¹H NMR (300 MHz, | IR (thin film) |
| | | ([M + H]⁺) | DMSO-d₆) δ 8.52 (d, | 3436, 2925 |
| | | | J = 8.1 Hz, 1H), | 1644, 1117, |
| | | | 7.99 (s, 1H), | 747 cm⁻¹ |
| | | | 7.90-7.85 (m, 4H), 7.48 (d, J = 8.1 Hz, | |
| | | | 1H), | |
| | | | 7.09 (dd, J = 15.9, 9.3 Hz, | |
| | | | 1H), 6.88 (d, J = 15.6 Hz, | |
| | | | 1H), | |
| | | | 4.84-4.78 (m, 1H), | |
| | | | 4.10-4.01 (m, 1H), | |
| | | | 3.58-3.47 (m, 2H), | |
| | | | 2.82-2.78 (m, 2H), | |
| | | | 1.20 (d, J = 6.6 Hz, 3H) | |
| F57 | | ESIMS 632 | ¹H NMR (300 MHz, | IR (thin film) |
| | | ([M + H]⁺) | DMSO-d₆) δ 8.22 (d, | 3433, 2928 |
| | | | J = 7.8 Hz, 1H), | 1634, 1115 |
| | | | 7.88 (s, 1H), 7.81 (s, 2H), | 746 cm⁻¹ |
| | | | 7.46 (s, 1H), 7.40 (d, | |
| | | | J = 8.1 Hz, 1H), | |
| | | | 7.29 (d, J = 7.8 Hz, 1H), | |
| | | | 6.86 (dd, J = 15.3, | |
| | | | 8.4 Hz, 1H), 6.74 (d, | |
| | | | J = 15.9 Hz, 1H), | |
| | | | 4.81-4.75 (m, 1H), | |
| | | | 4.16-3.99 (m, 1H), | |
| | | | 3.58-3.47 (m, 2H), | |
| | | | 2.82 (d, J = 6.9 Hz, | |
| | | | 2H), 2.35 (s, 3H), | |
| | | | 1.21 (d, J = 6.9 Hz, | |
| | | | 3H) | |
| F58 | 95-99 | ESIMS 631 | ¹H NMR (400 MHz, | |
| | | ([M + H]⁺) | DMSO-d₆) δ 8.67 (dd, | |
| | | | J = 8.0 Hz, 1H), | |
| | | | 7.90 (s, 2H), 7.79 (s, 1H), | |
| | | | 7.57 (d, J = 8.0 Hz, | |
| | | | 1H), 7.43-7.39 (m, | |
| | | | 1H), 7.90 (dd, J = 16.0, | |
| | | | 8.7 Hz, 1H), | |
| | | | 6.78 (d, J = 16.4 Hz, | |
| | | | 1H), 4.85-4.80 (m, | |
| | | | 1H), 4.76-4.69 (m, | |
| | | | 2H), 4.55-4.52 (m, | |
| | | | 1H), 3.60-3.57 (m, | |
| | | | 1H), 3.49-3.31 (m, | |
| | | | 1H), 1.32 (d, J = 6.4 Hz, | |
| | | | 3H) | |
| F59 | 139-141 | ESIMS 752 | ¹H NMR (400 MHz, | |
| | | ([M + H]⁺) | DMSO-d₆) δ 8.74 (d, | |
| | | | J = 7.6 Hz, 1H), | |
| | | | 8.05 (s, 2H), 8.00 (s, 1H), | |
| | | | 7.92 (d, J = 8.0 Hz, | |
| | | | 1H), 7.52 (d, J = 8.0 Hz, | |
| | | | 1H), 7.09 (dd, J = 15.6, | |
| | | | 9.2 Hz, 1H), | |
| | | | 6.87 (d, J = 15.6 Hz, | |
| | | | 1H), 4.84-4.77 (m, | |
| | | | 1H), 4.74-4.69 (m | |
| | | | 2H), 4.54-4.51 (m, | |
| | | | 1H), 3.57-3.31 (m, | |
| | | | 2H), 1.30 (d, J = 6.4 Hz, | |
| | | | 3H) | |
| F60 | 139-143 | ESIMS 704 | ¹H NMR (400 MHz, | |
| | | ([M + H]⁺) | DMSO-d₆) δ 8.78 (d, | |
| | | | J = 8.4 Hz, 1H), | |
| | | | 8.01 (s, 1H), | |
| | | | 7.91-7.85 (m, 4H), | |
| | | | 7.52-7.46 (m, 1H), 7.08 (dd, J = 16.0, | |
| | | | 9.6 Hz, 1H), | |
| | | | 6.88 (d, J = 15.6 Hz, | |
| | | | 1H), 4.84-4.79 (m, | |
| | | | 1H), 4.40-4.37 (m, | |
| | | | 1H), 4.13-3.95 (m, | |
| | | | 2H), 3.21-3.05 (m, | |
| | | | 2H), 1.30 (d, J = 6.8 Hz, | |
| | | | 3H) | |
| F61 | 135-138 | ESIMS 716 | ¹H NMR (400 MHz, | |
| | | ([M − H]⁻) | DMSO-d₆) δ 8.74 (d, | |
| | | | J = 8.4 Hz, 1H), | |
| | | | 8.00 (s, 1H), | |
| | | | 7.92-7.89 (m, 2H), 7.85 (s, | |
| | | | 2H), 7.52 (d, J = 8.0 Hz, | |
| | | | 1H), 7.09 (dd, J = 15.6, | |
| | | | 9.2 Hz, 1H), | |
| | | | 6.88 (d, J = 15.6 Hz, | |
| | | | 1H), 4.84-4.72 (m, | |
| | | | 3H), 4.55-4.51 (m, | |
| | | | 1H), 3.59-3.45 (m, | |
| | | | 2H), 1.30 (d, J = 6.8 Hz, | |
| | | | 3H) | |
| F62 | | ESIMS 576 | ¹H NMR (300 MHz, | IR (thin film) |
| | | ([M − H]⁻) | DMSO-d₆) δ 8.46 (d, | 3418, 2926, |
| | | | J = 8.1 Hz, 1H), | 1646, 1115 cm⁻¹ |
| | | | 7.98 (s, 2H), 7.92 (s, 1H), | |
| | | | 7.90 (d, J = 8.1 Hz, | |
| | | | 1H), 7.49 (d, J = 7.8 Hz, | |
| | | | 1H), 7.03 (dd, J = 9.3, | |
| | | | 15.9 Hz, 1H), | |
| | | | 6.88 (d, J = 15.9 Hz, | |
| | | | 1H), 4.86 (t, J = 6.6 Hz, | |
| | | | 1H), | |
| | | | 4.05-4.01 (m, 1H), | |
| | | | 2.72-2.65 (m, 1H), | |
| | | | 2.59-2.48 (m, 3H), | |
| | | | 1.23-1.17 (m, 6H) | |
| F63 | | ESIMS 660 | ¹H NMR (300 MHz, | IR (thin film) |
| | | ([M + H]⁺) | DMSO-d₆) δ 9.07 (s, | 3422, 1664, |
| | | | 1H), 7.97 (s, 1H), | 1114, 808 cm⁻¹ |
| | | | 7.91 (s, 2H), 7.90 (d, | |
| | | | J = 9.0 Hz, 1H), | |
| | | | 7.49 (d, J = 7.8 Hz, 1H), | |
| | | | 7.08 (dd, J = 15.6, | |
| | | | 8.7 Hz, 1H), 6.87 (d, | |
| | | | J = 15.6 Hz, 1H), | |
| | | | 4.88-4.82 (m, 1H), | |
| | | | 4.09-3.98 (m, 2H), | |
| | | | 3.41 (d, J = 13.5 Hz, | |
| | | | 1H), 3.27 (d, J = 13.5 Hz, | |
| | | | 1H), | |
| | | | 1.01-0.81 (m, 4H) | |
| F64 | | ESIMS 652 | ¹H NMR (300 MHz, | IR (thin film) |
| | | ([M + H]⁺) | DMSO-d₆) δ 8.44 (d, | 3430, 2928, |
| | | | J = 8.1 Hz, 1H), | 1644, 1116, |
| | | | 7.89-7.78 (m, 4H), | 746 cm⁻¹ |
| | | | 7.56-7.53 (m, 1H), | |
| | | | 7.38 (d, J = 7.5 Hz, 1H), | |
| | | | 7.01 (dd, J = 15.6, | |
| | | | 9.0 Hz, 1H), 6.77 (d, | |
| | | | J = 15.3 Hz, 1H), | |
| | | | 4.82-4.76 (m, 1H), | |
| | | | 4.12-4.07 (m, 1H), | |
| | | | 3.58-3.47 (m, 2H), | |
| | | | 2.81 (d, J = 5.7 Hz, | |
| | | | 2H), 1.21 (d, J = 6.6 Hz, | |
| | | | 3H) | |
| F65 | 107-109 | ESIMS 684 | ¹H NMR (400 MHz, | |
| | | ([M + H]⁺) | DMSO-d₆) δ 8.67 (d, | |
| | | | J = 8.4 Hz, 1H), | |
| | | | 7.89-7.79 (m, 4H), | |
| | | | 7.58 (d, J = 8.0 Hz, 1H), | |
| | | | 7.41 (d, J = 7.6 Hz, | |
| | | | 1H), 7.00 (dd, J = 16.0, | |
| | | | 9.6 Hz, 1H), | |
| | | | 6.77 (d, J = 16.0 Hz, | |
| | | | 1H), 4.82-4.69 (m, | |
| | | | 3H), 4.57-4.50 (m, | |
| | | | 1H), 3.61-3.44 (m, | |
| | | | 2H), 1.32 (d, J = 6.8 Hz, | |
| | | | 3H) | |
| F66 | 158-160 | ESIMS 664 | ¹H NMR (400 MHz, | |
| | | ([M + H]⁺) | DMSO-d₆) δ 8.44 (d, | |
| | | | J = 8.4 Hz, 1H), | |
| | | | 7.89 (s, 1H), | |
| | | | 7.84-7.81 (m, 2H), 7.46 (s, | |
| | | | 1H), 7.41 (d, J = 7.6 Hz, | |
| | | | 1H), 7.31 (d, J = 7.6 Hz, | |
| | | | 1H), | |
| | | | 6.87 (dd, J = 15.9, 8.7 Hz, | |
| | | | 1H), 6.74 (d, J = 15.6 Hz, | |
| | | | 1H), | |
| | | | 4.78-4.68 (m, 3H), | |
| | | | 4.58-4.55 (m, 1H), | |
| | | | 3.57-3.44 (m, 2H), | |
| | | | 2.34 (s, 3H), 1.31 (d, J = 6.8 Hz, | |
| | | | 3H) | |
| F67 | | ESIMS 598 | ¹H NMR (400 MHz, | IR (thin film) |
| | | ([M + H]⁺) | DMSO-d₆) δ 8.44 (d, | 3428, 2925, |
| | | | J = 7.6 Hz 1H), | 1640, 1115 cm⁻¹ |
| | | | 7.93 (s, 2H), 7.78 (s, 1H), | |
| | | | 7.56-7.54 (m, 1H), | |
| | | | 7.39 (d, J = 8.0 Hz, | |
| | | | 1H), 7.00 (dd, J = 9.2, | |
| | | | 15.6 Hz, 1H), | |
| | | | 6.77 (d, J = 15.6 Hz, | |
| | | | 1H), 4.85 (t, J = 10.4 Hz, | |
| | | | 1H), | |
| | | | 4.11-4.08 (m, 1H), | |
| | | | 3.57-3.48 (m, 2H), | |
| | | | 2.82-2.79 (m, 2H), 1.23 (d, J = 5.2 Hz, | |
| | | | 3H) | |
| F68 | 102-103 | ESIMS 610 | ¹H NMR (300 MHz, | |
| | | ([M + H]⁺) | DMSO-d₆) δ 8.66 (dd, | |
| | | | J = 8.1 Hz, 1H), | |
| | | | 8.00 (s, 1H), | |
| | | | 7.92-7.89 (m, 3H), 7.54 (d, J = 7.8 Hz, | |
| | | | 1H), | |
| | | | 7.09 (dd, J = 15.6, 8.7 Hz, | |
| | | | 1H), 6.88 (d, J = 15.6 Hz, | |
| | | | 1H), 4.88 (t, | |
| | | | J = 9.0 Hz, 1H), | |
| | | | 4.47-4.43 (m, 1H), | |
| | | | 3.18-3.10 (m, 2H), | |
| | | | 1.28-1.21 (m, 8H) | |
| F69 | 88-90 | ESIMS 594 | ¹H NMR (300 MHz, | |
| | | ([M + H]⁺) | DMSO-d₆) δ 8.68 (dd, | |
| | | | J = 8.4, 8.1 Hz, 1H), | |
| | | | 8.00 (s, 1H), 7.92 (s, | |
| | | | 2H), 7.87 (s, 1H), | |
| | | | 7.54-7.46 (m, 1H), | |
| | | | 7.09 (dd, J = 15.9, | |
| | | | 9.3 Hz, 1H), 6.89 (d, | |
| | | | J = 16.2 Hz, 1H), | |
| | | | 4.85 (t, J = 9.0 Hz, | |
| | | | 1H), 4.35-4.30 (m, | |
| | | | 1H), 2.92-2.78 (m, | |
| | | | 4H), 1.33-1.23 (m, | |
| | | | 6H) | |
| F70 | | ESIMS 592 | ¹H NMR (400 MHz, | IR (thin film) |
| | | ([M + H]⁺) | DMSO-d₆) δ 8.45 (d, | 3322, 2927, |
| | | | J = 8.0 Hz 1H), | 1650, 1115 cm⁻¹ |
| | | | 7.98 (s, 1H), 7.92 (s, 1H), | |
| | | | 7.89 (d, J = 9.2 Hz, | |
| | | | 2H), 7.49 (d, J = 7.6 Hz, | |
| | | | 1H), 7.07 (dd, J = 15.6, | |
| | | | 9.2 Hz, 1H), | |
| | | | 6.88 (d, J = 15.6 Hz, | |
| | | | 1H), 4.76 (t, J = 9.2 Hz, | |
| | | | 1H), | |
| | | | 4.09-4.01 (m, 1H), | |
| | | | 2.70-2.65 (m, 2H), | |
| | | | 2.58-2.56 (m, 2H), | |
| | | | 1.56-1.51 (m, 2H), 1.21 (d, J = 4.2 Hz, | |
| | | | 3H), 0.95 (t, | |
| | | | J = 4.4 Hz, 3H) | |
| F71 | | ESIMS 623 | ¹H NMR (400 MHz, | IR (thin film) |
| | | ([M + H]⁺) | DMSO-d₆) δ 8.48 (d, | 3297, 1643, |
| | | | J = 8.4 Hz, 1H), | 1141, 809 cm⁻¹ |
| | | | 7.89 (s, 1H), | |
| | | | 7.47-7.41 (m, 2H), | |
| | | | 7.30-7.27 (m, 1H), 6.83 (dd, J = 15.6, | |
| | | | 8.8 Hz, 1H), | |
| | | | 6.76 (d, J = 15.6 Hz, | |
| | | | 1H), 4.82-4.71 (m, | |
| | | | 3H), 4.67-4.57 (m, | |
| | | | 2H), 3.57-3.47 (m, | |
| | | | 2H), 2.73-2.70 (m, | |
| | | | 2H), 1.31 (m, 3H), | |
| | | | 1.15 (m, 3H) | |
| F72 | 142-144 | ESIMS 668 | ¹H NMR (300 MHz, | |
| | | ([M + H]⁺) | DMSO-d₆) δ 8.73 (d, | |
| | | | J = 8.1 Hz, 1H), | |
| | | | 7.89-7.80 (m, 4H), | |
| | | | 7.57 (m, J = 8.1 Hz, 1H), | |
| | | | 7.41-7.37 (m, 1H), | |
| | | | 7.01 (dd, J = 15.3, | |
| | | | 9.0 Hz, 1H), 6.78 (d, | |
| | | | J = 15.3 Hz, 1H), | |
| | | | 4.82-4.76 (m, 1H), | |
| | | | 4.41-4.37 (m, 1H), | |
| | | | 4.14-3.98 (m, 2H), | |
| | | | 3.22-3.04 (m, 2H), | |
| | | | 1.31 (d, J = 6.6 Hz, | |
| | | | 3H) | |
| F73 | 163-165 | ESIMS 648 | ¹H NMR (400 MHz, | |
| | | ([M + H]⁺) | DMSO-d₆) δ 8.51 (d, | |
| | | | J = 8.4 Hz, 1H), | |
| | | | 7.88 (s, 1H), 7.81 (s, 2H), | |
| | | | 7.47 (s, 1H), 7.41 (d, | |
| | | | J = 7.6 Hz, 1H), | |
| | | | 7.29 (t, J = 6.8 Hz, 1H), | |
| | | | 6.85 (dd, J = 15.6, | |
| | | | 8.8 Hz, 1H), 6.74 (d, | |
| | | | J = 15.6 Hz, 1H), | |
| | | | 4.81-4.76 (m, 1H), | |
| | | | 4.41-4.38 (m, 1H), | |
| | | | 4.12-3.94 (m, 2H), | |
| | | | 3.21-3.06 (m, 2H), | |
| | | | 2.34 (s, 3H), 1.25 (d, | |
| | | | J = 7.6 Hz, 3H) | |
| F75 | | | ¹H NMR (300 MHz, | IR (thin film) |
| | | | DMSO-d₆) δ 8.52 (d, | 3423, 2925, |
| | | | J = 8.1 Hz, 1H), | 1644, 1262, |
| | | | 7.97 (s, 1H), 7.89 (d, J = 8.1 Hz, | 749 cm⁻¹ |
| | | | 1H), 7.47 (d, | |
| | | | J = 7.8 Hz, 1H), | |
| | | | 7.16 (s, 2H), 7.02 (dd, J = 15.9, | |
| | | | 9.0 Hz, 1H), | |
| | | | 6.84 (d, J = 15.9 Hz, | |
| | | | 1H), 6.16 (s, 2H), | |
| | | | 4.68-4.61 (m, 1H), | |
| | | | 4.10-4.04 (m, 1H), | |
| | | | 3.58-3.47 (m, 2H), | |
| | | | 2.82-2.78 (m, 2H), | |
| | | | 1.20 (d, J = 6.6 Hz, | |
| | | | 3H) | |
| F76 | 135-137 | ESIMS 641 | ¹H NMR (400 MHz, | |
| | | ([M + H]⁺) | DMSO-d₆) δ 8.74 (d, | |
| | | | J = 8.1 Hz, 1H), | |
| | | | 7.98 (s, 1H), 7.91 (d, J = 8.1 Hz, | |
| | | | 1H), 7.51 (d, | |
| | | | J = 7.8 Hz, 1H), | |
| | | | 7.16 (s, 2H), 7.03 (dd, J = 15.6, | |
| | | | 8.7 Hz, 1H), | |
| | | | 6.84 (d, J = 15.9 Hz, | |
| | | | 1H), 6.16 (s, 2H), | |
| | | | 4.78-4.51 (m, 4H), | |
| | | | 3.55-3.45 (m, 2H), | |
| | | | 1.30 (d, J = 6.6 Hz, | |
| | | | 3H) | |
| F79 | 95-99 | ESIMS 631 | ¹H NMR (300 MHz, | |
| | | ([M + H]⁺) | DMSO-d₆) δ 8.66 (d, | |
| | | | J = 8.1 Hz, 1H), | |
| | | | 8.00 (s, 1H), 7.92 (d, J = 7.5 Hz, | |
| | | | 3H), 7.54 (d, | |
| | | | J = 8.1 Hz, 1H), | |
| | | | 7.09 (dd, J = 15.9, 9.3 Hz, | |
| | | | 1H), 6.88 (d, J = 15.6 Hz, | |
| | | | 1H), 4.89 (t, | |
| | | | J = 9.6 Hz, 1H), | |
| | | | 4.47-4.43 (m, 1H), | |
| | | | 3.31-3.20 (m, 2H), | |
| | | | 3.15-3.10 (m, 2H), | |
| | | | 1.77-1.69 (m, 2H), | |
| | | | 1.28 (d, J = 6.6 Hz, 3H), | |
| | | | 1.02 (t, J = 7.2 Hz, | |
| | | | 3H) | |
| F80 | 78-80 | ESIMS 624 | ¹H NMR (400 MHz, | |
| | | ([M + H]⁺) | DMSO-d₆) δ 8.65 (d, | |
| | | | J = 8.4 Hz, 1H), | |
| | | | 8.00 (s, 1H), | |
| | | | 7.92-7.89 (m, 3H), 7.55 (d, J = 7.8 Hz, | |
| | | | 1H), | |
| | | | 7.09 (dd, J = 15.6, 8.4 Hz, | |
| | | | 1H), 6.88 (d, J = 15.3 Hz, | |
| | | | 1H), | |
| | | | 4.85-4.80 (m, 1H), | |
| | | | 4.50-4.45 (m, 1H), | |
| | | | 3.23-3.18 (m, 3H), | |
| | | | 1.29-1.13 (m, 9H) | |
| F81 | 85-88 | ESIMS 640 | ¹H NMR (300 MHz, | |
| | | ([M + H]⁺) | DMSO-d₆) δ 8.66 (d, | |
| | | | J = 8.4 Hz, 1H), | |
| | | | 8.00 (s, 1H), 7.91 (s, 2H), | |
| | | | 7.89 (s, 1H), 7.54 (d, | |
| | | | J = 7.8 Hz, 1H), | |
| | | | 7.09 (dd, J = 15.3, 8.4 Hz, | |
| | | | 1H), 6.88 (d, J = 15.9 Hz, | |
| | | | 1H), 4.89 (t, | |
| | | | J = 9.6 Hz, 1H), | |
| | | | 4.47-4.45 (m, 1H), | |
| | | | 3.26-3.21 (m, 2H), | |
| | | | 3.08 (d, J = 6.6 Hz, 2H), | |
| | | | 2.25-2.21 (m, 1H), | |
| | | | 1.27 (d, J = 6.6 Hz, | |
| | | | 3H), 1.06-1.03 (m, | |
| | | | 6H) | |
| F82 | 92-96 | ESIMS 608 | ¹H NMR (300 MHz, | |
| | | ([M + H]⁺) | DMSO-d₆) δ 8.66 (dd, | |
| | | | J = 8.1, 7.8 Hz, 1H), | |
| | | | 8.00 (s, 1H), | |
| | | | 7.92-7.88 (m, 3H), | |
| | | | 7.56 (dd, J = 8.1, 8.1 Hz, | |
| | | | 1H), 7.09 (dd, J = 15.6, | |
| | | | 9.0 Hz, 1H), | |
| | | | 6.88 (d, J = 15.6 Hz, | |
| | | | 1H), 4.89 (t, J = 9.6 Hz, | |
| | | | 1H), | |
| | | | 4.36-4.31 (m, 1H), | |
| | | | 3.31-2.79 (m, 2H), | |
| | | | 2.72-2.66 (m, 1H), | |
| | | | 1.29-1.16 (m, 9H) | |
| F83 | | ESIMS 660 | ¹H NMR (300 MHz, | IR (thin film) |
| | | ([M + H]⁺) | DMSO-d₆) δ 8.88 (br | 3291, 2930, |
| | | | s, 1H), 8.05-8.00 (m, | 1651, 1132 cm⁻¹ |
| | | | 1H), 7.92-7.90 (m, | |
| | | | 3H), 7.54 (d, J = 7.8 Hz, | |
| | | | 1H), 7.11 (dd, J = 15.6, | |
| | | | 9.0 Hz, 1H), | |
| | | | 6.90 (d, J = 15.6 Hz, | |
| | | | 1H), 4.89-4.83 (m, | |
| | | | 1H), 4.70 (d, J = 7.2 Hz, | |
| | | | 2H), 4.47 (d, J = 6.9 Hz, | |
| | | | 2H), | |
| | | | 3.80-3.76 (m, 2H), | |
| | | | 3.71-3.66 (m, 2H) | |
| F84 | | ESIMS 662 | ¹H NMR (400 MHz, | ¹⁹F NMR (376 MHz, |
| | | ([M + H]⁺) | CDCl₃) δ 7.67 (s, 1H), | CDCl₃) δ |
| | | | 7.59 (d, J = 8.1 Hz, | −58.86, −61.13, |
| | | | 1H), 7.55 (d, J = 8.0 Hz, | 92.42-−96.08 (m) |
| | | | 1H), 7.40 (s, 2H), | |
| | | | 6.57-6.51 (m, 2H), | |
| | | | 6.09 (d, J = 8.0 Hz, | |
| | | | 1H), 4.69 (p, J = 6.6 Hz, | |
| | | | 1H), | |
| | | | 4.20-3.91 (m, 2H), | |
| | | | 3.86-3.72 (m, 1H), 3.54 (dd, J = 14.6, | |
| | | | 6.2 Hz, 1H), | |
| | | | 3.39 (dd, J = 14.5, | |
| | | | 5.2 Hz, 1H), 1.62 (t, | |
| | | | J = 18.5 Hz, 3H), | |
| | | | 1.26 (d, J = 4.2 Hz, | |
| | | | 3H) | |
| F85 | | ESIMS 606 | ¹H NMR (300 MHz, | IR (thin film) |
| | | ([M + H]⁺) | DMSO-d₆) δ 8.45 (d, | 3422, 2925, |
| | | | J = 8.1 Hz, 1H), | 1645, 1115 cm⁻¹ |
| | | | 7.98 (s, 1H), 7.92 (s, 2H), | |
| | | | 7.87 (s, 1H), 7.49 (d, | |
| | | | J = 7.8 Hz, 1H), | |
| | | | 7.08 (dd, J = 15.9, 9.0 Hz, | |
| | | | 1H), 6.88 (d, J = 15.9 Hz, | |
| | | | 1H), 4.88 (t, | |
| | | | J = 6.6 Hz, 1H), | |
| | | | 4.10-4.00 (m, 1H), | |
| | | | 2.72-2.56 (m, 2H), | |
| | | | 2.49-2.43 (m, 3H), | |
| | | | 1.23-1.17 (m, 9H) | |
| F86 | | ESIMS 592 | ¹H NMR (300 MHz, | IR (thin film) |
| | | ([M + H]⁺) | DMSO-d₆) δ 8.46 (d, | 3431, 2926, |
| | | | J = 8.1 Hz, 1H), | 1650, 1115 cm⁻¹ |
| | | | 7.98 (s, 1H), 7.92 (s, 2H), | |
| | | | 7.90 (d, 8.1 Hz, 1H), | |
| | | | 7.49 (d, J = 7.8 Hz, | |
| | | | 1H), 7.08 (dd, J = 15.9, | |
| | | | 9.0 Hz, 1H), | |
| | | | 6.88 (d, J = 15.9 Hz, | |
| | | | 1H), 4.88 (t, J = 6.6 Hz, | |
| | | | 1H), | |
| | | | 4.04-3.99 (m, 1H), | |
| | | | 3.02-2.93 (m, 1H), | |
| | | | 2.74-2.68 (m, 2H), | |
| | | | 1.29-1.07 (m, 9H) | |
| F87 | 111-115 | ESIMS 622 | ¹H NMR (400 MHz, | |
| | | ([M + H]⁺) | DMSO-d₆) δ 8.46 (dd, | |
| | | | J = 8.4, 8.0 Hz, 1H), | |
| | | | 7.98 (s, 1H), 7.92 (s, | |
| | | | 2H), 7.88 (d, J = 8.4 Hz, | |
| | | | 1H), 7.49 (d, J = 7.6 Hz, | |
| | | | 1H), | |
| | | | 7.01 (dd, J = 15.6, 9.2 Hz, | |
| | | | 1H), 6.88 (d, J = 15.6 Hz, | |
| | | | 1H), 4.87 (t, | |
| | | | J = 7.2 Hz, 1H), | |
| | | | 4.15-4.01 (m, 1H), | |
| | | | 4.13-3.98 (m, 2H), | |
| | | | 2.99-2.90 (m, 2H), | |
| | | | 2.73-2.66 (m, 3H), | |
| | | | 1.27-1.17 (m, 9H) | |
| F88 | | ESIMS 598 | ¹H NMR (400 MHz, | IR (thin film) |
| | | ([M + H]⁺) | DMSO-d₆) δ 8.50 (d, | 3271, 1646 cm⁻¹ |
| | | | J = 8.0 Hz, 1H), | |
| | | | 7.99 (s, 1H), 7.90 (d, J = 8.4 Hz, | |
| | | | 1H), | |
| | | | 7.69-7.66 (m, 2H), | |
| | | | 7.48 (d, J = 8.4 Hz, 1H), | |
| | | | 7.14 (d, J = 8.4 Hz, | |
| | | | 1H), 7.08 (dd, J = 16.0, | |
| | | | 9.6 Hz, 1H), | |
| | | | 6.88 (d, J = 15.6 Hz, | |
| | | | 1H), 4.87-4.78 (m, | |
| | | | 1H), 4.12-4.02 (m, | |
| | | | 1H), 3.58-3.47 (m, | |
| | | | 2H), 3.31-2.78 (m, | |
| | | | 2H), 1.23-1.15 (m, | |
| | | | 3H) | |
| F89 | | ESIMS 544 | ¹H NMR (300 MHz, | IR (thin film) |
| | | ([M + H]⁺) | DMSO-d₆) δ 8.23 (d, | 3433, 1635 cm⁻¹ |
| | | | J = 8.7 Hz, 1H), | |
| | | | 7.66 (s, 2H), 7.46 (s, 1H), | |
| | | | 7.40 (d, J = 8.1 Hz, | |
| | | | 1H), 7.29 (d, J = 7.8 Hz, | |
| | | | 1H), 7.15 (d, J = 7.8 Hz, | |
| | | | 1H), | |
| | | | 7.08 (dd, J = 15.6, 9.0 Hz, | |
| | | | 1H), 6.88 (d, J = 15.6 Hz, | |
| | | | 1H), | |
| | | | 4.83-4.77 (m, 1H), | |
| | | | 4.14-4.07 (m, 1H), | |
| | | | 3.58-3.54 (m, 2H), | |
| | | | 2.82-2.78 (m, 2H), | |
| | | | 2.30 (s, 3H), | |
| | | | 1.23-1.15 (m, 3H) | |
| F90 | | ESIMS 615 | ¹H NMR (300 MHz, | IR (thin film) |
| | | ([M + H]⁺) | DMSO-d₆) δ 8.70 (d, | 3291, 1651 cm⁻¹ |
| | | | J = 8.1 Hz, 1H), | |
| | | | 8.01 (s, 1H), 7.91 (d, J = 7.5 Hz, | |
| | | | 1H), | |
| | | | 7.69-7.66 (m, 3H), | |
| | | | 7.52 (d, J = 7.8 Hz, 1H), | |
| | | | 7.01 (dd, J = 15.9, | |
| | | | 9.1 Hz, 1H), 6.89 (d, | |
| | | | J = 15.9 Hz, 1H), | |
| | | | 4.87-4.78 (m, 1H), | |
| | | | 4.42-4.31 (m, 1H), | |
| | | | 4.01-3.98 (m, 2H), | |
| | | | 3.18-3.08 (m, 2H), | |
| | | | 1.23-1.15 (m, 3H) | |
| F91 | | ESIMS 561 | ¹H NMR (300 MHz, | IR (thin film) |
| | | ([M + H]⁺) | DMSO-d₆) δ 8.41 (d, | 3292, 1640 cm⁻¹ |
| | | | J = 8.4 Hz, 1H), | |
| | | | 7.66 (s, 2H), 7.47 (s, 1H), | |
| | | | 7.41 (d, J = 8.1 Hz, | |
| | | | 1H), 7.31-7.24 (m, | |
| | | | 1H), 7.15 (d, J = 7.8 Hz, | |
| | | | 1H), 7.08 (dd, J = 15.6, | |
| | | | 9.0 Hz, 1H), | |
| | | | 6.88 (d, J = 15.6 Hz, | |
| | | | 1H), 4.83-4.77 (m, | |
| | | | 1H), 4.41-4.37 (m, | |
| | | | 1H), 4.14-3.93 (m, | |
| | | | 2H), 3.31-3.19 (m, | |
| | | | 2H), 2.34 (s, 3H), | |
| | | | 1.31-1.25 (m, 3H) | |
| F92 | | ESIMS 577 | ¹H NMR (300 MHz, | IR (thin film) |
| | | ([M + H]⁺) | DMSO-d₆) δ 8.55 (d, | 3291, |
| | | | J = 8.4 Hz, 1H), | 1643, 1314, |
| | | | 7.66 (s, 2H), 7.47 (s, 1H), | 1144 cm⁻¹ |
| | | | 7.41 (d, J = 8.1 Hz, | |
| | | | 1H), 7.32 (d, J = 8.1 Hz, | |
| | | | 1H), 7.15 (d, J = 7.8 Hz, | |
| | | | 1H), | |
| | | | 7.01 (dd, J = 15.6, 9.0 Hz, | |
| | | | 1H), 6.74 (d, J = 15.6 Hz, | |
| | | | 1H), | |
| | | | 4.83-4.71 (m, 3H), | |
| | | | 4.59-4.54 (m, 1H), | |
| | | | 3.61-3.47 (m, 2H), | |
| | | | 2.34 (s, 3H), 1.31 (d, J = 6.6 Hz, | |
| | | | 3H) | |
| F93 | | ESIMS 631 | ¹H NMR (300 MHz, | IR (thin film) |
| | | ([M + H]⁺) | DMSO-d₆) δ 8.74 (d, | 3275, 1654, |
| | | | J = 8.1 Hz, 1H), | 1325, 1245 cm⁻¹ |
| | | | 8.00 (s, 1H), 7.92 (d, J = 7.8 Hz, | |
| | | | 1H), 7.69 (m, | |
| | | | 2H), 7.52 (d, J = 7.8 Hz, | |
| | | | 1H), 7.21 (d, J = 7.2 Hz, | |
| | | | 1H), | |
| | | | 7.01 (dd, J = 15.9, 9.1 Hz, | |
| | | | 1H), 6.89 (d, J = 15.9 Hz, | |
| | | | 1H), | |
| | | | 4.86-4.68 (m, 3H), | |
| | | | 4.55-4.50 (m, 1H), | |
| | | | 3.57-3.45 (m, 2H), | |
| | | | 1.23-1.15 (m, 3H) | |
| F94 | | ESIMS 580 | ¹H NMR (300 MHz, | IR (thin film) |
| | | ([M + H]⁺) | DMSO-d₆) δ 8.62 (d, | 3263, 1651, |
| | | | J = 8.4 Hz, 1H), | 804 cm⁻¹ |
| | | | 7.79 (s, 1H), 7.68 (s, 3H), | |
| | | | 7.57 (d, J = 8.1 Hz, | |
| | | | 1H), 7.41-7.31 (m, | |
| | | | 1H), 7.01 (dd, J = 15.9, | |
| | | | 9.3 Hz, 1H), | |
| | | | 6.78 (d, J = 15.9 Hz, | |
| | | | 1H), 4.84-4.78 (m, | |
| | | | 1H), 4.39-4.37 (m, | |
| | | | 1H), 4.00-3.98 (m, | |
| | | | 2H), 3.14-2.98 (m, | |
| | | | 2H), 1.32-1.15 (m, | |
| | | | 3H) | |
| F95 | | ESIMS 598 | ¹H NMR (300 MHz, | IR (thin film) |
| | | ([M + H]⁺) | DMSO-d₆) δ 8.68 (d, | 3287, 1652, |
| | | | J = 8.1 Hz, 1H), | 1346, 1247 cm⁻¹ |
| | | | 7.80 (s, 1H), 7.68 (s, 3H), | |
| | | | 7.57 (d, J = 8.1 Hz, | |
| | | | 1H), 7.41 (d, J = 7.8 Hz, | |
| | | | 1H), 7.02 (dd, J = 15.6, | |
| | | | 9.0 Hz, 1H), | |
| | | | 6.78 (d, J = 15.6 Hz, | |
| | | | 1H), 4.84-4.68 (m, | |
| | | | 3H), 4.56-4.51 (m, | |
| | | | 1H), 3.57-3.39 (m, | |
| | | | 2H), 1.32 (d, J = 6.8 Hz, | |
| | | | 3H) | |
| F96 | | ESIMS 647 | ¹H NMR (300 MHz, | IR (thin film) |
| | | ([M + H]⁺) | DMSO-d₆) δ 8.36 (d, | 3430, 1647, |
| | | | J = 8.1 Hz, 1H), | 750 cm⁻¹ |
| | | | 7.98 (s, 1H), 7.92 (s, 2H), | |
| | | | 7.90 (d, J = 8.1 Hz, | |
| | | | 1H), 7.48 (d, J = 8.1 Hz, | |
| | | | 1H), 7.09 (dd, J = 15.9, | |
| | | | 9.3 Hz, 1H), | |
| | | | 6.88 (d, J = 15.6 Hz, | |
| | | | 1H), 4.88-4.84 (m, | |
| | | | 1H), 4.06-3.96 (m, | |
| | | | 1H), 3.54-3.39 (m, | |
| | | | 2H), 2.72-2.68 (m, | |
| | | | 2H), 1.76-1.68 (m, | |
| | | | 2H), 1.13 (d, J = 6.4 Hz, | |
| | | | 3H) | |
| F97 | | ESIMS 596 | ¹H NMR (300 MHz, | IR (thin film) |
| | | ([M + H]⁺) | DMSO-d₆) δ 8.52 (d, | 3420, 2924, |
| | | | J = 7.8 Hz, 1H), | 1641, 1115 cm⁻¹ |
| | | | 7.89 (s, 2H), 7.37 (d, J = 10.5 Hz, | |
| | | | 1H), 7.27 (s, | |
| | | | 1H), 7.39 (d, J = 8.0 Hz, | |
| | | | 1H), 6.95 (dd, J = 9.0, | |
| | | | 15.6 Hz, 1H), | |
| | | | 6.73 (d, J = 15.9 Hz, | |
| | | | 1H), 4.86 (t, J = 9.6 Hz, | |
| | | | 1H), | |
| | | | 4.15-4.10 (m, 1H), | |
| | | | 3.58-3.47 (m, 2H), | |
| | | | 2.81-2.78 (d, J = 6.6 Hz, 2H), | |
| | | | 2.28 (s, 3H), 1.22 (d, | |
| | | | J = 6.6 Hz, 3H) | |
| F98 | 90-93 | ESIMS 628 | ¹H NMR (400 MHz, | |
| | | ([M + H]⁺) | DMSO-d₆) δ 8.76 (d, | |
| | | | J = 8.0 Hz, 1H), | |
| | | | 7.89 (s, 2H), 7.39 (d, J = 10.8 Hz, | |
| | | | 1H), 7.27 (s, | |
| | | | 1H), 6.93 (dd, J = 15.6, | |
| | | | 9.2 Hz, 1H), | |
| | | | 6.72 (d, J = 15.6 Hz, | |
| | | | 1H), 4.85 (t, J = 9.2 Hz, | |
| | | | 1H), | |
| | | | 4.75-4.70 (m, 2H), | |
| | | | 4.59-4.52 (m, 1H), | |
| | | | 3.59 (dd, J = 7.2, | |
| | | | 14.4 Hz, 1H) | |
| | | | 3.48 (dd, J = 7.8, | |
| | | | 14.0 Hz, 1H), 2.27 (s, | |
| | | | 3H), 1.31 (d, J = 6.8 Hz, | |
| | | | 3H) | |
| F99 | 108-110 | ESIMS 612 | ¹H NMR (400 MHz, | |
| | | ([M + H]⁺) | DMSO-d₆) δ 8.82 (dd, | |
| | | | J = 8.4, 7.6 Hz, 1H), | |
| | | | 7.89 (s, 2H), 7.39 (d, | |
| | | | J = 10.8 Hz, 1H), | |
| | | | 7.28 (s, 1H), | |
| | | | 6.93 (dd, J = 16.0, 9.2 Hz, | |
| | | | 1H), 6.73 (d, J = 15.6 Hz, | |
| | | | 1H), 4.85 (t, | |
| | | | J = 10.0 Hz, 1H), | |
| | | | 4.42-4.37 (m, 1H), | |
| | | | 4.14-4.01 (m, 2H), | |
| | | | 3.21-3.13 (m, 2H), | |
| | | | 2.27 (s, 3H), 1.23 (d, | |
| | | | J = 3.6 Hz, 3H) | |
| F100 | | ESIMS 626 | ¹H NMR (300 MHz, | IR (thin film) |
| | | ([M + H]⁺) | DMSO-d₆) δ 10.32 (s, | 3440, 1644, |
| | | | 1H), 8.52 (d, J = 8.4 Hz, | 750 cm⁻¹ |
| | | | 1H), 8.23 (s, 1H), | |
| | | | 8.02-7.88 (m, 3H), | |
| | | | 7.48 (d, J = 7.8 Hz, | |
| | | | 1H), 7.09 (dd, J = 15.6, | |
| | | | 8.7 Hz, 1H), | |
| | | | 6.89 (d, J = 15.6 Hz, | |
| | | | 1H), 5.03-4.97 (m, | |
| | | | 1H), 4.10-4.01 (m, | |
| | | | 1H), 3.58-3.47 (m, | |
| | | | 2H), 2.81-2.79 (m, | |
| | | | 2H), 1.20 (d, J = 6.6 Hz, | |
| | | | 3H) | |
| F101 | | ESIMS 590 | ¹H NMR (300 MHz, | IR (thin film) |
| | | ([M + H]⁺) | DMSO-d₆) δ 8.47 (d, | 3431, 2924, |
| | | | J = 8.1 Hz, 1H), | 1650, 1260, |
| | | | 7.98 (s, 1H), 7.92 (s, 2H), | 750 cm⁻¹ |
| | | | 7.89 (d, J = 7.5 Hz, | |
| | | | 1H), 7.49 (d, J = 7.5 Hz, | |
| | | | 1H), 7.09 (dd, J = 8.7, | |
| | | | 15.3 Hz, 1H), | |
| | | | 6.88 (d, J = 16.2 Hz, | |
| | | | 1H), 5.82-5.73 (m, | |
| | | | 1H), 5.18-5.07 (m, | |
| | | | 2H), 4.88 (t, J = 9.0 Hz, | |
| | | | 1H), | |
| | | | 4.06-4.01 (m, 1H), 3.20 (d, J = 7.5 Hz, | |
| | | | 2H), | |
| | | | 2.65-2.58 (m, 2H), | |
| | | | 1.19 (d, J = 6.6 Hz, 3H) | |
| F102 | | ESIMS 622 | ¹H NMR (300 MHz, | IR (thin film) |
| | | ([M + H]⁺) | DMSO-d₆) δ 8.67 (d, | 3423, 2925, |
| | | | J = 8.4 Hz, 1H), | 1651, 1260, |
| | | | 8.00 (s, 2H), 7.92 (d, J = 8.4 Hz, | 750 cm⁻¹ |
| | | | 1H), 7.54 (d, | |
| | | | J = 8.1 Hz, 1H), | |
| | | | 7.09 (dd, J = 15.6, 8.4 Hz, | |
| | | | 1H), 6.88 (d, J = 15.6 Hz, | |
| | | | 1H), | |
| | | | 5.87-5.82 (m, 1H), | |
| | | | 5.50-5.44 (m, 2H), 4.89 (t, | |
| | | | J = 8.4 Hz, 1H), | |
| | | | 4.49-4.45 (m, 1H), | |
| | | | 3.99 (d, J = 7.2 Hz, 2H), | |
| | | | 3.39-3.24 (m, 2H), | |
| | | | 1.28 (d, J = 6.6 Hz, | |
| | | | 3H) | |
| F103 | | ESIMS 606 | ¹H NMR (300 MHz, | IR (thin film) |
| | | ([M + H]⁺) | DMSO-d₆) δ 8.70 (dd, | 3422, 2924, |
| | | | J = 8.4, 7.5 Hz, 1H), | 1650, 1171 cm⁻¹ |
| | | | 8.00 (s, 2H), | |
| | | | 7.92-7.87 (m, 2H), | |
| | | | 7.53-7.45 (m, 1H), | |
| | | | 7.09 (dd, J = 15.9, 8.7 Hz, | |
| | | | 1H), 6.88 (d, J = 15.6 Hz, | |
| | | | 1H), | |
| | | | 5.93-5.85 (m, 1H), | |
| | | | 5.40-5.35 (m, 2H), 4.88 (t, | |
| | | | J = 9.6 Hz, 1H), | |
| | | | 4.36-4.31 (m, 1H), | |
| | | | 3.66-3.60 (m, 1H), | |
| | | | 3.52-3.45 (m, 1H), | |
| | | | 2.95-2.89 (m, 2H), | |
| | | | 1.28 (d, J = 3.9 Hz, 3H) | |
| F104 | | ESIMS 564 | ¹H NMR (400 MHz, | IR (thin film) |
| | | ([M + H]⁺) | DMSO-d₆) δ 8.43 (d, | 3433, 1635 cm⁻¹ |
| | | | J = 8.4 Hz, 1H), | |
| | | | 7.78 (s, 1H), 7.67 (s, 3H), | |
| | | | 7.55 (t, J = 6.8 Hz, | |
| | | | 1H), 7.38 (d, J = 8.0 Hz, | |
| | | | 1H), 6.99 (dd, J = 15.6, | |
| | | | 9.2 Hz, 1H), | |
| | | | 6.77 (d, J = 15.6 Hz, | |
| | | | 1H), 4.82-4.78 (m, | |
| | | | 1H), 4.11-4.08 (m, | |
| | | | 1H), 3.56-3.51 (m, | |
| | | | 2H), 2.82-2.78 (m, | |
| | | | 2H), 1.23-1.15 (d, J = 8.8 Hz, | |
| | | | 3H) | |
| F105 | | ESIMS 664 | ¹H NMR (300 MHz, | IR (thin film) |
| | | ([M + H]⁺) | DMSO-d₆) δ 8.46 (d, | 3422, 1647, |
| | | | J = 8.4 Hz 1H), | 810 cm⁻¹ |
| | | | 8.00 (s, 1H), 7.92 (s, 2H), | |
| | | | 7.90 (d, J = 7.8 Hz, | |
| | | | 1H), 7.54 (d, J = 7.5 Hz, | |
| | | | 1H), 7.09 (dd, J = 15.9, | |
| | | | 9.3 Hz, 1H), | |
| | | | 6.89 (d, J = 15.3 Hz, | |
| | | | 1H), 4.88-4.26 (m, | |
| | | | 1H), 4.10-3.88 (m, | |
| | | | 3H), 2.98-2.95 (m, | |
| | | | 2H), 1.88-1.86 (m, | |
| | | | 2H), 1.13 (d, J = 6.4 Hz, | |
| | | | 3H) | |
| F106 | | ESIMS 680 | ¹H NMR (400 MHz, | IR (thin film) |
| | | ([M + H]⁺) | CDCl₃) δ 7.68 (s, 1H), | 3430, 1647, |
| | | | 7.62 (d, J = 8.0 Hz | 764 cm⁻¹ |
| | | | 1H), 7.51 (d, J = 5.4 Hz | |
| | | | 1H), 7.41 (s, 2H), | |
| | | | 6.64 (d, J = 16.0 Hz, | |
| | | | 1H), 6.47 (dd, J = 15.6, | |
| | | | 8.0 Hz 1H), | |
| | | | 5.69 (d, J = 8.8 Hz | |
| | | | 1H), 4.34 (br s, 1H), | |
| | | | 4.14-4.10 (m, 1H), | |
| | | | 3.85-3.79 (m, 2H), | |
| | | | 3.49-3.30 (m, 2H), | |
| | | | 2.21-2.04 (m, 2H), | |
| | | | 1.35 (d, J = 6.8 Hz, | |
| | | | 3H) | |
| F107 | | ESIMS 676 | ¹H NMR (300 MHz, | IR (thin film) |
| | | ([M + H]⁺) | DMSO-d₆) δ 9.06 (br | 3434, 2924, |
| | | | s, 1H), 8.03 (s, 1H), | 1275, 764 cm⁻¹ |
| | | | 7.93-7.91 (m, 3H), | |
| | | | 7.54-7.51 (m, 1H), | |
| | | | 7.08 (dd, J = 15.9, | |
| | | | 8.7 Hz, 1H), 6.90 (d, | |
| | | | J = 15.9 Hz, 1H), | |
| | | | 4.94-4.92 (m, 2H), | |
| | | | 4.86-4.83 (m, 1H), | |
| | | | 4.72-4.57 (m, 2H), | |
| | | | 4.03-3.89 (m, 4H) | |
| F108 | | ESIMS 631 | ¹H NMR (400 MHz, | ¹⁹F NMR (376 MHz, |
| | | ([M + H]⁺) | CDCl₃) δ 7.66 (s, 1H), | CDCl₃) δ |
| | | | 7.58 (d, J = 8.0 Hz, | −58.82, −66.50, |
| | | | 1H), 7.52 (d, J = 7.9 Hz, | −91.61-−96.84 (m) |
| | | | 1H), 7.40 (s, 2H), | |
| | | | 6.56-6.51 (m, 2H), | |
| | | | 5.80 (d, J = 8.2 Hz, | |
| | | | 1H), 4.47-4.30 (m, | |
| | | | 1H), 3.79 (tt, J = 14.5, | |
| | | | 3.6 Hz, 1H), | |
| | | | 3.28-3.07 (m, 2H), | |
| | | | 2.98-2.81 (m, 2H), | |
| | | | 1.62 (t, J = 18.5 Hz, | |
| | | | 3H), 1.34 (d, J = 6.7 Hz, | |
| | | | 3H) | |
| F109 | | ESIMS 623 | | |
| | | ([M + H]⁺) | | |
| F110 | | ESIMS 646 | ¹H NMR (300 MHz, | IR (thin film) |
| | | ([M + H]⁺) | DMSO-d₆) δ 8.88 (s, | 3422, 2925, |
| | | | 1H), 7.98 (s, 1H), | 1656, 750 cm⁻¹ |
| | | | 7.91 (m, 3H), | |
| | | | 7.46 (d, J = 8.1 Hz, 1H), | |
| | | | 7.09 (dd, J = 15.6, | |
| | | | 9.0 Hz, 1H), 6.88 (d, | |
| | | | J = 15.6 Hz, 1H), | |
| | | | 4.88-4.82 (m, 1H), | |
| | | | 3.58-3.47 (m, 2H), | |
| | | | 3.05 (s, 2H), | |
| | | | 1.23-1.15 (m, 2H), | |
| | | | 0.85-0.80 (m, 2H) | |
| F111 | 151-156 | ESIMS 608 | ¹H NMR (300 MHz, | |
| | | ([M + H]⁺) | DMSO-d₆) δ 8.58 (dd, | |
| | | | J = 6.6, 7.8 Hz, 1H), | |
| | | | 8.00 (s, 1H), | |
| | | | 7.92-7.88 (m, 2H), | |
| | | | 7.53 (d, J = 8.1 Hz, 1H), | |
| | | | 7.40-7.36 (m, 1H), | |
| | | | 7.09 (dd, J = 15.9, | |
| | | | 9.3 Hz, 1H), 6.88 (d, | |
| | | | J = 15.6 Hz, 1H), | |
| | | | 4.89 (t, J = 9.3 Hz, | |
| | | | 1H), 4.35-4.31 (m, | |
| | | | 1H), 2.97-2.88 (m, | |
| | | | 1H), 2.79-2.67 (m, | |
| | | | 3H), 1.71-1.66 (m, | |
| | | | 2H), 1.28 (d, J = 3.9 Hz, | |
| | | | 3H), 1.03 (t, J = 6.6 Hz, | |
| | | | 3H) | |
| F112 | | ESIMS 674 | ¹H NMR (300 MHz, | IR (thin film) |
| | | ([M − H]⁻) | DMSO-d₆) δ 9.03 (s, | 3422, 2925, |
| | | | 1H), 7.97 (s, 1H), | 1656, 750 cm⁻¹ |
| | | | 7.91 (s, 3H), 7,52 (d, | |
| | | | J = 7.8 Hz, 1H), | |
| | | | 7.09 (dd, J = 15.6, 9.0 Hz, | |
| | | | 1H), 6.87 (d, J = 15.6 Hz, | |
| | | | 1H), | |
| | | | 4.88-4.82 (m, 1H), | |
| | | | 4.73-4.63 (m, 2H), | |
| | | | 3.68 (s, 2H), | |
| | | | 1.23-1.15 (m, 2H), | |
| | | | 0.87-0.80 (m, 2H) | |
| F113 | | HRMS-FAB | ¹H NMR (400 MHz, | |
| | | calcd for | CDCl₃) δ 7.68 (s, 1H), | |
| | | C₂₄H₂₂Cl₃F₆NOS₂, | 7.60 (dd, J = 8.0, 1.6 Hz, | |
| | | 623.0113 | 1H), 7.55 (d, J = 8.0 Hz, | |
| | | found, | 1H), 7.42 (s, | |
| | | 623.0131. | 2H), 6.63 (d, J = 15.9 Hz, | |
| | | | 1H), | |
| | | | 6.44 (dd, J = 15.9, 7.9 Hz, | |
| | | | 1H), 6.38 (t, J = 5.8 Hz, | |
| | | | 1H), | |
| | | | 4.22-4.05 (m, 1H), 4.00 (t, J = 6.9 Hz, | |
| | | | 1H), 3.74 (t, | |
| | | | J = 6.4 Hz, 2H), | |
| | | | 2.81-2.59 (m, 4H), | |
| | | | 1.29 (t, J = 7.4 Hz, 6H) | |
| F114 | | ESIMS 709 | ¹H NMR (400 MHz, | ¹⁹F NMR (376 MHz, |
| | | ([M − H]⁻) | CDCl₃) δ 7.65 (d, J = 1.5 Hz, | CDCl₃) δ |
| | | | 1H), | −58.97, −61.12, |
| | | | 7.60-7.50 (m, 3H), | −68.56 |
| | | | 7.46 (d, J = 2.0 Hz, 1H), | |
| | | | 6.62 (d, J = 15.9 Hz, | |
| | | | 1H), 6.44 (dd, J = 15.9, | |
| | | | 7.9 Hz, 1H), | |
| | | | 6.34 (d, J = 8.1 Hz, | |
| | | | 1H), 4.67 (ddd, J = 13.5, | |
| | | | 9.5, 6.0 Hz, | |
| | | | 1H), 4.20-4.03 (m, | |
| | | | 2H), 3.97 (dt, J = 15.5, | |
| | | | 9.3 Hz, 1H), | |
| | | | 3.53 (dd, J = 14.5, | |
| | | | 6.5 Hz, 1H), | |
| | | | 3.38 (dd, J = 14.5, 5.1 Hz, | |
| | | | 1H), 1.50 (d, J = 6.9 Hz, | |
| | | | 3H) | |
| F115 | | ESIMS 655 | ¹H NMR (400 MHz, | ¹⁹F NMR (376 MHz, |
| | | ([M − H]⁻) | CDCl₃) δ 7.66 (d, J = 1.7 Hz, | CDCl₃) δ |
| | | | 1H), | −57.06-−60.12 (m), −61.12, |
| | | | 7.61-7.48 (m, 3H), | −66.25-−70.73 (m) |
| | | | 7.48-7.39 (m, 2H), | |
| | | | 7.18-7.05 (m, 1H), | |
| | | | 6.61 (d, J = 15.9 Hz, | |
| | | | 1H), 6.54-6.42 (m, 1H), | |
| | | | 6.23 (d, J = 8.1 Hz, | |
| | | | 1H), 5.84-5.65 (m, | |
| | | | 1H), 5.54-5.37 (m, | |
| | | | 1H), 4.67 (dq, J = 13.2, | |
| | | | 6.5 Hz, 1H), | |
| | | | 4.34-3.90 (m, 2H), | |
| | | | 3.54 (dd, J = 14.6, | |
| | | | 6.3 Hz, 1H), | |
| | | | 3.39 (dd, J = 14.5, 5.2 Hz, | |
| | | | 1H), 1.52 (d, J = 6.9 Hz, | |
| | | | 3H) | |
| F116 | | ESIMS 675 | ¹H NMR (400 MHz, | ¹⁹F NMR (376 MHz, |
| | | ([M − H]⁻) | CDCl₃) δ 7.66 (d, J = 1.6 Hz, | CDCl₃) δ |
| | | | 1H), | −58.92, −66.51, |
| | | | 7.62-7.55 (m, 2H), | −68.19 |
| | | | 7.51 (d, J = 8.0 Hz, | |
| | | | 1H), | |
| | | | 7.46 (d, J = 2.0 Hz, | |
| | | | 1H), 6.62 (d, J = 15.9 Hz, | |
| | | | 1H), | |
| | | | 6.43 (dd, J = 15.9, 7.9 Hz, | |
| | | | 1H), 5.99 (d, J = 8.2 Hz, | |
| | | | 1H), 4.36 (dq, J = 8.2, | |
| | | | 6.3 Hz, 1H), | |
| | | | 4.14 (d, J = 8.1 Hz, | |
| | | | 1H), 3.28-3.04 (m, | |
| | | | 2H), 2.97-2.79 (m, | |
| | | | 2H), 1.33 (d, J = 6.7 Hz, | |
| | | | 3H) | |
| F117 | | ESIMS 623.9 | ¹H NMR (300 MHz, | ¹⁹F NMR (471 MHz, |
| | | ([M − H]⁻) | Methanol-d₄) δ | CDCl₃) δ |
| | | | 7.74 (d, J = 1.5 Hz, 1H), | −60.71, −68.71 |
| | | | 7.67 (d, J = 2.0 Hz, | |
| | | | 1H), 7.61-7.45 (m, | |
| | | | 2H), 7.40 (ddd, J = 9.4, | |
| | | | 8.1, 1.8 Hz, 2H), | |
| | | | 6.79-6.60 (m, 2H), | |
| | | | 4.71-4.37 (m, 1H), | |
| | | | 4.13-3.66 (m, 2H), | |
| | | | 3.40-3.04 (m, 3H), | |
| | | | 1.44 (dd, J = 6.8, 5.2 Hz, | |
| | | | 3H) | |
| F118 | | ESIMS 646.3 | ¹H NMR (500 MHz, | ¹⁹F NMR (471 MHz, |
| | | ([M + H]⁺) | CDCl₃) δ 7.67 (s, 1H), | CDCl₃) δ |
| | | | 7.62-7.57 (m, 1H), | −58.90, −61.12, |
| | | | 7.55 (d, J = 8.0 Hz, | −116.70-−122.10 (m); |
| | | | 1H), 7.37 (s, 2H), | IR (thin film) |
| | | | 6.58 (d, J = 16.0 Hz, | 3269, 2978, |
| | | | 1H), 6.45 (dd, J = 16.0, | 1650, 1533 cm⁻¹ |
| | | | 7.6 Hz, 1H), | |
| | | | 6.16 (d, J = 7.9 Hz, | |
| | | | 1H), 6.02 (td, J = 55.7, | |
| | | | 3.4 Hz, 1H), | |
| | | | 4.69 (dt, J = 13.3, | |
| | | | 6.6 Hz, 1H), | |
| | | | 4.17-4.05 (m, 1H), | |
| | | | 4.05-3.96 (m, 1H), | |
| | | | 3.96-3.80 (m, 1H), | |
| | | | 3.54 (dd, J = 14.5, 6.3 Hz, | |
| | | | 1H), 3.40 (dd, J = 14.5, | |
| | | | 5.2 Hz, 1H), | |
| | | | 1.54 (d, J = 6.9 Hz, | |
| | | | 3H) | |
| F119 | | HRMS-ESI | ¹H NMR (300 MHz, | ¹⁹F NMR (471 MHz, |
| | | (m/z) [M + H]⁺ | CDCl₃) δ | DMSO-d₆) |
| | | calcd for | 7.69-7.62 (m, 1H), 7.57 (d, J = 8.3 Hz, | δ −57.78, |
| | | C₂₄H₂₂Cl₃F₆NOS, | 1H), 7.51 (d, | −92.20 (td, J = 19.7, |
| | | 594.0438; | J = 7.9 Hz, 1H), | 14.7 Hz), |
| | | found, | 7.40 (s, 2H), | −211.57-−212.01 (m); |
| | | 594.0444 | 6.62-6.44 (m, 2H), 5.93 (d, J = 8.1 Hz, | IR (thin film) |
| | | | 1H), | 1646, 1550 cm⁻¹ |
| | | | 4.58 (dtd, J = 47.0, 6.3, | |
| | | | 1.4 Hz, 2H), | |
| | | | 4.35 (hept, J = 6.5 Hz, | |
| | | | 1H), 3.79 (tt, J = 14.4, | |
| | | | 3.6 Hz, 1H), | |
| | | | 2.99-2.69 (m, 4H), | |
| | | | 1.62 (t, J = 18.5 Hz, | |
| | | | 3H), 1.32 (d, J = 6.7 Hz, | |
| | | | 3H) | |
| F120 | | ESIMS 734.1 | ¹H NMR (400 MHz, | ¹⁹F NMR (376 MHz, |
| | | ([M + H]⁺) | CDCl₃) δ 7.61 (s, 1H), | CDCl₃) δ |
| | | | 7.53 (d, J = 8.0 Hz, | −58.90, −61.13, |
| | | | 1H), 7.48 (d, J = 8.0 Hz, | −73.82, |
| | | | 1H), 7.21 (d, J = 1.5 Hz, | −88.60-−97.49 (m); |
| | | | 1H), 7.06 (t, | IR (thin film) |
| | | | J = 1.9 Hz, 1H), | 3269, 1650, |
| | | | 6.92 (t, J = 1.8 Hz, 1H), | 1573 cm⁻¹ |
| | | | 6.57 (dd, J = 15.9, | |
| | | | 7.8 Hz, 1H), 6.49 (d, | |
| | | | J = 16.0 Hz, 1H), | |
| | | | 6.35 (d, J = 8.0 Hz, | |
| | | | 1H), 4.67 (dt, J = 13.4, | |
| | | | 6.7 Hz, 1H), | |
| | | | 4.36 (q, J = 8.0 Hz, | |
| | | | 2H), 4.17-3.93 (m, | |
| | | | 2H), 3.79 (ddd, J = 16.8, | |
| | | | 12.2, 7.8 Hz, | |
| | | | 1H), 3.54 (dd, J = 14.6, | |
| | | | 6.3 Hz, 1H), | |
| | | | 3.37 (dd, J = 14.5, | |
| | | | 5.2 Hz, 1H), 1.60 (t, | |
| | | | J = 18.5 Hz, 3H), | |
| | | | 1.51 (d, J = 6.9 Hz, | |
| | | | 3H) | |
| F121 | | HRMS-ESI | ¹H NMR (500 MHz, | ¹⁹F NMR (471 MHz, |
| | | (m/z) [M + H]⁺ | DMSO-d₆, mixture of | DMSO-d₆, |
| | | calcd for | diastereomers) δ | mixture of |
| | | C₂₄H₂₂Cl₃F₆NO₂S, | 8.79-8.52 (m, 1H), | diastereomers) |
| | | 610.0387; | 8.00-7.93 (m, 1H), | δ −57.76, |
| | | found, | 7.87 (dt, J = 7.8, 2.3 Hz, | −57.78, |
| | | 610.0383 | 1H), 7.81 (s, 2H), | −92.06-−92.33 (m), |
| | | | 7.53-7.44 (m, 1H), | −218.82 (tdd, J = 46.9, |
| | | | 7.02 (dd, J = 15.8, | 34.6, |
| | | | 9.4 Hz, 1H), 6.74 (d, | 21.6 Hz), |
| | | | J = 15.7 Hz, 1H), | −219.22 (tdd, J = 47.1, |
| | | | 4.98-4.71 (m, 2H), | 34.1, |
| | | | 4.43-4.21 (m, 2H), | 21.8 Hz); |
| | | | 3.31-3.19 (m, 1H), | IR (thin film) |
| | | | 3.17-2.91 (m, 3H), | 3245, 2973, |
| | | | 1.62 (t, J = 19.0 Hz, | 1650, 1550 cm⁻¹ |
| | | | 3H), 1.32-1.24 (m, | |
| | | | 3H) | |
| F122 | | HRMS-ESI | ¹H NMR (500 MHz, | ¹⁹F NMR (471 MHz, |
| | | (m/z) [M + H]⁺ | DMSO-d₆) δ 8.66 (d, | DMSO-d₆) |
| | | calcd for | J = 8.2 Hz, 1H), | δ −57.80, |
| | | C₂₄H₂₂Cl₃F₆NO₃S, | 7.96 (t, J = 2.0 Hz, 1H), | −92.20 (td, J = 18.8, |
| | | 626.0337; | 7.88 (d, J = 8.1 Hz, | 13.8 Hz), |
| | | found, | 1H), 7.81 (s, 2H), | −219.25 (tt, J = 47.0, |
| | | 626.0334 | 7.52 (d, J = 7.9 Hz, | 26.4 Hz); |
| | | | 1H), 7.02 (dd, J = 15.8, | IR (thin film) |
| | | | 9.4 Hz, 1H), | 3250, 2978, |
| | | | 6.74 (d, J = 15.7 Hz, | 1651, 1550 cm⁻¹ |
| | | | 1H), 4.89 (dd, J = 6.0, | |
| | | | 4.5 Hz, 1H), | |
| | | | 4.79 (dd, J = 6.1, 4.4 Hz, | |
| | | | 1H), 4.51 (hept, J = 6.8 Hz, | |
| | | | 1H), 4.29 (td, | |
| | | | J = 14.5, 9.4 Hz, 1H), | |
| | | | 3.78-3.57 (m, 2H), | |
| | | | 3.40 (dd, J = 14.2, | |
| | | | 7.2 Hz, 1H), | |
| | | | 3.32-3.28 (m, 1H), 1.62 (t, | |
| | | | J = 18.9 Hz, 3H), | |
| | | | 1.32-1.25 (m, 3H) | |
| F123 | | ESIMS 644.2 | ¹H NMR (400 MHz, | ¹⁹F NMR (376 MHz, |
| | | ([M + H]⁺) | CDCl₃) δ 7.66 (s, 1H), | CDCl₃) δ |
| | | | 7.66-7.58 (m, 2H), | −58.88, −61.13, |
| | | | 7.55 (t, J = 8.8 Hz, | −61.41 (d, J = 12.4 Hz), |
| | | | 2H), 7.24 (d, J = 3.2 Hz, | −92.67-−95.42 (m), |
| | | | 1H), 6.60 (dd, J = 15.9, | −113.13 (q, J = 12.4 Hz); |
| | | | 7.4 Hz, 1H), | IR (thin film) |
| | | | 6.54 (d, J = 16.0 Hz, | 3264, 1648, |
| | | | 1H), 6.08 (d, J = 8.2 Hz, | 1537 cm⁻¹ |
| | | | 1H), 4.68 (dd, J = 13.6, | |
| | | | 6.6 Hz, 1H), | |
| | | | 4.17-3.84 (m, 3H), | |
| | | | 3.54 (dd, J = 14.6, | |
| | | | 6.2 Hz, 1H), | |
| | | | 3.39 (dd, J = 14.5, 5.1 Hz, | |
| | | | 1H), 1.62 (t, J = 18.5 Hz, | |
| | | | 3H), 1.54 (d, J = 6.9 Hz, | |
| | | | 3H) | |
| F124 | | ESIMS 640.1 | ¹H NMR (300 MHz, | ¹⁹F NMR (471 MHz, |
| | | ([M − H]⁻) | Methanol-d₄) δ | CDCl₃) δ |
| | | | 7.73 (d, J = 1.5 Hz, 1H), | −61.04, −68.71 |
| | | | 7.67 (d, J = 2.1 Hz, | |
| | | | 1H), 7.57 (d, J = 8.4 Hz, | |
| | | | 1H), 7.52 (dd, J = 8.0, | |
| | | | 1.6 Hz, 1H), | |
| | | | 7.48-7.33 (m, 3H), | |
| | | | 6.79-6.56 (m, 2H), | |
| | | | 4.69 (td, J = 7.2, 5.6 Hz, | |
| | | | 1H), | |
| | | | 4.58-4.31 (m, 3H), 3.60 (dd, J = 14.3, | |
| | | | 7.4 Hz, 1H), | |
| | | | 3.47 (dd, J = 14.3, | |
| | | | 5.4 Hz, 1H), 1.43 (d, | |
| | | | J = 6.8 Hz, 3H) | |
| F125 | | HRMS-ESI | ¹H NMR (400 MHz, | ¹⁹F NMR (376 MHz, |
| | | (m/z) [M + H]⁺ | CDCl₃) δ 7.69 (d, J = 1.5 Hz, | CDCl₃) δ |
| | | calcd for | 1H), | −59.00, −66.49, |
| | | C₂₃H₁₆Cl₃F₉N₂O₂S, | 7.60 (dd, J = 8.0, 1.6 Hz, | −68.56; |
| | | 662.9900; | 1H), 7.53 (d, J = 8.0 Hz, | IR (thin film) |
| | | found, | 1H), 7.42 (s, 2H), | 3276, 1656, |
| | | 662.9904 | 6.94 (d, J = 7.7 Hz, | 1552, 1520 cm⁻¹ |
| | | | 1H), 6.71-6.57 (m, | |
| | | | 2H), 6.45 (dd, J = 15.9, | |
| | | | 7.8 Hz, 1H), | |
| | | | 5.85-5.66 (m, 1H), | |
| | | | 4.87 (td, J = 7.3, 5.6 Hz, | |
| | | | 1H), | |
| | | | 4.21-4.06 (m, 1H), | |
| | | | 3.35-3.12 (m, 3H), 3.04 (dd, J = 14.1, | |
| | | | 7.0 Hz, 1H) | |
| F126 | | HRMS-ESI | ¹H NMR (300 MHz, | IR (thin film) |
| | | (m/z) [M + H]⁺ | CDCl₃) δ 7.67 (d, J = 1.4 Hz, | 1662 cm⁻¹ |
| | | calcd for | 1H), | |
| | | C₂₃H₁₈Cl₃F₈NO₃S, | 7.62-7.56 (m, 1H), | |
| | | 647.9991; | 7.51 (d, J = 8.0 Hz, 1H), | |
| | | found, | 7.40 (s, 2H), | |
| | | 647.9989 | 6.57-6.51 (m, 2H), 6.44 (t, | |
| | | | J = 6.2 Hz, 1H), | |
| | | | 4.03 (q, J = 6.0 Hz, 2H), | |
| | | | 3.89 (q, J = 9.1 Hz, | |
| | | | 2H), 3.84-3.71 (m, | |
| | | | 1H), 3.54-3.46 (m, | |
| | | | 2H), 1.61 (t, J = 18.6 Hz, | |
| | | | 3H) | |
| F128 | | HRMS-ESI | ¹H NMR (500 MHz, | ¹⁹F NMR (471 MHz, |
| | | (m/z) [M + H]⁺ | DMSO-d₆) δ 8.63 (t, J = 5.7 Hz, | DMSO-d₆) |
| | | calcd for | 1H), | δ −57.80, |
| | | C₂₃H₂₀Cl₃F₆NOS, | 7.95 (d, J = 1.7 Hz, 1H), | −91.55-−92.86 (m), |
| | | 580.02810; | 7.88 (dd, J = 8.0, 1.7 Hz, | −211.95 (tt, J = 47.3, |
| | | found, | 1H), 7.81 (s, 2H), | 22.0 Hz); |
| | | 580.02810 | 7.49 (d, J = 7.9 Hz, | IR (thin film) |
| | | | 1H), 7.02 (dd, J = 15.8, | 3257, 3059, |
| | | | 9.4 Hz, 1H), | 1656, 1614, |
| | | | 6.75 (d, J = 15.7 Hz, | 1550 cm⁻¹ |
| | | | 1H), 4.62 (t, J = 6.2 Hz, | |
| | | | 1H), 4.53 (t, J = 6.2 Hz, | |
| | | | 1H), 4.29 (td, | |
| | | | J = 14.5, 9.4 Hz, 1H), | |
| | | | 3.40 (dt, J = 7.8, 6.1 Hz, | |
| | | | 2H), 2.90 (t, J = 6.2 Hz, | |
| | | | 1H), 2.85 (t, | |
| | | | J = 6.2 Hz, 1H), | |
| | | | 2.72 (t, J = 7.1 Hz, 2H), | |
| | | | 1.63 (t, J = 19.0 Hz, | |
| | | | 3H) | |
| F129 | | HRMS-ESI | ¹H NMR (500 MHz, | ¹⁹F NMR (471 MHz, |
| | | (m/z) [M + H]⁺ | DMSO-d₆) δ 8.80 (t, J = 5.6 Hz, | DMSO-d₆) |
| | | calcd for | 1H), | δ −57.79, |
| | | C₂₃H₂₀Cl₃F₆NO₂S, | 7.99-7.94 (m, 1H), | −91.50-−92.87 (m), |
| | | 596.0231; | 7.88 (dd, J = 7.9, 1.6 Hz, | −219.11 (tdd, J = 46.9, |
| | | found, | 1H), 7.81 (s, 2H), | 34.4, |
| | | 596.0231 | 7.51 (d, J = 7.9 Hz, | 21.6 Hz); |
| | | | 1H), 7.02 (dd, J = 15.8, | IR (thin film) |
| | | | 9.4 Hz, 1H), | 3427, 3252, |
| | | | 6.74 (d, J = 15.7 Hz, | 3057, 1661, |
| | | | 1H), 4.96-4.70 (m, | 1550 cm⁻¹ |
| | | | 2H), 4.29 (td, J = 14.5, | |
| | | | 9.4 Hz, 1H), | |
| | | | 3.70-3.52 (m, 2H), | |
| | | | 3.37-3.23 (m, 1H), | |
| | | | 3.17-3.03 (m, 2H), | |
| | | | 2.96 (dt, J = 12.7, | |
| | | | 6.0 Hz, 1H), 1.62 (t, | |
| | | | J = 19.0 Hz, 3H) | |
| F130 | 92-94 | ESIMS 670.00 | ¹H NMR (300 MHz, | |
| | | ([M + H]⁺) | DMSO-d₆) δ 8.76 (d, | |
| | | | J = 7.8 Hz, 1H), | |
| | | | 7.88 (s, 1H), 7.79 (s, 2H), | |
| | | | 7.57 (d, J = 8.1 Hz, | |
| | | | 1H), 7.33 (d, J = 7.8 Hz, | |
| | | | 1H), 6.94 (dd, J = 6.3, | |
| | | | 15.9 Hz, 1H), | |
| | | | 6.64 (d, J = 13.5 Hz, | |
| | | | 1H), 4.96-4.91 (m, | |
| | | | 2H), 4.32-4.10 (m, | |
| | | | 4H), 1.67 (t, J = 18.9 Hz, | |
| | | | 3H), | |
| | | | 1.28 (d, J = 6.9 Hz, 3H) | |
| F131 | | ESIMS 644.10 | ¹H NMR (300 MHz, | IR (thin film) |
| | | ([M + H]⁺) | DMSO-d₆) δ 8.72 (d, | 3435, 1652 cm⁻¹ |
| | | | J = 7.8 Hz, 1H), | |
| | | | 7.96 (s, 1H), 7.90 (d, J = 8.1 Hz, | |
| | | | 1H), 7.75 (d, J = 6.6 Hz, | |
| | | | 2H), | |
| | | | 7.51 (d, J = 8.1 Hz, 1H), | |
| | | | 7.06 (dd, J = 9.0, | |
| | | | 15.6 Hz, 1H), | |
| | | | 6.75 (d, J = 15.6 Hz, 1H), | |
| | | | 4.78-4.68 (m, 2H), | |
| | | | 4.55-4.00 (m, 1H), | |
| | | | 4.29-4.25 (m, 1H), | |
| | | | 3.60-3.43 (m, 2H), | |
| | | | 1.67 (t, J = 19.2 Hz, | |
| | | | 3H), 1.30 (d, J = 6.6 Hz, | |
| | | | 3H) | |
| F132 | | ESIMS 589.90 | ¹H NMR (300 MHz, | IR (thin film) |
| | | ([M − H]⁻) | DMSO-d₆) δ 8.65 (d, | 3292, 2925 |
| | | | J = 8.1 Hz, 1H), | 1650, 1132 cm⁻¹ |
| | | | 7.76 (s, 1H), 7.56 (s, 2H), | |
| | | | 7.55 (d, J = 8.7 Hz, | |
| | | | 2H), 7.39 (d, J = 7.8 Hz, | |
| | | | 1H), 6.98 (dd, J = 9.0, | |
| | | | 15.3 Hz, 1H), | |
| | | | 6.65 (d, J = 15.9 Hz, | |
| | | | 1H), 4.78-4.67 (m, | |
| | | | 2H), 4.56-4.51 (m, | |
| | | | 1H), 4.26-4.21 (m, | |
| | | | 1H), 3.61-3.54 (m, | |
| | | | 1H), 3.49-3.41 (m, | |
| | | | 1H), 1.66 (t, J = 19.2 Hz, | |
| | | | 3H), 1.32 (d, J = 6.6 Hz, | |
| | | | 3H) | |
| F133 | | ESIMS 637.70 | ¹H NMR (400 MHz, | IR (thin film) |
| | | ([M + H]⁺) | DMSO-d₆) δ 8.64 (d, | 3292, 1676 cm⁻¹ |
| | | | J = 8.0 Hz, 1H), | |
| | | | 7.90 (s, 1H), 7.54 (s, 4H), | |
| | | | 7.33 (d, J = 7.6 Hz, | |
| | | | 1H), 6.96 (dd, J = 9.2, | |
| | | | 15.6 Hz, 1H), | |
| | | | 6.64 (d, J = 15.6 Hz, | |
| | | | 1H), 4.79-4.69 (m, | |
| | | | 2H), 4.54-4.51 (m, | |
| | | | 1H), 4.25-4.22 (m, | |
| | | | 1H), 3.60-3.57 (m, | |
| | | | 1H), 3.48-3.43 (m, | |
| | | | 1H), 1.64 (t, J = 19.2 Hz, | |
| | | | 3H), 1.32 (d, J = 6.8 Hz, | |
| | | | 3H) | |
| F134 | | ESIMS 626.40 | ¹H NMR (400 MHz, | IR (thin film) |
| | | ([M + H]⁺) | DMSO-d₆) δ 8.72 (d, | 3445, 1651 cm⁻¹ |
| | | | J = 8.0 Hz, 1H), | |
| | | | 7.96 (s, 1H), 7.89 (d, J = 7.6 Hz, | |
| | | | 1H), 7.59 (s, | |
| | | | 3H), 7.50 (d, J = 7.6 Hz, | |
| | | | 1H), 7.05 (dd, J = 12.0, | |
| | | | 16.4 Hz, 1H), | |
| | | | 6.75 (d, J = 15.6 Hz, | |
| | | | 1H), 4.77-4.69 (m, | |
| | | | 1H), 4.54-4.51 (m, | |
| | | | 1H), 4.27-4.25 (m, | |
| | | | 2H), 3.59-3.55 (m, | |
| | | | 2H), 1.65 (t, J = 19.2 Hz, | |
| | | | 3H), 1.30 (d, J = 7.2 Hz, | |
| | | | 3H) | |
| F135 | | ESIMS 643.88 | ¹H NMR (300 MHz, | IR (thin film) |
| | | ([M + H]⁺) | DMSO-d₆) δ 8.72 (d, | 3423, 2925 cm⁻¹ |
| | | | J = 7.8 Hz, 1H), | |
| | | | 8.24-8.17 (m, 1H), | |
| | | | 7.94 (s, 2H), 7.89 (d, J = 7.6 Hz, | |
| | | | 1H), | |
| | | | 7.58-7.47 (m, 2H), | |
| | | | 7.07 (dd, J = 9.3, 15.9 Hz, | |
| | | | 1H), 6.76 (d, J = 15.9 Hz, | |
| | | | 1H), | |
| | | | 4.78-4.68 (m, 2H), | |
| | | | 4.57-4.47 (m, 2H), | |
| | | | 4.63-4.37 (m, 2H), 1.68 (t, | |
| | | | J = 19.2 Hz, 3H), | |
| | | | 1.32 (d, J = 6.9 Hz, | |
| | | | 3H) | |
| F136 | | ESIMS 611.81 | ¹H NMR (400 MHz, | IR (thin film) |
| | | ([M + H]⁺) | DMSO-d₆) δ 8.70 (d, | 3445, 1275, |
| | | | J = 8.0 Hz, 1H), | 750 cm⁻¹ |
| | | | 7.95 (s, 1H), 7.87 (d, J = 8.0 Hz, | |
| | | | 1H), 7.48 (t, | |
| | | | J = 7.6 Hz, 3H), | |
| | | | 7.01-6.94 (m, 1H), | |
| | | | 6.72 (d, J = 15.6 Hz, 1H), | |
| | | | 4.77-4.69 (m, 2H), | |
| | | | 4.25-4.18 (m, 1H), | |
| | | | 4.25-4.18 (m, 1H), | |
| | | | 3.59-3.30 (m, 2H), | |
| | | | 1.60 (t, J = 18.4 Hz, | |
| | | | 1H), 1.38-1.24 (m, | |
| | | | 5H) | |
| F137 | | ESIMS 608.0 | ¹H NMR (300 MHz, | ¹⁹F NMR (471 MHz, |
| | | ([M − H]⁻) | CDCl₃) δ 7.59 (d, J = 1.6 Hz, | DMSO-d₆) |
| | | | 1H), 7.53 (d, | δ −66.46, |
| | | | J = 8.0 Hz, 1H), | −68.73 |
| | | | 7.51-7.45 (m, 2H), | |
| | | | 7.35 (dd, J = 8.1, 1.6 Hz, | |
| | | | 1H), 7.22 (dd, J = 8.3, | |
| | | | 2.2 Hz, 1H), | |
| | | | 6.51 (d, J = 15.9 Hz, 1H), | |
| | | | 6.41 (dd, J = 15.9, | |
| | | | 7.2 Hz, 1H), 6.07 (d, | |
| | | | J = 8.2 Hz, 1H), | |
| | | | 4.53-4.29 (m, 1H), | |
| | | | 4.26-3.99 (m, 1H), | |
| | | | 3.19 (qt, J = 9.9, 4.8 Hz, | |
| | | | 2H), 2.91 (d, J = 5.9 Hz, | |
| | | | 2H), 1.37 (d, J = 6.7 Hz, | |
| | | | 3H) | |

TABLE 4
Structure and Preparation Method for FC Series Compounds

| No. | Structure | Prep.* |
|---|---|---|
| FC74 | 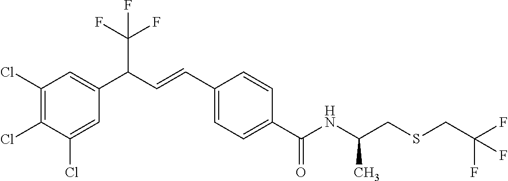 [SMILES: C[C@H](CSCC(F)(F)F)NC(=O)c1ccc(/C=C/C(c2cc(Cl)c(Cl)c(Cl)c2)C(F)(F)F)cc1] | 10 |
| FC77 | 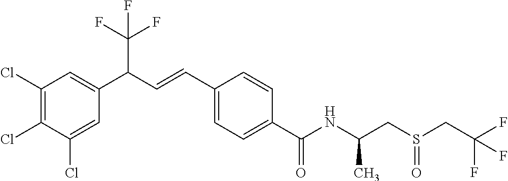 [SMILES: C[C@H](CS(=O)CC(F)(F)F)NC(=O)c1ccc(/C=C/C(c2cc(Cl)c(Cl)c(Cl)c2)C(F)(F)F)cc1] | 3 |
| FC78 | 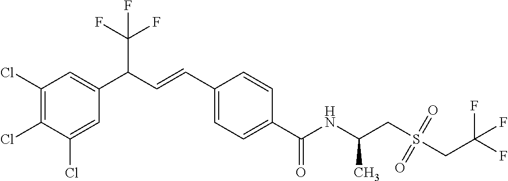 [SMILES: C[C@H](CS(=O)(=O)CC(F)(F)F)NC(=O)c1ccc(/C=C/C(c2cc(Cl)c(Cl)c(Cl)c2)C(F)(F)F)cc1] | 3 |

*prepared according to example number

TABLE 5
Analytical Data for Compounds in Table 5

| | Mp | | | ¹³C NMR; |
|---|---|---|---|---|
| No. | (° C.) | Mass (m/z) | ¹H NMR | ¹⁹F NMR; IR |
| FC74 | | ESIMS 563 | ¹H NMR (400 MHz, | ¹⁹F NMR (376 |
| | | ([M − H]⁻) | CDCl₃) δ 7.78-7.69 | MHz, CDCl₃) δ |
| | | | (m, 2H), 7.48-7.43 | −66.50, −68.68 |
| | | | (m, 2H), 7.42 (s, 2H), | |
| | | | 6.62 (d, J = 15.8 Hz, | |
| | | | 1H), 6.41 (dd, J = | |
| | | | 15.9, 8.0 Hz, 1H), 6.12 | |
| | | | (d, J = 8.0 Hz, 1H), | |
| | | | 4.41 (dt, J = 13.9, 6.3 | |
| | | | Hz, 1H), 4.20-4.04 | |
| | | | (m, 1H), 3.26-3.06 | |
| | | | (m, 2H), 2.97-2.87 | |
| | | | (m, 2H), rotamers | |
| | | | 1.37 (d, J = 6.7 Hz) & | |
| | | | 1.32 (d, J = 6.7 Hz) | |
| | | | (3H) | |
| FC77 | | ESIMS 580 | mixture of | mixture of |
| | | ([M − H]⁻) | diastereomers: ¹H NMR | diastereomers: |
| | | | (400 MHz, CDCl₃) δ | ¹⁹F NMR (376 |
| | | | 7.82-7.70 (m, 2H), | MHz, CDCl₃) δ |
| | | | 7.48-7.40 (m, 2H), | −60.54 & −60.76, |
| | | | 7.41 (s, 2H), 6.61 (d, J = | −68.68 |
| | | | 15.9 Hz, 1H), 6.40 | |
| | | | (ddd, J = 15.9, 8.0, | |
| | | | 3.9 Hz, 1H), 4.61 (td, J = | |
| | | | 7.0, 5.8 Hz, 1H), | |
| | | | 4.10 (q, J = 8.6 Hz, | |
| | | | 1H), 3.71 (dq, J = | |
| | | | 14.2, 9.9 Hz, 1H), 3.65- | |
| | | | 3.48 (m, 1H), 3.33- | |
| | | | 3.10 (m, 2H), 1.60- | |
| | | | 1.53 (m, 3H) | |
| FC78 | | ESIMS 596 | ¹H NMR (300 MHz, | ¹⁹F NMR (376 |
| | | ([M − H]⁻) | CDCl₃) δ 7.73 (d, J = | MHz, CDCl₃) δ |
| | | | 8.3 Hz, 2H), 7.43 (d, J = | −61.00, −68.67 |
| | | | 9.1 Hz, 4H), 6.71- | |
| | | | 6.55 (m, 2H), 6.40 | |
| | | | (dd, J = 15.9, 7.9 Hz, | |
| | | | 1H), 4.70 (p, J = 6.4 | |
| | | | Hz, 1H), 4.20-4.07 | |
| | | | (m, 1H), 4.12-3.87 | |
| | | | (m, 2H), 3.57 (dd, J = | |
| | | | 14.5, 5.9 Hz, 1H), 3.40 | |
| | | | (dd, J = 14.5, 5.2 Hz, | |
| | | | 1H), 1.55 (d, J = 6.9 | |
| | | | Hz, 3H) | |

| | |
|---|---|
| % Control (or Mortality) | Rating |

BAW, CEW, & CL Rating Table

| | |
|---|---|
| 50-100 | A |
| More than 0-Less than 50 | B |
| Not Tested | C |
| No activity noticed in this bioassay | D |

GPA & YFM Rating Table

| | |
|---|---|
| 80-100 | A |
| More than 0-Less than 80 | B |
| Not Tested | C |
| No activity noticed in this bioassay | D |

TABLE ABC
Biological Results

PESTS

| No. | BAW | CL | GPA | YFM |
|---|---|---|---|---|
| F1 | A | A | C | A |
| F2 | A | A | C | A |
| F3 | A | A | C | B |
| F4 | A | A | C | C |
| F10 | A | A | C | C |
| F11 | A | A | C | B |
| F12 | A | A | C | A |
| F13 | A | A | C | A |
| F14 | A | A | C | A |
| F15 | A | A | C | C |
| F16 | A | A | C | C |
| F17 | A | A | C | C |
| F18 | A | A | C | C |
| F19 | A | A | C | C |
| F20 | A | A | C | C |
| F21 | A | A | C | A |
| F22 | A | A | A | A |
| F23 | A | A | C | C |
| F24 | A | A | C | A |
| F25 | A | A | B | A |
| F26 | A | A | B | B |
| F27 | A | A | C | A |
| F28 | A | A | C | A |
| F29 | A | D | C | C |
| F30 | A | A | C | C |
| F31 | A | A | C | C |
| F32 | A | A | C | C |
| F33 | A | A | C | C |
| F34 | A | A | C | A |
| F35 | A | A | B | A |
| F36 | A | A | C | A |
| F37 | A | A | C | A |
| F38 | A | A | B | A |
| F39 | A | A | C | A |
| F40 | A | A | C | A |
| F41 | A | A | C | A |
| F42 | A | A | C | A |
| F43 | A | A | C | C |
| F44 | A | A | C | A |
| F45 | A | A | C | A |
| F46 | A | A | C | A |
| F47 | A | A | C | C |
| F48 | A | A | C | A |
| F49 | A | A | D | A |
| F50 | A | A | C | A |
| F51 | A | A | C | A |
| F52 | A | A | C | A |
| F53 | A | A | C | A |
| F54 | A | A | C | A |
| F55 | A | A | C | C |
| F56 | A | A | D | A |
| F57 | A | A | C | A |
| F58 | A | A | D | A |
| F59 | A | A | D | C |
| F60 | A | A | C | A |
| F61 | A | A | C | A |
| F62 | A | A | C | A |
| F63 | A | A | C | A |
| F64 | A | A | C | A |
| F65 | A | A | C | A |
| F66 | A | A | C | A |
| F67 | A | A | C | A |
| F68 | A | A | B | A |
| F69 | A | A | C | A |
| F70 | A | A | C | A |
| F71 | A | A | C | A |
| F72 | A | A | C | A |
| F73 | A | A | C | A |
| FC74 | A | A | C | C |
| F75 | A | A | C | C |
| F76 | A | A | C | C |
| FC77 | A | A | C | C |
| FC78 | A | A | C | C |
| F79 | A | A | C | C |
| F80 | A | A | C | C |
| F81 | A | A | C | C |
| F82 | A | A | C | C |
| F83 | A | A | C | C |
| F84 | A | A | C | C |
| F85 | A | A | C | A |
| F86 | A | A | C | A |
| F87 | A | A | C | A |
| F88 | A | A | C | A |
| F89 | A | A | C | A |
| F90 | A | A | C | A |
| F91 | A | A | C | A |
| F92 | A | A | C | A |
| F93 | A | A | C | A |
| F94 | A | A | C | A |
| F95 | A | A | C | A |
| F96 | A | A | C | A |
| F97 | A | A | C | A |
| F98 | A | A | C | A |
| F99 | A | A | C | A |
| F100 | A | A | C | B |
| F101 | A | A | C | A |
| F102 | A | A | C | A |
| F103 | A | A | C | A |
| F104 | A | A | C | A |
| F105 | A | A | C | A |
| F106 | A | A | C | A |
| F107 | A | A | C | C |
| F108 | A | A | C | C |
| F109 | A | A | C | A |
| F110 | A | A | C | A |
| F111 | A | A | B | A |
| F112 | A | A | C | A |
| F113 | A | A | C | A |
| F114 | A | A | C | C |
| F115 | A | A | C | C |
| F116 | A | A | C | C |
| F117 | A | A | C | C |
| F118 | A | A | C | A |
| F119 | A | A | B | A |
| F120 | A | A | C | C |
| F121 | A | A | C | C |
| F122 | A | A | C | C |
| F123 | A | A | C | C |
| F124 | A | A | C | C |
| F125 | A | A | C | A |
| F126 | A | A | C | B |
| F128 | A | A | C | C |
| F129 | A | A | C | C |
| F130 | A | A | C | C |
| F131 | A | A | C | D |
| F132 | A | A | C | B |
| F133 | A | A | C | C |
| F134 | A | A | C | A |
| F135 | A | A | C | C |
| F136 | A | A | C | C |
| F137 | A | A | C | C |

### Comparative Data

Bioassays on BAW and CL were conducted according to the procedures outlined in Example A: Bioassays on Beet Armyworm (“BAW”) and Cabbage Looper (“CL”) using the indicated concentrations. The results are indicated in Table CD1.

TABLE CD1

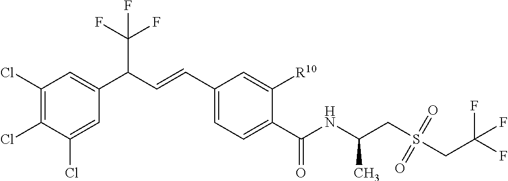

| | | 5 μg/cm² | | 0.5 μg/cm² | | 0.05 μg/cm² | |
|---|---|---|---|---|---|---|---|
| No. | R¹⁰ | BAW | CL | BAW | CL | BAW | CL |
| FC78 | H | 100* | 100 | 19 | 7 | 0 | 0 |
| F58 | Cl | 100 | 100 | 100 | 100 | 100 | 100 |
| F40 | CH₃ | 100 | 100 | 100 | 100 | 60 | 100 |
| F24 | CF₃ | 100 | 100 | 100 | 100 | 100 | 100 |

*Percent control (or mortality)

1. A molecule having the following formula 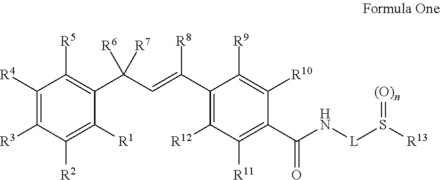
wherein:
(A) R¹, R⁵, R⁶, R⁹, and R¹² are each independently selected from the group consisting of H, F, Cl, Br, I, CN, (C₁-C₄)alkyl, (C₁-C₄)haloalkyl, (C₁-C₄)alkoxy, and (C₁-C₄)haloalkoxy;
(B) R² is selected from the group consisting of H, F, Cl, Br, I, CN, (C₁-C₄)alkyl, (C₁-C₄)haloalkyl, (C₁-C₄)alkoxy, and (C₁-C₄)haloalkoxy;
(C) R³ and R⁴ are each independently selected from the group consisting of (D), H, F, Cl, Br, I, CN, C(O)H, (C₁-C₄)alkyl, (C₂-C₄)alkenyl, (C₂-C₄)alkynyl, (C₁-C₄)haloalkyl, (C₁-C₄)alkoxy, and (C₁-C₄)haloalkoxy;
(D) R³ and R⁴ together can optionally form a 3- to 5-membered saturated or unsaturated, heterohydrocarbyl link, which may contain one or more heteroatoms selected from the group consisting of nitrogen, sulfur, and oxygen,
wherein said heterohydrocarbyl link may optionally be substituted with one or more substituents independently selected from the group consisting of H, F, Cl, Br, I, CN, and OH;
(E) R⁷ is (C₁-C₆)haloalkyl;
(F) R⁸ is selected from the group consisting of H, (C₁-C₄)alkyl, (C₁-C₄)haloalkyl, and (C₁-C₄)alkoxy;
(G) R¹⁰ is selected from the group consisting of F, Cl, Br, I, (C₁-C₄)alkyl, (C₂-C₄)alkenyl, (C₂-C₄)alkynyl, (C₁-C₄)haloalkyl, (C₁-C₄)alkoxy, and (C₁-C₄)haloalkoxy;
(H) R¹¹ is selected from the group consisting of H, F, Cl, Br, I, (C₁-C₄)alkyl, or (C₁-C₄)haloalkyl;
(I) L is a linker that is selected from the group consisting of (C₁-C₈)alkyl, (C₁-C₄)alkoxy, (C₃-C₆)cycloalkyl-(C₁-C₄)alkyl, (C₁-C₄)alkyl-(C₃-C₆)cycloalkoxy, (C₁-C₄)alkyl-S—(C₁-C₄)alkyl, (C₁-C₄)alkyl-S(O)—(C₁-C₄)alkyl, and (C₁-C₄)alkyl-S(O)₂—(C₁-C₄)alkyl,
wherein each alkyl, alkoxy, and cycloalkyl may optionally be substituted with one or more substituents independently selected from the group consisting of F, Cl, Br, I, CN, OH, oxetanyl, C(═O)NH(C₁-C₄)haloalkyl, and (C₁-C₄)alkoxy;
(J) n is selected from the group consisting of 0, 1, and 2;
(K) R¹³ is selected from the group consisting of (C₁-C₄)alkyl, (C₂-C₄)alkenyl, (C₁-C₄)haloalkyl, (C₁-C₄)alkoxy, (C₁-C₄)haloalkoxy, phenyl, benzyl, (C₁-C₄)alkyl-(C₃-C₆)cycloalkyl, and NH(C₁-C₄)haloalkyl,
wherein each alkyl, alkenyl, haloalkyl, alkoxy, haloalkoxy, phenyl, and cycloalkyl, may optionally be substituted with one or more substituents independently selected from the group consisting of F, Cl, Br, I, CN, and OH; and
agriculturally acceptable acid addition salts, salt derivatives, solvates, ester derivatives, crystal polymorphs, isotopes, resolved stereoisomers, and tautomers, of the molecules of Formula One.

2. A molecule according to claim 1 wherein R¹, R⁵, R⁶, R⁹, and R¹² are H.

3. A molecule according to claim 1 wherein R² is Cl or Br.

4. A molecule according to claim 1 wherein R³ is H, F, Cl, or CN.

5. A molecule according to claim 1 wherein R⁴ is Cl, Br, or C(O)H.

6. A molecule according to claim 1 wherein R³ and R⁴ together are —OCH₂O—.

7. A molecule according to claim 1 wherein R², R³, and R⁴ are Cl.

8. A molecule according to claim 1 wherein R⁷ is CF₃ or CF₂CH₃.

9. A molecule according to claim 1 wherein R⁸ is H, OCH₃, or OCH₂CH₃.

10. A molecule according to claim 1 wherein R¹⁰ is F, Cl, Br, CH₃, CH₂CH₃, CHF₂, or CF₃.

11. A molecule according to claim 1 wherein R¹¹ is H or CH₃.

12. A molecule according to claim 1 wherein L is —CH₂CH₂—, —CH(CH₃)CH₂—, —CH(CH₂CH₃)CH₂—, —CH(CH(CH₃)₂)CH₂—, —C(CH₃)₂CH₂—, —CH(CH₃)CH₂CH₂—, —CH(CH₂OCH₃)CH₂—, —C(cyclopropyl)CH₂—, —CH₂C(3,3-oxetanyl)-, or —CH₂CH(SCH₂CH₃)—.

13. A molecule according to claim 1 wherein n is 0, 1, or 2.

14. A molecule according to claim 1 wherein R¹³ is CH₃, CH₂CH₃, CH₂CH₂CH₃, CH(CH₃)₂, CH₂CH(CH₃)₂, CH₂CH═CH₂, CH₂CF₃, CH₂CH₂CF₃, phenyl, CH₂phenyl, CH(CH₃)phenyl, CH₂cyclopropyl, or NHCH₂CF₃, wherein each phenyl and cyclopropyl is optionally substituted with one or more substituents selected from the group consisting of F, Cl, Br, and CN.

15. A molecule according to claim 1 wherein
(A) R¹, R⁵, R⁶, R⁹, and R¹² are H;
(B) R² is selected from the group consisting of Cl and Br;
(C) R³ and R⁴ are, each independently selected from the group consisting of (D), H, F, Cl, Br, I, CN, and C(O)H;
(D) R³ and R⁴ together can optionally form a 3- to 5-membered saturated or unsaturated, heterohydrocarbyl link, which may contain one or more heteroatoms selected from the group consisting of nitrogen, sulfur, and oxygen,
wherein said heterohydrocarbyl link may optionally be substituted with one or more substituents independently selected from the group consisting of H, F, Cl, Br, I, CN, and OH;
(E) R⁷ is (C₁-C₆)haloalkyl;
(F) R⁸ is selected from the group consisting of H and (C₁-C₄)alkoxy;
(G) R¹⁰ is selected from the group consisting of F, Cl, Br, I, (C₁-C₄)alkyl, and (C₁-C₄)haloalkyl;
(H) R¹¹ is selected from the group consisting of H and (C₁-C₄)alkyl;
(I) L is a linker that is selected from the group consisting of (C₁-C₈)alkyl, (C₁-C₄)alkoxy, (C₃-C₆)cycloalkyl-(C₁-C₄)alkyl, (C₁-C₄)alkyl-(C₃-C₆)cycloalkoxy, and (C₁-C₄)alkyl-S—(C₁-C₄)alkyl,
wherein each alkyl, alkoxy, cycloalkyl, and haloalkyl may optionally be substituted with one or more (C₁-C₄)alkoxy substituents;
(J) n is selected from the group consisting of 0, 1, and 2; and
(k) R13 is selected from the group consisting of (C₁-C₄)alkyl, (C₂-C₄)alkenyl, (C₁-C₄)haloalkyl, phenyl, (C₁-C₄)alkyl-phenyl, (C₁-C₄)alkyl-(C₃-C₆)cycloalkyl, and NH(C₁-C₄)haloalkyl,
wherein each alkyl, alkenyl, haloalkyl, alkoxy, haloalkoxy, phenyl, and cycloalkyl, may optionally be substituted with one or more substituents independently selected from the group consisting of F, Cl, Br, I, and CN.

16. A molecule according to claim 1 wherein
(A) R¹, R⁵, R⁶, R⁹, and R¹² are H;
(B) R² is selected from the group consisting of Cl and Br;
(C) R³ and R⁴ are, each independently selected from the group consisting of H, F, Cl, Br, I, and CN.
(E) R⁷ is (C₁-C₆)haloalkyl;
(F) R⁸ is H;
(G) R¹⁰ is selected from the group consisting of F, Cl, Br, I, (C₁-C₄)alkyl, and (C₁-C₄)haloalkyl;
(H) R¹¹ is selected from the group consisting of H and (C₁-C₄)alkyl;
(I) L is a linker that is selected from the group consisting of (C₁-C₅)alkyl, (C₃-C₆)cycloalkyl-(C₁-C₄)alkyl, and (C₁-C₄)alkyl-S—(C₁-C₄)alkyl;
(J) n is selected from the group consisting of 0, 1, and 2; and
(K) R¹³ is selected from the group consisting of (C₁-C₄)alkyl, (C₂-C₄)alkenyl, (C₁-C₄)haloalkyl, (C₁-C₄)alkyl-phenyl, (C₁-C₄)alkyl-(C₃-C₆)cycloalkyl, and NH(C₁-C₄)haloalkyl,
wherein each alkyl, alkenyl, haloalkyl, alkoxy, haloalkoxy, and cycloalkyl, may optionally be substituted with one or more substituents independently selected from the group consisting of F, Cl, Br, and I.

17. A molecule according to claim 1 wherein said molecule is selected from one of the molecules in Table 1

18. A pesticidal composition comprising a molecule according to claim 1 further comprising one or more active ingredients.

19. A pesticidal composition according to claim 18 wherein said active ingredient is from AIGA.

20. A pesticidal composition according to claim 18 wherein said active ingredient is selected from the group consisting of AI-1, 1,3-dichloropropene, chlorpyrifos, chlorpyrifos-methyl, hexaflumuron, methoxyfenozide, noviflumuron, spinetoram, spinosad, sulfoxaflor, and sulfuryl fluoride.

21. A pesticidal composition comprising a molecule according to claim 1 further comprising one or more MoA Materials.

22. A pesticidal composition according to claim 21 wherein said MoA Material is from MoAMGA.

23. A pesticidal composition comprising a molecule according to claim 1 and a seed.

24. A process to control a pest said process comprising applying to a locus, a pesticidally effective amount of a pesticidal composition wherein said pesticidal composition comprises a molecule according to claim 1.
